(19) United States
(12) Patent Application Publication
(10) Pub. No.: US 2007/0111214 A1
(43) Pub. Date: May 17, 2007

(54) 20685, 579,17114, 23821, 33894 and 32613, novel human transporters

(75) Inventors: Maria Alexandra Glucksmann, Lexington, MA (US); Inmaculada Silos-Santiago, Jamaica Plain, MA (US)

Correspondence Address: Intellectual Property Group;MILLENNIUM PHARMACEUTICALS, INC., 75 Sidney Street, Cambridge, MA, 02139, US

(73) Assignee: Millennium Pharmaceuticals, Inc.

(21) Appl. No.: 10/913,257

(22) Filed: Aug. 6, 2004

Related U.S. Application Data
(63) Continuation of application No. 10/199,485, filed on Jul. 18, 2002, now abandoned.
Application No. 10/199,485 is a continuation-in-part of application No. 09/795,693, filed on Feb. 28, 2001, now abandoned.
(60) Provisional application No. 60/185,906, filed on Feb. 29, 2000.

(51) Int. Cl. C12Q 1/68; G01N 33/567; A61K 38/17; C07K 14/705; C07K 16/28; C07H 21/04; C12P 21/06

(52) U.S. Cl. 435/6; 435/69.1; 435/320.1; 435/325; 435/456; 530/350; 530/388.22; 536/23.5; 514/12; 435/7.2

(57) ABSTRACT

The present invention relates to newly identified human transporters. In particular, the invention relates to transporter polypeptides and polynucleotides, methods of detecting the transporter polypeptides and polynucleotides, and methods of diagnosing and treating transporter-related disorders. Also provided are vectors, host cells, and recombinant methods for making and using the novel molecules.

### CROSS-REFERENCE TO RELATED APPLICATION

This application is a continuation-in-part of copending U.S. patent application Ser. No. 09/795,693, filed on Feb. 28, 2001, which claims the benefit of U.S. Provisional Application Ser. No. 60/185,906, filed Feb. 29, 2000, the contents of which are hereby incorporated in their entireties herein by reference.

### FIELD OF THE INVENTION

The present invention relates to newly identified human transporters. In particular, the invention relates to transporter polypeptides and polynucleotides, methods of detecting the transporter polypeptides and polynucleotides, and methods of diagnosing and treating transporter-related disorders. Also provided are vectors, host cells, and recombinant methods for making and using the novel molecules.

### BACKGROUND OF THE INVENTION

Transporters throughout the body control the solute composition of the cerebrospinal fluid, urine, plasma, and other extracellular fluids. Cloning of genes encoding transporters is facilitating the elucidation of the role of transport proteins in health and disease. The availability of cloned transporters provides the opportunity to define the pharmacological profiles of specific gene products, map their patterns of distribution, and make correlations with in vivo observations to better understand their biological functions.

Neurotransmitter Transporters

In the brain, neurotransmitter transporters serve specialized functions related to the modulation of synaptic transmission. Neurotransmitter transporters, their molecular biology, function, and regulation have recently been reviewed in Borowsky et al., (1995) International Review of Neurobiology, 38:139-199 (summarized below). There are four processes that are integral to synaptic neurotransmission. The transmitter must be synthesized and stored in the neuron. An action potential must stimulate release of the neurotransmitter from the pre-synaptic terminal. The released neurotransmitter must enter the synaptic cleft and interact with both post- and pre-synaptic receptors. Then the neurotransmitter must be removed from the synapse. Active transport into pre-synaptic neurons or into surrounding glia via membrane-bound transport proteins is the predominant mechanism for removing the released neurotransmitters. The results of pharmacological studies suggest that there are specific transporters for each of the monoaminergic and amino acid neurotransmitters. The site of action for anti-depressants and psychostimulants is the monominergic transporter.

Accordingly, inactivation and recycling of released neurotransmitter are the major functions of these plasma membrane bound transporters.

In addition to being useful for understanding basic neurochemistry and developing drugs, transporter nucleic acids have also proven to be useful for mapping the anatomic distribution of neurotransmitter systems which lack other specific markers. Interest in transporters has also resulted from their potential role at the site of action of both anti-depressants and psycho-motor stimulants. Numerous monoamine uptake inhibitors have been targeted as anti-depressants. Serotonin and dopamine uptake inhibitors have also been shown to be effective for treating depression.

A large and growing number of transporters have been cloned and identified. These transporters have been classified into several families on the basis of sequence homology, ion dependence, and predicted topology (see FIG. 2 in Borowsky et al., cited above). The sodium/chloride-dependent family functions at the plasma membrane, is sodium- and chloride-dependent and has twelve potential transmembrane domains. This subfamily has been designated the monoamine family and its members include transporters for dopamine, serotonin, and norepinephine. The subfamily designated “amino acid” includes GABA, glycine, proline, taurine, betaine, and creatine. A second family contains sodium-dependent transporters with 8 to 10 potential membrane domains that function at the plasma membrane. This family includes transporters for glutamate and a neutral amino acid transporter. The glutamate transporters depend upon both sodium and potassium. The neutral amino acid transporter is dependent on sodium. A third family contains vesicular transporters that package neurotransmitters into synaptic or neuroendocrine vesicles by transporting neurotransmitters from the site of the plasma membrane into the vesical lumen. This family can contain a large first intralumenal loop. The family is dependent on H⁺. Examples in this family include two vesicular monoamine transporters (for example, for serotonin, dopamine, norepinephrine, epinephrine, and histamine) and the vesicular acetylcholine transporter.

Table I in Borowsky et al. shows various sodium/chloride-dependent transporters, such as DAT with the substrate dopamine, 5-HHT, with serotonin as the substrate, NET with norepinephrine as the substrate; GAT-1, with GABA as the substrate; GAT-2, with GABA as the substrate; GAT-3, with GABA as the substrate; BGT-1, with betaine and GABA as the substrate; GLYT-1a, GLYT-1b, GLYT-1c, and GLYT-2, with glycine as the substrate; PROT, with proline as the substrate; and TAUT, with taurine as the substrate. These have all been shown to inhibited by the specific inhibitors in Table 1 in Borowsky et al., incorporated herein for these corresponding inhibitors. Tissue distribution of these members is also shown in this table, which is incorporated herein for this distribution.

The role of the vesicular transporters is to repackage the cytoplasmic pool of neurotransmitters into presynaptic vesicles. During synaptic transmission these vesicles fuse with the plasma membrane and release their contents into the synapse. Vesicular amine transporters can be proton-dependent. Substrates include, but are not limited to, serotonin, dopamine, norepinephrine, epinephrine, and histamine. They are inhibited by reserpine and tetrabenazine. Localization can be in components including, but not limited to, chromaffin granules and monoaminergic neurons in the central nervous system and in peripheral tissues. Table III in Borowsky et al. shows various vesicular transporter family members. These include VMAT1, with the substrates serotonin, epinephrine, dopamine, and norepinephrine; VMAT2, with the substrates serotonin, dopamine, norepinephrine, epinephrine, and histamine; UNC-17, with the substrate acetylcholine; and VAChT, with the substrate acetylcholine. VMAT1 is inhibited by reserpine, tetrabenazine, and ketanserin; VMAT2 is inhibited by reserpine, tetrabenazine, ketanserin; UNC-17 and VAChT are inhibited by vesicamol. The tissue distribution of these members is also shown in this table, which is incorporated herein for this distribution.

The glutamate family of transporters defines a family with no significant homology to sodium/chloride dependent transporter family members. Several members of the glutamate family of transporters are shown in Table II of Borowsky et al. These include EEAC1, with glutamate as the substrate, and THA, AAD, and DHK as an inhibitor; GLAST, with glutamate as the substrate and THA as an inhibitor; GLT1, with glutamate as the substrate and THA, AAD, and DHK as an inhibitor; ASCT1, with alanine, serine, and cysteine as substrates; EAAT1, with glutamate as the substrate and THA, PDC, SOS, DHK and KA as an inhibitor; EAAT2, with glutamate as the substrate and PDC, THA, DHK, KA and SOS as an inhibitor; and EAAT3, with glutamate as the substrate and THA, PDC, SOS, DHK and KA as an inhibitor. Tissue distribution of these members is also shown on this table, which is incorporated herein for this distribution.

Regarding the regulation of transporters, changes in the activity or the availability of transporters is important for the amount and duration of neurotransmitter available in the synapse. These changes can alter the response of both pre- and post-synaptic receptors to release neurotransmitters. Changes in the level of transport can result from changes in the affinity of the transporter for substrates or changes in the maximal velocity of the transporter. Changes in the maximal velocity can result by either changes in the rate of transporter cycling from occupied to unoccupied or changes in the number of active transporters at the cell surface. Borowsky et al. suggests that regulation of transport can occur directly by phosphorylation of the transport protein or indirectly by phosphorylation of other proteins, such as sodium/potassium-ATPase, phosphates, or proteins affecting ion gradients. Phosphorylation could also affect the distribution of transporters in active and inactive pools. A schematic model illustrating potential mechanisms for phosphorylation-mediated regulation of neurotransmitter transport is shown in FIG. 8 of Borowsky et al. The regulation of transport by hypertonicity is another example of physiologic regulation. There is also evidence that transporters on glial cells are regulated by monoamine receptors.

As indicated, members of the sodium/chloride dependent transporter family are dependent on extracellular sodium and chloride. Studies demonstrating the ionic dependence of neurotransmitter transport are cited in Borowsky et al., above. The energy necessary for the active transport of substrates, which may be against the substrate concentration gradient, derives from energy stored in the ion gradient generated by the sodium-potassium ATPase. The ionic requirements for members of the glutamate transport family is distinct from that of the sodium/chloride dependent family. In retinal glia, high affinity glutamate transport is coupled to co-transport of sodium and potassium ions as well as OH⁻ ion.

Vesicular amine transport depends on the electrochemical gradient generated by the vacuolar H⁺-ATPase (studies cited in Borowsky et al.). The transport of a cytoplasmic amine into the vesicle lumen may be coupled with the transport of a proton out of the vesicle. Further, in chromaffin granules and permeabilized CV-1 cells expressing VMAT2, dependence on ATP and transmembrane electrochemical proton gradient has been shown.

Sugar Transport Proteins

In mammalian cells the uptake of glucose is mediated by a family of closely related transport proteins which are called the glucose transporters [1,2,3]. At least seven of these transporters are currently known to exist (in human they are encoded by the GLUT1 to GLUT7 genes).

These integral membrane proteins are predicted to comprise twelve membrane spanning domains. The glucose transporters show sequence similarities [4,5] with a number of other sugar or metabolite transport proteins including but are not limited to those listed below.

Escherichia coli arabinose-proton symport (araE).

Escherichia coli galactose-proton symport (galP).

Escherichia coli and Klebsiella pneumoniae citrate-proton symport (also known as citrate utilization determinant) (gene cit).

Escherichia coli alpha-ketoglutarate permease (gene kgtP).

Escherichia coli proline/betaine transporter (gene proP) [6].

Escherichia coli xylose-proton symport (xylE).

Zymomonas mobilis glucose facilitated diffusion protein (gene glf).

Yeast high and low affinity glucose transport proteins (genes SNF3, HXT1 to HXT14).

Yeast galactose transporter (gene GAL2).

Yeast maltose permeases (genes MAL3T and MAL6T).

Yeast myo-inositol transporters (genes ITR1 and ITR2).

Yeast carboxylic acid transporter protein homolog JEN1.

Yeast inorganic phosphate transporter (gene PHO84).

Kluyveromyces lactis lactose permease (gene LAC12).

Neurospora crassa quinate transporter (gene Qa-y), and Emericella nidulans quinate permease (gene qutD).

Chlorella hexose carrier (gene HUP1).

Arabidopsis thaliana glucose transporter (gene STP1).

Spinach sucrose transporter.

Leishmania donovani transporters D1 and D2.

Leishmania enriettii probable transport protein (LTP).

Yeast hypothetical proteins YBR241c, YCR98c and YFL040w.

Caenorhabditis elegans hypothetical protein ZK637.1.

Escherichia coli hypothetical proteins yabE, ydjE and yhjE.

Haemophilus influenzae hypothetical proteins HI0281 and HI0418.

Bacillus subtilis hypothetical proteins yxbC and yxdF.
[1] Silverman Annu. Rev. Biochem. 60:757-794 (1991).
[2] Gould et al. Trends Biochem. Sci. 15:18-23 (1990).
[3] Baldwin Biochim. Biophys. Acta 1154:17-49 (1993).
[4] Maiden et al. Nature 325:641-643 (1987).
[5] Henderson Curr. Opin. Struct. Biol. 1:590-601 (1991).
[6] Culham et al. J. Mol. Biol. 229:268-276 (1993).
   ABC Transporters

On the basis of sequence similarities, a family of related ATP-binding proteins has been characterized [1 to 5]. These proteins are associated with a variety of distinct biological processes in both prokaryotes and eukaryotes, but a majority of them are involved in active transport of small hydrophilic molecules across the cytoplasmic membrane. All these proteins share a conserved domain of some two hundred amino acid residues, which includes an ATP-binding site. These proteins are collectively known as ABC transporters. Proteins known to belong to this family include but are not limited to those listed below.

In prokaryotes:

Active transport systems components: alkylphosphonate uptake (phnC/phnK/phnL); arabinose (araG); arginine (artP); dipeptide (dciAD;dppD/dppF); ferric enterobactin (fepC); ferrichrome (fhuC); galactoside (mglA); glutamine (glnQ); glycerol-3-phosphate (ugpC); glycine betaine/L-proline (proV); glutamate/aspatate (gltL); histidine (his P); iron(III) (sfuC), iron(III) dicitrate (fecE); lactose (lacK); leucine/isoleucine/valine (braF/braG;livF/livG); maltose (malK); molybdenum (modC); nickel (nikD/nikE); oligopeptide (amiE/amiF;oppD/oppF); peptide (sapD/sapF); phosphate (pstB); putrescine (potG); ribose (rbsA); spermidine/putrescine (potA); sulfate (cysA); vitamin B₁₂ (btuD).

Hemolysin/leukotoxin export proteins hlyB, cyaB and lktB.

Colicin V export protein cvaB.

Lactococcin export protein IcnC [6].

Lantibiotic transport proteins nisT (nisin) and spaT (subtilin).

Extracellular proteases B and C export protein prtD.

Alkaline protease secretion protein aprD.

Beta-(1,2)-glucan export proteins chvA and ndvA.

Haemophilus influenzae capsule-polysaccharide export protein bexA.

Cytochrome c biogenesis proteins ccmA (also known as cycV and helA).

Polysialic acid transport protein kpsT.

Cell division associated ftsE protein.

Copper processing protein nosF from Pseudomonas stutzeri.

Nodulation protein nodI from Rhizobium.

Escherichia coli proteins cydC and cydD.

Subunit A of the ABC excision nuclease (gene uvrA).

Erythromycin resistance protein from Staphylococcus epidermidis (gene msrA).

Tylosin resistance protein from Streptomyces fradiae (gene tlrC) [7].

Heterocyst differentiation protein (gene hetA) from Anabaena PCC 7120.

Protein P29 from Mycoplasma hyorhinis, a probable component of a high affinity transport system.

yhbG, a putative protein whose gene is linked with ntrA in many bacteria such as Escherichia coli, Klebsiella pneumoniae, Pseudomonas putida, Rhizobium meliloti and Thiobacillus ferrooxidans.

Escherichia coli and related bacteria hypothetical proteins yabJ, yadG, yagC, ybbA, ycjW, yddA, yehX, yejF, yheS, yhiG, yhiH, yjcW, yjjK, yojI, yrbF and ytfR.

In eukaryotes:

The multidrug transporters (Mdr) (P-glycoprotein), a family of closely related proteins which extrude a wide variety of drugs out of the cell (for a review see [8]).

Cystic fibrosis transmembrane conductance regulator (CFTR), which is most probably involved in the transport of chloride ions.

Antigen peptide transporters 1 (TAP1, PSF1, RING4, HAM-1, mtp1) and 2 (TAP2, PSF2, RING11, HAM-2, mtp2), which are involved in the transport of antigens from the cytoplasm to a membrane-bound compartment for association with MHC class I molecules.

70 Kd peroxisomal membrane protein (PMP70).

ALDP, a peroxisomal protein involved in X-linked adrenoleukodystrophy [9].

Sulfonylurea receptor [10], a putative subunit of the B-cell ATP-sensitive potassium channel.

Drosophila proteins white (w) and brown (bw), which are involved in the import of ommatidium screening pigments.

Fungal elongation factor 3 (EF-3).

Yeast STE6 which is responsible for the export of the a-factor pheromone.

Yeast mitochondrial transporter ATM1.

Yeast MDL1 and MDL2.

Yeast SNQ2.

Yeast sporidesmin resistance protein (gene PDR5 or STS1 or YDR1).

Fission yeast heavy metal tolerance protein hmt1. This protein is probably involved in the transport of metal-bound phytochelatins.

Fission yeast brefeldin A resistance protein (gene bfr1 or hba2).

Fission yeast leptomycin B resistance protein (gene pmd1).

mbpX, a hypothetical chloroplast protein from Liverwort.

Prestalk-specific protein tagB from slime mold. This protein consists of two domains: a N-terminal subtilase catalytic domain and a C-terminal ABC transporter domain.
[1] Higgins et al., Biomembr. 22:571-592 (1990).
[2] Higgins et al., BioEssays 8:111-116 (1988).
[3] Higgins et al., Nature 323:448-450 (1986).
[4] Doolittle et al., Nature 323:451-453 (1986).
[5] Blight et al., Mol. Microbiol. 4:873-880 (1990).
[6] Stoddard et al., Appl. Environ. Microbiol. 58:1952-1961 (1992).
[7] Rosteck Jr. et al., Gene 102:27-32 (1991).
[8] Gottesman et al., J. Biol. Chem. 263:12163-12166 (1988).
[9] Valle et al., J. Nature 361:682-683 (1993).
[10] Aguilar-Bryan et al., Science 268:423-426 (1995).
[E1] http://gdbdoc.gdb.org/˜avoltz/abcintro.html

Accordingly, transporters are a major target for drug action and development. Therefore, it is valuable to the field of pharmaceutical development to identify and characterize previously unknown transporters. The present invention advances the state of the art by providing previously unidentified human transporters.

### SUMMARY OF THE INVENTION

Novel transporter nucleotide sequences, and the deduced transporter polypeptides are described herein. Accordingly, the invention provides isolated transporter nucleic acid molecules having the sequences shown in SEQ ID NOS:1, 3, 4, 6, 7, 9, 10, 12, 13, 15, 16, or 18, and variants and fragments thereof.

It is also an object of the invention to provide nucleic acid molecules encoding the transporter polypeptides, and variants and fragments thereof. Such nucleic acid molecules are useful as targets and reagents in transporter expression assays, applicable to treatment and diagnosis of transporter-related disorders and are useful for producing novel transporter polypeptides by recombinant methods.

The invention thus further provides nucleic acid constructs comprising the nucleic acid molecules described herein. In a preferred embodiment, the nucleic acid molecules of the invention are operatively linked to a regulatory sequence. The invention also provides vectors and host cells for expressing the transporter nucleic acid molecules and polypeptides, and particularly recombinant vectors and host cells.

In another aspect, it is an object of the invention to provide isolated transporter polypeptides and fragments and variants thereof, including a polypeptide having the amino acid sequence shown in SEQ ID NOS:2, 5, 8, 11, 14, or 17 or the amino acid sequences encoded by the deposited cDNAs. The disclosed transporter polypeptides are useful as reagents or targets in transporter assays and are applicable to treatment and diagnosis of transporter-related disorders.

The invention also provides assays for determining the activity of or the presence or absence of the transporter polypeptides or nucleic acid molecules in a biological sample, including for disease diagnosis. In addition, the invention provides assays for determining the presence of a mutation in the polypeptides or nucleic acid molecules, including for disease diagnosis.

A further object of the invention is to provide compounds that modulate expression of the transporter for treatment and diagnosis of transporter-related disorders. Such compounds may be used to treat conditions related to aberrant activity or expression of the transporter polypeptides or nucleic acids.

The disclosed invention further relates to methods and compositions for the study, modulation, diagnosis and treatment of transporter related disorders. The compositions include transporter polypeptides, nucleic acids, vectors, transformed cells and related variants thereof. In particular, the invention relates to the diagnosis and treatment of transporter-related disorders including, but not limited to, disorders as described in the background above, further herein, or involving a tissue shown in the figures herein.

In yet another aspect, the invention provides antibodies or antigen-binding fragments thereof that selectively bind the transporter polypeptides and fragments. Such antibodies and antigen binding fragments have use in the detection of the transporter polypeptide, and in the prevention, diagnosis and treatment of transporter related disorders.

The transporters disclosed herein are designated as follows: 20685, 579, 17114, 23821, 32613, and 33894.

### DESCRIPTION OF THE DRAWINGS

FIGS. 1A & B shows the 20685 transporter cDNA sequence (SEQ ID NO:1), the predicted coding sequence (SEQ ID NO:3), and the deduced amino acid sequence (SEQ ID NO:2).

FIG. 2 shows a 20685 transporter hydrophobicity plot and domains. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:2) of human 20685 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIG. 3 shows an analysis of the 20685 transporter amino acid sequence: αβturn and coil regions; hydrophilicity; amphipathic regions; flexible regions; antigenic index; and surface probability plot.

FIG. 4 shows an analysis of the 20685 transporter open reading frame for amino acids corresponding to specific functional sites and predicted transmembrane segments of SEQ ID NO:2. For the cAMP- and cGMP-dependent protein kinase phosphorylation site, the actual modified residue is the last amino acid. For protein kinase C phosphorylation sites, the actual modified residue is the first amino acid. For casein kinase II phosphorylation sites, the actual modified residue is the first amino acid. For N-myristoylation sites, the actual modified residue is the first amino acid.

FIG. 5 shows PSORT prediction of protein localization for the 20685 transporter.

FIGS. 6A, B,C1 and C2 shows a search for complete domains in PFAM for the 20685 transporter. This Figure includes alignments of the transporter domains of human 20685 with consensus amino acid sequences derived from hidden Markov models. In all the alignments the upper sequence is the consensus amino acid sequence, while the lower amino acid sequence corresponds to amino acids of SEQ ID NO:2. In the first alignment the upper sequence is SEQ ID NO:19 and the lower sequence corresponds to amino acids 344 to 357 of SEQ ID NO:2. In the second alignment the upper sequence is SEQ ID NO:20 and the lower sequence corresponds to amino acids 35 to 434 of SEQ ID NO:2. In the third alignment the upper sequence is SEQ ID NO:21 and the lower sequence corresponds to amino acids 39 to 446 of SEQ ID NO:2. In the fourth alignment the upper sequence is SEQ ID NO:22 and the lower sequence corresponds to amino acids 10 to 456 of SEQ ID NO:2.

FIGS. 7A and B shows expression of the 20685 in various human tissues and cells. The abbreviations of the various tissues and cells are as follows: LF: liver fibrosis; Grans: granulocytes; PBMC: peripheral blood mononuclear cells; BM-MNC: bone marrow mononuclear cells; mPB: mobilized peripheral blood cells; ABM: adult bone marrow; mBM: mobilized bone marrow; Meg: megakaryocytes; BM: bone marrow; HepG2: hepatocyte specific cell line; NHLF: normal human lung fibroblasts; TH1: Th1 cells; TH2: Th2 cells; NHBE: normal human bronchial epithelial; HepG2 2.2.15-A: HepG2 cell line stably transfected with HBV. Tissues and cells are analyzed for expression of the 20685 mRNA from left to right as follows: Lung MPI 188, Kidney MPI 58, Brain 167, Heart Pit 273, Colon MPI 383, Tonsil MPI 37, Spleen MPI 380, Fetal Liver MPI, Liver NDR 154, Stellate D3#1, Stellate FBS, NHLF CTN 48 hr, NHLF TGF 10 ng, HepG2 CTN 48 hr, HepG2 TGF 10 ng, NHLH resting, NHLF activated, LF NDR 190, LF NDR 191, LF NDR 194, LF NDR 113, TH1 48 hr M4, TH1 48 hr M5, TH2 48 hr M5, Grans, CD19, CD14, CD14 activated, PBMC mock, PBMC PHA, PBMC IL10, PBMC IL13, NHBE IL13-1, BM-MNC, mPB CD34+, ABM CD34+, mBM CD34+, Erythroid, Megs, Neutrophils, mBM CD11b+, mBM CD15+, mBM CD11b−, BM CD71, HepG2A, HepG2 2.21-a.

FIG. 8 shows mapping information for the 20685 transporter gene.

FIGS. 9A, B & C shows the 579 transporter cDNA sequence (SEQ ID NO:4), the predicted coding sequence (SEQ ID NO:6), and the deduced amino acid sequence (SEQ ID NO:5).

FIG. 10 shows a 579 transporter hydrophobicity plot and analysis of the domains. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:5) of human 579 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIGS. 11A & B shows predicted MEMSAT 579 transmembrane segments and an analysis of the 579 transporter open reading frame for amino acids corresponding to specific functional sites of SEQ ID NO:5. For the N-glycosylation sites, the actual modified residue is the first amino acid. For the cAMP- and cGMP-dependent protein kinase phophorylation sites, the actual modified residue is the last amino acid. For protein kinase C phosphorylation sites, the actual modified residue is the first amino acid. For casein kinase II phosphorylation sites, the actual modified residue is the first amino acid. For the tyrosine kinase phosphorylation site, the actual modified residue is the last amino acid residue. For N-myristoylation sites, the actual modified residue is the first amino acid. In addition, a sodium neurotransmitter symporter family signature is found from about amino acids 85 to 99.

FIG. 12 shows PSORT prediction of protein localization for the 579 transporter.

FIGS. 13A & B shows a search for complete domains in PFAM for the 579 transporter. This Figure includes an alignment of the transporter domain of human 579 with a consensus amino acid sequence derived from a hidden Markov model. In all the alignments the upper sequence is the consensus amino acid sequence, while the lower amino acid sequence corresponds to amino acids of SEQ ID NO:5. In the first alignment the upper sequence is SEQ ID NO:23 and the lower sequence corresponds to amino acids 409 to 641 of SEQ ID NO:5. In the second alignment the upper sequence is SEQ ID NO:24 and the lower sequence corresponds to amino acids 61 to 659 of SEQ ID NO:5.

FIGS. 14A, B, C, D, E, F & G shows the 17114 transporter cDNA sequence (SEQ ID NO:7), the predicted coding sequence (SEQ ID NO:9), and the deduced amino acid sequence (SEQ ID NO:8).

FIG. 15 shows a 17114 transporter hydrophobicity plot and domains. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:8) of human 17114 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIGS. 16A, B, C, D, E & F shows 17114 signal peptide predictions, predicted MEMSAT 17114 transmembrane segments, and an analysis of the 17114 transporter open reading frame for amino acids corresponding to specific functional sites of SEQ ID NO:8. For the N-glycosylation sites, the actual modified residue is the first amino acid. For cAMP- and cGMP-dependent protein kinase phosphorylation sites, the actual modified residue is the last amino acid. For protein kinase C phosphorylation sites, the actual modified residue is the first amino acid. For casein kinase II phosphorylation sites, the actual modified residue is the first amino acid. For the tyrosine kinase phosphorylation site, the actual modified residue is the last amino acid residue. For N-myristoylation sites, the actual modified residue is the first amino acid. In addition, an ABC transporter family signature is found from about amino acids 1124-1138.

FIG. 17 shows PSORT prediction of protein localization for the 17114 transporter.

FIGS. 18A, B & C shows a search for complete domains in PFAM for the 17114 transporter. This Figure includes alignments of the transporter domains of human 17114 with consensus amino acid sequences derived from hidden Markov models. In all the alignments the upper sequence is the consensus amino acid sequence, while the lower amino acid sequence corresponds to amino acids of SEQ ID NO:8. In the first alignment the upper sequence is SEQ ID NO:25 and the lower sequence corresponds to amino acids 1018 to 1198 of SEQ ID NO:8. In the second alignment the upper sequence is SEQ ID NO:26 and the lower sequence corresponds to amino acids 1733 to 1755 of SEQ ID NO:8. In the third alignment the upper sequence is SEQ ID NO:27 and the lower sequence corresponds to amino acids 1542 to 1963 of SEQ ID NO:8. In the fourth alignment the upper sequence is SEQ ID NO:28 and the lower sequence corresponds to amino acids 2081 to 2262 of SEQ ID NO:8. In the fifth alignment the upper sequence is SEQ ID NO:29 and the lower sequence corresponds to amino acids 1017 to 1199 of SEQ ID NO:8. In the sixth alignment the upper sequence is SEQ ID NO:30 and the lower sequence corresponds to amino acids 2080 to 2265 of SEQ ID NO:8.

FIGS. 19A & B shows the 23821 transporter cDNA sequence (SEQ ID NO:10), the predicted coding sequence (SEQ ID NO:12), and the deduced amino acid sequence (SEQ ID NO:11).

FIG. 20 shows a 23821 transporter hydrophobicity plot. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:11) of human 23821 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIG. 21 shows predictions for 23821 signal peptides, predicted MEMSAT 23821 transmembrane segments, and an analysis of the 23821 transporter open reading frame for amino acids corresponding to specific functional sites of SEQ ID NO:11. For the N-glycosylation sites, the actual modified residue is the first amino acid. For protein kinase C phosphorylation sites, the actual modified residue is the first amino acid. For the casein kinase II phosphorylation site, the actual modified residue is the first amino acid. For N-myristoylation sites, the actual modified residue is the first amino acid. In addition, a neurotransmitter-gated ion-channel signature is found from about amino acids 154-168.

FIG. 22 shows PSORT prediction of protein localization for the 23821 transporter.

FIG. 23 shows a search for complete domains in PFAM for the 23821 transporter. This Figure includes an alignment of the transporter domain of human 23821 with a consensus amino acid sequence derived from a hidden Markov model. In the alignment the upper sequence is SEQ ID NO:31 and the lower sequence corresponds to amino acids 30 to 446 of SEQ ID NO:11.

FIGS. 24A, B, & C shows the 32613 transporter cDNA sequence (SEQ ID NO:13), the predicted coding sequence (SEQ ID NO:15), and the deduced amino acid sequence (SEQ ID NO:14).

FIG. 25 shows a 32613 hydrophobicity plot and domains. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:14) of human 32613 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIG. 26 shows an analysis of the 32613 transporter amino acid sequence: αβturn and coil regions; hydrophilicity; amphipathic regions; flexible regions; antigenic index; and surface probability plot.

FIGS. 27A & B shows predicted MEMSAT 32613 transmembrane segments and an analysis of the 32613 transport open reading frame for amino acids corresponding to specific functional sites of SEQ ID NO:14. For the N-glycosylation sites, the actual modified residue is the first amino acid. For protein kinase C phosphorylation sites, the actual modified residue is the first amino acid. For the casein kinase II phosphorylation site, the actual modified residue is the first amino acid. For N-myristoylation sites, the actual modified residue is the first amino acid. For the tyrosine kinase phosphorylation site, the actual modified residue is the last amino acid.

FIG. 28 shows PSORT prediction of protein localization for the 32613 transporter.

FIGS. 29A, B & C shows a search of complete domains in PFAM for the 32613 transporter. This Figure includes alignments of the transporter domains of human 32613 with consensus amino acid sequences derived from hidden Markov models. In all the alignments the upper sequence is the consensus amino acid sequence, while the lower amino acid sequence corresponds to amino acids of SEQ ID NO:14. In the first alignment the upper sequence is SEQ ID NO:32 and the lower sequence corresponds to amino acids 120 to 399 of SEQ ID NO:14. In the second alignment the upper sequence is SEQ ID NO:33 and the lower sequence corresponds to amino acids 148 to 434 of SEQ ID NO:14. In the third alignment the upper sequence is SEQ ID NO:34 and the lower sequence corresponds to amino acids 161 to 512 of SEQ ID NO:14. In the fourth alignment the upper sequence is SEQ ID NO:35 and the lower sequence corresponds to amino acids 209 to 519 of SEQ ID NO:14. In the fifth alignment the upper sequence is SEQ ID NO:36 and the lower sequence corresponds to amino acids 356 to 548 of SEQ ID NO:14.

FIGS. 30A, B & C shows the 33894 transporter cDNA sequence (SEQ ID NO:16), the predicted coding sequence (SEQ ID NO:18), and the deduced amino acid sequence (SEQ ID NO:17).

FIG. 31 shows a 33894 transporter hydrophobicity plot and domains. Relative hydrophobic residues are shown above the dashed horizontal line, and relative hydrophilic residues are below the dashed horizontal line. The cysteine residues (cys) and N glycosylation site (Ngly) are indicated by short vertical lines just below the hydropathy trace. The numbers corresponding to the amino acid sequence (shown in SEQ ID NO:17) of human 33894 are indicated. Polypeptides of the invention include fragments which include: all or a part of a hydrophobic sequence (a sequence above the dashed line); or all or part of a hydrophilic fragment (a sequence below the dashed line). Other fragments include a cysteine residue or an N-glycosylation site.

FIG. 32 shows an analysis of the 33894 transporter amino acid sequence: αβturn and coil regions; hydrophilicity; amphipathic regions; flexible regions; antigenic index; and surface probability plot.

FIGS. 33A & B shows predictions for 33894 signal peptide, predicted MEMSAT 33894 transmembrane segments, and an analysis of the 33894 transporter open reading frame for amino acids corresponding to specific functional sites of SEQ ID NO:17. For the N-glycosylation site, the actual modified residue is the first amino acid. For the cAMP and cGMP-dependent protein kinase phosphorylation site, the actual modified residue is the last amino acid. For protein kinase C phosphorylation sites, the actual modified residue is the first amino acid. For casein kinase II phosphorylation sites, the actual modified residue is the first amino acid. For the tyrosine kinase phosphorylation site, the actual modified residue is the last amino acid. For N-myristoylation sites, the actual modified residue is the first amino acid. In addition, an ABC transporter family signature is found at about amino acid 643-657.

FIGS. 34A, B and C shows a search for complete domains in PFAM for the 33894 transporter. This Figure includes alignments of the transporter domains of human 33894 with consensus amino acid sequences derived from hidden Markov models. In all the alignments the upper sequence is the consensus amino acid sequence, while the lower amino acid sequence corresponds to amino acids of SEQ ID NO:17. In the first alignment the upper sequence is SEQ ID NO:37 and the lower sequence corresponds to amino acids 1 to 227 of SEQ ID NO:17. In the second alignment the upper sequence is SEQ ID NO:38 and the lower sequence corresponds to amino acids 388 to 409 of SEQ ID NO:17. In the third alignment the upper sequence is SEQ ID NO:39 and the lower sequence corresponds to amino acids 188 to 459 of SEQ ID NO:17. In the fourth alignment the upper sequence is SEQ ID NO:40 and the lower sequence corresponds to amino acids 531 to 650 of SEQ ID NO:17. In the fifth alignment the upper sequence is SEQ ID NO:41 and the lower sequence corresponds to amino acids 532 to 716 of SEQ ID NO:17. In the sixth alignment the upper sequence is SEQ ID NO:42 and the lower sequence corresponds to amino acids 531 to 717 of SEQ ID NO:17.

FIG. 35 shows expression of the 33894 transporter in various human tissues and cells. Tissues and cells are from right to left as follows: Aorta, Lymph Node, Tonsil, Thymus, Spinal Cord, Spleen, Cervix, Fet Spinal Cord, Osteoblasts Primary Culture, Osteoblasts Differentiated, Osteoblasts Undifferentiated, Fetal Heart (columns 12-13), Fetal Liver (columns 14-15), Placenta, Teste, Skin (columns 18-19), Thyroid (columns 20-21), Small Intestine, Adipose (columns 23-24), Trachea, Vein (columns 26-27), Lung (columns 28-29), Kidney (columns 30-31), Ovary (columns 32-33), Heart (columns 34-35), Colon (columns 36-37), Brain (columns 38-39), Skeletal Muscle (columns 40-41), Breast (columns 42-43), Liver (columns 44-45), Osteoclasts, Prostate.

FIGS. 36A & B shows expression of 20685 in various virus infected human tissues and cells. A) The tissues and cells analyzed for 20685 mRNA expression are listed from left to right: Normal Liver (NDR 200), Normal Liver (Pit 260), HBV Liver (MAI 01), HBV Liver (MAI 04), HBV Liver (MAI 10), HepC+ Liver (Pit 519) (Hepatitis C infected liver), HepC+ Liver (Pit 519), HepG2-B (liver specific cell line), HepG2.2.15-B (HepG2-B stably transfected with HBV), HepG2 no treat #1, HepG2-B IC50 #2, HepG2-B IC100 #3, HepG2.2.15 no treat #4, HepG2.2.15-B IC50 #5, HepG2.2.15-B IC100 #6, HepG2.2.15 no treat old #11, HepG2.2.15 3TC IC 100 old #12, HepG2.2.15 3TC IC50 old #13, HepG2 control, HepG2 transfected, HuH7 control (human hepatocellular carcinoma cell line), HuH7 transfected, Old 1. B) The tissues and cells analyzed for 20685 mRNA expression are listed from left to right: Normal Liver (260), Normal Liver (200), HBV+ Liver (MA101) (HBV infected liver), HBV+ Liver (MAI 10), HepC+ Liver (518) (Hepatitis C virus infected liver), HepC+ Liver (519), HepG2, HepG2.2.15, HepG2 control (#1), HepG2 transfected HBV-X (#2), HuH7 control (#3), HuH7 transfected HBV-X (#4), HSV-ganglia 287 (Herpes simplex virus), HSV+ ganglia 290, NT2/KOS 0 hr. #9 (embryonal carcinoma cell line transfected with live avirulent HSV-1), NT2/KOS 2.5 hr. #10, NT2/KOS 5 hr. #11, NT2/KOS 7 hr. #12, MRC/VZV Mock (human embryo lung cells mock transfected with varicella-zoster virus), MRC/VZV 18 hr., MRC/VZV 72 hr.

FIGS. 37A1, 37A2, 37B1 and 37B2 shows expression of 579 in various human tissues and cells. A) The tissues and cells analyzed for 579 mRNA expression from left to right include: Lung (MPI 131), Kidney (MPI 58), Brain (MPI 167), Heart (PIT 272), Colon (MPI 383), Tonsil (MPI 37), Lymph Nodes (NDR 173), Spleen (MPI 380), Fetal Liver (MPI 133), Pooled Liver, Stellate, Stellate-FBS, NHLF Mock, NHLF TGF, HepG2 Mock, HepG2 TGF, NHLH Resting, NHLH Activated, Liver Fibrosis (NDR 190), Liver Fibrosis (NDR 191), Liver Fibrosis (NDR 194), Th1 48 hr (M4), Th2 48 hr. (M4), Th1 48 hr (M5), Th2 48 hr (M5), Grans (Donor 8), CD19 (LP031999), CD 14 #7 (CG0006), CD14 LPS (CG0010), PBMC Mock, PBMC PHA, PBMC IL10, IL4, PBMC IFN g, TNF, NHBE Mock, NHBE IL13-1, Th0 24 hr (L67), Th2 24 (RLD63), BM-MNC, mPB CD34+, ABM CD34+, mBM CD34+, Erythroid, and Megakaryocytes. B) The tissues and cells analyzed for 579 mRNA expression are listed from left to right: Prostate MPI 242, Osteoclasts, Liver MPI 154, Breast CLN 734, Breast CLN 736, Skeletal Muscle MPI 166, Skeletal Muscle MPI 570, Brain MPI 515, Colon MPI 176, Colon MPI 383/411, Heart MPI 664, Heart MPI 53, Kidney, Ovary MPI 415, Lung MPI 28, Vein MPI 134, Vein MPI 135, Adipose MPI 621, Adipose MPI 620, Small Intestine MPI 376, Thyroid MPI 54, Skin MPI 572, Testis MPI 33/78, Placenta MPI 391/76, Fetal Liver MPI 425, Fetal Liver MPI 133, Fetal Heart MPI 32, Fetal Heart MPI 164, Undifferentiated Osteoblast, Differentiated Osteoblast, Prim Cult Osteoblast, Spinal Cord MPI 655, Cervix MPI 567, Spleen MPI 380, Spinal Cord MPI 651, Thymus MPI 388, Tonsil MPI 396, Lymph Node MPI 158, and Aorta CLN 618.

FIGS. 38A and B shows expression of 17114 in various human tissues and cells. The tissues and cells analyzed for 17114 mRNA expression from left to right include: Artery Normal, Aorta Diseased, Vein Normal, Coronary SMC, HUVEC, Hemangioma, Heart Normal, Heart CHF, Kidney, Skeletal Muscle, Adipose Normal, Pancreas, Primary Osteoblasts, Osteoclasts (Diff), Skin Normal, Spinal Cord Normal, Brain Cortex Normal, Brain Hypothalamus Normal, Nerve, DRG (Dorsal Root Ganglion), Breast Normal, Breast Tumor, Ovary Normal, Ovary Tumor, Prostate Normal, Prostate Tumor, Salivary Glands, Colon Normal, Colon Tumor, Lung Normal, Lung Tumor, Lung COPD, Colon IBD, Liver Normal, Liver Fibrosis, Spleen Normal, Tonsil Normal, Lymph Node Normal, Small Intestine Normal, Skin-Decubitus, Synovium, BM-MNC, Activated PBMC, Neutrophils, Megakaryocytes, and Erythroid.

FIG. 39 shows expression of 579 in various human tissues and cells in a human pain panel phase I experiment. The tissues and cells analyzed for 579 mRNA expression from left to right are: Adrenal Gland, Brain, Heart, Kidney, Liver, Lung, Mammary Gland, Pancreas, Placenta, Prostate, Salivary Gland, Muscle, Small Intestine, Spleen, Bladder, Prostate BPH, Thymus, Trachea, Uterus, Spinal Cord, DRG (Dorsal Root Ganglion), Skin, and Ureter.

FIG. 40 shows expression of the r16854 rat ortholog of 579 in various rat tissues and cells in a rat pain panel phase I experiment. The tissues and cells analyzed for r16854 mRNA expression from left to right are: Brain, Spinal Cord, DRG (Dorsal Root Ganglion), SCG (Superior Cervical Ganglion), Ovary, Hairy Skin, Gastro Muscle, Heart, Kidney, Liver, Lung, Spleen, Aorta, Adrenal Gland, Salivary Gland, Thyroid, Prostate, Thymus, Trachea, Esophagus, Duodenum, Diaphram, and Colon.

FIG. 41 shows expression of the r16854 rat ortholog of 579 in a rat pain panel phase II experiment in rat DRG (Dorsal Root Ganglion) as a function of exposure to CCI (chronic constriction injury), CFA (complete Freund's adjuvant), and AXT (axotomy). Columns 1-6 show naïve DRG (column 1) and DRG at 3, 7, 10, 14, and 28 days after exposure to CCI (columns 2-6, respectively). Columns 7-13 show naïve DRG (column 7) and DRG at 1, 3, 7, 10, 14, and 28 days after exposure to CFA (columns 8-13, respectively). Columns 14-19 show naïve DRG (column 14) and DRG at 1, 3, 7, 14, and 28 days after exposure to AXT (columns 2-6, respectively).

### DETAILED DESCRIPTION OF THE INVENTION

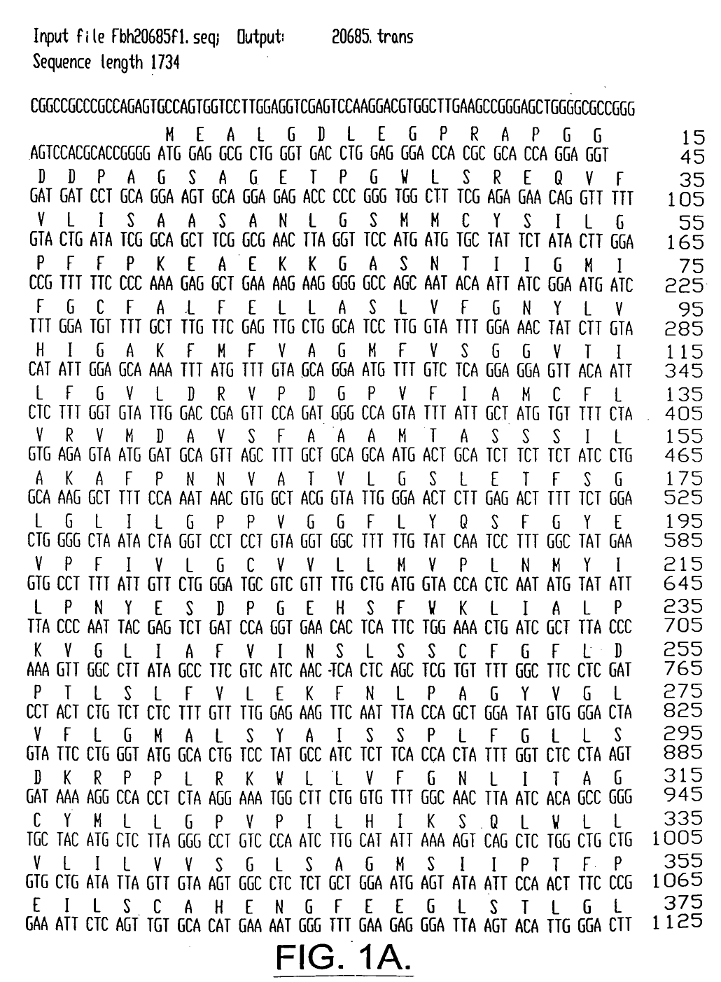

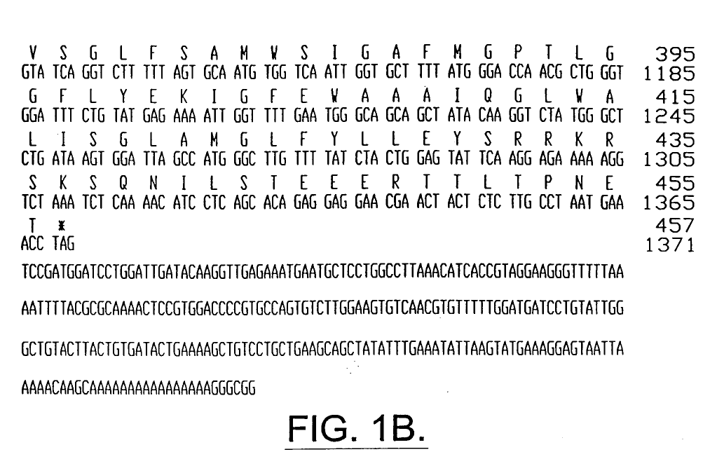

The invention is based on the identification of six novel human cDNA molecules that encode transporter proteins. These molecules and the encoded polypeptides are designated 20685, 579, 17114, 23821, 32613, and 33894. The transporter cDNA was identified in human cDNA libraries. Specifically, expressed sequence tags (EST) found in human cDNA libraries, were selected based on homology to known transporter sequences. Based on such EST sequences, primers were designed to identify a full length clone from a human cDNA library. Positive clones were sequenced and the overlapping fragments were assembled. The 20685, 579, 17114, 23821, 32613, and 33894 transporter amino acid sequences are shown in FIGS. 1A & B, 9A-C, 14A-G, 19A & B, 24A-C, and 30A-C, respectively, and SEQ ID NOS:2, 5, 8, 11, 14, and 17, respectively. The cDNA sequences are also shown in these figures (SEQ ID NOS:1 and 3, 4 and 6, 7 and 9, 10 and 12, 13 and 15, 16 and 18, respectively). Identification of the cDNA molecules was based upon consensus motifs or protein domains that are characteristic of transporter proteins. Transporter proteins are defined as polypeptides that are capable of transporting a substrate molecule or ion across a cell membrane.

To identify the presence of consensus motifs or protein domains in a protein sequence that are characteristic of transporter proteins, and make the determination that a polypeptide or protein of interest has a particular profile, the amino acid sequence of the protein can be searched against a database of hidden Markov models (HMMs) (e.g., the Pfam database, release 2.1) using the default parameters (http://www.sanger.ac.uk/Software/Pfam/HMM_search). For example, the hmmsf program, which is available as part of the HMMER package of search programs, is a family specific default program for MILPAT0063 and a score of 15 is the default threshold score for determining a hit. Alternatively, the threshold score for determining a hit can be lowered (e.g., to 8 bits). A description of the Pfam database can be found in Sonhammer et al. (1997) Proteins 28(3):405-420 and a detailed description of HMMs can be found, for example, in Gribskov et al. (1990) Meth. Enzymol. 183:146-159; Gribskov et al. (1987) Proc. Natl. Acad. Sci. USA 84:4355-4358; Krogh et al. (1994) J. Mol. Biol. 235:1501-1531; and Stultz et al. (1993) Protein Sci. 2:305-314, the contents of which are incorporated herein by reference. For general information regarding PFAM identifiers, PS prefix and PF prefix domain identification numbers, refer to Sonnhammer et al. (1997) Protein 28:405-420 and http//www.psc.edu/general/software/packages/pfam/pfam.html.

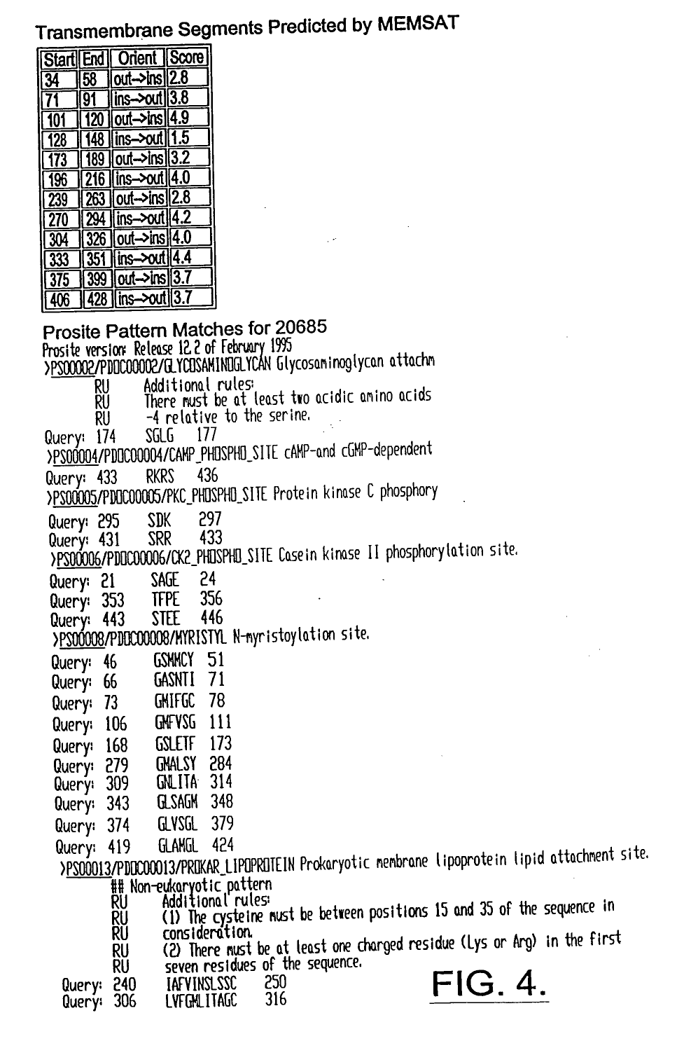

One molecule upon which the invention is based is the 20685 transporter. The 20685 transporter gene encodes an approximately 1734 nucleotide mRNA transcript with an open reading frame that encodes a 456 amino acid protein. Prosite program analysis was used to predict various sites within the 20685 transporter protein as shown in FIG. 4.

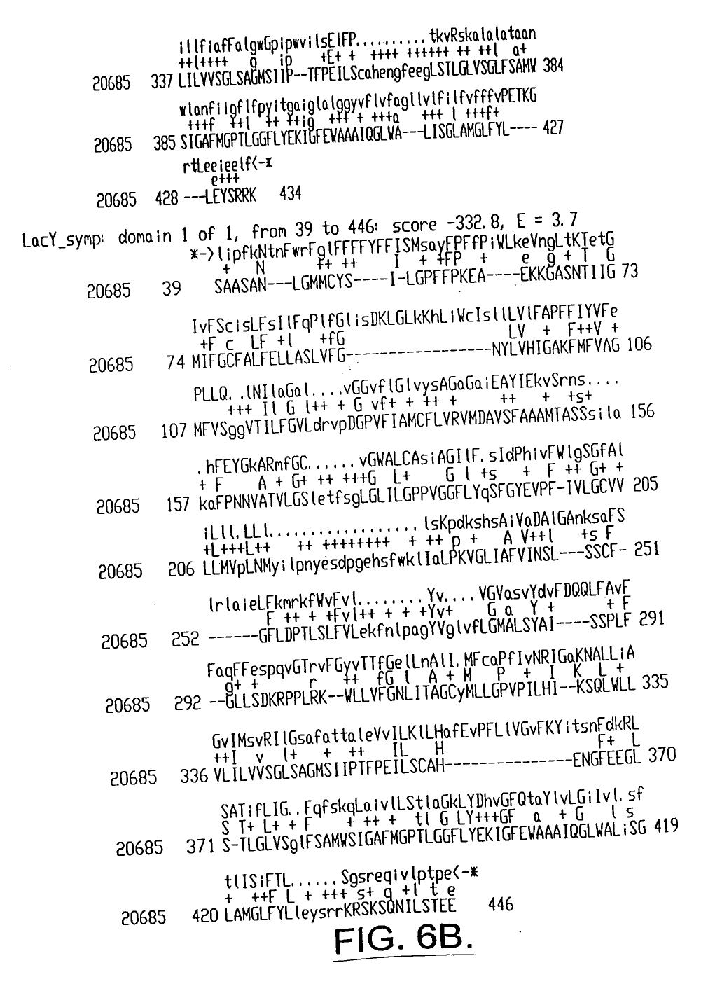

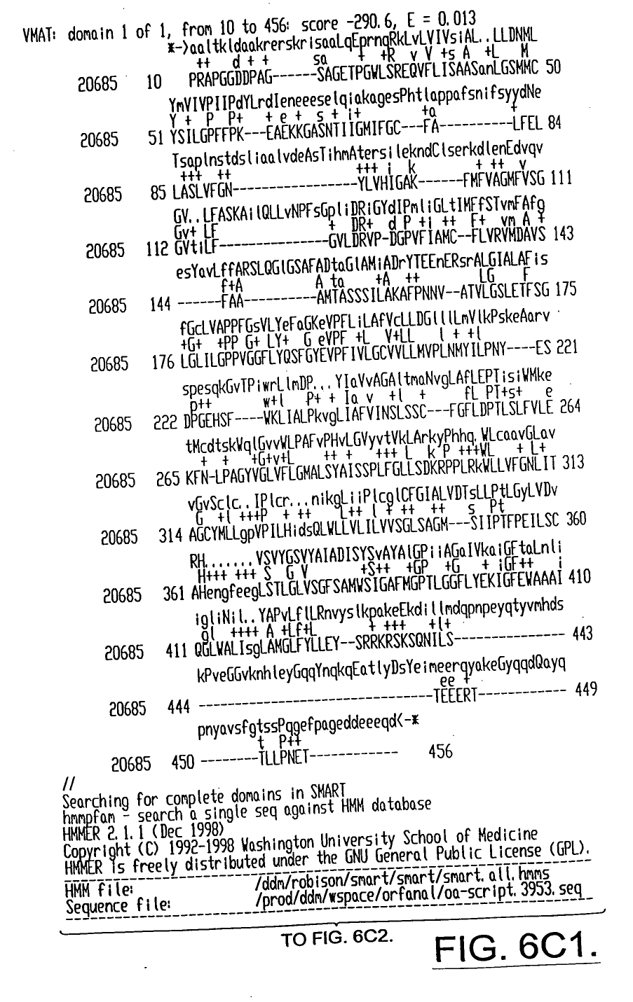

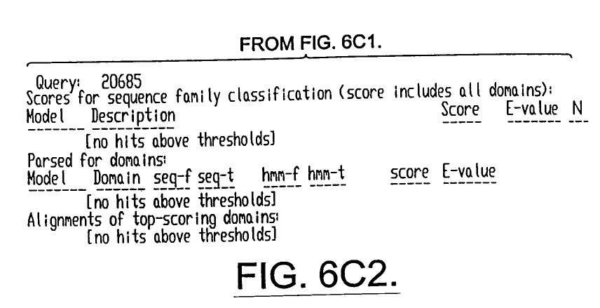

Pfam analysis indicates that this polypeptide shares sequence similarity with the sugar (and other) transporters and the vesicular monoamine transporters (FIG. 6). The sugar (and other) transporter domain (HMM) (PS00216 and PS00217) aligns with amino acids 35 to 434 of SEQ ID NO:2. The vesicular monoamine transporter domain (HMM) (PF01703) aligns with amino acids 10 to 456 of SEQ ID NO:2.

In one embodiment a 20685-like polypeptide or protein has a “sugar (and other) transporter domain” or a region which has at least about 60%, 70%, 80%, 90%, 95%, 99%, or 100% sequence identity with a “sugar (and other) transporter domain,” e.g., the sugar (and other) transporter domain of human 20685 (e.g., amino acid residues 35 to 434 of SEQ ID NO:2).

In one embodiment a 20685-like polypeptide or protein has a “vesicular monoamine transporter domain” or a region which has at least about 60%, 70%, 80%, 90%, 95%, 99%, or 100% sequence identity with a “vesicular monoamine transporter domain,” e.g., the vesicular monoamine transporter domain of human 20685 (e.g., amino acid residues 10 to 456 of SEQ ID NO:2).

ProDom matches for the 20685 transporter show similarity to vesicular monoamine transporters.

MEMSAT analysis of 20685 transporter protein predicts 12 transmembrane seqments or domains (FIG. 4). As used herein, the term “transmembrane domain” includes an amino acid sequence of about 15-30 amino acid residues in length that spans a phospholipid membrane. Transmembrane domains are rich in hydrophobic residues, and typically have an α-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, http://pfam.wustl.edu/cgi-bin/getdesc?name=7tm-1, and Zagotta W. N. et al. (1996) Annual Rev. Neuronsci. 19:235-63, the contents of which are incorporated herein by reference.

In one embodiment, a 20685-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of human 20685 (e.g., amino acid residues 34-58, 71-91, 101-120, 128-148, 173-189, 196-216, 239-263, 270-294, 304-326, 333-351, 375-399, 406-428 of SEQ ID NO:2).

In another embodiment, a 20685-like protein includes at least one “non-transmembrane domain.” As used herein, “non-transmembrane domains” are domains that reside outside of the membrane. When referring to plasma membranes, non-transmembrane domains include extracellular domains (i.e., outside of the cell) and intracellular domains (i.e., within the cell). When referring to membrane-bound proteins found in intracellular organelles (e.g., mitochondria, endoplasmic reticulum, peroxisomes and microsomes), non-transmembrane domains include those domains of the protein that reside in the cytosol (i.e., the cytoplasm), the lumen of the organelle, or the matrix or the intermembrane space (the latter two relate specifically to mitochondria organelles). The C-terminal amino acid residue of a non-transmembrane domain is adjacent to an N-terminal amino acid residue of a transmembrane domain in a naturally occurring 20685 protein, or 20685-like protein.

The 20685 gene has been mapped (TIGR-A006R06) to chromosome 16 with a location between D16S401 and D16S411 (45.5-57cM).

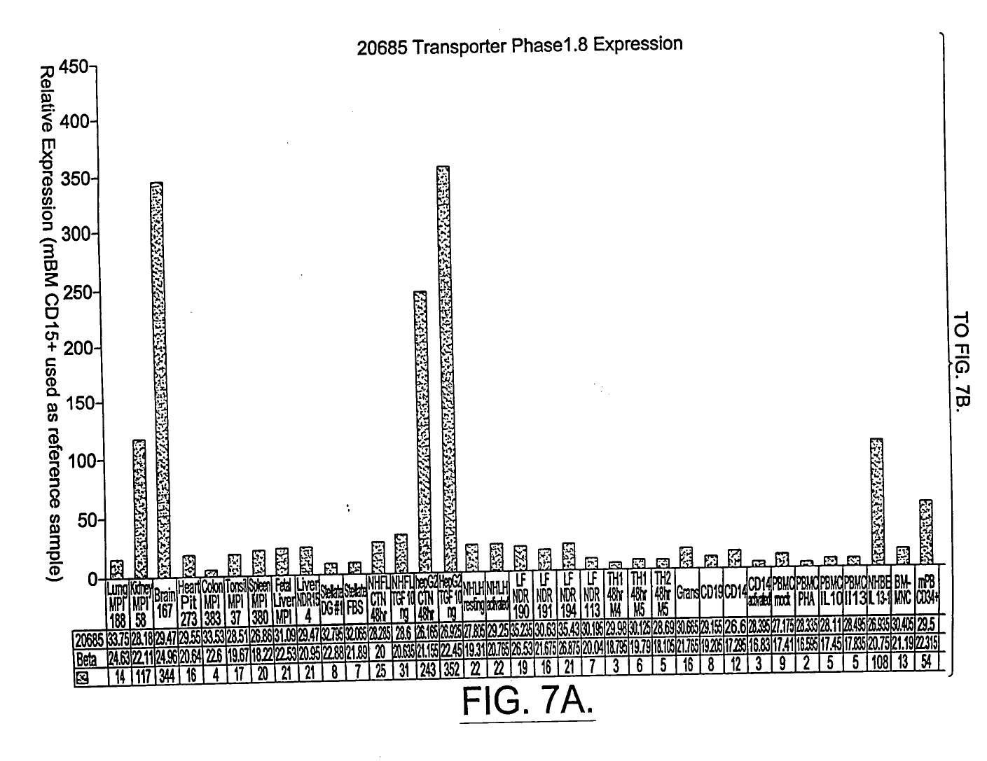

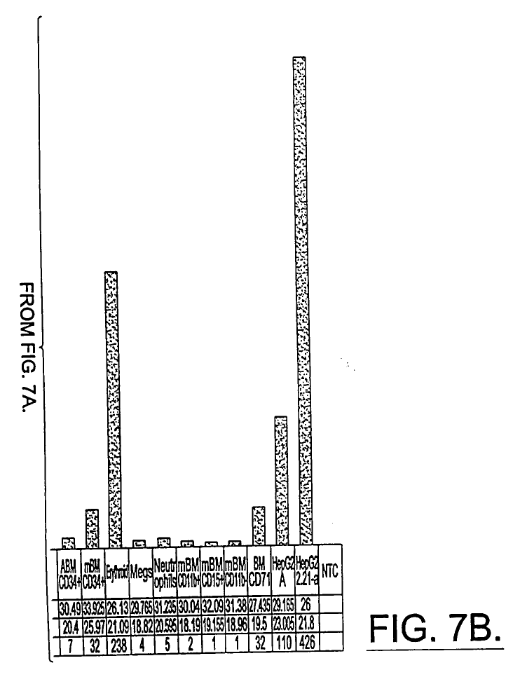

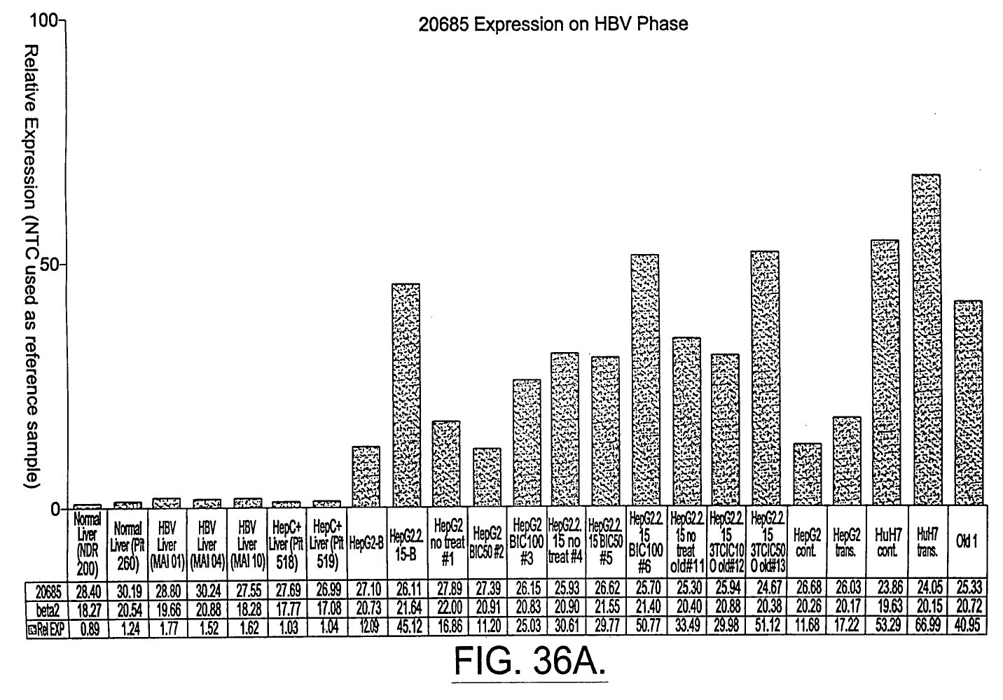

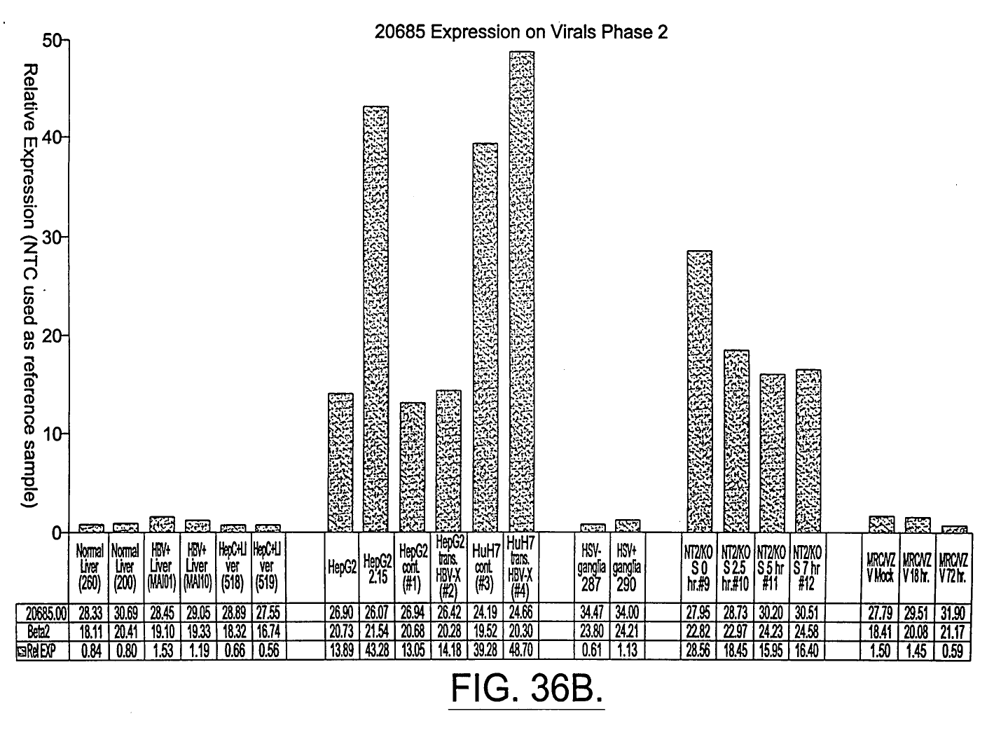

The 20685 gene is expressed in various human tissues and cells including, but not limited to, those shown in FIG. 7. This panel shows the highest levels of 20685 expression in HepG2 cells, brain, and erythroid cells. FIGS. 36A & B shows expression of 20685 in various virus infected human tissues and cells. Expression levels of 20685 in hepatocytes and in is hepatocytes transfected with HBV is shown in FIG. 36A. The 20685 gene is also expressed in various other tissues, including adrenal gland, blood, brain, breast: colon to liver metastases, D8 dendritic cells, epithelial cells, fibroblasts, heart keratinocytes, lung lymphocytes, lymphoma, megakaryocytes, neurons, osteoblasts, pituitary, prostate, skin, T-cells and thymus.

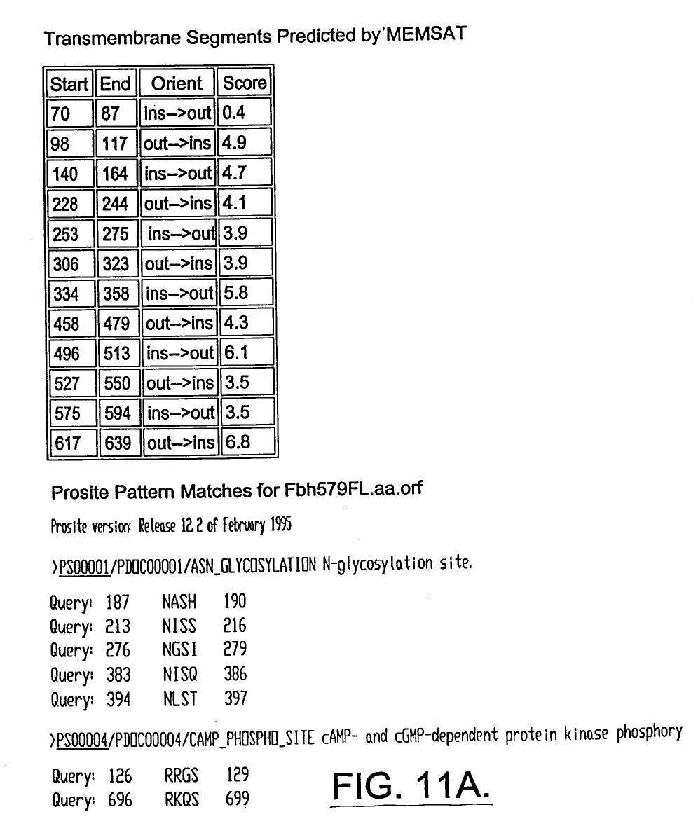

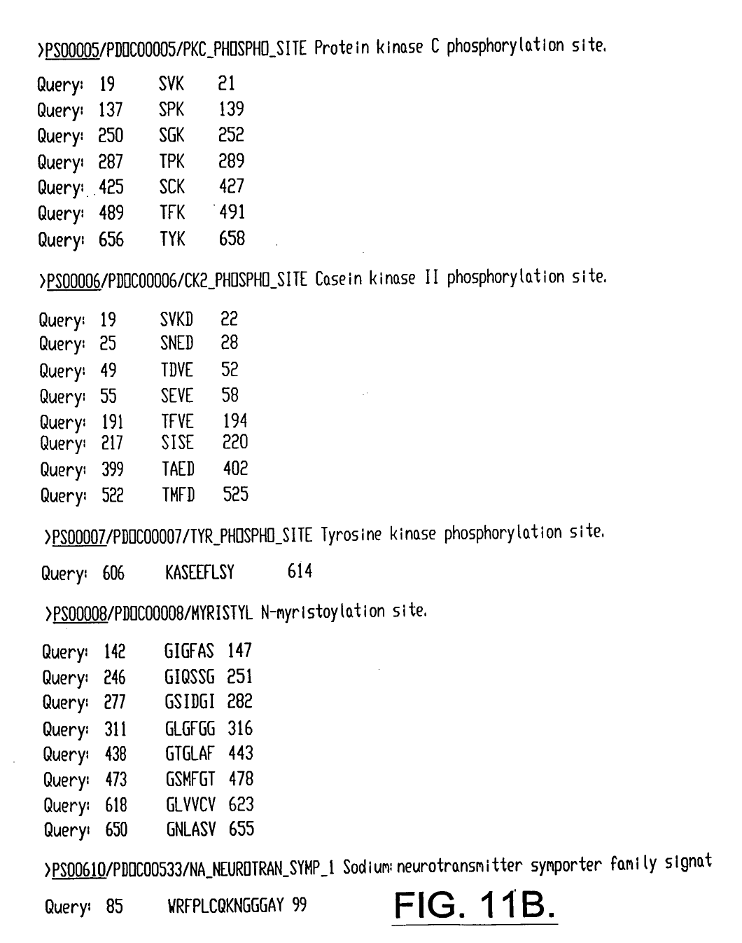

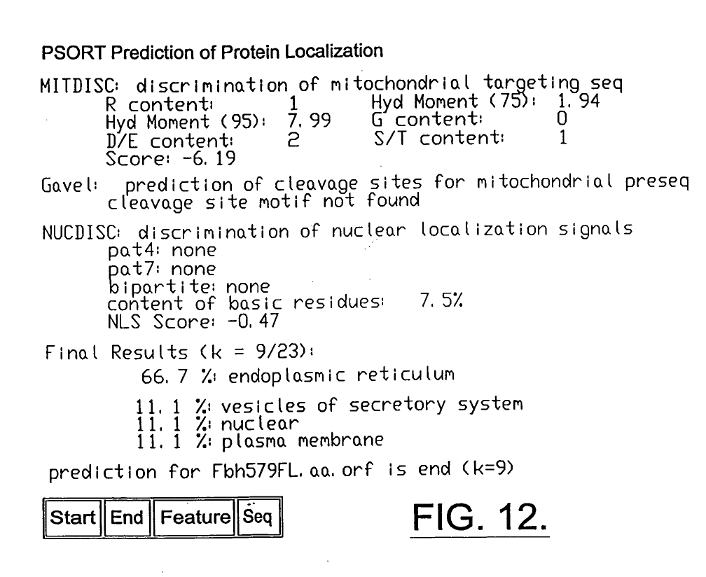

The 20685 transporter is useful for the diagnosis and treatment of vesicular monoamine transporter- and sugar (and other) transporter-related disorders. Where 20685 transporter is diferentially expressed in a virally-infected cell, modulation of the gene is especially relevant in such cells for treatment of the viral disorder and also useful for diagnosis of such a disorder. Further, expression is relevant to prevent, treat, or diagnose the effects of viral infection, particularly HBV infection, such as tissue fibrosis and especially liver fibrosis. The 20685 transporter is also useful for the diagnosis and treatment of neurological and central nervous system disorders, including Parkinson's disease, depression, and pain; infectious disease, particularly viral; cell proliferative disorders, including cancer; blood disorders, and immune and inflammatory disorders. The invention is also based on the identification of the novel human transporter designated 579. The 579 transporter gene encodes an approximately 3103 nucleotide mRNA transcript with an open reading frame that encodes a 730 amino acid protein. Prosite program analysis was used to predict various sites within the 579 transporter protein as shown in FIG. 11.

A plasmid containing the 579 transporter cDNA insert was deposited with the Patent Depository of the American Type Culture Collection (ATCC), 10801 University Boulevard, Manassas, Va., on Jun. 9, 2000, and assigned Patent Deposit Number PTA-2016. This deposit will be maintained under the terms of the Budapest Treaty on the International Recognition of the Deposit of Microorganisms for the Purposes of Patent Procedure. This deposit was made merely as a convenience for those of skill in the art and is not an admission that a deposit is required under 35 U.S.C. § 112.

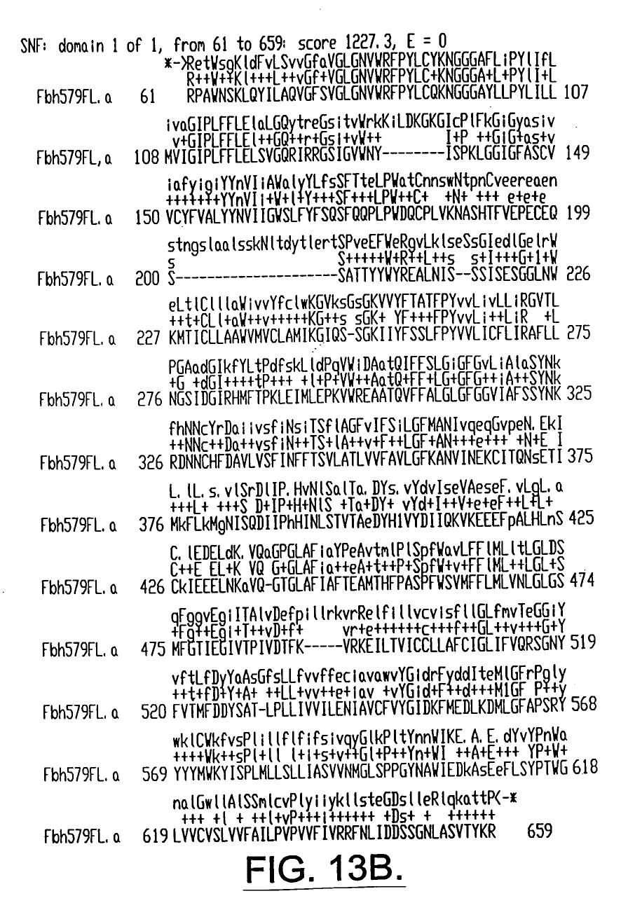

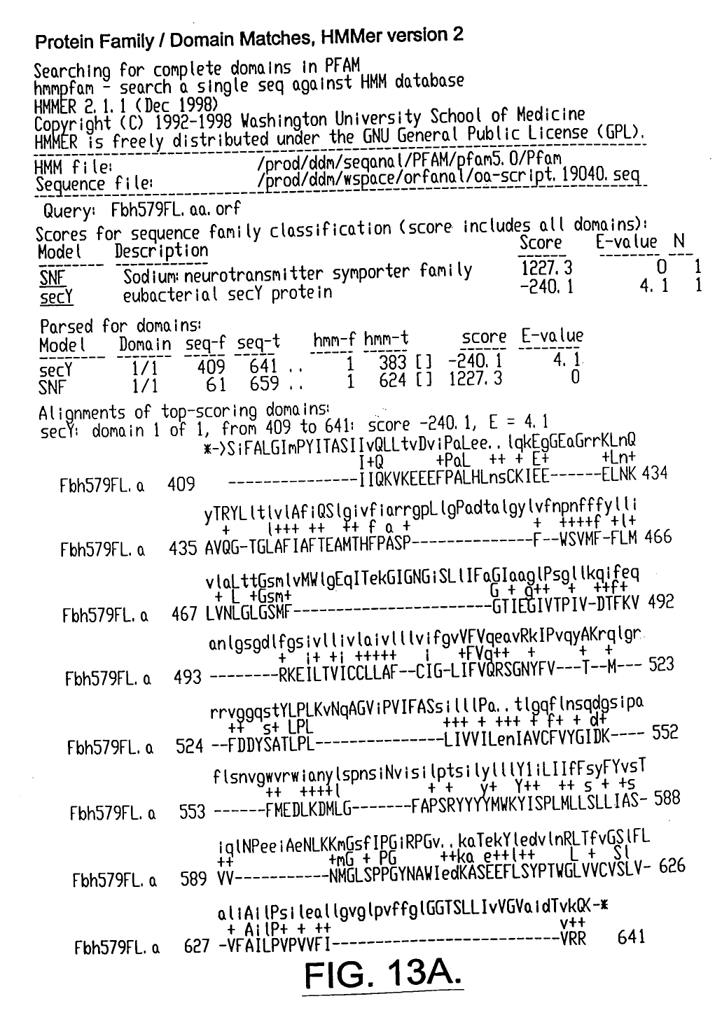

The 579 cDNA was identified based on consensus motifs or protein domains characteristic of transporters, and in particular, neurotransmitters. Pfam analysis indicates that this polypeptide shares a high degree of sequence similarity with the sodium: neurotransmitter-symporter family (FIGS. 13A & B). The sodium: neurotransmitter-symporter domain (HMM) (PS00610 and PS00754) aligns with amino acids 61 to 659 of SEQ ID NO:5.

In one embodiment a 579-like polypeptide or protein has a “sodium: neurotransmitter-symporter domain” or a region which has at least about 60%, 70%, 80%, 90%, 95%, 99%, or 100% sequence identity with a “sodium: neurotransmitter-symporter domain,” e.g., the sodium: neurotransmitter-symporter domain of human 579 (e.g., amino acid residues 61 to 659 of SEQ ID NO:5).

PropDom matches for the 579 transporter show similarity to sodium and chloride dependent neurotransmitter transporters. BLASTX analysis of 579 transporter reveals that the amino acid sequence of 579 polypeptide (SEQ ID NO:5) from about amino acid 1 to 730 is about 90% identical and 96% similar to that of rat sodium and chloride dependent transporter (Genbank Accession No: Q08469).

MEMSAT analysis of 579 transporter protein predicts 12 transmembrane seqments or domains (FIG. 11A). In one embodiment, a 579-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of human 579 (e.g., amino acid residues 70-87, 98-117, 140-164, 228-244, 253-275, 306-323, 334-358, 458-479, 496-513, 527-550, 575-594, 617-639 of SEQ ID NO:5).

In another embodiment, a 579-like protein includes at least one “non-transmembrane domain.” The C-terminal amino acid residue of a non-transmembrane domain is adjacent to an N-terminal amino acid residue of a transmembrane domain in a naturally occurring 579 protein, or 579-like protein.

With STS being Cda1db04, the gene has been mapped to chromosome 12 between D12S319 and D12S322 (95.8-97cM). With STS being SGC30472, the gene has been mapped to chromosome 12 between D12S88 and D12S82 (95.8-96cM).

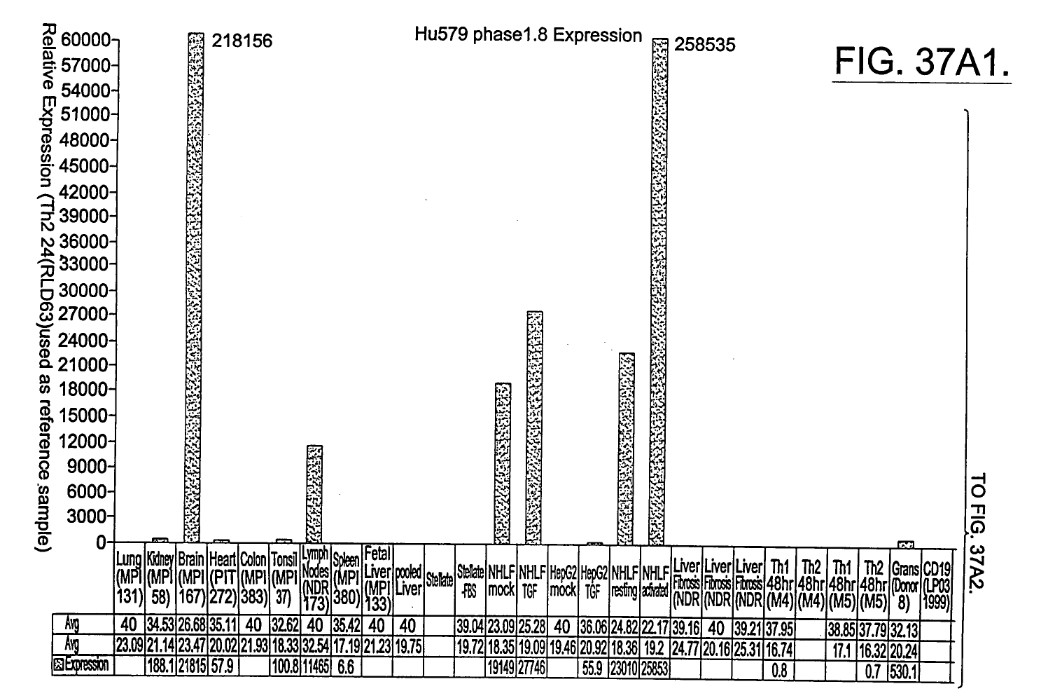

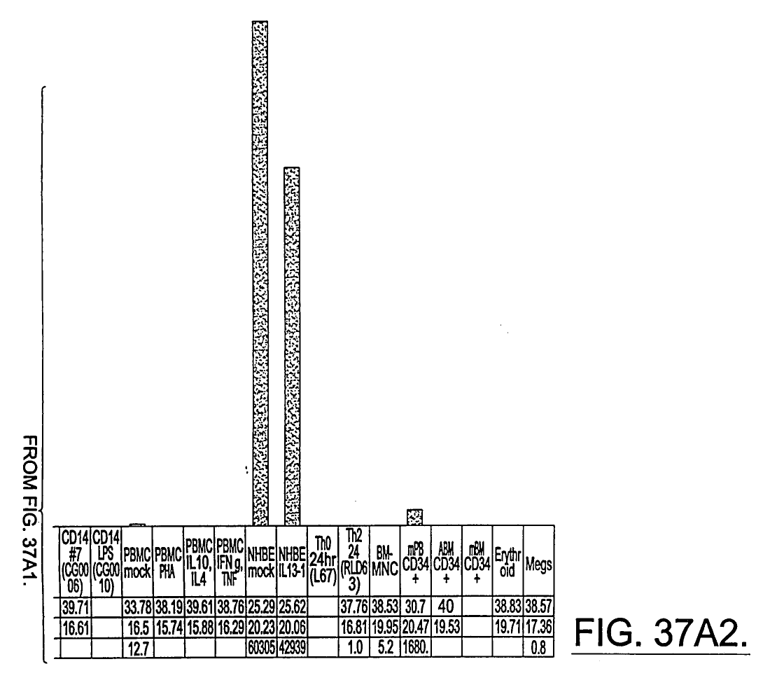

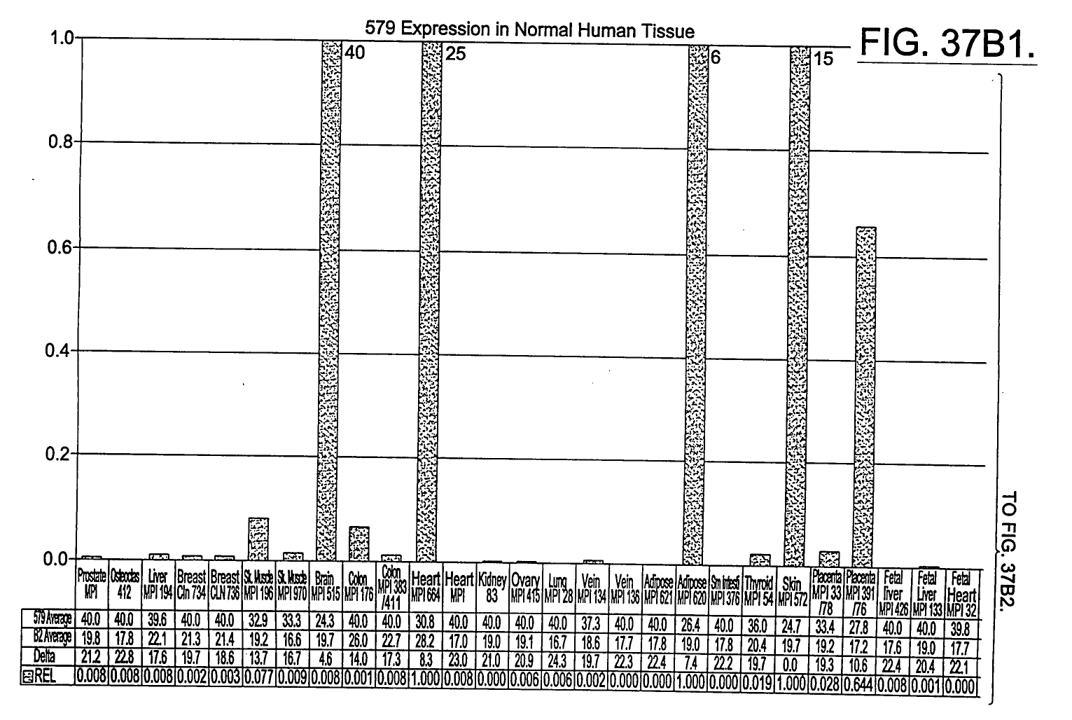

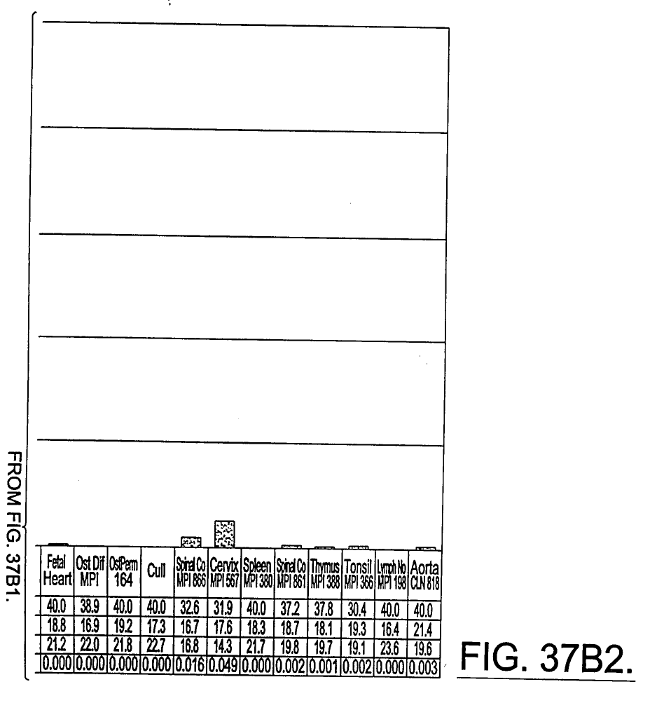

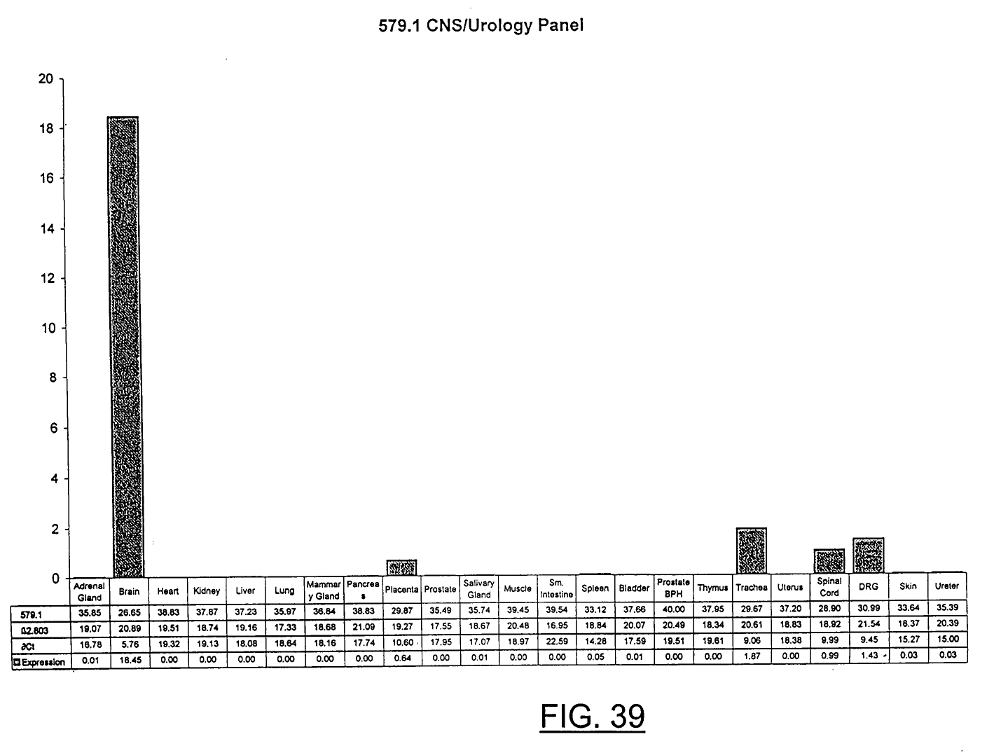

The 579 gene is expressed in various human tissues and cells including, but not limited to, those shown in FIGS. 37A & B and 39. The highest expression shown in FIGS. 37A & B is observed in brain, lung, heart, adipose, placenta, and skin. The human pain panel phase I experiment data of FIG. 39 show that the expression of the 579 mRNA is almost exclusively directed to nervous tissue, with FIG. 39 additionally demonstrating that the highest levels of the mRNA occur in the brain, followed by spinal cord, DRG, and placenta.

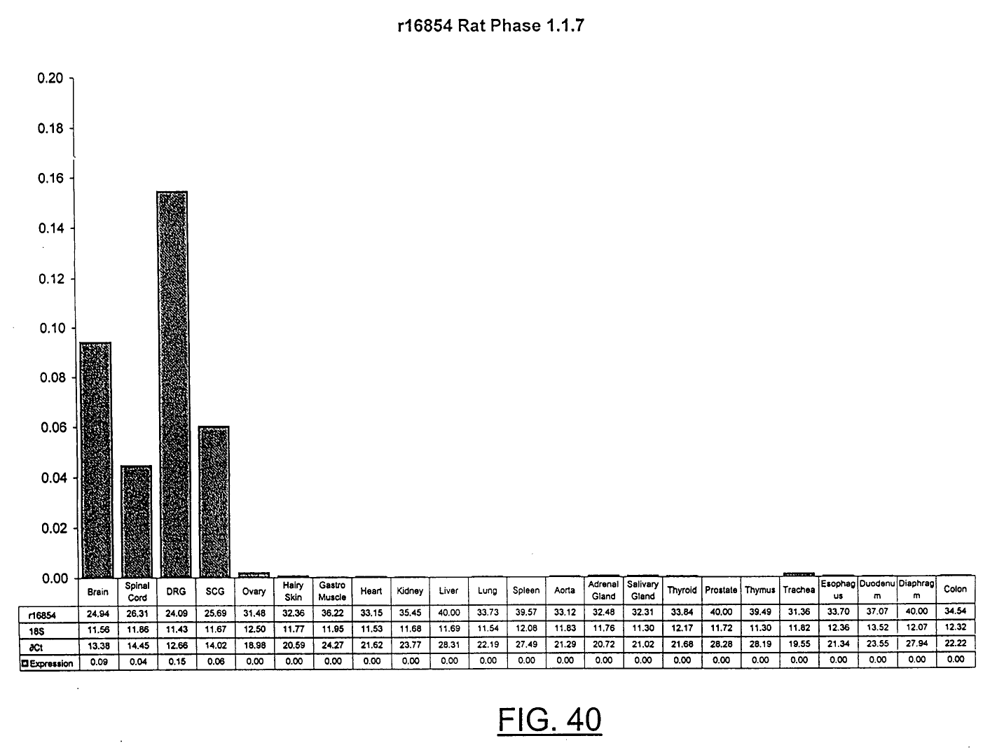

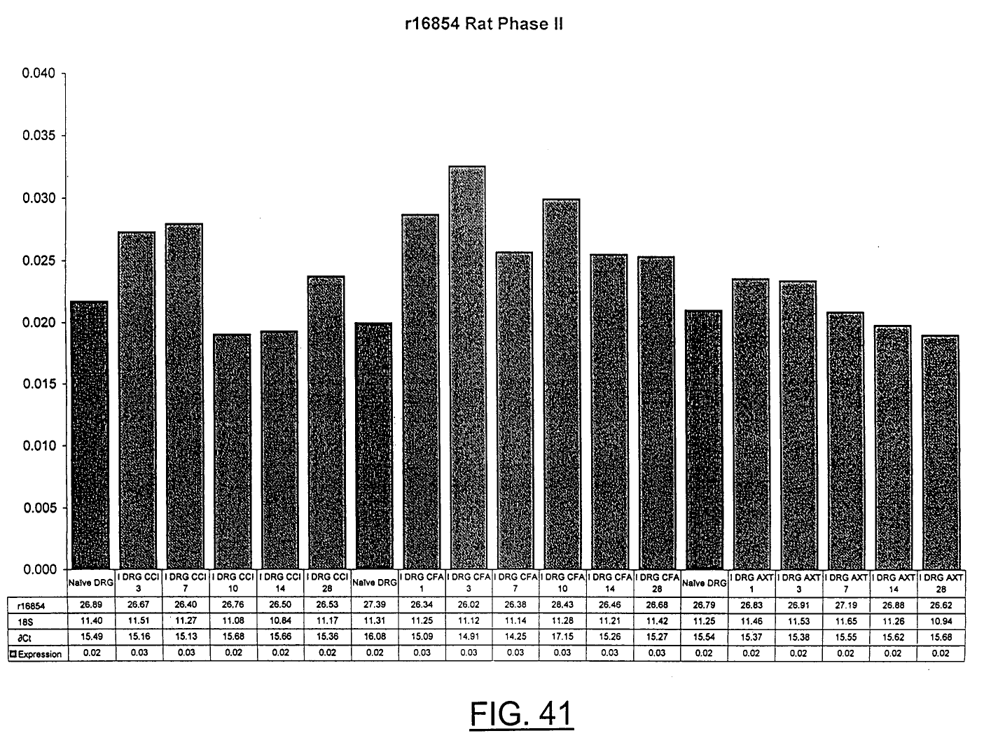

Further data on the role of the 579 gene is presented for the r16854 rat ortholog of 579 in FIGS. 40 and 41. Taqman® data for a rat pain panel phase I experiment (FIG. 40) show that the rat ortholog exhibits the same pattern of expression as the human 579 gene and further characterizes the gene as being additionally expressed in the SCG (superior cervical ganglion). ISH hybridization (data not shown) confirmed the exclusive expression of the 579 gene and rat ortholog in the brain, DRG and spinal cord using both human and rat probes. Taqman® data for a rat pain panel phase II experiment (FIG. 41) show up-regulation of r16854 in the DRG at 1, 3, 7, 10, 14, and 28 days after exposure to CFA. Taqman® data for a rat pain panel phase III experiment (data not shown) show no regulation of r16854 in spinal cords of any of the models studied, except for some down-regulation one month after CFA and axotomy.

The 579 transporter is useful for the diagnosis and treatment of sodium and chloride dependent neurotransmitter transporter-related disorders. The 579 transporter is useful for the diagnosis and treatment of neurological and central nervous system disorders, including pain, stroke, and depression; disorders of the lung, including cancer; immune and inflammatory disorders; and disorders of the vascular system.

Additionally, the 579 gene may play an important role in the treatment of pain disorders, for example in pain disorders associated with inflammatory pain. For example, the 579 gene may be important for regulating the physiology of neurons involved in nociceptive pathways. See, for example, the observation presented in FIG. 41 and discussed above that the rat ortholog of 579, r16854, is upregulated in neurons of the DRG (Dorsal Root Ganglion) in the CFA (Complete Freund's Adjuvant) model. Examples of pain disorders include, but are not limited to, pain response elicited during various forms of tissue injury, e.g., inflammation, infection, and ischemia, usually referred to as hyperalgesia (described in, for example, Fields, H. L. (1987) Pain, New York: McGraw-Hill); pain associated with muscoloskeletal disorders, e.g., joint pain; tooth pain; headaches; pain associated with surgery; pain related to irritable bowel syndrome; or chest pain. Additional pain disorders or conditions include, for example, vascular pain, including angina, ischemic muscle pain, migraine, lumbar pain, pelvic pain, and sympathetic nerve activity including inflammation associated with arthritis.

As used herein, a “pain modulatory ability” refers to the ability of the molecule of the invention to alter pain levels by, for example, increasing or decreasing neurotransmitter levels.

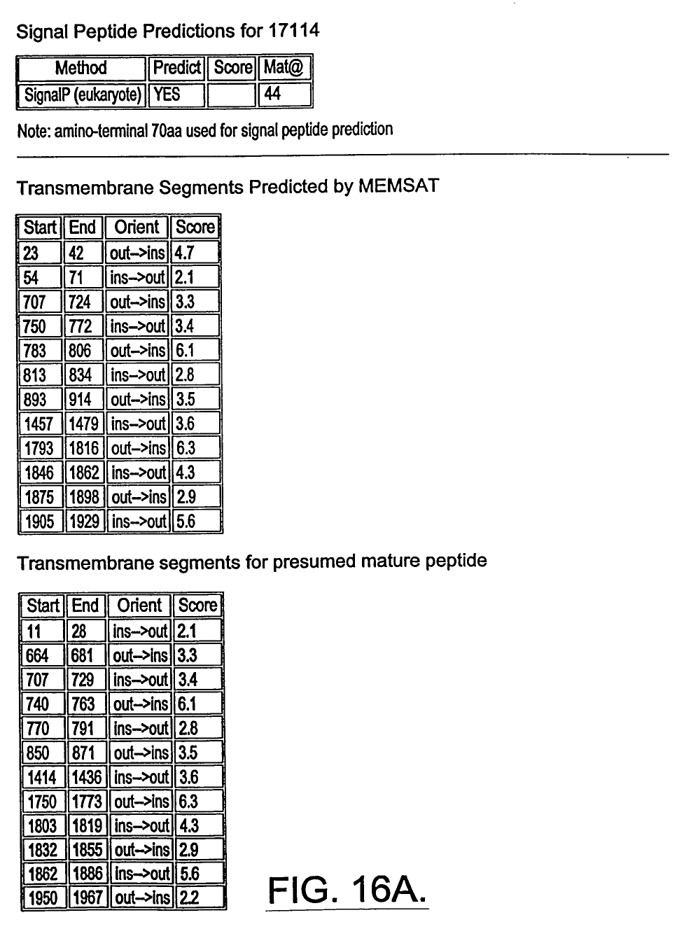

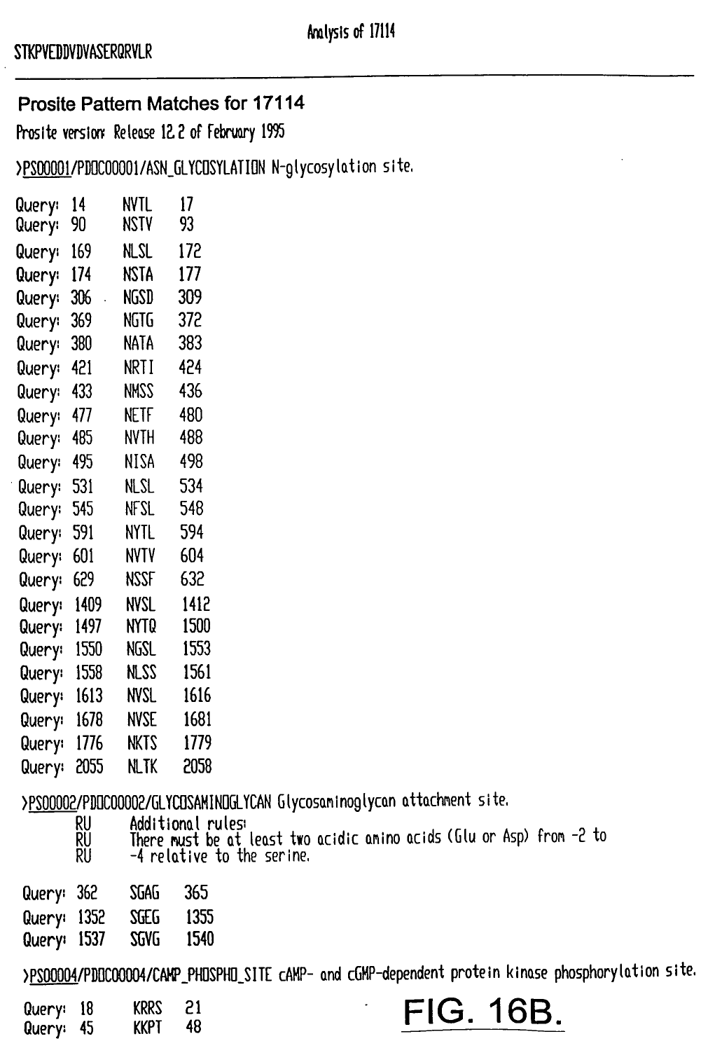

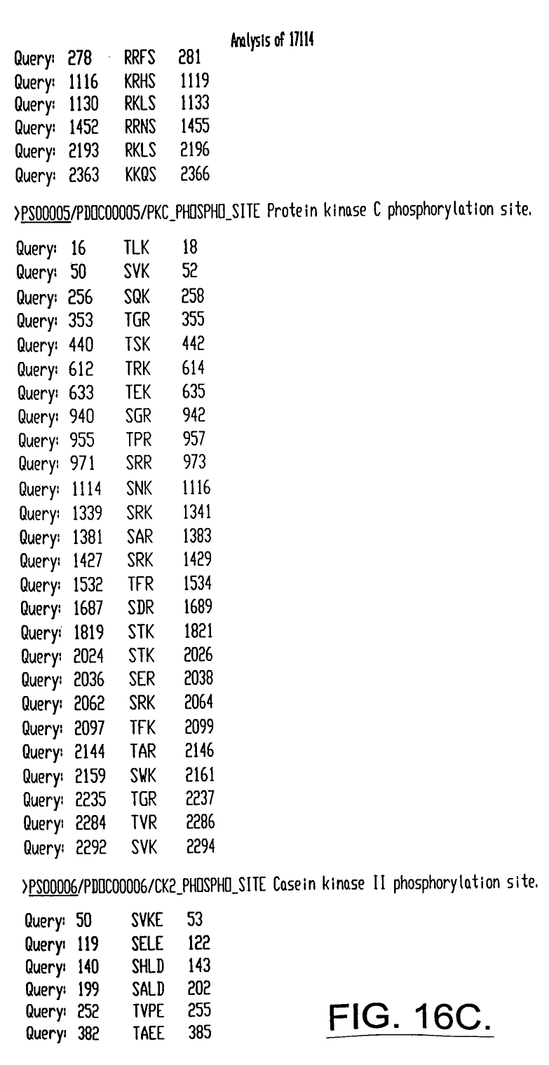

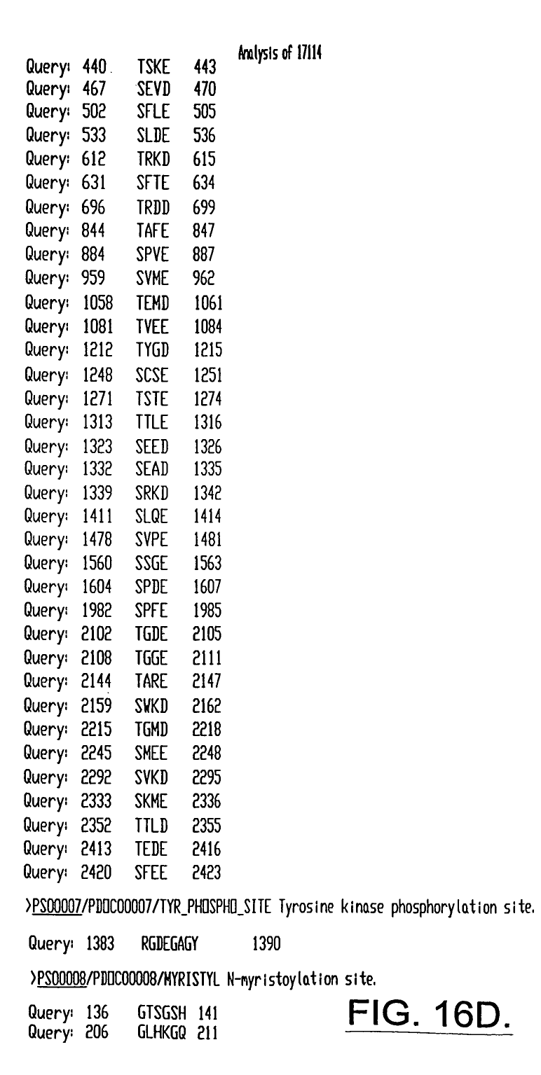

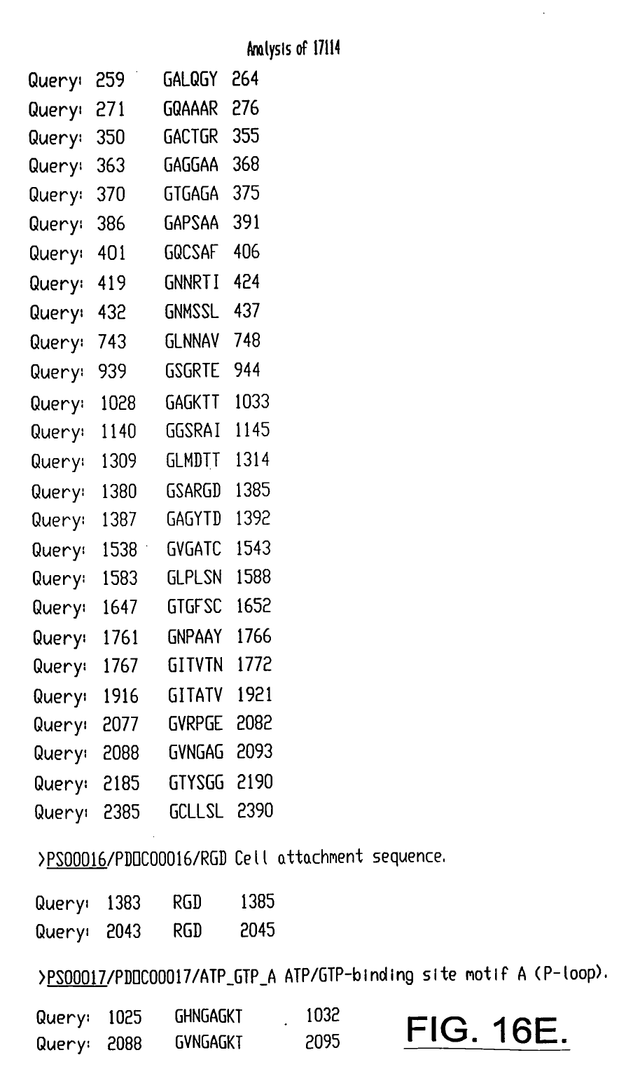

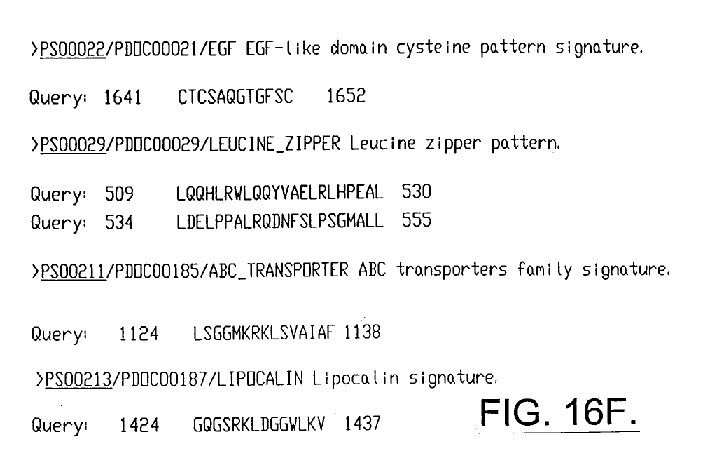

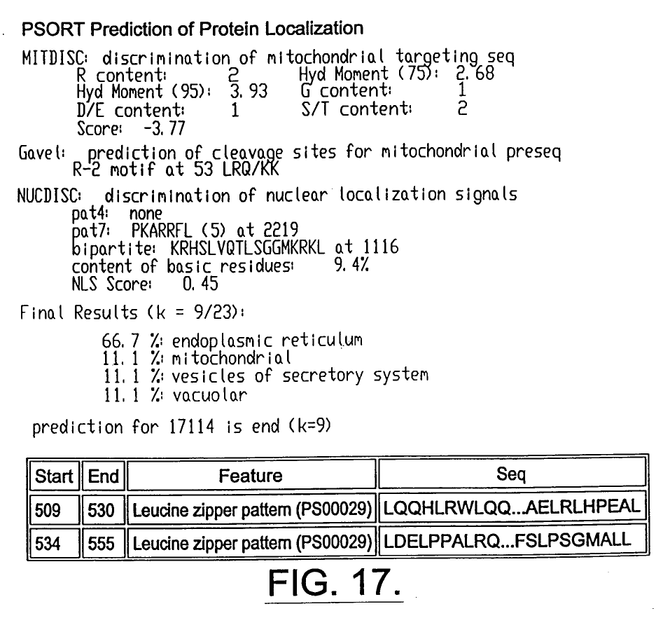

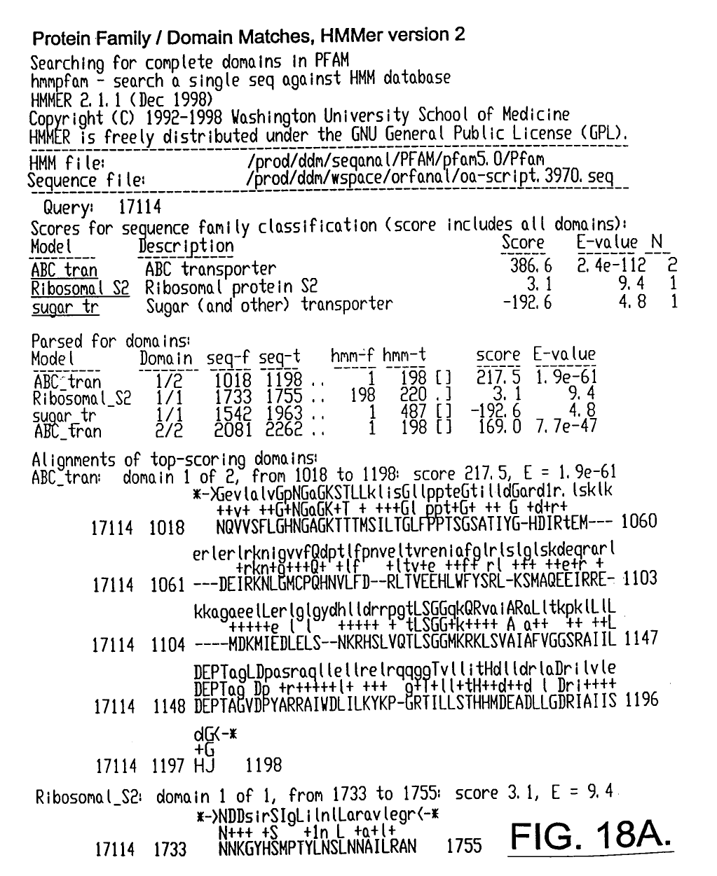

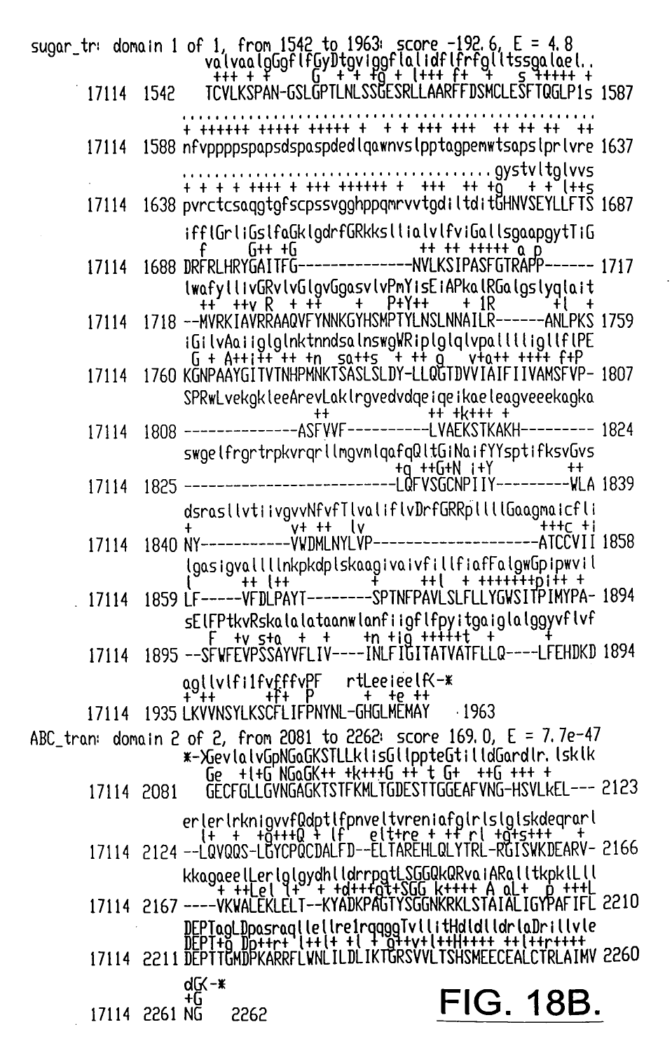

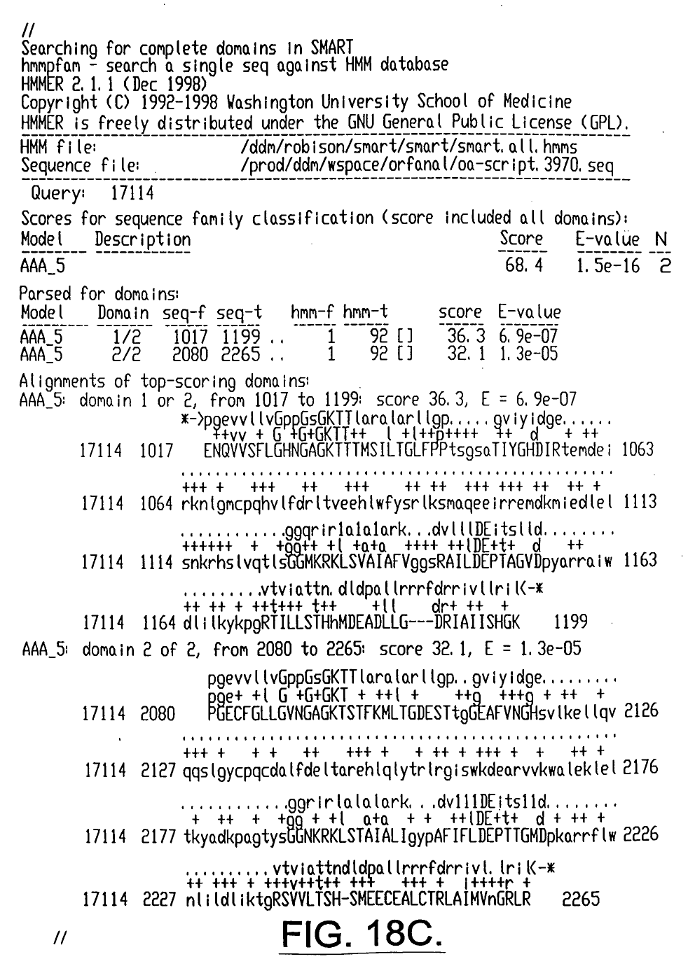

The invention is also based on the identification of the novel human transporter 17114. The cDNA was identified based on consensus motifs or protein domains characteristic of transporters, particularly ABC transporters (ATP-binding transporter cassette). The 17114 transporter gene encodes an approximately 8195 nucleotide mRNA transcript with an open reading frame that encodes a 2436 amino acid protein. Prosite program analysis was used to predict various sites within the 17114 transporter protein as shown in FIG. 16.

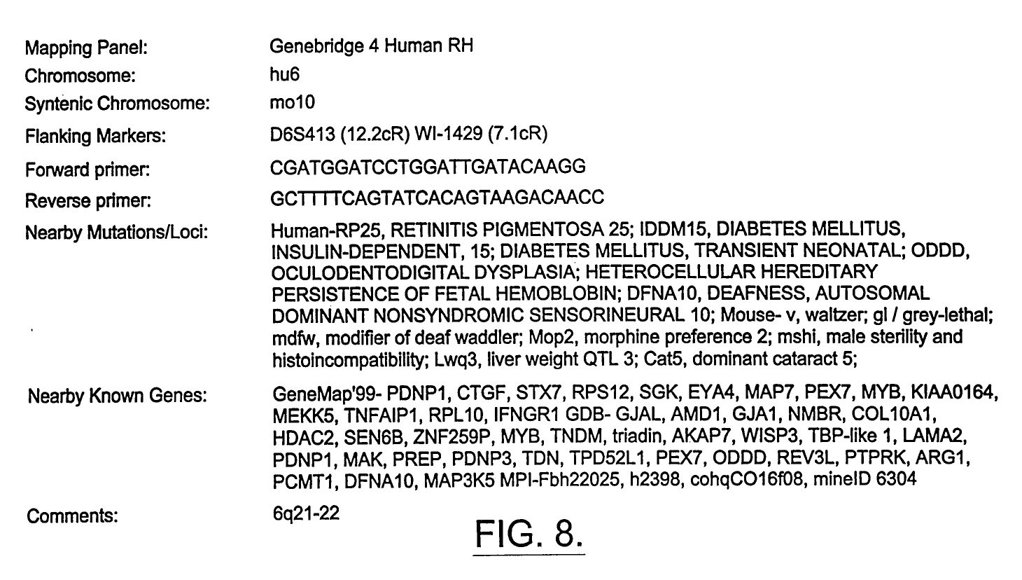

Pfam analysis indicates that this polypeptide shares sequence similarity with the ABC transporter family (FIGS. 8A & B). The ABC transporter domain 1 (HMM) (PS00211) aligns with amino acids 1018 to 1198 of SEQ ID NO:8 and domain 2 aligns with amino acids 2081 to 2262.

In one embodiment a 17114-like polypeptide or protein has an “ABC transporter domain” or a region which has at least about 60%, 70%, 80%, 90%, 95%, 99%, or 100% sequence identity with an “ABC transporter domain,” e.g., the ABC transporter domains of human 17114 (e.g., amino acid residues 1018 to 1198 and 2081 to 2262 of SEQ ID NO:8).

ProDom matches for the 17114 transporter show similarity to ABC transporters.

MEMSAT analysis of 17114 transporter protein predicts 12 transmembrane seqments or domains (FIG. 16A). In one embodiment, a 17114-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of human 17114 (e.g., amino acid residues 23-42, 54-71, 707-724, 750-772, 783-806, 813-834, 893-914, 1457-1479, 1793-1816, 1846-1862, 1875-1898, 1905-1929 of SEQ ID NO:8).

In another embodiment, a 17114-like protein includes at least one “non-transmembrane domain.” The C-terminal amino acid residue of a non-transmembrane domain is adjacent to an N-terminal amino acid residue of a transmembrane domain in a naturally occurring 17114 protein, or 17114-like protein.

A 17114-like molecule can further include a signal sequence. As used herein, a “signal sequence” refers to a peptide of about 20-80 amino acid residues in length which occurs at the N-terminus of secretory and integral membrane proteins and which contains a majority of hydrophobic amino acid residues. For example, a signal sequence contains at least 24 amino acid residues, and has at least about 40-70%, preferably about 50-65%, and more preferably about 55-60% hydrophobic amino acid residues (e.g., alanine, valine, leucine, isoleucine, phenylalanine, tyrosine, tryptophan, or proline). Such a “signal sequence”, also referred to in the art as a “signal peptide”, serves to direct a protein containing such a sequence to a lipid bilayer. For example, in one embodiment, a 17114-like protein contains a signal sequence of about amino acids 1-44 of SEQ ID NO:8 (FIG. 16A). The “signal sequence” is cleaved during processing of the mature protein. The mature 17114 protein corresponds to amino acids 45-2436 of SEQ ID NO:8.

MEMSAT analysis of mature 17114 transporter protein predicts 12 transmembrane seqments or domains (FIG. 16A). In one embodiment, a 17114-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of mature human 17114 (e.g., amino acid residues 11-28, 664-681, 707-729, 740-763, 770-791, 850-871, 1414-1436, 1750-1773, 1803-1819, 1832-1855, 1062-1886, 1950-1967 of amino acids 45-2436 of SEQ ID NO:8 wherein residue number 45 of SEQ ID NO:8 is designated residue number 1).

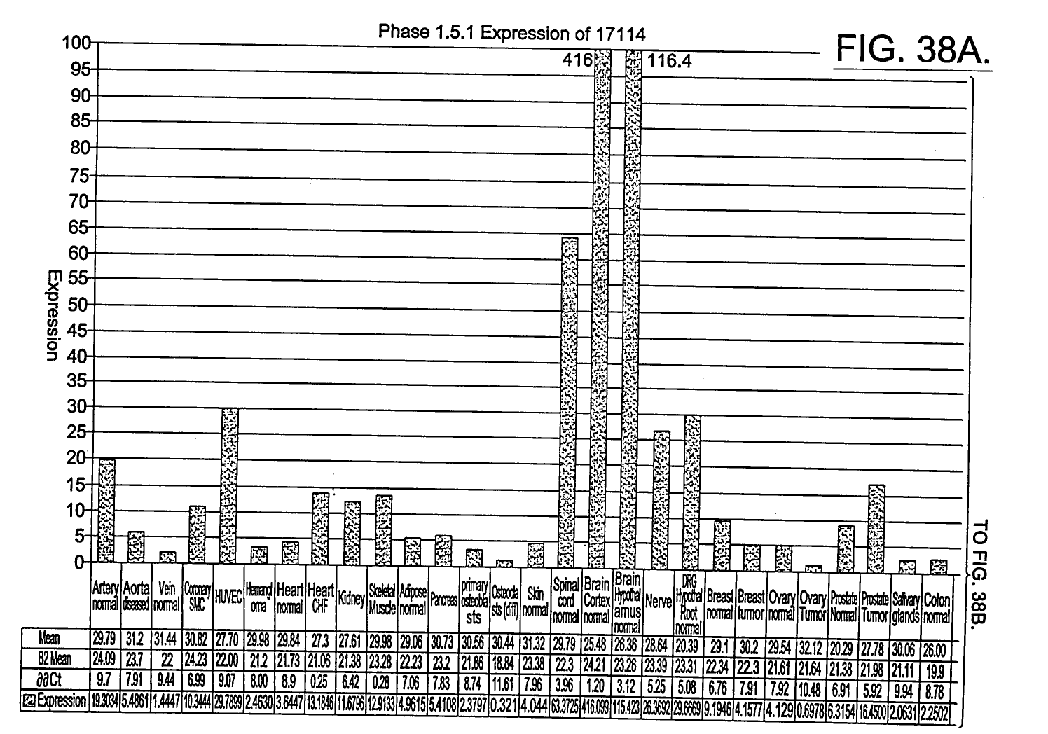

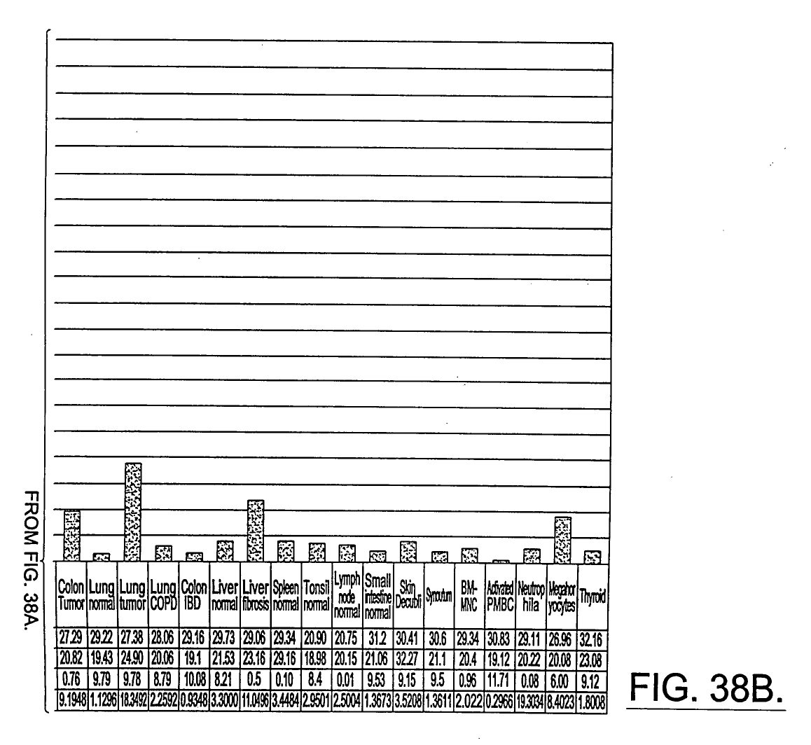

The 17114 gene is expressed in various human tissues and cells including, but not limited to, those shown in FIG. 38. The highest expression is observed in brain, spinal cord, nerve, artery, and umbilical vein endothelial cells. 17114 is more highly expressed in prostrate, lung, and colon tumors than in the respective normal tissues. In addition, 17114 is more highly expressed in liver fibrosis than in normal liver tissue.

The 17114 transporter is useful for the diagnosis and treatment of ABC transporter-related disorders. The 17114 transporter is useful for the diagnosis and treatment of neurological and central nervous system disorders; immune and inflammatory disorders including multiple sclerosis; disorders of the lung, prostrate, and colon, particularly cancer; disorders of the liver, particularly liver fibrosis; and disorders of the vascular system, particularly atherosclerosis.

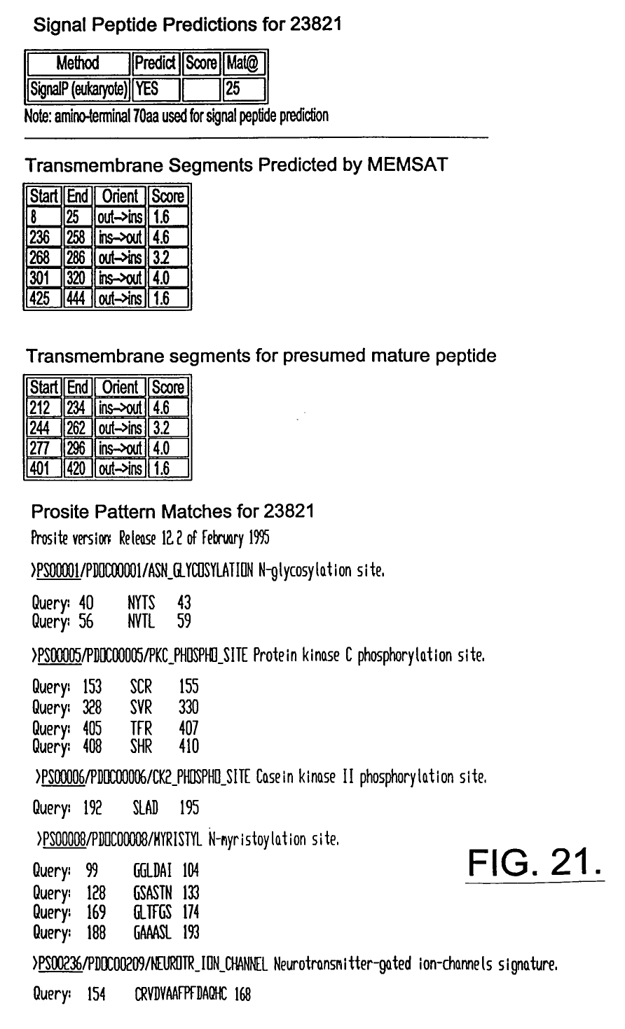

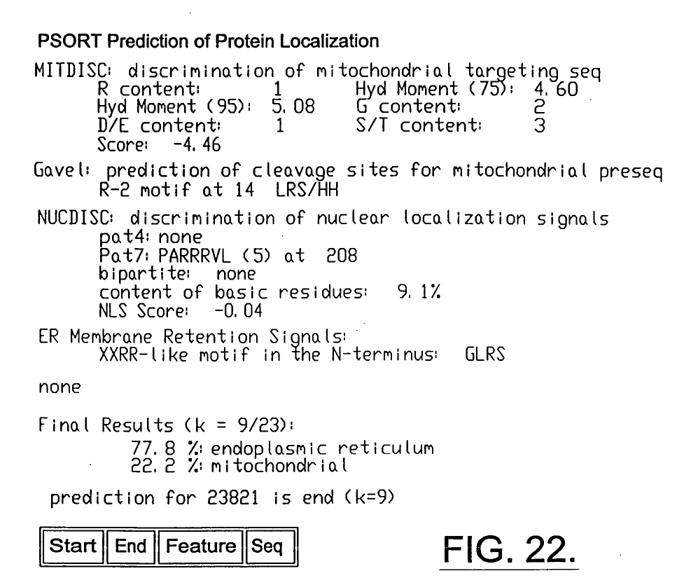

The invention is also based on the identification of the novel human transporter 23821. The cDNA was identified based on consensus motifs or protein domains characteristic of transporters, particularly neurotransmitter-gated ion channels. The 23821 transporter gene encodes an approximately 2150 nucleotide mRNA transcript with an open reading frame that encodes a 450 amino acid protein. Prosite program analysis was used to predict various sites within the 23812 transporter protein as shown in FIG. 21.

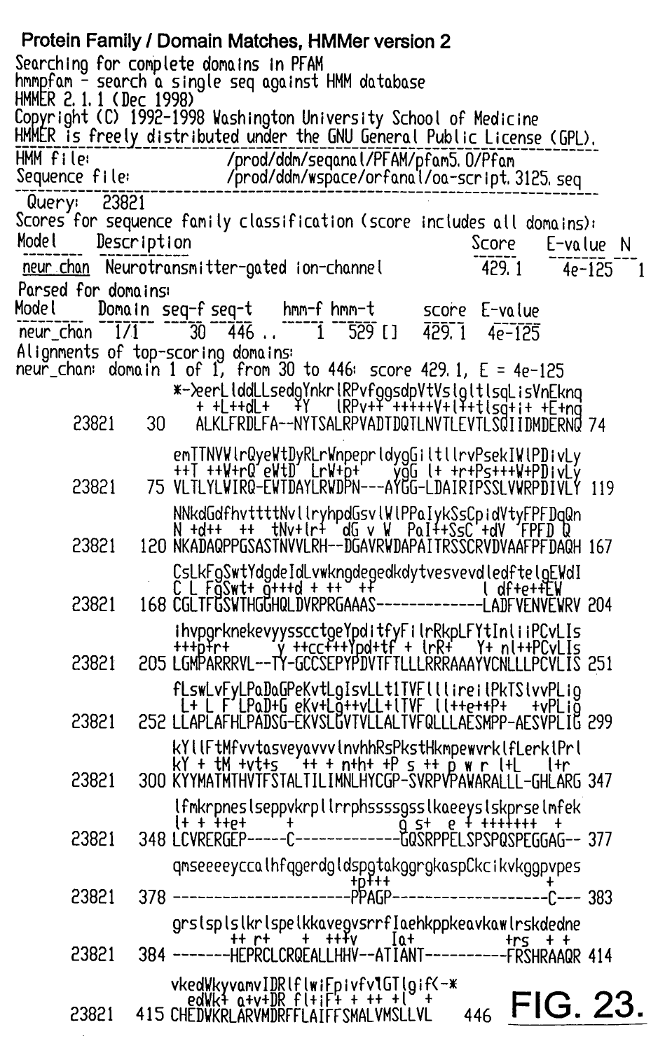

Pfam analysis indicates that this polypeptide shares sequence similarity with the neurotransmitter-gated ion channel family (FIG. 23). The neurotransmitter-gated ion channel domain (HMM) (PS00236) (PSaligns with amino acids 30 to 446 of SEQ ID NO:11.

In one embodiment a 23821-like polypeptide or protein has a “neurotransmitter-gated ion channel domain” or a region which has at least about 60%, 70%, 80%, 90%, 95%, 99%, or 100% sequence identity with a “neurotransmitter-gated ion channel domain,” e.g., the neurotransmitter-gated ion channel domain of human 23821 (e.g., amino acid residues 30 to 446 of SEQ ID NO:11).

ProDom matches for the 23821 transporter show similarity to the acetylcholine receptor subunit subclass of neurotransmitter-gated ion channel transporters. BLASTX analysis of 23821 transporter reveals that the amino acid sequence of the 23821 polypeptide (SEQ ID NO:11) is 90% identical and 92% similar to that of rat neuronal nicotinic acetylcholine receptor subunit (alpha10) (Genbank Accession No:AF196344).

MEMSAT analysis of 23821 transporter protein predicts 5 transmembrane seqments or domains (FIG. 21). In one embodiment, a 23821-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of human 23821 (e.g., amino acid residues 8-25, 236-258, 268-286, 301-320, 425-444 of SEQ ID NO:11).

In another embodiment, a 23821-like protein includes at least one “non-transmembrane domain.” The C-terminal amino acid residue of a non-transmembrane domain is adjacent to an N-terminal amino acid residue of a transmembrane domain in a naturally occurring 23821 protein, or 23821-like protein.

A 23821-like molecule can further include a signal sequence. For example, in one embodiment, a 23821-like protein contains a signal sequence of about amino acids 1-25 of SEQ ID NO:11 (FIG. 21). The “signal sequence” is cleaved during processing of the mature protein. The mature 23821 protein corresponds to amino acids 26-450 of SEQ ID NO:11.

MEMSAT analysis of mature 23821 transporter protein predicts 4 transmembrane seqments or domains (FIG. 21). In one embodiment, a 23821-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of mature human 23821 (e.g., amino acid residues 212-234, 244-262, 277-296, 401-420 of amino acids 26-450 of SEQ ID NO:11 wherein residue number 26 of SEQ ID NO:11 is designated residue number 1).

The 23821 transporter is useful for the diagnosis and treatment of neuronal nicotinic acetylcholine receptor subunit-related disorders. The 23821 transporter is useful for the diagnosis and treatment of neurological and central nervous system disorders including, but not limited to, Alzheimer's Disease, Parkinson's Disease, epilepsy, schizophrenia, Lewy body diseases, and stroke; inflammatory and autoimmune disorders; and vascular disorders.

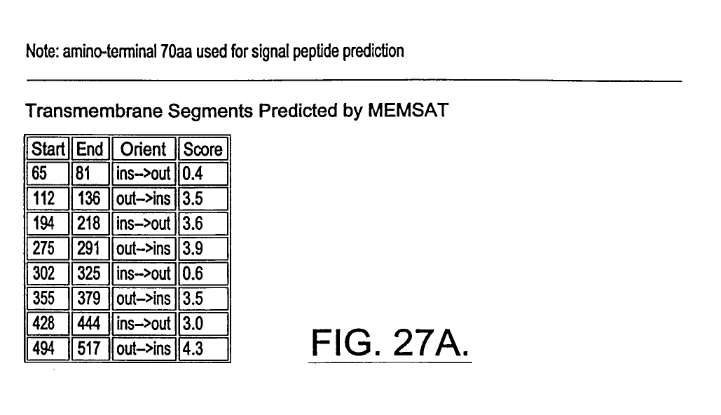

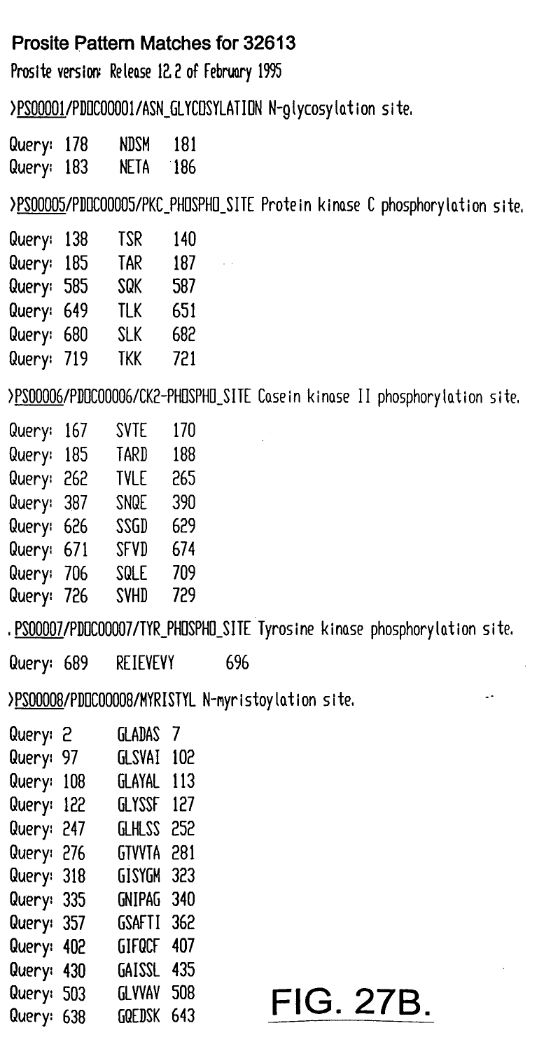

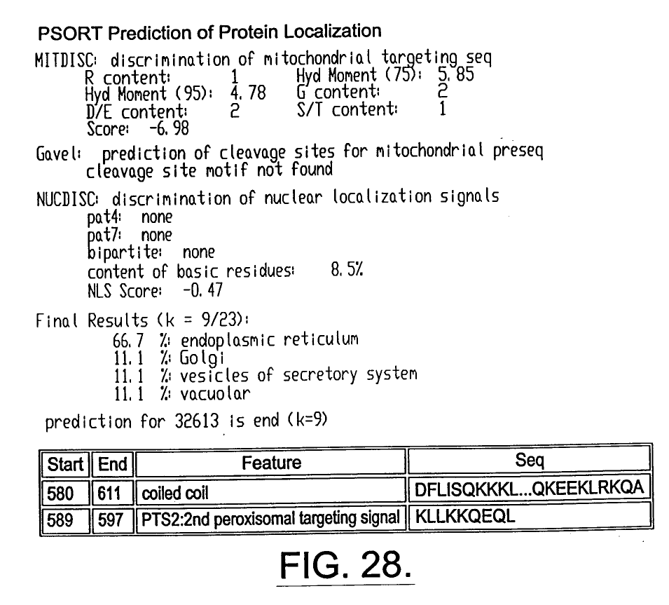

The invention is also based on the identification of the novel human transporter designated 32613. The 32613 transporter gene encodes an approximately 2593 nucleotide mRNA transcript with an open reading frame that encodes a 751 amino acid protein. The cDNA was identified based on consensus motifs or protein domains characteristic of transporters particularly sulfate transporters. Prosite program analysis was used to predict various sites within the 32613 transporter protein as shown in FIG. 27.

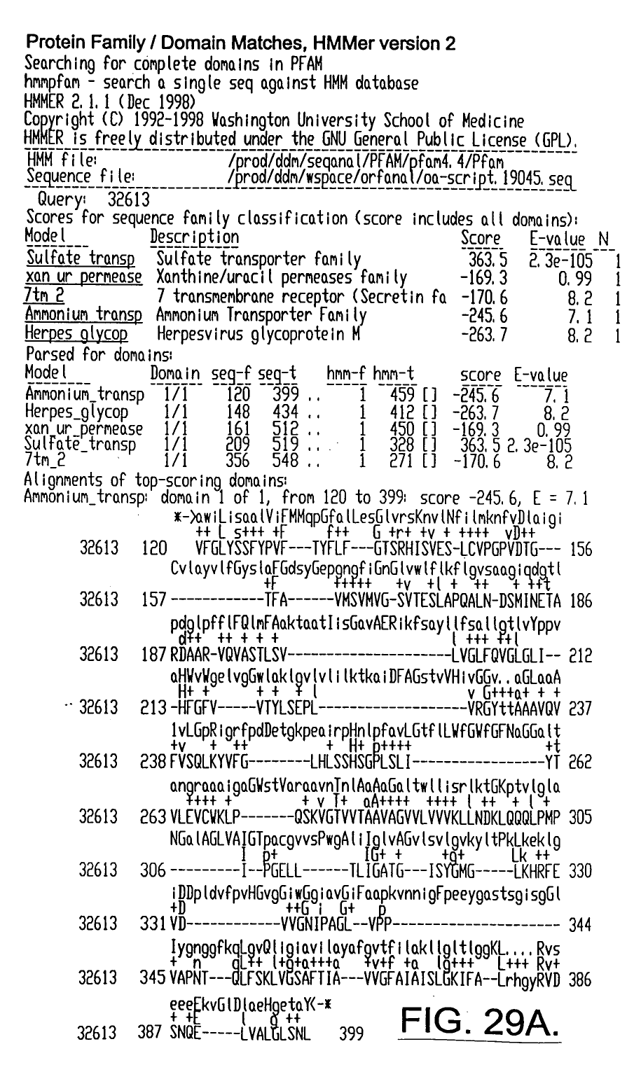

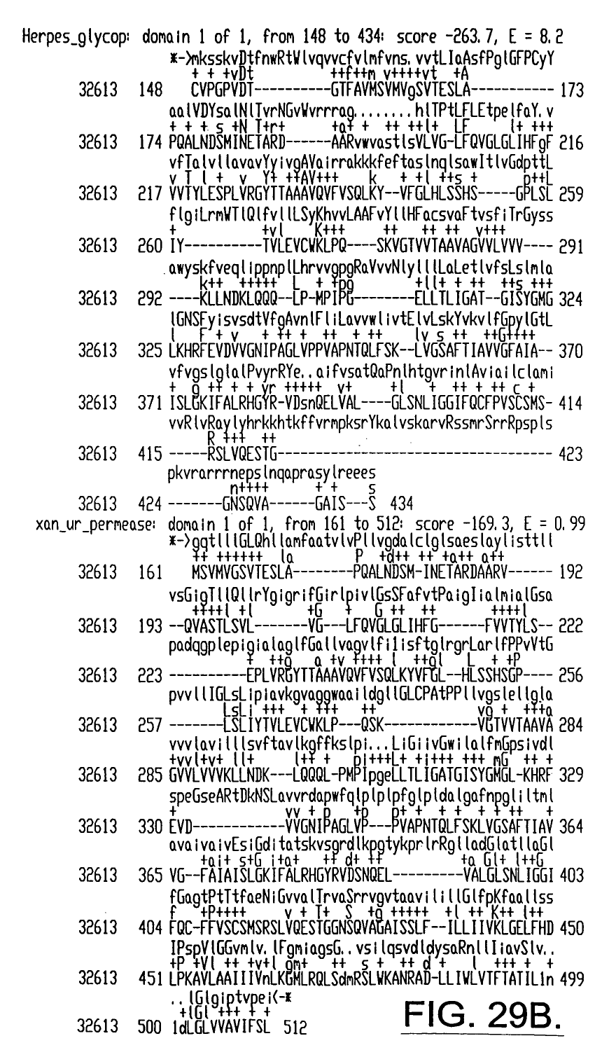

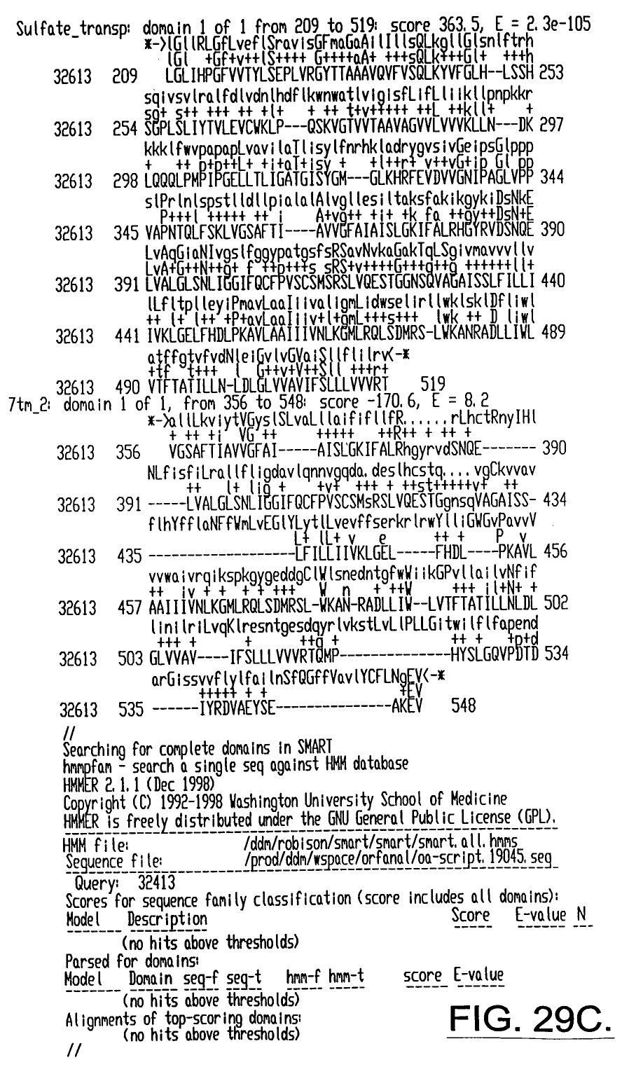

Pfam analysis indicates that this polypeptide shares sequence similarity with the sulfate transporter family (FIGS. 29A, B & C). The sulfate transporter domain (HMM) (PS001130) aligns with amino acids 209 to 519 of SEQ ID NO:14. The sulfate transporter family of proteins as defined by Pfam include proteins that transport anions other than sulfate. These anions include chloride, iodide, and formate (Scott and Karniski (2000) Am. J. Cell Physiol. 278:C207-211; Scott et al. (1999) Nat. Genet. 21:440-443; Royaux et al. (2000) Endocrinology 141:839-845). “Sulfate transporter” as defined by Pfam is herein defined as a polypeptide capable of transporting an anion across a membrane or an “anion transporter”.

In one embodiment a 32613-like polypeptide or protein has a “sulfate transporter domain” or a region which has at least about 60%, 70%, 80%, 90%, 95%, 99%, or 100% sequence identity with a “sulfate transporter domain,” e.g., the sulfate transporter domain of human 32613 (e.g., amino acid residues 209 to 519 of SEQ ID NO:14).

ProDom matches for the 32613 transporter show similarity to sulfate transporters. In addition, BLAST analysis reveals amino acids from about 176 to about 579 of the 32613 transporter (SEQ ID NO:16) shares approximately 42% sequence identity to amino acids 171 to 591 of the Pedrin polypeptide from Homo sapiens (Genbank Accession No. 043511). In addition, amino acids 62 to 145 of SEQ ID NO: 16 share approximately 55% identity to amino acids 56 to 138 of Genbank Accession No. 043511. Furthermore, amino acids 151 to 603 of SEQ ID NO:16 share approximately 40% identity to amino acids 128 to 579 from the mouse DRA polypeptide (Genbank Accession No. AF136751). Both of these proteins are members of the sulfate transporter family. The human DRA protein is down-regulated in adenoma. Human Pendrin protein, a chloride-iodide transporter protein, is involved in a number of hearing loss genetic diseases (Scott et al. (1999) Nat. Genet. 21:440-443; Royaux et al. (2000) Endocrinology 141:839-845). Another member of the sulfate transporter family, human DTDST, is involved in the genetic disease, diastrophic dysplasia.

MEMSAT analysis of 32613 transporter protein predicts 8 transmembrane seqments or domains (FIG. 27A). In one embodiment, a 32613-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of human 32613 (e.g., amino acid residues 65-81, 112-136, 194-218, 275-291, 302-325, 355-379, 428-444, 494-517 of SEQ ID NO:14).

In another embodiment, a 32613-like protein includes at least one “non-transmembrane domain.” The C-terminal amino acid residue of a non-transmembrane domain is adjacent to an N-terminal amino acid residue of a transmembrane domain in a naturally occurring 32613 protein, or 32613-like protein.

The 32613 gene is expressed in tissues and cells including, but not limited to: fibroblasts, keratinocytes, lung, lymphoma, muscle, osteoblast, pituitary, and T-cells.

The 32613 transporter is useful for the diagnosis and treatment of sulfate transporter family-related disorders of the tissues including, but not limited to, those listed above. The 32613 transporter is particularly useful for the diagnosis and treatment of diastrophic dysplasia, congenital chloride diarrhea, and Pendred syndrome; immune, inflammatory, and cell proliferative disorders including cancer, particularly those of bone, colon, thyroid, and glandular tissue; skeletal dysplasia; goitre; Graves' disease; disorders of electrolyte imbalance, particularly diarrhea; and deafness.

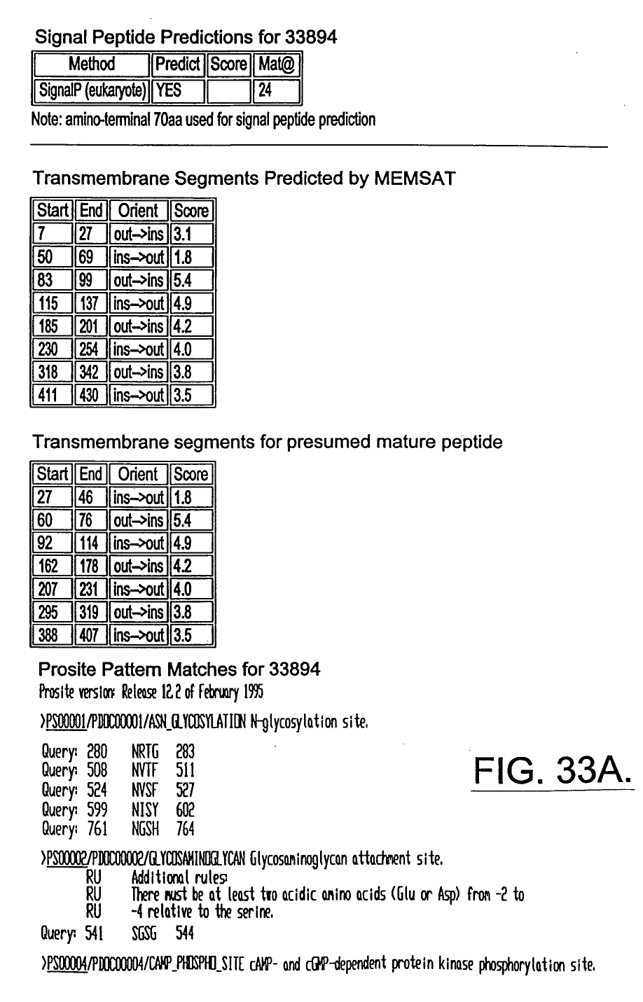

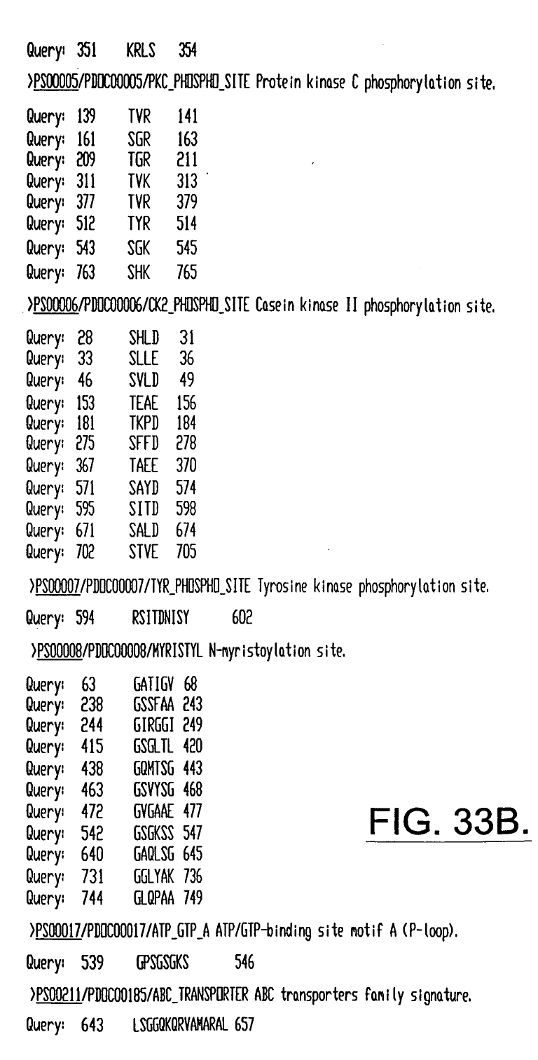

The invention is also based on the identification of the novel human transporter 33894. The 33894 transporter gene encodes an approximately 3408 nucleotide mRNA transcript with an open reading frame that encodes a 766 amino acid protein. The cDNA was identified based on consensus motifs or protein domains characteristic of transporters particularly, ABC transporters. Prosite program analysis was used to predict various sites within the 33894 transporter protein as shown in FIG. 33A-B.

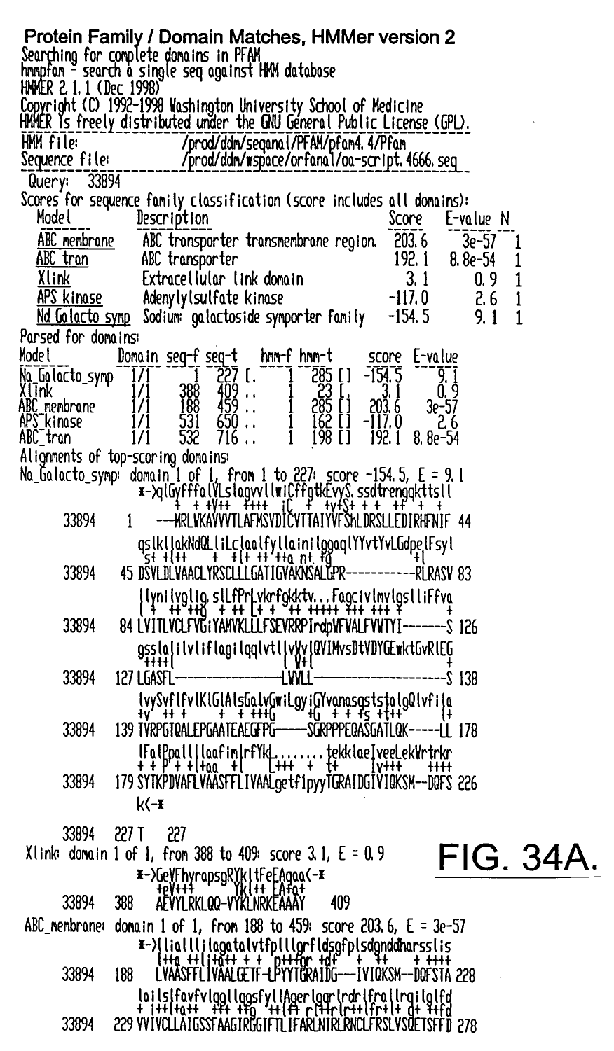

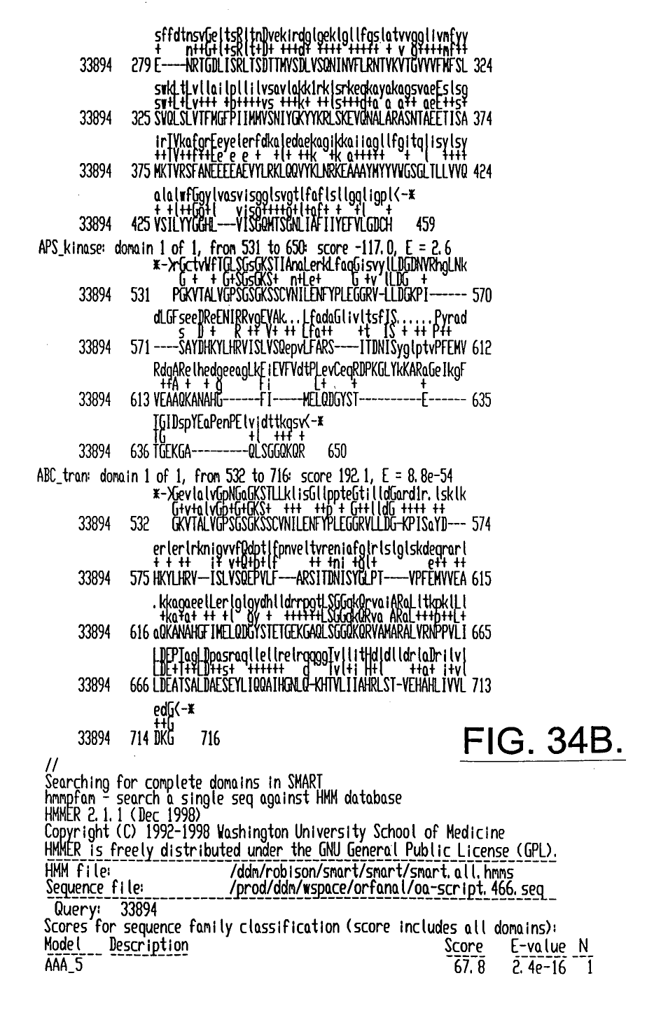

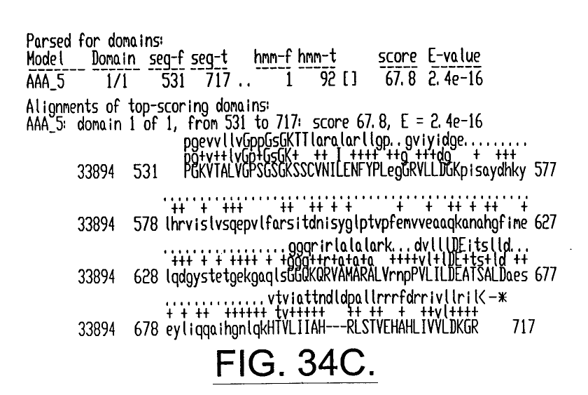

Pfam analysis indicates that this polypeptide shares a high degree of sequence similarity with the ABC transporter family (FIGS. 34A & B). The ABC transporter domain (HMM) (PS00211) aligns with amino acids 532 to 716 of SEQ ID NO:17. The ABC transporter transmembrane region domain (HMM) aligns with amino acids 188 to 459 of SEQ ID NO:17.

In one embodiment a 33894-like polypeptide or protein has an “ABC transporter domain” or a region which has at least about 60%, 70%, 80%, 90%, 95%, 99%, or 100% sequence identity with an “ABC transporter domain,” e.g., the ABC transporter domain of human 33894 (e.g., amino acid residues 532 to 716 of SEQ ID NO:17).

In one embodiment a 33894-like polypeptide or protein has an “ABC transporter transmembrane region domain” or a region which has at least about 60%, 70%, 80%, 90%, 95%, 99%, or 100% sequence identity with an “ABC transporter transmembrane region domain,” e.g., the ABC transporter transmembrane region domain of human 33894 (e.g., amino acid residues 188 to 459 of SEQ ID NO:17).

ProDom matches for the 33894 transporter show similarity to ABC transporters. BLASTX analysis of 33894 transporter reveals that amino acids 1 to 150 of 33894 polypeptide (SEQ ID NO:17) are about 92% identical to amino acids 1 to 150 of rat TAP-like ABC transporter polypeptide (Accession No: AB027520), and amino acids 158 to 766 of SEQ ID NO:17 are about 94% identical to amino acids 152 to 762 of rat TAP-like ABC transporter polypeptide (Accession No: AB027520).

MEMSAT analysis of 33894 transporter protein predicts 8 transmembrane seqments or domains (FIG. 33A). In one embodiment, a 33894-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of human 33894 (e.g., amino acid residues 7-27, 50-69, 83-99, 115-137, 185-201, 230-254, 318-342, 411-430 of SEQ ID NO:17).

In another embodiment, a 23821-like protein includes at least one “non-transmembrane domain.” The C-terminal amino acid residue of a non-transmembrane domain is adjacent to an N-terminal amino acid residue of a transmembrane domain in a naturally occurring 33894 protein, or 33894-like protein.

A 33894-like molecule can further include a signal sequence. For example, in one embodiment, a 33894-like protein contains a signal sequence of about amino acids 1-24 of SEQ ID NO:17 (FIG. 33A). The “signal sequence” is cleaved during processing of the mature protein. The mature 33894 protein corresponds to amino acids 25-766 of SEQ ID NO:17.

MEMSAT analysis of mature 33894 transporter protein predicts 7 transmembrane seqments or domains (FIG. 33A). In one embodiment, a 33894-like polypeptide or protein has at least one transmembrane domain or a region which includes at least 16-27, amino acid residues and has at least about 60%, 70% 80% 90% 95%, 99%, or 100% sequence identity with a “transmembrane domain,” e.g., at least one transmembrane domain of mature human 33894 (e.g., amino acid residues 27-46, 60-76, 92-114, 162-178, 207-231, 295-319, 388-407 of amino acids 25-766 of SEQ ID NO:17 wherein residue number 25 of SEQ ID NO:17 is designated residue number 1).

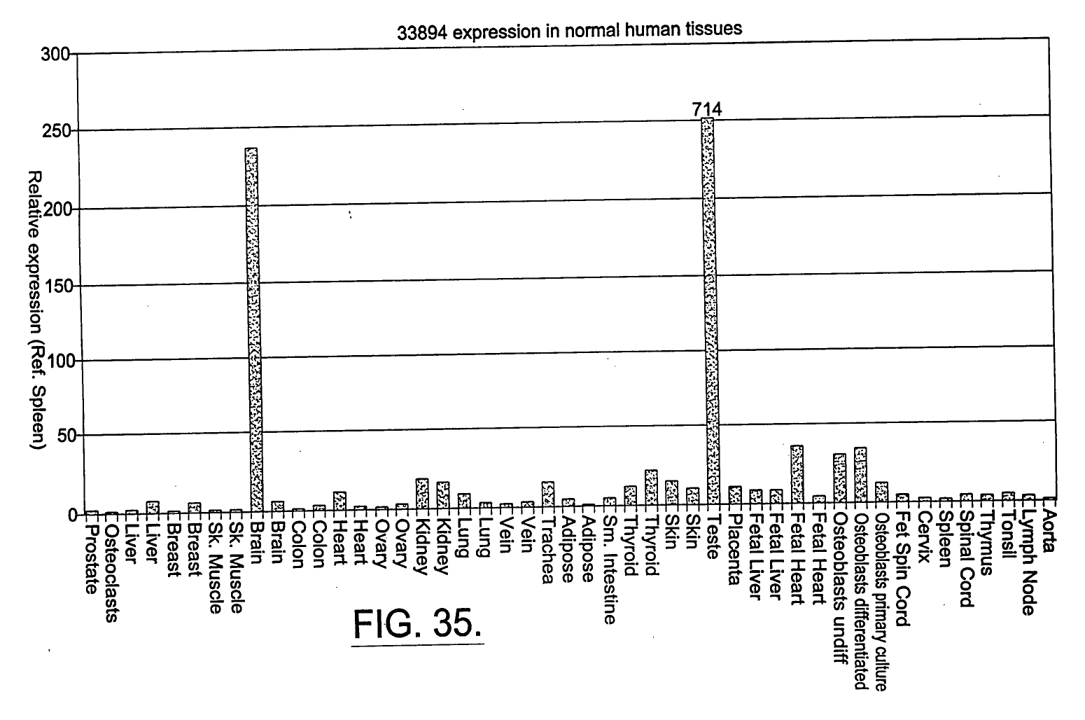

The 33894 transporter gene is expressed in various human tissues and cells including, but not limited to, those shown in FIG. 35. Highest expression is in brain and testes.

The 33894 transporter is useful for the diagnosis and treatment of ABC transporter-related disorders of the tissues including, but not limited to, those listed above. The 33894 transporter is particularly useful for the diagnosis and treatment of neurological and central nervous system disorders, particularly Alzheimer's disease; immune and inflammatory disorders including multiple sclerosis, Graves' disease, allergy, and arthritis; cell proliferative disorders including cancer; and disorders of the vascular system, particularly atherosclerosis.

These gene sequences, and other nucleotide sequences encoding the transporter proteins or fragments and variants thereof, are referred to as “20685, 579, 17114, 23821, 32613, and 33894 transporter sequences.”

The transporter sequences of the invention belong to the transporter family of molecules having conserved functional features. The term “family” when referring to the proteins and nucleic acid molecules of the invention is intended to mean two or more proteins or nucleic acid molecules having sufficient amino acid or nucleotide sequence identity as defined herein to provide a specific function. Such family members can be naturally-occurring and can be from either the same or different species. For example, a family can contain a first protein of murine origin and an ortholog of that protein of human origin, as well as a second, distinct protein of human origin and a murine ortholog of that protein.

Expression of the transporter mRNAs in the cells and tissues mentioned above indicates that the transporter is likely to be involved in the proper function of and in disorders involving these tissues. Accordingly, the disclosed invention further relates to methods and compositions for the study, modulation, diagnosis and treatment of transporter related disorders, especially disorders of these tissues that include, but are not limited to those disclosed herein.

For example, the fact that a transporter is expressed in a malignant cell, such as lymphoma or colonic metastases, means that the gene is relevant to these disorders. Moreover, if the transporter is expressed in megakaryocytes, this means that the expression is relevant to the formation of mature platelets and, accordingly, can be used to treat or diagnose thrombocytopenia. A transporter expressed in osteoblasts can be used to treat disorders of bone mass, such as osteoporosis or osteopetrosis. A transporter expressed in T cells can be used to treat inflammation. A transporter involved in neurotransmission can be used to treat disorders involving motor skills, cognitive function, and other disorders involving proper neurological function. Moreover, neurotransmitters are also relevant to the treatment of pain.

In addition, expression is particularly relevant in disorders involving tissues or cells in which a transporter gene is highly expressed. Still, further, where a transporter is diferentially expressed in a virally-infected cell, modulation of the gene is especially relevant in such cells or treatment of the viral disorder and also useful for diagnosis of such a disorder. Further, expression is relevant to prevent, treat, or diagnose the effects of viral infection, such as tissue fibrosis and especially liver fibrosis.

The compositions include transporter polypeptides, nucleic acids, vectors, transformed cells and related variants and fragments thereof, as well as agents that modulate expression of the polypeptides and polynucleotides. In particular, the invention relates to the modulation, diagnosis and treatment of transporter related disorders as described herein. Treatment is defined as the application or administration of a therapeutic agent to a patient, or application or administration of a therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease or the predisposition toward disease. “Subject”, as used herein, can refer to a mammal, e.g. a human, or to an experimental or animal or disease model. The subject can also be a non-human animal, e.g. a horse, cow, goat, or other domestic animal. A therapeutic agent includes, but is not limited to, small molecules, peptides, antibodies, ribozymes and antisense oligonucleotides.

Disorders involving the spleen include, but are not limited to, splenomegaly, including nonspecific acute splenitis, congestive spenomegaly, and spenic infarcts; neoplasms, congenital anomalies, and rupture. Disorders associated with splenomegaly include infections, such as nonspecific splenitis, infectious mononucleosis, tuberculosis, typhoid fever, brucellosis, cytomegalovirus, syphilis, malaria, histoplasmosis, toxoplasmosis, kala-azar, trypanosomiasis, schistosomiasis, leishmaniasis, and echinococcosis; congestive states related to partial hypertension, such as cirrhosis of the liver, portal or splenic vein thrombosis, and cardiac failure; lymphohematogenous disorders, such as Hodgkin disease, non-Hodgkin lymphomas/leukemia, multiple myeloma, myeloproliferative disorders, hemolytic anemias, and thrombocytopenic purpura; immunologic-inflammatory conditions, such as rheumatoid arthritis and systemic lupus erythematosus; storage diseases such as Gaucher disease, Niemann-Pick disease, and mucopolysaccharidoses; and other conditions, such as amyloidosis, primary neoplasms and cysts, and secondary neoplasms.

Disorders involving the lung include, but are not limited to, congenital anomalies; atelectasis; diseases of vascular origin, such as pulmonary congestion and edema, including hemodynamic pulmonary edema and edema caused by microvascular injury, adult respiratory distress syndrome (diffuse alveolar damage), pulmonary embolism, hemorrhage, and infarction, and pulmonary hypertension and vascular sclerosis; chronic obstructive pulmonary disease, such as emphysema, chronic bronchitis, bronchial asthma, and bronchiectasis; diffuse interstitial (infiltrative, restrictive) diseases, such as pneumoconioses, sarcoidosis, idiopathic pulmonary fibrosis, desquamative interstitial pneumonitis, hypersensitivity pneumonitis, pulmonary eosinophilia (pulmonary infiltration with eosinophilia), Bronchiolitis obliterans-organizing pneumonia, diffuse pulmonary hemorrhage syndromes, including Goodpasture syndrome, idiopathic pulmonary hemosiderosis and other hemorrhagic syndromes, pulmonary involvement in collagen vascular disorders, and pulmonary alveolar proteinosis; complications of therapies, such as drug-induced lung disease, radiation-induced lung disease, and lung transplantation; tumors, such as bronchogenic carcinoma, including paraneoplastic syndromes, bronchioloalveolar carcinoma, neuroendocrine tumors, such as bronchial carcinoid, miscellaneous tumors, and metastatic tumors; pathologies of the pleura, including inflammatory pleural effusions, noninflammatory pleural effusions, pneumothorax, and pleural tumors, including solitary fibrous tumors (pleural fibroma) and malignant mesothelioma.

Disorders involving the colon include, but are not limited to, congenital anomalies, such as atresia and stenosis, Meckel diverticulum, congenital aganglionic megacolon-Hirschsprung disease; enterocolitis, such as diarrhea and dysentery, infectious enterocolitis, including viral gastroenteritis, bacterial enterocolitis, necrotizing enterocolitis, antibiotic-associated colitis (pseudomembranous colitis), and collagenous and lymphocytic colitis, miscellaneous intestinal inflammatory disorders, including parasites and protozoa, acquired immunodeficiency syndrome, transplantation, drug-induced intestinal injury, radiation enterocolitis, neutropenic colitis (typhlitis), and diversion colitis; idiopathic inflammatory bowel disease, such as Crohn disease and ulcerative colitis; tumors of the colon, such as non-neoplastic polyps, adenomas, familial syndromes, colorectal carcinogenesis, colorectal carcinoma, and carcinoid tumors.

Disorders involving the liver include, but are not limited to, hepatic injury; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, a₁-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

Disorders involving the uterus and endometrium include, but are not limited to, endometrial histology in the menstrual cycle; functional endometrial disorders, such as anovulatory cycle, inadequate luteal phase, oral contraceptives and induced endometrial changes, and menopausal and postmenopausal changes; inflammations, such as chronic endometritis; adenomyosis; endometriosis; endometrial polyps; endometrial hyperplasia; malignant tumors, such as carcinoma of the endometrium; mixed Müllerian and mesenchymal tumors, such as malignant mixed Müllerian tumors; tumors of the myometrium, including leiomyomas, leiomyosarcomas, and endometrial stromal tumors.

Disorders involving the brain include, but are not limited to, disorders involving neurons, and disorders involving glia, such as astrocytes, oligodendrocytes, ependymal cells, and microglia; cerebral edema, raised intracranial pressure and herniation, and hydrocephalus; malformations and developmental diseases, such as neural tube defects, forebrain anomalies, posterior fossa anomalies, and syringomyelia and hydromyelia; perinatal brain injury; cerebrovascular diseases, such as those related to hypoxia, ischemia, and infarction, including hypotension, hypoperfusion, and low-flow states—global cerebral ischemia and focal cerebral ischemia—infarction from obstruction of local blood supply, intracranial hemorrhage, including intracerebral (intraparenchymal) hemorrhage, subarachnoid hemorrhage and ruptured berry aneurysms, and vascular malformations, hypertensive cerebrovascular disease, including lacunar infarcts, slit hemorrhages, and hypertensive encephalopathy; infections, such as acute meningitis, including acute pyogenic (bacterial) meningitis and acute aseptic (viral) meningitis, acute focal suppurative infections, including brain abscess, subdural empyema, and extradural abscess, chronic bacterial meningoencephalitis, including tuberculosis and mycobacterioses, neurosyphilis, and neuroborreliosis (Lyme disease), viral meningoencephalitis, including arthropod-borne (Arbo) viral encephalitis, Herpes simplex virus Type 1, Herpes simplex virus Type 2, Varicalla-zoster virus (Herpes zoster), cytomegalovirus, poliomyelitis, rabies, and human immunodeficiency virus 1, including HIV-1 meningoencephalitis (subacute encephalitis), vacuolar myelopathy, AIDS-associated myopathy, peripheral neuropathy, and AIDS in children, progressive multifocal leukoencephalopathy, subacute sclerosing panencephalitis, fungal meningoencephalitis, other infectious diseases of the nervous system; transmissible spongiform encephalopathies (prion diseases); demyelinating diseases, including multiple sclerosis, multiple sclerosis variants, acute disseminated encephalomyelitis and acute necrotizing hemorrhagic encephalomyelitis, and other diseases with demyelination; degenerative diseases, such as degenerative diseases affecting the cerebral cortex, including Alzheimer disease and Pick disease, degenerative diseases of basal ganglia and brain stem, including Parkinsonism, idiopathic Parkinson disease (paralysis agitans), progressive supranuclear palsy, corticobasal degeneration, multiple system atrophy, including striatonigral degeneration, Shy-Drager syndrome, and olivopontocerebellar atrophy, and Huntington disease; spinocerebellar degenerations, including spinocerebellar ataxias, including Friedreich ataxia, and ataxia-telanglectasia, degenerative diseases affecting motor neurons, including amyotrophic lateral sclerosis (motor neuron disease), bulbospinal atrophy (Kennedy syndrome), and spinal muscular atrophy; inborn errors of metabolism, such as leukodystrophies, including Krabbe disease, metachromatic leukodystrophy, adrenoleukodystrophy, Pelizaeus-Merzbacher disease, and Canavan disease, mitochondrial encephalomyopathies, including Leigh disease and other mitochondrial encephalomyopathies; toxic and acquired metabolic diseases, including vitamin deficiencies such as thiamine (vitamin B₁) deficiency and vitamin B₁₂ deficiency, neurologic sequelae of metabolic disturbances, including hypoglycemia, hyperglycemia, and hepatic encephatopathy, toxic disorders, including carbon monoxide, methanol, ethanol, and radiation, including combined methotrexate and radiation-induced injury; tumors, such as gliomas, including astrocytoma, including fibrillary (diffuse) astrocytoma and glioblastoma multiforme, pilocytic astrocytoma, pleomorphic xanthoastrocytoma, and brain stem glioma, oligodendroglioma, and ependymoma and related paraventricular mass lesions, neuronal tumors, poorly differentiated neoplasms, including medulloblastoma, other parenchymal tumors, including primary brain lymphoma, germ cell tumors, and pineal parenchymal tumors, meningiomas, metastatic tumors, paraneoplastic syndromes, peripheral nerve sheath tumors, including schwannoma, neurofibroma, and malignant peripheral nerve sheath tumor (malignant schwannoma), and neurocutaneous syndromes (phakomatoses), including neurofibromotosis, including Type 1 neurofibromatosis (NF1) and TYPE 2 neurofibromatosis (NF2), tuberous sclerosis, and Von Hippel-Lindau disease.

Disorders involving T-cells include, but are not limited to, cell-mediated hypersensitivity, such as delayed type hypersensitivity and T-cell-mediated cytotoxicity, and transplant rejection; autoimmune diseases, such as systemic lupus erythematosus, Sjögren syndrome, systemic sclerosis, inflammatory myopathies, mixed connective tissue disease, and polyarteritis nodosa and other vasculitides; immunologic deficiency syndromes, including but not limited to, primary immunodeficiencies, such as thymic hypoplasia, severe combined immunodeficiency diseases, and AIDS; leukopenia; reactive (inflammatory) proliferations of white cells, including but not limited to, leukocytosis, acute nonspecific lymphadenitis, and chronic nonspecific lymphadenitis; neoplastic proliferations of white cells, including but not limited to lymphoid neoplasms, such as precursor T-cell neoplasms, such as acute lymphoblastic leukemia/lymphoma, peripheral T-cell and natural killer cell neoplasms that include peripheral T-cell lymphoma, unspecified, adult T-cell leukemia/lymphoma, mycosis fungoides and Sézary syndrome, and Hodgkin disease.

Diseases of the skin, include but are not limited to, disorders of pigmentation and melanocytes, including but not limited to, vitiligo, freckle, melasma, lentigo, nevocellular nevus, dysplastic nevi, and malignant melanoma; benign epithelial tumors, including but not limited to, seborrheic keratoses, acanthosis nigricans, fibroepithelial polyp, epithelial cyst, keratoacanthoma, and adnexal (appendage) tumors; premalignant and malignant epidermal tumors, including but not limited to, actinic keratosis, squamous cell carcinoma, basal cell carcinoma, and merkel cell carcinoma; tumors of the dermis, including but not limited to, benign fibrous histiocytoma, dermatofibrosarcoma protuberans, xanthomas, and dermal vascular tumors; tumors of cellular immigrants to the skin, including but not limited to, histiocytosis X, mycosis fungoides (cutaneous T-cell lymphoma), and mastocytosis; disorders of epidermal maturation, including but not limited to, ichthyosis; acute inflammatory dermatoses, including but not limited to, urticaria, acute eczematous dermatitis, and erythema multiforme; chronic inflammatory dermatoses, including but not limited to, psoriasis, lichen planus, and lupus erythematosus; blistering (bullous) diseases, including but not limited to, pemphigus, bullous pemphigoid, dermatitis herpetiformis, and noninflammatory blistering diseases: epidermolysis bullosa and porphyria; disorders of epidermal appendages, including but not limited to, acne vulgaris; panniculitis, including but not limited to, erythema nodosum and erythema induratum; and infection and infestation, such as verrucae, molluscum contagiosum, impetigo, superficial fungal infections, and arthropod bites, stings, and infestations.

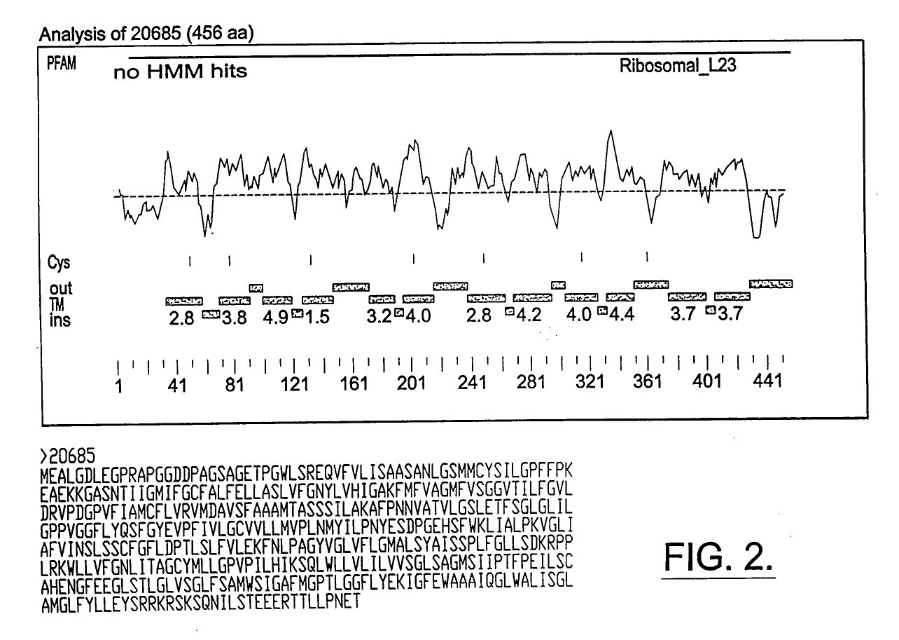

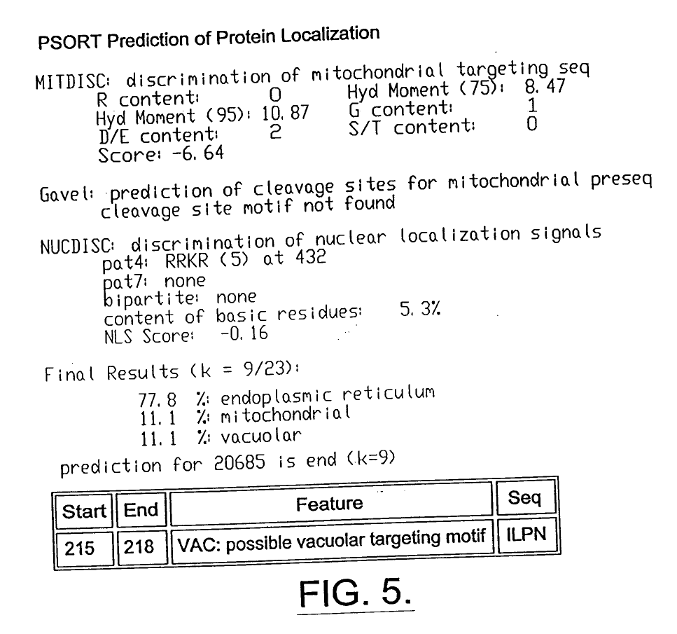

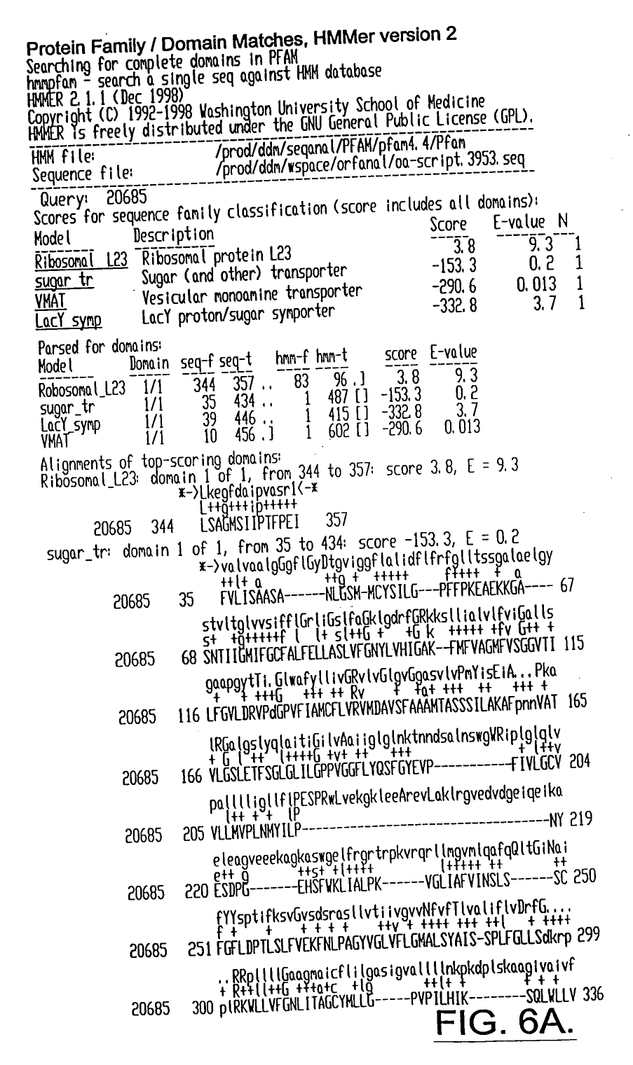

In normal bone marrow, the myelocytic series (polymorphoneuclear cells) make up approximately 60% of the cellular elements, and the erythrocytic series, 20-30%. Lymphocytes, monocytes, reticular cells, plasma cells and megakaryocytes together constitute 10-20%. Lymphocytes make up 5-15% of normal adult marrow. In the bone marrow, cell types are add mixed so that precursors of red blood cells (erythroblasts), macrophages (monoblasts), platelets (megakaryocytes), polymorphoneuclear leucocytes (myeloblasts), and lymphocytes (lymphoblasts) can be visible in one microscopic field. In addition, stem cells exist for the different cell lineages, as well as a precursor stem cell for the committed progenitor cells of the different lineages. The various types of cells and stages of each would be known to the person of ordinary skill in the art and are found, for example, on page 42 (FIG. 2-8) of Immunology, Imunopathology and Immunity, Fifth Edition, Sell et al. Simon and Schuster (1996), incorporated by reference for its teaching of cell types found in the bone marrow. According, the invention is directed to disorders arising from these cells. These disorders include but are not limited to the following: diseases involving hematopoeitic stem cells; committed lymphoid progenitor cells; lymphoid cells including B and T-cells; committed myeloid progenitors, including monocytes, granulocytes, and megakaryocytes; and committed erythroid progenitors. These include but are not limited to the leukemias, including B-lymphoid leukemias, T-lymphoid leukemias, undifferentiated leukemias; erythroleukemia, megakaryoblastic leukemia, monocytic; [leukemias are encompassed with and without differentiation]; chronic and acute lymphoblastic leukemia, chronic and acute lymphocytic leukemia, chronic and acute myelogenous leukemia, lymphoma, myelo dysplastic syndrome, chronic and acute myeloid leukemia, myelomonocytic leukemia; chronic and acute myeloblastic leukemia, chronic and acute myelogenous leukemia, chronic and acute promyelocytic leukemia, chronic and acute myelocytic leukemia, hematologic malignancies of monocyte-macrophage lineage, such as juvenile chronic myelogenous leukemia; secondary AML, antecedent hematological disorder; refractory anemia; aplastic anemia; reactive cutaneous angioendotheliomatosis; fibrosing disorders involving altered expression in dendritic cells, disorders including systemic sclerosis, E-M syndrome, epidemic toxic oil syndrome, eosinophilic fasciitis localized forms of scleroderma, keloid, and fibrosing colonopathy; angiomatoid malignant fibrous histiocytoma; carcinoma, including primary head and neck squamous cell carcinoma; sarcoma, including kaposi's sarcoma; fibroadanoma and phyllodes tumors, including mammary fibroadenoma; stromal tumors; phyllodes tumors, including histiocytoma; erythroblastosis; neurofibromatosis; diseases of the vascular endothelium; demyelinating, particularly in old lesions; gliosis, vasogenic edema, vascular disease, Alzheimer's and Parkinson's disease; T-cell lymphomas; B-cell lymphomas.

Disorders involving the heart, include but are not limited to, heart failure, including but not limited to, cardiac hypertrophy, left-sided heart failure, and right-sided heart failure; ischemic heart disease, including but not limited to angina pectoris, myocardial infarction, chronic ischemic heart disease, and sudden cardiac death; hypertensive heart disease, including but not limited to, systemic (left-sided) hypertensive heart disease and pulmonary (right-sided) hypertensive heart disease; valvular heart disease, including but not limited to, valvular degeneration caused by calcification, such as calcific aortic stenosis, calcification of a congenitally bicuspid aortic valve, and mitral annular calcification, and myxomatous degeneration of the mitral valve (mitral valve prolapse), rheumatic fever and rheumatic heart disease, infective endocarditis, and noninfected vegetations, such as nonbacterial thrombotic endocarditis and endocarditis of systemic lupus erythematosus (Libman-Sacks disease), carcinoid heart disease, and complications of artificial valves; myocardial disease, including but not limited to dilated cardiomyopathy, hypertrophic cardiomyopathy, restrictive cardiomyopathy, and myocarditis; pericardial disease, including but not limited to, pericardial effusion and hemopericardium and pericarditis, including acute pericarditis and healed pericarditis, and rheumatoid heart disease; neoplastic heart disease, including but not limited to, primary cardiac tumors, such as myxoma, lipoma, papillary fibroelastoma, rhabdomyoma, and sarcoma, and cardiac effects of noncardiac neoplasms; congenital heart disease, including but not limited to, left-to-right shunts—late cyanosis, such as atrial septal defect, ventricular septal defect, patent ductus arteriosus, and atrioventricular septal defect, right-to-left shunts—early cyanosis, such as tetralogy of fallot, transposition of great arteries, truncus arteriosus, tricuspid atresia, and total anomalous pulmonary venous connection, obstructive congenital anomalies, such as coarctation of aorta, pulmonary stenosis and atresia, and aortic stenosis and atresia, and disorders involving cardiac transplantation.

Disorders involving blood vessels include, but are not limited to, responses of vascular cell walls to injury, such as endothelial dysfunction and endothelial activation and intimal thickening; vascular diseases including, but not limited to, congenital anomalies, such as arteriovenous fistula, atherosclerosis, and hypertensive vascular disease, such as hypertension; inflammatory disease—the vasculitides, such as giant cell (temporal) arteritis, Takayasu arteritis, polyarteritis nodosa (classic), Kawasaki syndrome (mucocutaneous lymph node syndrome), microscopic polyanglitis (microscopic polyarteritis, hypersensitivity or leukocytoclastic anglitis), Wegener granulomatosis, thromboanglitis obliterans (Buerger disease), vasculitis associated with other disorders, and infectious arteritis; Raynaud disease; aneurysms and dissection, such as abdominal aortic aneurysms, syphilitic (luetic) aneurysms, and aortic dissection (dissecting hematoma); disorders of veins and lymphatics, such as varicose veins, thrombophlebitis and phlebothrombosis, obstruction of superior vena cava (superior vena cava syndrome), obstruction of inferior vena cava (inferior vena cava syndrome), and lymphangitis and lymphedema; tumors, including benign tumors and tumor-like conditions, such as hemangioma, lymphangioma, glomus tumor (glomangioma), vascular ectasias, and bacillary angiomatosis, and intermediate-grade (borderline low-grade malignant) tumors, such as Kaposi sarcoma and hemangloendothelioma, and malignant tumors, such as angiosarcoma and hemangiopericytoma; and pathology of therapeutic interventions in vascular disease, such as balloon angioplasty and related techniques and vascular replacement, such as coronary artery bypass graft surgery.

Disorders involving red cells include, but are not limited to, anemias, such as hemolytic anemias, including hereditary spherocytosis, hemolytic disease due to erythrocyte enzyme defects: glucose-6-phosphate dehydrogenase deficiency, sickle cell disease, thalassemia syndromes, paroxysmal nocturnal hemoglobinuria, immunohemolytic anemia, and hemolytic anemia resulting from trauma to red cells; and anemias of diminished erythropoiesis, including megaloblastic anemias, such as anemias of vitamin B₁₂ deficiency: pernicious anemia, and anemia of folate deficiency, iron deficiency anemia, anemia of chronic disease, aplastic anemia, pure red cell aplasia, and other forms of marrow failure.

Disorders involving the thymus include developmental disorders, such as DiGeorge syndrome with thymic hypoplasia or aplasia; thymic cysts; thymic hypoplasia, which involves the appearance of lymphoid follicles within the thymus, creating thymic follicular hyperplasia; and thymomas, including germ cell tumors, lynphomas, Hodgkin disease, and carcinoids. Thymomas can include benign or encapsulated thymoma, and malignant thymoma Type I (invasive thymoma) or Type II, designated thymic carcinoma.

Disorders involving B-cells include, but are not limited to precursor B-cell neoplasms, such as lymphoblastic leukemia/lymphoma. Peripheral B-cell neoplasms include, but are not limited to, chronic lymphocytic leukemia/small lymphocytic lymphoma, follicular lymphoma, diffuse large B-cell lymphoma, Burkitt lymphoma, plasma cell neoplasms, multiple myeloma, and related entities, lymphoplasmacytic lymphoma (Waldenström macroglobulinemia), mantle cell lymphoma, marginal zone lymphoma (MALToma), and hairy cell leukemia.

Disorders involving the kidney include, but are not limited to, congenital anomalies including, but not limited to, cystic diseases of the kidney, that include but are not limited to, cystic renal dysplasia, autosomal dominant (adult) polycystic kidney disease, autosomal recessive (childhood) polycystic kidney disease, and cystic diseases of renal medulla, which include, but are not limited to, medullary sponge kidney, and nephronophthisis-uremic medullary cystic disease complex, acquired (dialysis-associated) cystic disease, such as simple cysts; glomerular diseases including pathologies of glomerular injury that include, but are not limited to, in situ immune complex deposition, that includes, but is not limited to, anti-GBM nephritis, Heymann nephritis, and antibodies against planted antigens, circulating immune complex nephritis, antibodies to glomerular cells, cell-mediated immunity in glomerulonephritis, activation of alternative complement pathway, epithelial cell injury, and pathologies involving mediators of glomerular injury including cellular and soluble mediators, acute glomerulonephritis, such as acute proliferative (poststreptococcal, postinfectious) glomerulonephritis, including but not limited to, poststreptococcal glomerulonephritis and nonstreptococcal acute glomerulonephritis, rapidly progressive (crescentic) glomerulonephritis, nephrotic syndrome, membranous glomerulonephritis (membranous nephropathy), minimal change disease (lipoid nephrosis), focal segmental glomerulosclerosis, membranoproliferative glomerulonephritis, IgA nephropathy (Berger disease), focal proliferative and necrotizing glomerulonephritis (focal glomerulonephritis), hereditary nephritis, including but not limited to, Alport syndrome and thin membrane disease (benign familial hematuria), chronic glomerulonephritis, glomerular lesions associated with systemic disease, including but not limited to, systemic lupus erythematosus, Henoch-Schonlein purpura, bacterial endocarditis, diabetic glomerulosclerosis, amyloidosis, fibrillary and immunotactoid glomerulonephritis, and other systemic disorders; diseases affecting tubules and interstitium, including acute tubular necrosis and tubulointerstitial nephritis, including but not limited to, pyelonephritis and urinary tract infection, acute pyelonephritis, chronic pyelonephritis and reflux nephropathy, and tubulointerstitial nephritis induced by drugs and toxins, including but not limited to, acute drug-induced interstitial nephritis, analgesic abuse nephropathy, nephropathy associated with nonsteroidal anti-inflammatory drugs, and other tubulointerstitial diseases including, but not limited to, urate nephropathy, hypercalcemia and nephrocalcinosis, and multiple myeloma; diseases of blood vessels including benign nephrosclerosis, malignant hypertension and accelerated nephrosclerosis, renal artery stenosis, and thrombotic microangiopathies including, but not limited to, classic (childhood) hemolytic-uremic syndrome, adult hemolytic-uremic syndrome/thrombotic thrombocytopenic purpura, idiopathic HUS/TTP, and other vascular disorders including, but not limited to, atherosclerotic ischemic renal disease, atheroembolic renal disease, sickle cell disease nephropathy, diffuse cortical necrosis, and renal infarcts; urinary tract obstruction (obstructive uropathy); urolithiasis (renal calculi, stones); and tumors of the kidney including, but not limited to, benign tumors, such as renal papillary adenoma, renal fibroma or hamartoma (renomedullary interstitial cell tumor), angiomyolipoma, and oncocytoma, and malignant tumors, including renal cell carcinoma (hypernephroma, adenocarcinoma of kidney), which includes urothelial carcinomas of renal pelvis.

Disorders of the breast include, but are not limited to, disorders of development; inflammations, including but not limited to, acute mastitis, periductal mastitis, periductal mastitis (recurrent subareolar abscess, squamous metaplasia of lactiferous ducts), mammary duct ectasia, fat necrosis, granulomatous mastitis, and pathologies associated with silicone breast implants; fibrocystic changes; proliferative breast disease including, but not limited to, epithelial hyperplasia, sclerosing adenosis, and small duct papillomas; tumors including, but not limited to, stromal tumors such as fibroadenoma, phyllodes tumor, and sarcomas, and epithelial tumors such as large duct papilloma; carcinoma of the breast including in situ (noninvasive) carcinoma that includes ductal carcinoma in situ (including Paget's disease) and lobular carcinoma in situ, and invasive (infiltrating) carcinoma including, but not limited to, invasive ductal carcinoma, no special type, invasive lobular carcinoma, medullary carcinoma, colloid (mucinous) carcinoma, tubular carcinoma, and invasive papillary carcinoma, and miscellaneous malignant neoplasms.

Disorders in the male breast include, but are not limited to, gynecomastia and carcinoma.

Disorders involving the testis and epididymis include, but are not limited to, congenital anomalies such as cryptorchidism, regressive changes such as atrophy, inflammations such as nonspecific epididymitis and orchitis, granulomatous (autoimmune) orchitis, and specific inflammations including, but not limited to, gonorrhea, mumps, tuberculosis, and syphilis, vascular disturbances including torsion, testicular tumors including germ cell tumors that include, but are not limited to, seminoma, spermatocytic seminoma, embryonal carcinoma, yolk sac tumor choriocarcinoma, teratoma, and mixed tumors, tumore of sex cord-gonadal stroma including, but not limited to, Leydig (interstitial) cell tumors and sertoli cell tumors (androblastoma), and testicular lymphoma, and miscellaneous lesions of tunica vaginalis.

Disorders involving the prostate include, but are not limited to, inflammations, benign enlargement, for example, nodular hyperplasia (benign prostatic hypertrophy or hyperplasia), and tumors such as carcinoma.

Disorders involving the thyroid include, but are not limited to, hyperthyroidism; hypothyroidism including, but not limited to, cretinism and myxedema; thyroiditis including, but not limited to, hashimoto thyroiditis, subacute (granulomatous) thyroiditis, and subacute lymphocytic (painless) thyroiditis; Graves disease; diffuse and multinodular goiter including, but not limited to, diffuse nontoxic (simple) goiter and multinodular goiter; neoplasms of the thyroid including, but not limited to, adenomas, other benign tumors, and carcinomas, which include, but are not limited to, papillary carcinoma, follicular carcinoma, medullary carcinoma, and anaplastic carcinoma; and cogenital anomalies.

Disorders involving the skeletal muscle include tumors such as rhabdomyosarcoma.

Disorders involving the pancreas include those of the exocrine pancreas such as congenital anomalies, including but not limited to, ectopic pancreas; pancreatitis, including but not limited to, acute pancreatitis; cysts, including but not limited to, pseudocysts; tumors, including but not limited to, cystic tumors and carcinoma of the pancreas; and disorders of the endocrine pancreas such as, diabetes mellitus; islet cell tumors, including but not limited to, insulinomas, gastrinomas, and other rare islet cell tumors.

Disorders involving the small intestine include the malabsorption syndromes such as, celiac sprue, tropical sprue (postinfectious sprue), whipple disease, disaccharidase (lactase) deficiency, abetalipoproteinemia, and tumors of the small intestine including adenomas and adenocarcinoma.

Disorders related to reduced platelet number, thrombocytopenia, include idiopathic thrombocytopenic purpura, including acute idiopathic thrombocytopenic purpura, drug-induced thrombocytopenia, HIV-associated thrombocytopenia, and thrombotic microangiopathies: thrombotic thrombocytopenic purpura and hemolytic-uremic syndrome.

Disorders involving precursor T-cell neoplasms include precursor T lymphoblastic leukemia/lymphoma. Disorders involving peripheral T-cell and natural killer cell neoplasms include T-cell chronic lymphocytic leukemia, large granular lymphocytic leukemia, mycosis fungoides and Sézary syndrome, peripheral T-cell lymphoma, unspecified, angioimmunoblastic T-cell lymphoma, angiocentric lymphoma (NK/T-cell lymphoma^{4a}), intestinal T-cell lymphoma, adult T-cell leukemia/lymphoma, and anaplastic large cell lymphoma.

Disorders involving the ovary include, for example, polycystic ovarian disease, Stein-leventhal syndrome, Pseudomyxoma peritonei and stromal hyperthecosis; ovarian tumors such as, tumors of coelomic epithelium, serous tumors, mucinous tumors, endometeriod tumors, clear cell adenocarcinoma, cystadenofibroma, brenner tumor, surface epithelial tumors; germ cell tumors such as mature (benign) teratomas, monodermal teratomas, immature malignant teratomas, dysgerminoma, endodermal sinus tumor, choriocarcinoma; sex cord-stomal tumors such as, granulosa-theca cell tumors, thecoma-fibromas, androblastomas, hill cell tumors, and gonadoblastoma; and metastatic tumors such as Krukenberg tumors.

Bone-forming cells include the osteoprogenitor cells, osteoblasts, and osteocytes. The disorders of the bone are complex because they may have an impact on the skeleton during any of its stages of development. Hence, the disorders may have variable manifestations and may involve one, multiple or all bones of the body. Such disorders include, congenital malformations, achondroplasia and thanatophoric dwarfism, diseases associated with abnormal matix such as type 1 collagen disease, osteoporosis, Paget disease, rickets, osteomalacia, high-turnover osteodystrophy, low-turnover of aplastic disease, osteonecrosis, pyogenic osteomyelitis, tuberculous osteomyelitism, osteoma, osteoid osteoma, osteoblastoma, osteosarcoma, osteochondroma, chondromas, chondroblastoma, chondromyxoid fibroma, chondrosarcoma, fibrous cortical defects, fibrous dysplasia, fibrosarcoma, malignant fibrous histiocytoma, Ewing sarcoma, primitive neuroectodermal tumor, giant cell tumor, and metastatic tumors.

Furthermore, as disclosed in the background hereinabove, specific disorders have been associated with function of the various transporters. Accordingly, the transporters disclosed herein, having homology to specific transporters as disclosed herein, are useful for diagnosis and treatment of the disorders associated with transporter dysfunction as disclosed herein and for modulation of gene expression in the affected tissues.

The sequences of the invention find use in diagnosis of disorders involving altered transporter expression. The sequences also find use in modulating transporter-related responses. By “modulating” is intended the upregulating or downregulating of a response. That is, the compositions of the invention affect the targeted activity in either a positive or negative fashion.

For diagnosis of a disorder involving aberrant transporter expression, results obtained with a biological sample from a test subject may be compared to results obtained with a biological sample from a control subject. “Misexpression or aberrant expression”, as used herein, refers to a non-wild type pattern of gene expression, at the RNA or protein level. It includes: expression at non-wild type levels, i.e., over or under expression; a pattern of expression that differs from wild type in terms of the time or stage at which the gene is expressed, e.g., increased or decreased expression (as compared with wild type) at a predetermined developmental period or stage; a pattern of expression that differs from wild type in terms of decreased expression (as compared with wild type) in a predetermined cell type or tissue type; a pattern of expression that differs from wild type in terms of the splicing size, amino acid sequence, post-transitional modification, or biological activity of the expressed polypeptide; a pattern of expression that differs from wild type in terms of the effect of an environmental stimulus or extracellular stimulus on expression of the gene, e.g., a pattern of increased or decreased expression (as compared with wild type) in the presence of an increase or decrease in the strength of the stimulus.

The present invention provides isolated or purified transporter polypeptides and variants and fragments thereof. “Transporter polypeptide” or “transporter protein” refers to the polypeptide in SEQ ID NOS:2, 5, 8, 11, 14, or 17, or encoded by the deposited cDNAs. The term “transporter protein” or “transporter polypeptide,” however, further includes the numerous variants described herein, as well as fragments derived from the full-length transporter and variants.

Transporter polypeptides can be purified to homogeneity. It is understood, however, that preparations in which the polypeptide is not purified to homogeneity are useful and considered to contain an isolated form of the polypeptide. The critical feature is that the preparation allows for the desired function of the polypeptide, even in the presence of considerable amounts of other components. Thus, the invention encompasses various degrees of purity.

As used herein, a polypeptide is said to be “isolated” or “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell and still be considered “isolated” or “purified.”

In one embodiment, the language “substantially free of cellular material” includes preparations of transporter having less than about 30% (by dry weight) other proteins (i.e., contaminating protein), less than about 20% other proteins, less than about 10% other proteins, or less than about 5% other proteins. When the polypeptide is recombinantly produced, it can also be substantially free of culture medium, i.e., culture medium represents less than about 20%, less than about 10%, or less than about 5% of the volume of the protein preparation.

The transporter polypeptide is also considered to be isolated when it is part of a membrane preparation or is purified and then reconstituted with membrane vesicles or liposomes.

The language “substantially free of chemical precursors or other chemicals” includes preparations of the transporter polypeptide in which it is separated from chemical precursors or other chemicals that are involved in its synthesis. The language “substantially free of chemical precursors or other chemicals” includes, but is not limited to, preparations of the polypeptide having less than about 30% (by dry weight) chemical precursors or other chemicals, less than about 20% chemical precursors or other chemicals, less than about 10% chemical precursors or other chemicals, or less than about 5% chemical precursors or other chemicals.

In one embodiment, the transporter polypeptide comprises the amino acid sequence shown in SEQ ID NOS:2, 5, 8, 11, 14, or 17. However, the invention also encompasses sequence variants. By “variants” is intended proteins or polypeptides having an amino acid sequence that is at least about 60%, 65%, or 70%, preferably about 75%, 85%, 90%, 91%, 92%, 93%, 94%, 95%, 96%, 97%, 98%, or 99% identical to the amino acid sequence of SEQ ID NOS:2, 5, 8, 11, 14, or 17. Variants retain the biological activity (e.g. the transporter activity) of the reference polypeptide set forth in SEQ ID NOS:2, 5, 8, 11, 14, or 17. Variants also include polypeptides encoded by a nucleic acid molecule that hybridizes to the nucleic acid molecule of SEQ ID NOS:1, 3, 4, 6, 7, 9, 10, 12, 13, 15, 16, or 18, or a complement thereof, under stringent conditions.

In another embodiment, a variant of an isolated polypeptide of the present invention differs, by at least 1, but less than 5, 10, 20, 50, or 100 amino acid residues from the sequence shown in SEQ ID NOS:2, 5, 8, 11, 14, or 17. If alignment is needed for this comparison the sequences should be aligned for maximum identity. “Looped” out sequences from deletions or insertions, or mismatches, are considered differences. Such variants generally retain the functional activity of the transporter-like proteins of the invention. Variants include polypeptides that differ in amino acid sequence due to natural allelic variation or mutagenesis. It is understood, however, that variants exclude any amino acid sequences disclosed prior to the invention.

Preferred transporter polypeptides of the present invention have an amino acid sequence sufficiently identical to the amino acid sequence of SEQ ID NOS:2, 5, 8, 11, 14 or 17. The term “sufficiently identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences that contain a common structural domain having at least about 60%, 65%, 70%, 75%, 80%, 85%, 90%, 91%, 92%, 93%, 94%, 95%, 96%, 97%, 98% or 99% identity are defined herein as sufficiently identical.

As used herein, two proteins (or a region of the proteins) are substantially homologous when the amino acid sequences are at least about 60%, 65%, 70%, 75%, 80%, 85%, 90%, 91%, 92%, 93%, 94%, 95%, 96%, 97%, 98%, 99% or more identical. A substantially homologous amino acid sequence, according to the present invention, will be encoded by a nucleic acid sequence hybridizing to the nucleic acid sequence, or portion thereof, of the sequence shown in SEQ ID NOS:1, 3, 4, 6, 7, 9, 10, 12, 13, 15, 16, or 18, under stringent conditions as more fully described below.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (1970) J. Mol. Biol. 48:444-453 algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) is using a Blossum 62 scoring matrix with a gap open penalty of 12, a gap extend penalty of 4, and a frameshift gap penalty of 5.

The percent identity between two amino acid or nucleotide sequences can be determined using the algorithm of Meyers and Miller (1989) CABIOS 4:11-17 which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other family members or related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to transporter nucleic acid molecules of the invention. BLAST protein searches can be performed with the XBLAST program, score=50, wordlength=3 to obtain amino acid sequences homologous to transporter protein molecules of the invention. To obtain gapped alignments for comparison purposes, Gapped BLAST can be utilized as described in Altschul et al. (1997) Nucleic Acids Res. 25(17):3389-3402. When utilizing BLAST and Gapped BLAST programs, the default parameters of the respective programs (e.g., XBLAST and NBLAST) can be used. See http://www.ncbi.nlm.nih.gov.

The invention also encompasses polypeptides having sufficient similarity so as to perform one or more of the same functions performed by the transporter. Similarity is determined by conservative amino acid substitution, as shown in Table 1. Such substitutions are those that substitute a given amino acid in a polypeptide by another amino acid of like characteristics. Conservative substitutions are likely to be phenotypically silent. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr. Guidance concerning which amino acid changes are likely to be phenotypically silent are found in Bowie et al., Science 247:1306-1310 (1990).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

A variant polypeptide can differ in amino acid sequence by one or more substitutions, deletions, insertions, inversions, fusions, and truncations or a combination of any of these. Variant polypeptides can be fully functional or can lack function in one or more activities. Thus, in the present case, variations can affect the transporter function, membrane association or subcellular localization, regions involved in post-translational modification, for example, by phosphorylation, and regions that are important for effector function (i.e., agents that act upon the protein).

Fully functional variants typically contain only conservative variation or variation in non-critical residues or in non-critical regions. Functional variants can also contain substitution of similar amino acids, which results in no change or an insignificant change in function. Alternatively, such substitutions may positively or negatively affect function to some degree.

Non-functional variants typically contain one or more non-conservative amino acid substitutions, deletions, insertions, inversions, or truncation or a substitution, insertion, inversion, or deletion in a critical residue or critical region.

As indicated, variants can be naturally-occurring or can be made by recombinant means or chemical synthesis to provide useful and novel characteristics for the transporter polypeptide. This includes preventing immunogenicity from pharmaceutical formulations by preventing protein aggregation.

Useful variations further include alteration of functional activity. For example, one embodiment involves a variation that results in binding but not transport or more or less transport of the substrate than wild type. A further useful variation at the same site can result in altered affinity for the substrate. Useful variations also include changes that provide for affinity for another substrate. Useful variations further include the ability to bind an effector molecule with greater or lesser affinity, such as not to bind or to bind but not release it. Further useful variations include alteration in the ability of the propeptide to be cleaved by a cleavage protein, including alteration in the binding or recognition site. Further, the cleavage site can also be modified so that recognition and cleavage are by a different protease.

Another useful variation provides a fusion protein in which one or more domains or subregions are operationally fused to one or more domains, subregions, or motifs from another transporter. For example, a transmembrane domain from a protein can be introduced into the transporter such that the protein is anchored in the cell surface. Other permutations include changing the number of transporter domains, and mixing of transporter domains from different transporter families, so that substrate specificity is altered. Mixing these various domains can allow the formation of novel transporter molecules with different host cell, subcellular localization, substrate, and effector molecule (one that acts on the transporter) specificity.

The term “substrate” is intended to refer not only to the transported substrate that but also to refer to any component with which the polypeptide interacts in order to produce an effect on that component or a subsequent biological effect that is a result of interacting with that component.

Amino acids that are essential for function can be identified by methods known in the art, such as site-directed mutagenesis or alanine-scanning mutagenesis (Cunningham et al. (1985) Science 244:1081-1085). The latter procedure introduces single alanine mutations at every residue in the molecule. The resulting mutant molecules are then tested for biological activity, such as peptide bond hydrolysis in vitro or related biological activity, such as proliferative activity. Sites that are critical for binding can also be determined by structural analysis such as crystallization, nuclear magnetic resonance or photoaffinity labeling (Smith et al. (1992) J. Mol. Biol. 224:899-904; de Vos et al. (1992) Science 255:306-312).

The invention thus also includes polypeptide fragments of the transporters. Fragments can be derived from the amino acid sequence shown in SEQ ID NOS:2, 5, 8, 11, 14 or 17. However, the invention also encompasses fragments of the variants of the transporter polypeptides as described herein. The fragments to which the invention pertains, however, are not to be construed as encompassing fragments that may be disclosed prior to the present invention.

A fragment can comprise at least about 5-10, 10-15, 15-20, 20-25, 25-30, 30-35, 35-40, 40-45, 45-50, 50-60, 60-70, 70-80, 80-90, 90-100, 200, 300, 400, 500, 600, 700, 800, 900, 1000, 1100, 1200, 1300, 1400, 1500, 1600, 1700, 1800, 1900, 2000, 2100, 2200, 2300 or more contiguous amino acids. Fragments can retain one or more of the biological activities of the protein, for example as discussed above, as well as fragments that can be used as an immunogen to generate transporter antibodies.

Alternatively, an amino acid sequence that is a fragment of a transporter-like amino acid sequence of the present invention comprises an amino acid sequence consisting of amino acid residues 1-100, 100-200, 200-300, 300-400, 400-456 of SEQ ID NO:2.

Alternatively, an amino acid sequence that is a fragment of a transporter-like amino acid sequence of the present invention comprises an amino acid sequence consisting of amino acid residues 1-100, 100-200, 200-300, 300-400, 400-500, 500-600, 600-700, 700-730 of SEQ ID NO:5.

Alternatively, an amino acid sequence that is a fragment of a transporter-like amino acid sequence of the present invention comprises an amino acid sequence consisting of amino acid residues 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2000-2100, 2100-2200, 2200-2300, 2300-2400, 2400-2436 of SEQ ID NO:8.

Alternatively, an amino acid sequence that is a fragment of a transporter-like amino acid sequence of the present invention comprises an amino acid sequence consisting of amino acid residues 1-100, 100-200, 200-300, 300-400, 400-450 of SEQ ID NO:11.

Alternatively, an amino acid sequence that is a fragment of a transporter-like amino acid sequence of the present invention comprises an amino acid sequence consisting of amino acid residues 1-100, 100-200, 200-300, 300-400, 400-500, 500-600, 600-700, 700-751 of SEQ ID NO:14.

Alternatively, an amino acid sequence that is a fragment of a transporter-like amino acid sequence of the present invention comprises an amino acid sequence consisting of amino acid residues 1-100, 100-200, 200-300, 300-400, 400-500, 500-600, 600-700, 700-766 of SEQ ID NO:17.

Biologically active fragments (peptides which are, for example, about 5, 10, 15, 20, 25, 30, 35, 40, 50, 100, 200, 300, 400, 500, 600, 700, 800, 900, 1000, 1100, 1200, 1300, 1400, 1500, 1600, 1700, 1800, 1900, 2000 or more amino acids in length) can comprise a functional site. Such sites include but are not limited to those discussed above, such as a regulatory site, site important for substrate recognition, binding or transport, regions containing a transporter domain or motif, phosphorylation sites, glycosylation sites, and other functional sites disclosed herein.

Fragments, for example, can extend in one or both directions from the functional site to encompass 5, 10, 15, 20, 30, 40, 50, 100, 200, 300, 400, 500, 600, 700, 800, 900, or up to 1000 amino acids. Further, fragments can include sub-fragments of the specific sites or regions disclosed herein, which sub-fragments retain the function of the site or region from which they are derived.

The invention also provides fragments with immunogenic properties. These contain an epitope-bearing portion of the transporter polypeptide and variants. These epitope-bearing peptides are useful to raise antibodies that bind specifically to a transporter polypeptide or region or fragment. These peptides can contain at least 10, 12, at least 14, or between at least about 15 to about 30 amino acids. The epitope-bearing transporter polypeptides may be produced by any conventional means (Houghten, R. A. (1985) Proc. Natl. Acad. Sci. USA 82:5131-5135). Simultaneous multiple peptide synthesis is described in U.S. Pat. No. 4,631,211.

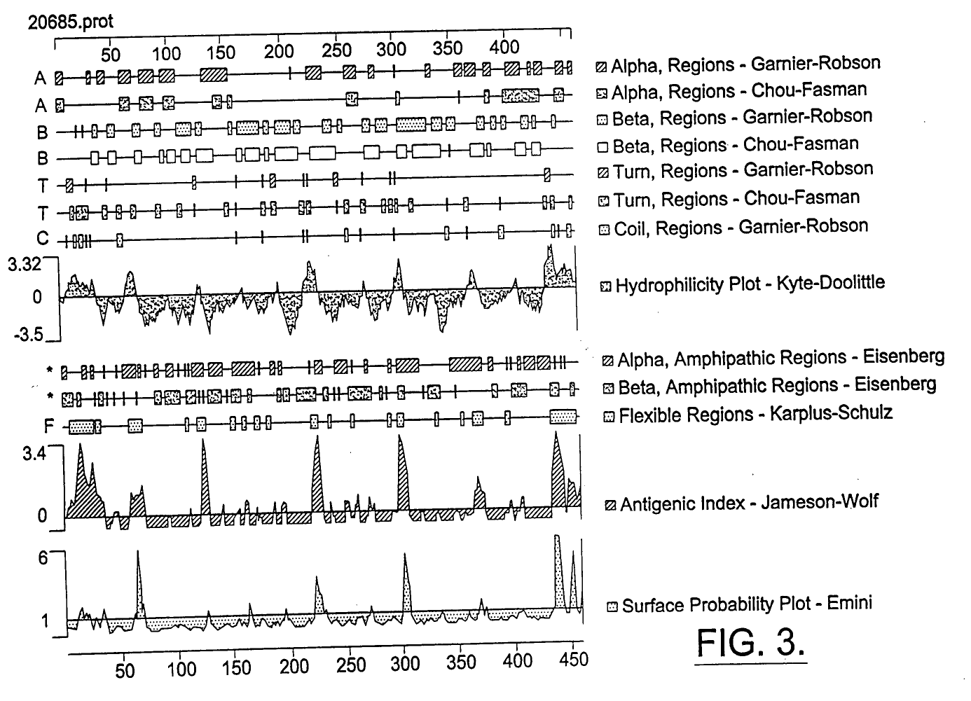

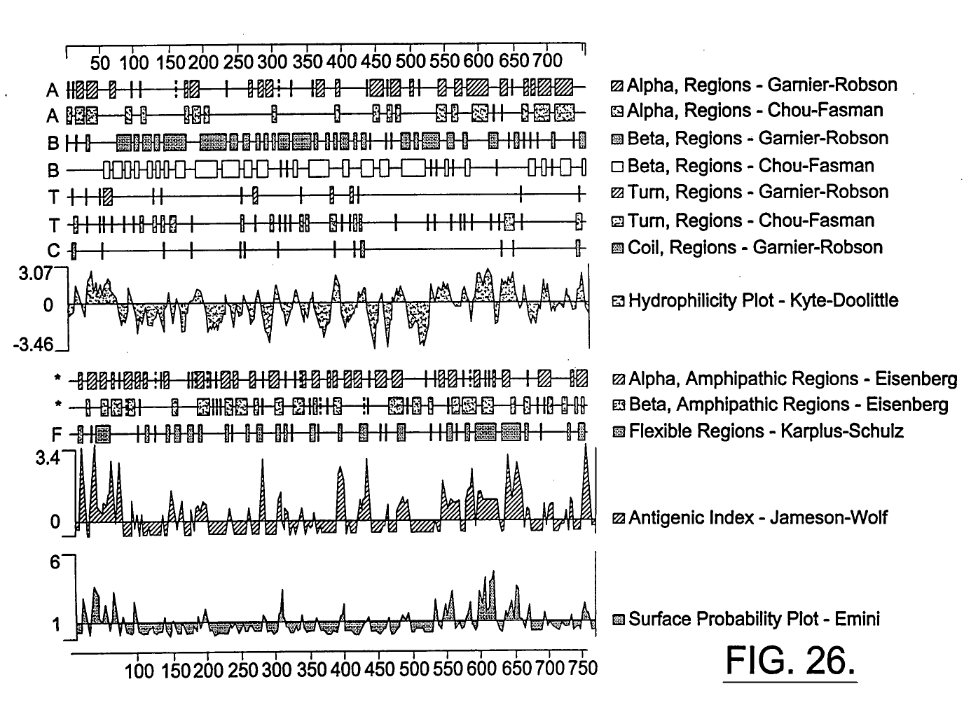

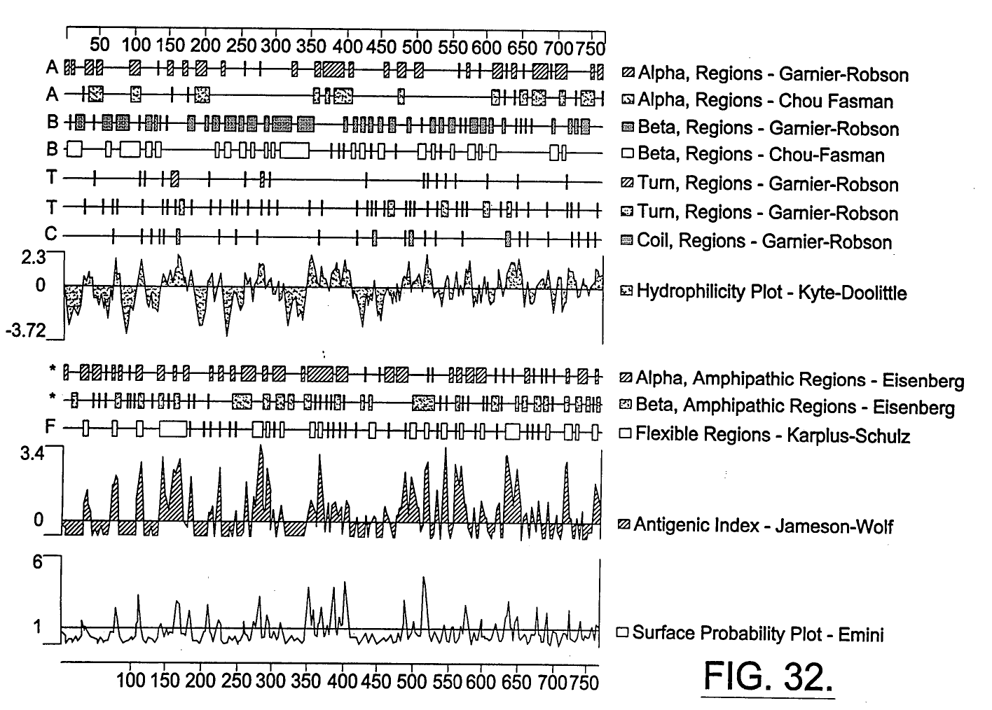

Non-limiting examples of antigenic polypeptides that can be used to generate antibodies include but are not limited to peptides derived from extracellular regions. Regions having a high antigenicity index are shown in FIGS. 3, 26, and 32. However, intracellularly-made antibodies (“intrabodies”) are also encompassed, which would recognize intracellular peptide regions.

Fragments can be discrete (not fused to other amino acids or polypeptides) or can be within a larger polypeptide. Further, several fragments can be comprised within a single larger polypeptide. In one embodiment a fragment designed for expression in a host can have heterologous pre- and pro-polypeptide regions fused to the amino terminus of the transporter polypeptide fragment and an additional region fused to the carboxyl terminus of the fragment.

The invention thus provides chimeric or fusion proteins. These comprise a transporter peptide sequence operatively linked to a heterologous peptide having an amino acid sequence not substantially homologous to the transporter polypeptide. “Operatively linked” indicates that the transporter polypeptide and the heterologous peptide are fused in-frame. The heterologous peptide can be fused to the N-terminus or C-terminus of the transporter polypeptide or can be internally located.

In one embodiment the fusion protein does not affect transporter function per se. For example, the fusion protein can be a GST-fusion protein in which transporter sequences are fused to the N- or C-terminus of the GST sequences. Other types of fusion proteins include, but are not limited to, enzymatic fusion proteins, for example beta-galactosidase fusions, yeast two-hybrid GAL4 fusions, poly-His fusions and Ig fusions. Such fusion proteins, particularly poly-His fusions, can facilitate the purification of recombinant transporter polypeptide. In certain host cells (e.g., mammalian host cells), expression and/or secretion of a protein can be increased by using a heterologous signal sequence. Therefore, in another embodiment, the fusion protein contains a heterologous signal sequence at its C- or N-terminus.

EP-A-O 464 533 discloses fusion proteins comprising various portions of immunoglobulin constant regions. The Fc is useful in therapy and diagnosis and thus results, for example, in improved pharmacokinetic properties (EP-A 0232 262). In drug discovery, for example, human proteins have been fused with Fc portions for the purpose of high-throughput screening assays to identify antagonists (Bennett et al. (1995) J. Mol. Recog. 8:52-58 (1995) and Johanson et al. J. Biol. Chem. 270:9459-9471). Thus, this invention also encompasses soluble fusion proteins containing a transporter polypeptide and various portions of the constant regions of heavy or light chains of immunoglobulins of various subclass (IgG, IgM, IgA, IgE). Preferred as immunoglobulin is the constant part of the heavy chain of human IgG, particularly IgG1, where fusion takes place at the hinge region. For some uses it is desirable to remove the Fc after the fusion protein has been used for its intended purpose, for example when the fusion protein is to be used as antigen for immunizations. In a particular embodiment, the Fc part can be removed in a simple way by a cleavage sequence, which is also incorporated and can be cleaved with factor Xa.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al. (1992) Current Protocols in Molecular Biology). Moreover, many expression vectors are commercially available that already encode a fusion moiety (e.g., a GST protein). A transporter-encoding nucleic acid can be cloned into such an expression vector such that the fusion moiety is linked in-frame to transporter.

Another form of fusion protein is one that directly affects transporter functions. Accordingly, a transporter polypeptide is encompassed by the present invention in which one or more of the transporter regions (or parts thereof) has been replaced by heterologous or homologous regions (or parts thereof) from another transporter. Accordingly, various permutations are possible, for example, as discussed above. Thus, chimeric transporters can be formed in which one or more of the native domains or subregions has been duplicated, removed, or replaced by another. This includes but is not limited to substrate binding domains and regions involved in transport.

It is understood however that such regions could be derived from a transporter that has not yet been characterized. Moreover, transporter function can be derived from peptides that contain these functions but are not in a transporter family.

The isolated transporter protein can be purified from cells that naturally express it, especially purified from cells that have been altered to express it (recombinant), or synthesized using known protein synthesis methods.

In one embodiment, the protein is produced by recombinant DNA techniques. For example, a nucleic acid molecule encoding the transporter polypeptide is cloned into an expression vector, the expression vector introduced into a host cell and the protein expressed in the host cell. The protein can then be isolated from the cells by an appropriate purification scheme using standard protein purification techniques.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code, in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretory sequence or a sequence for purification of the mature polypeptide or a pro-protein sequence.

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well-known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd ed., T. E. Creighton, W.H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (1990) Meth. Enzymol. 182: 626-646) and Rattan et al. (1992) Ann. N.Y. Acad. Sci. 663:48-62).

As is also well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods.

Modifications can occur anywhere in a polypeptide, including the peptide backbone, the amino acid side-chains and the amino or carboxyl termini. Blockage of the amino or carboxyl group in a polypeptide, or both, by a covalent modification, is common in naturally-occurring and synthetic polypeptides. For instance, the aminoterminal residue of polypeptides made in E. coli, prior to proteolytic processing, almost invariably will be N-formylmethionine.

The modifications can be a function of how the protein is made. For recombinant polypeptides, for example, the modifications will be determined by the host cell posttranslational modification capacity and the modification signals in the polypeptide amino acid sequence. Accordingly, when glycosylation is desired, a polypeptide should be expressed in a glycosylating host, generally a eukaryotic cell. Insect cells often carry out the same posttranslational glycosylations as mammalian cells and, for this reason, insect cell expression systems have been developed to efficiently express mammalian proteins having native patterns of glycosylation. Similar considerations apply to other modifications.

The same type of modification may be present in the same or varying degree at several sites in a given polypeptide. Also, a given polypeptide may contain more than one type of modification.

Polypeptide Uses

Transporter polypeptides are useful for producing antibodies specific for transporter, regions, or fragments. Regions having a high antigenicity index score are shown in FIGS. 3, 26 and 32.

Transporter polypeptides are useful for biological assays related to transporters. Such assays involve any of the known transporter functions or activities or properties useful for diagnosis and treatment of transporter-related conditions, including those in the references cited herein, which are incorporated by reference for these assays, functions, and disorders.

Substrates also include any in the references cited herein, which are incorporated herein by reference for these substrates. Accordingly the assays include, but are not limited to, these transported substrates and biochemical, cellular, or phenotypic effects of transport. Further, assays may relate to changes in the protein, per se, and on the effects of these changes, for example, activation of the transporter by modification as disclosed herein, induction of expression of the protein in vivo, inhibition of function, as well as any other effects on the protein mentioned herein or cited in any reference herein, which are incorporated herein by reference for these effects and for the subsequent biological consequences of these effects.

Transporter polypeptides are also useful in drug screening assays, in cell-based or cell-free systems. Cell-based systems can be native, i.e., cells that normally express transporter, such as those discussed above, as a biopsy, or expanded in cell culture. In one embodiment, however, cell-based assays involve recombinant host cells expressing transporter. Accordingly, these drug-screening assays can be based on effects on protein function as described above for biological assays useful for diagnosis and treatment.

Determining the ability of the test compound to interact with a transporter can also comprise determining the ability of the test compound to preferentially bind to the polypeptide as compared to the ability of a known binding molecule to bind to the polypeptide.

The polypeptides can be used to identify compounds that modulate transporter activity. Such compounds, for example, can increase or decrease affinity or rate of binding to substrate, compete with substrate for binding to transporter, or displace substrate bound to transporter. Both transporter and appropriate variants and fragments can be used in high-throughput screens to assay candidate compounds for the ability to bind to transporter. These compounds can be further screened against a functional transporter to determine the effect of the compound on transporter activity. Compounds can be identified that activate (agonist) or inactivate (antagonist) transporter to a desired degree. Modulatory methods can be performed in vitro (e.g., by culturing the cell with the agent) or, alternatively, in vivo (e.g., by administering the agent to a subject).

Transporter polypeptides can be used to screen a compound for the ability to stimulate or inhibit interaction between transporter protein and a target molecule that normally interacts with the transporter, for example, substrate of the transporter domain. The assay includes the steps of combining transporter protein with a candidate compound under conditions that allow the transporter protein or fragment to interact with the target molecule, and to detect the formation of a complex between the transporter protein and the target or to detect the biochemical consequence of the interaction with the transporter and the target.

Determining the ability of the transporter to bind to a target molecule can also be accomplished using a technology such as real-time Bimolecular Interaction Analysis (BIA). Sjolander et al. (1991) Anal. Chem. 63:2338-2345 and Szabo et al. (1995) Curr. Opin. Struct. Biol. 5:699-705. As used herein, “BIA” is a technology for studying biospecific interactions in real time, without labeling any of the interactants (e.g., BIAcore™). Changes in the optical phenomenon surface plasmon resonance (SPR) can be used as an indication of real-time reactions between biological molecules.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam, K. S. (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. 97:6378-6382); (Felici (1991) J. Mol. Biol. 222:301-310); (Ladner supra).

Candidate compounds include, for example, 1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries); substrate analogs including, but not limited to, substrates disclosed herein.

One candidate compound is a soluble full-length transporter or fragment that competes for substrate. Other candidate compounds include mutant transporters or appropriate fragments containing mutations that affect transporter function and compete for substrate. Accordingly, a fragment that competes for substrate, for example with a higher affinity, or a fragment that binds substrate but does not process or otherwise affect it, is encompassed by the invention.

The invention provides other end points to identify compounds that modulate (stimulate or inhibit) transporter activity. The assays typically involve an assay of cellular events that indicate transporter activity. Thus, the expression of genes that are up- or down-regulated in response to transporter activity can be assayed. In one embodiment, the regulatory region of such genes can be operably linked to a marker that is easily detectable, such as luciferase. Alternatively, modification of the transporter could also be measured.

Any of the biological or biochemical functions mediated by the transporter can be used as an endpoint assay. These include any of the biochemical or biochemical/biological events described herein, in any reference cited herein, incorporated by reference for these endpoint assay targets, and other functions known to those of ordinary skill in the art. Specific end points can include, but are not limited to, the events resulting from expression (or lack thereof) of transporter activity. With respect to disorders, this would include, but not be limited to, effects on function, differentiation, and proliferation, which can be assayed, as well as the biological effects of function, such as disorders discussed hereinabove and in the references cited hereinabove which are incorporated herein by reference for the disorders disclosed in those references and other disorders and pathology. For example, models of pain, tumor progression, viral infection, bone formation or loss, inflammation, or blood clotting can be used as an end point.

Binding and/or activating compounds can also be screened by using chimeric transporter proteins in which one or more regions, segments, sites, and the like, as disclosed herein, or parts thereof, can be replaced by heterologous and homologous counterparts derived from other transporters. For example, a catalytic region can be used that interacts with a different substrate specificity and/or affinity than the native transporter. Accordingly, a different set of components is available as an end-point assay for activation. As a further alternative, the site of modification by an effector protein, for example, activation or phosphorylation, can be replaced with the site for a different effector protein. Activation can also be detected by a reporter gene containing an easily detectable coding region operably linked to a transcriptional regulatory sequence that is part of the native pathway in which transporter is involved.

Transporter polypeptides are also useful in competition binding assays in methods designed to discover compounds that interact with the transporter. Thus, a compound is exposed to a transporter polypeptide under conditions that allow the compound to bind or to otherwise interact with the polypeptide. Soluble transporter polypeptide is also added to the mixture. If the test compound interacts with the soluble transporter polypeptide, it decreases the amount of complex formed or activity from the transporter target. This type of assay is particularly useful in cases in which compounds are sought that interact with specific regions of the transporter. Thus, the soluble polypeptide that competes with the target transporter region is designed to contain peptide sequences corresponding to the region of interest.

Another type of competition-binding assay can be used to discover compounds that interact with specific functional sites. As an example, bindable substrate analog and a candidate compound can be added to a sample of the transporter. Compounds that interact with the transporter at the same site as the substrate or analog will reduce the amount of complex formed between the transporter and the substrate or analog. Accordingly, it is possible to discover a compound that specifically prevents interaction between the transporter and the component. Another example involves adding a candidate compound to a sample of transporter and transportable substrate. A compound that competes with the substrate will reduce the amount of binding or transport of the substrate to the transporter. Accordingly, compounds can be discovered that directly interact with the transporter and compete with the substrate. Such assays can involve any other component that interacts with the transporter.

To perform cell free drug screening assays, it is desirable to immobilize either transporter, or fragment, or its target molecule to facilitate separation of complexes from uncomplexed forms of one or both of the proteins, as well as to accommodate automation of the assay.

Techniques for immobilizing proteins on matrices can be used in the drug screening assays. In one embodiment, a fusion protein can be provided which adds a domain that allows the protein to be bound to a matrix. For example, glutathione-S-transferase/transporter fusion proteins can be adsorbed onto glutathione sepharose beads (Sigma Chemical, St. Louis, Mo.) or glutathione derivatized microtitre plates, which are then combined with the cell lysates (e.g., ³⁵S-labeled) and the candidate compound, and the mixture incubated under conditions conducive to complex formation (e.g., at physiological conditions for salt and pH). Following incubation, the beads are washed to remove any unbound label, and the matrix immobilized and radiolabel determined directly, or in the supernatant after the complexes is dissociated. Alternatively, the complexes can be dissociated from the matrix, separated by SDS-PAGE, and the level of transporter-binding protein found in the bead fraction quantitated from the gel using standard electrophoretic techniques. For example, either the polypeptide or its target molecule can be immobilized utilizing conjugation of biotin and streptavidin using techniques well known in the art. Alternatively, antibodies reactive with the protein but which do not interfere with binding of the protein to its target molecule can be derivatized to the wells of the plate, and the protein trapped in the wells by antibody conjugation. Preparations of a transporter-binding target component, such as substrate or activating enzyme, and a candidate compound are incubated in transporter-presenting wells and the amount of complex trapped in the well can be quantitated. Methods for detecting such complexes, in addition to those described above for the GST-immobilized complexes, include immunodetection of complexes using antibodies reactive with the transporter target molecule, or which are reactive with the transporter and compete with the target molecule; as well as enzyme-linked assays which rely on detecting an enzymatic activity associated with the target molecule.

Modulators of transporter activity identified according to these drug screening assays can be used to treat a subject with a disorder related to the transporter, by treating cells that express the transporter. These methods of treatment include the steps of administering the modulators of transporter activity in a pharmaceutical composition as described herein, to a subject in need of such treatment.

Various transporters described herein are expressed in tumor cells. Accordingly, these transporters are relevant to these disorders and relevant as well to differentiation, function, and growth of the tissues giving rise to the tumors. Transporters are expressed as described above, and accordingly are relevant for disorders involving these tissues. Disorders include, but are not limited to, those discussed hereinabove.

Transporter polypeptides are thus useful for treating a transporter-associated disorder characterized by aberrant expression or activity of a transporter. In one embodiment, the method involves administering an agent (e.g., an agent identified by a screening assay described herein), or combination of agents that modulates (e.g., upregulates or downregulates) expression or activity of the protein. In another embodiment, the method involves administering transporter as therapy to compensate for reduced or aberrant expression or activity of the protein.

Methods for treatment include but are not limited to the use of soluble transporter or fragments of transporter protein that compete for substrate or any other component that directly interacts with transporter, or any of the enzymes that modify the transporter. These transporters or fragments can have a higher affinity for the target so as to provide effective competition.

Stimulation of activity is desirable in situations in which the protein is abnormally downregulated and/or in which increased activity is likely to have a beneficial effect. Likewise, inhibition of activity is desirable in situations in which the protein is abnormally upregulated and/or in which decreased activity is likely to have a beneficial effect. In one example of such a situation, a subject has a disorder characterized by aberrant development or cellular differentiation. In another example, the subject has a disorder characterized by an aberrant hematopoietic response. In another example, it is desirable to achieve tissue regeneration in a subject.

In yet another aspect of the invention, the proteins of the invention can be used as “bait proteins” in a two-hybrid assay or three-hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Biotechniques 14:920-924; Iwabuchi et al. (1993) Oncogene 8:1693-1696; and Brent WO 94/10300), to identify other proteins (captured proteins) which bind to or interact with the proteins of the invention and modulate their activity.

Transporter polypeptides also are useful to provide a target for diagnosing a disease or predisposition to disease mediated by the transporter, including, but not limited to, those diseases disclosed herein, in the references cited herein, and as disclosed above in the background. Accordingly, methods are provided for detecting the presence, or levels of the transporter in a cell, tissue, or organism. The method involves contacting a biological sample with a compound capable of interacting with the transporter such that the interaction can be detected. One agent for detecting a transporter is an antibody capable of selectively binding to the transporter. A biological sample includes tissues, cells and biological fluids isolated from a subject, as well as tissues, cells and fluids present within a subject.

The transporter also provides a target for diagnosing active disease, or predisposition to disease, in a patient having a variant transporter. Thus, transporter can be isolated from a biological sample and assayed for the presence of a genetic mutation that results in an aberrant protein. This includes amino acid substitution, deletion, insertion, rearrangement, (as the result of aberrant splicing events), and inappropriate post-translational modification. Analytic methods include altered electrophoretic mobility, altered tryptic peptide digest, altered transporter activity in cell-based or cell-free assays, such as by alteration in substrate binding or transport, or ability to be activated, altered antibody-binding pattern, altered isoelectric point, direct amino acid sequencing, and any other of the known assay techniques useful for detecting mutations in a protein in general or in a transporter specifically, such as are disclosed herein.

In vitro techniques for detection of transporter include enzyme linked immunosorbent assays (ELISAs), Western blots, immunoprecipitations and immunofluorescence. Alternatively, the protein can be detected in vivo in a subject by introducing into the subject a labeled anti-transporter antibody. For example, the antibody can be labeled with a radioactive marker whose presence and location in a subject can be detected by standard imaging techniques. Particularly useful are methods, which detect the allelic variant of transporter expressed in a subject, and methods, which detect fragments of transporter in a sample.

Transporter polypeptides are also useful in pharmacogenomic analysis. Pharmacogenomics deal with clinically significant hereditary variations in the response to drugs due to altered drug disposition and abnormal action in affected persons. See, e.g., Eichelbaum, M. (1996) Clin. Exp. Pharmacol. Physiol. 23(10-11):983-985, and Linder, M. W. (1997) Clin. Chem. 43(2):254-266. The clinical outcomes of these variations result in severe toxicity of therapeutic drugs in certain individuals or therapeutic failure of drugs in certain individuals as a result of individual variation in metabolism. Thus, the genotype of the individual can determine the way a therapeutic compound acts on the body or the way the body metabolizes the compound. Further, the activity of drug metabolizing enzymes affects both the intensity and duration of drug action. Thus, the pharmacogenomics of the individual permit the selection of effective compounds and effective dosages of such compounds for prophylactic or therapeutic treatment based on the individual's genotype. The discovery of genetic polymorphisms in some drug metabolizing enzymes has explained why some patients do not obtain the expected drug effects, show an exaggerated drug effect, or experience serious toxicity from standard drug dosages. Polymorphisms can be expressed in the phenotype of the extensive metabolizer and the phenotype of the poor metabolizer. Accordingly, genetic polymorphism may lead to allelic protein variants of transporter in which one or more of transporter functions in one population is different from those in another population. The polypeptides thus allow a target to ascertain a genetic predisposition that can affect treatment modality. Thus, in a peptide-based treatment, polymorphism may give rise to transporter regions that are more or less active. Accordingly, dosage would necessarily be modified to maximize the therapeutic effect within a given population containing the polymorphism. As an alternative to genotyping, specific polymorphic polypeptides could be identified.

Transporter polypeptides are also useful for monitoring therapeutic effects during clinical trials and other treatment. Thus, the therapeutic effectiveness of an agent that is designed to increase or decrease gene expression, protein levels or transporter activity can be monitored over the course of treatment using transporter polypeptides as an end-point target. The monitoring can be, for example, as follows: (i) obtaining a pre-administration sample from a subject prior to administration of the agent; (ii) detecting the level of expression or activity of the protein in the pre-administration sample; (iii) obtaining one or more post-administration samples from the subject; (iv) detecting the level of expression or activity of the protein in the post-administration samples; (v) comparing the level of expression or activity of the protein in the pre-administration sample with the protein in the post-administration sample or samples; and (vi) increasing or decreasing the administration of the agent to the subject accordingly.

Antibodies

The invention also provides antibodies that selectively bind to the transporter and its variants and fragments. An antibody is considered to selectively bind, even if it also binds to other proteins that are not substantially homologous with the transporter. These other proteins share homology with a fragment or domain of transporter. This conservation in specific regions gives rise to antibodies that bind to both proteins by virtue of the homologous sequence. In this case, it would be understood that antibody binding to the transporter is still selective.

Antibodies can be polyclonal or monoclonal. An intact antibody, or a fragment thereof (e.g. Fab or F(ab′)₂) can be used. An appropriate immunogenic preparation can be derived from native, recombinantly expressed, or chemically synthesized peptides.

To generate antibodies, an isolated transporter polypeptide is used as an immunogen to generate antibodies using standard techniques for polyclonal and monoclonal antibody preparation. Either the full-length protein or antigenic peptide fragment can be used. Regions having a high antigenicity index are disclosed hereinabove.

Antibodies are preferably prepared from these regions or from discrete fragments in these regions. However, antibodies can be prepared from any region of the peptide as described herein. A preferred fragment produces an antibody that diminishes or completely prevents substrate transport or binding. Antibodies can be developed against the entire transporter or domains of the transporter as described herein, for example, the substrate binding region, transporter motif, or subregions thereof. Antibodies can also be developed against other specific functional sites as disclosed herein.

The antigenic peptide can comprise a contiguous sequence of at least 12, 14, 15-20, 20-25, or 25-30 or more amino acid residues. In one embodiment, fragments correspond to regions that are located on the surface of the protein, e.g., hydrophilic regions. These fragments are not to be construed, however, as encompassing any fragments, which may be disclosed prior to the invention.

Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Uses

The antibodies can be used to isolate a transporter by standard techniques, such as affinity chromatography or immunoprecipitation. The antibodies can facilitate the purification of the natural transporter from cells and recombinantly produced transporter expressed in host cells.

The antibodies are useful to detect the presence of a transporter in cells or tissues to determine the pattern of expression of the transporter among various tissues in an organism and over the course of normal development. The antibodies can be used to detect a transporter in situ, in vitro, or in a cell lysate or supernatant in order to evaluate the abundance and pattern of expression. Antibody detection of circulating fragments of the full length transporter can be used to identify transporter turnover. In addition, the antibodies can be used to assess abnormal tissue distribution or abnormal expression during development.

Further, the antibodies can be used to assess transporter expression in disease states such as in active stages of the disease or in an individual with a predisposition toward disease related to transporter function. When a disorder is caused by an inappropriate tissue distribution, developmental expression, or level of expression of transporter protein, the antibody can be prepared against the normal transporter protein. If a disorder is characterized by a specific mutation in transporter, antibodies specific for this mutant protein can be used to assay for the presence of the specific mutant transporter. However, intracellularly-made antibodies (“intrabodies”) are also encompassed, which would recognize intracellular transporter peptide regions.

The antibodies can also be used to assess normal and aberrant subcellular localization of cells in the various tissues in an organism. Antibodies can be developed against the whole transporter or portions of the transporter.

The diagnostic uses can be applied, not only in genetic testing, but also in monitoring a treatment modality. Accordingly, where treatment is ultimately aimed at correcting transporter expression level or the presence of aberrant transporters and aberrant tissue distribution or developmental expression, antibodies directed against the transporter or relevant fragments can be used to monitor therapeutic efficacy.

Additionally, antibodies are useful in pharmacogenomic analysis. Thus, antibodies prepared against polymorphic transporter can be used to identify individuals that require modified treatment modalities.

The antibodies are also useful as diagnostic tools as an immunological marker for aberrant transporter analyzed by electrophoretic mobility, isoelectric point, tryptic peptide digest, and other physical assays known to those in the art.

The antibodies are also useful for tissue typing. Thus, where a specific transporter has been correlated with expression in a specific tissue, antibodies that are specific for this transporter can be used to identify a tissue type.

The antibodies are also useful in forensic identification. Accordingly, where an individual has been correlated with a specific genetic polymorphism resulting in a specific polymorphic protein, an antibody specific for the polymorphic protein can be used as an aid in identification.

The antibodies are also useful for inhibiting transporter function, for example, substrate binding, or transport.

These uses can also be applied in a therapeutic context in which treatment involves inhibiting transporter function. An antibody can be used, for example, to block substrate binding. Antibodies can be prepared against specific fragments containing sites required for function or against intact transporter associated with a cell.

Completely human antibodies are particularly desirable for therapeutic treatment of human patients. For an overview of this technology for producing human antibodies, see Lonberg et al. (1995) Int. Rev. Immunol. 13:65-93. For a detailed discussion of this technology for producing human antibodies and human monoclonal antibodies and protocols for producing such antibodies, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806.

The invention also encompasses kits for using antibodies to detect the presence of a transporter protein in a biological sample. The kit can comprise antibodies such as a labeled or labelable antibody and a compound or agent for detecting the transporter in a biological sample; means for determining the amount of transporter in the sample; and means for comparing the amount of transporter in the sample with a standard. The compound or agent can be packaged in a suitable container. The kit can further comprise instructions for using the kit to detect the transporter.

Polynucleotides

The nucleotide sequences in SEQ ID NOS:1, 4, 7, 10, 13, and 16, were obtained by sequencing the deposited human cDNAs. Accordingly, the sequences of the deposited clones are controlling as to any discrepancies between the two and any reference to a sequence of SEQ ID NOS:1, 4, 7, 10, 13, or 16 includes reference to the sequence of the deposited cDNA.

The specifically disclosed cDNA comprises the coding region and 5′ and 3′ untranslated sequences in SEQ ID NOS:1, 4, 7, 10, 13, or 16.

The invention provides isolated polynucleotides encoding the novel transporters. The term “transporter polynucleotide” or “transporter nucleic acid” refers to the sequences shown in SEQ ID NOS:1, 3, 4, 6, 7, 9, 10, 12, 13, 15, 16, or 18, or in the deposited cDNAs. The term “transporter polynucleotide” or “transporter nucleic acid” further includes variants and fragments of transporter polynucleotides.

An “isolated” transporter nucleic acid is one that is separated from other nucleic acid present in the natural source of transporter nucleic acid. Preferably, an “isolated” nucleic acid is free of sequences which naturally flank transporter nucleic acid (i.e., sequences located at the 5′ and 3′ ends of the nucleic acid) in the genomic DNA of the organism from which the nucleic acid is derived. However, there can be some flanking nucleotide sequences, for example up to about 5 KB. The important point is that the transporter nucleic acid is isolated from flanking sequences such that it can be subjected to the specific manipulations described herein, such as recombinant expression, preparation of probes and primers, and other uses specific to the transporter nucleic acid sequences. In one embodiment, the transporter nucleic acid comprises only the coding region.

Moreover, an “isolated” nucleic acid molecule, such as a cDNA or RNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

In some instances, the isolated material will form part of a composition (for example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

In some instances, the isolated material will form part of a composition (or example, a crude extract containing other substances), buffer system or reagent mix. In other circumstances, the material may be purified to essential homogeneity, for example as determined by PAGE or column chromatography such as HPLC. Preferably, an isolated nucleic acid comprises at least about 50, 80 or 90% (on a molar basis) of all macromolecular species present.

Transporter polynucleotides can encode the mature protein plus additional amino or carboxyterminal amino acids, or amino acids interior to the mature polypeptide (when the mature form has more than one polypeptide chain, for instance). Such sequences may play a role in processing of a protein from precursor to a mature form, facilitate protein trafficking, prolong or shorten protein half-life or facilitate manipulation of a protein for assay or production, among other things. As generally is the case in situ, the additional amino acids may be processed away from the mature protein by cellular enzymes.

Transporter polynucleotides include, but are not limited to, the sequence encoding the mature polypeptide alone, the sequence encoding the mature polypeptide and additional coding sequences, such as a leader or secretory sequence (e.g., a pre-pro or pro-protein sequence), the sequence encoding the mature polypeptide, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the polynucleotide may be fused to a marker sequence encoding, for example, a peptide that facilitates purification.

Transporter polynucleotides can be in the form of RNA, such as mRNA, or in the form DNA, including cDNA and genomic DNA obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The invention further provides variant transporter polynucleotides, and fragments thereof, that differ from the nucleotide sequence shown in SEQ ID NOS:1, 3, 4, 6, 7, 9, 10, 12, 13, 15, 16, or 18, due to degeneracy of the genetic code and thus encode the same protein as that encoded by a nucleotide sequence shown in SEQ ID NOS:1, 3, 4, 6, 7, 9, 10, 12, 13, 15, 16, or 18.

The invention also provides transporter nucleic acid molecules encoding the variant polypeptides described herein. Such polynucleotides may be naturally occurring, such as allelic variants (same locus), homologs (different locus), and orthologs (different organism), or may be constructed by recombinant DNA methods or by chemical synthesis. Such non-naturally occurring variants may be made by mutagenesis techniques, including those applied to polynucleotides, cells, or organisms. Accordingly, as discussed above, the variants can contain nucleotide substitutions, deletions, inversions and insertions.

Typically, variants have a substantial identity with nucleic acid molecules of SEQ ID NOS:1, 3, 4, 6, 7, 9, 10, 12, 13, 15, 16, or 18, and the complements thereof. Variation can occur in either or both the coding and non-coding regions. The variations can produce both conservative and non-conservative amino acid substitutions.

Orthologs, homologs, and allelic variants can be identified using methods well known in the art. These variants comprise a nucleotide sequence encoding a transporter that has typically at least about 60%, 65%, 70%, 75%, 80%, 85%, 90%, 91%, 92%, 93%, 94%, 95%, 96%, 97%, 98%, 99% or more identity to the nucleotide sequence shown in SEQ ID NOS:1, 3, 4, 6, 7, 9, 10, 12, 13, 15, 16, or 18, or a fragment of the sequence. Such nucleic acid molecules can readily be identified as being able to hybridize under stringent conditions, to the nucleotide sequence shown in SEQ ID NOS:1, 3, 4, 6, 7, 9, 10, 12, 13, 15, 16, or 18, or a fragment of the sequence.

Nucleic acid molecules that are fragments of the transporter nucleotide sequences are also encompassed by the present invention. By “fragment” is intended a portion of the transporter nucleic acid molecules of the invention. A fragment of a transporter nucleic acid molecule may encode a biologically active portion of a transporter protein, or it may be a fragment that can be used as a hybridization probe or PCR primer using methods disclosed below. A biologically active portion of a transporter protein can be prepared by isolating a portion of one of the transporter nucleotide sequences of the invention, expressing the encoded portion of the transporter polypeptide (e.g., by recombinant expression in vitro), and assessing the activity of the encoded portion of the transporter protein.

A nucleic acid molecule that is a fragment of a transporter-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1734 of SEQ ID NO:1.

A nucleic acid molecule that is a fragment of a transporter-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2100-2200, 2200-2300, 2300-2400, 2400-2500, 2500-2600, 2600-2700, 2700-2800, 2800-2900, 2900-3000, 3000-3103 of SEQ ID NO:4.

A nucleic acid molecule that is a fragment of a transporter-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, 4400-4500, 4500-4600, 4600-4700, 4700-4800, 4800-4900, 4900-5000, 5000-5100, 5100-5200, 5200-5300, 5300-5400, 5400-5500, 5500-5600, 5600-5700, 5700-5800, 5800-5900, 5900-6000, 6000-6100, 6100-6200, 6200-6300, 6300-6400, 6400-6500, 6500-6600, 6600-6700, 6700-6800, 6800-6900, 6900-7000, 7000-7100, 7100-7200, 7200-7300, 7300-7400, 7400-7500, 7500-7600, 7600-7700, 7700-7800, 7800-7900, 7900-8000, 8000-8100, 8100-8195 of SEQ ID NO:7.

A nucleic acid molecule that is a fragment of a transporter-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2100-2150 of SEQ ID NO:10.

A nucleic acid molecule that is a fragment of a transporter-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2100-2200, 2200-2300, 2300-2400, 2400-2500, 2500-2593 of SEQ ID NO:13.

A nucleic acid molecule that is a fragment of a transporter-like nucleotide sequence of the present invention comprises a nucleotide sequence consisting of nucleotides 1-100, 100-200, 200-300, 300-400, 400-500, 500-600, 600-700, 700-800, 800-900, 900-1000, 1000-1100, 1100-1200, 1200-1300, 1300-1400, 1400-1500, 1500-1600, 1600-1700, 1700-1800, 1800-1900, 1900-2000, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3408 of SEQ ID NO:16.

It is understood that stringent hybridization does not indicate substantial homology where it is due to general homology, such as polyA⁺ sequences, or sequences common to all or most proteins, transporters, neurotransmitters, sulfate transporters, ABC transporters, or any of the transporters to which the transporters of the present invention have shown homology, for example, by BLAST analysis. Moreover, it is understood that variants do not include any of the nucleic acid sequences that may have been disclosed prior to the invention.

As used herein, the term “hybridizes under stringent conditions” describes conditions for hybridization and washing. Stringent conditions are known to those skilled in the art and can be found in Current Protocols in Molecular Biology John Wiley & Sons, N.Y. (1989), 6.3.1-6.3.6. Aqueous and nonaqueous methods are described in that reference and either can be used. A preferred, example of stringent hybridization conditions are hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions are hybridization in 6× sodium chloride/sodium citrate (SSC) at about 45° C., followed by one or more washes in 0.2×SSC, 0.1% SDS at 60° C. Preferably, stringent hybridization conditions are hybridization in 6× sodium chloride/sodium citrate (SSC) at about 45° C., followed by one or more washes in 0.2×SSC, 0.1% SDS at 65° C. Particularly preferred stringency conditions (and the conditions that should be used if the practitioner is uncertain about what conditions should be applied to determine if a molecule is within a hybridization limitation of the invention) are 0.5M Sodium Phosphate, 7% SDS at 65° C., followed by one or more washes at 0.2×SSC, 1% SDS at 65° C. Preferably, an isolated nucleic acid molecule of the invention that hybridizes under stringent conditions to the sequence of SEQ ID NOS:1, 4, 7, 10, 13, or 16, or SEQ ID NOS:3, 6, 9, 12, 15, or 18, corresponds to a naturally-occurring nucleic acid molecule.

As used herein, a “naturally-occurring” nucleic acid molecule refers to an RNA or DNA molecule having a nucleotide sequence that occurs in nature (e.g., encodes a natural protein).

The present invention also provides isolated nucleic acids that contain a single or double stranded fragment or portion that hybridizes under stringent conditions to the nucleotide sequence of SEQ ID NOS:1, 3, 4, 6, 7, 9, 10, 12, 13, 15, 16, or 18, or the complements of SEQ ID NOS:1, 3, 4, 6, 7, 9, 10, 12, 13, 15, 16, or 18. In one embodiment, the nucleic acid consists of a portion of a nucleotide sequence of SEQ ID NOS:1, 3, 4, 6, 7, 9, 10, 12, 13, 15, 16, or 18, and the complements. The nucleic acid fragments of the invention are at least about 10-15, preferably at least about 15-20 or 20-25 contiguous nucleotides, and can be 30, 33, 35, 40, 50, 60, 70, 75, 80, 90, 100, 200, 500 or more nucleotides in length. Longer fragments, for example, 600 or more nucleotides in length, which encode antigenic proteins or polypeptides described herein are also useful.

The fragment can comprise DNA or RNA and can be derived from either the coding or the non-coding sequence.

In another embodiment an isolated transporter nucleic acid encodes the entire coding region. In another embodiment the isolated transporter nucleic acid encodes a sequence corresponding to the mature protein. Other fragments include nucleotide sequences encoding the amino acid fragments described herein.

Thus, transporter nucleic acid fragments further include sequences corresponding to the regions described herein, subregions also described, and specific functional sites. Transporter nucleic acid fragments also include combinations of the regions, segments, motifs, and other functional sites described above. It is understood that a transporter fragment includes any nucleic acid sequence that does not include the entire gene. A person of ordinary skill in the art would be aware of the many permutations that are possible. Nucleic acid fragments, according to the present invention, are not to be construed as encompassing those fragments that may have been disclosed prior to the invention.

Where the location of the regions or sites have been predicted by computer analysis, one of ordinary skill would appreciate that the amino acid residues constituting these regions can vary depending on the criteria used to define the regions.

Polynucleotide Uses

The nucleic acid fragments of the invention provide probes or primers in assays such as those described below. “Probes” are oligonucleotides that hybridize in a base-specific manner to a complementary strand of nucleic acid. Such probes include polypeptide nucleic acids, as described in Nielsen et al. (1991) Science 254:1497-1500. Typically, a probe comprises a region of nucleotide sequence that hybridizes under highly stringent conditions to at least about 15, typically about 20-25, and more typically about 30, 40, 50 or 75 consecutive nucleotides of the nucleic acid sequence shown in SEQ ID NO:5 and the complements thereof. More typically, the probe further comprises a label, e.g., radioisotope, fluorescent compound, enzyme, or enzyme co-factor.

As used herein, the term “primer” refers to a single-stranded oligonucleotide which acts as a point of initiation of template-directed DNA synthesis using well-known methods (e.g., PCR, LCR) including, but not limited to those described herein. The appropriate length of the primer depends on the particular use, but typically ranges from about 15 to 30 nucleotides. The term “primer site” refers to the area of the target DNA to which a primer hybridizes. The term “primer pair” refers to a set of primers including a 5′ (upstream) primer that hybridizes with the 5′ end of the nucleic acid sequence to be amplified and a 3′ (downstream) primer that hybridizes with the complement of the sequence to be amplified.

Transporter polynucleotides are thus useful for probes, primers, and in biological assays. Where the polynucleotides are used to assess transporter properties or functions, such as in the assays described herein, all or less than all of the entire cDNA can be useful. Assays specifically directed to transporter functions, such as assessing agonist or antagonist activity, encompass the use of known fragments. Further, diagnostic methods for assessing transporter function can also be practiced with any fragment, including those fragments that may have been known prior to the invention. Similarly, in methods involving treatment of transporter dysfunction, all fragments are encompassed including those, which may have been known in the art.

Transporter polynucleotides are useful as a hybridization probe for cDNA and genomic DNA to isolate a full-length cDNA and genomic clones encoding the polypeptides described in SEQ ID NOS:2, 5, 8, 11, 14, or 17 and to isolate cDNA and genomic clones that correspond to variants producing the same polypeptides shown in SEQ ID NOS:2, 5, 8, 11, 14, or 17, or the other variants described herein. Variants can be isolated from the same tissue and organism from which a polypeptide shown in SEQ ID NOS:2, 5, 8, 11, 14, or 17 was isolated, different tissues from the same organism, or from different organisms. This method is useful for isolating genes and cDNA that are developmentally-controlled and therefore may be expressed in the same tissue or different tissues at different points in the development of an organism.

The probe can correspond to any sequence along the entire length of the gene encoding the transporter polypeptide. Accordingly, it could be derived from 5′ noncoding regions, the coding region, and 3′ noncoding regions.

The nucleic acid probe can be, for example, the full-length cDNA of SEQ ID NOS:1, 4, 7, 10, 13, or 16, or a fragment thereof, such as an oligonucleotide of at least 5, 10, 15, 20, 25, 30, 50, 100, 250 or 500 nucleotides in length and sufficient to specifically hybridize under stringent conditions to mRNA or DNA.

Fragments of the polynucleotides described herein are also useful to synthesize larger fragments or full-length polynucleotides described herein, ribozymes or antisense molecules. For example, a fragment can be hybridized to any portion of an mRNA and a larger or full-length cDNA can be produced.

Antisense nucleic acids of the invention can be designed using the nucleotide sequences of SEQ ID NOS:1, 3, 4, 6, 7, 9, 10, 12, 13, 15, 16, or 18, and constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest).

Additionally, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4:5). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670. PNAs can be further modified, e.g., to enhance their stability, specificity or cellular uptake, by attaching lipophilic or other helper groups to PNA, by the formation of PNA-DNA chimeras, or by the use of liposomes or other techniques of drug delivery known in the art. The synthesis of PNA-DNA chimeras can be performed as described in Hyrup (1996), supra, Finn et al. (1996) Nucleic Acids Res. 24(17):3357-63, Mag et al. (1989) Nucleic Acids Res. 17:5973, and Peterser et al. (1975) Bioorganic Med. Chem. Lett. 5:1119.

The nucleic acid molecules and fragments of the invention can also include other appended groups such as peptides (e.g., for targeting host cell transporters in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/0918) or the blood brain barrier (see, e.g., PCT Publication No. WO 89/10134). In addition, oligonucleotides can be modified with hybridization-triggered cleavage agents (see, e.g., Krol et al. (1988) Bio-Techniques 6:958-976) or intercalating agents (see, e.g., Zon (1988) Pharm Res. 5:539-549).

Transporter polynucleotides are also useful as primers for PCR to amplify any given region of a transporter polynucleotide.

Transporter polynucleotides are also useful for constructing recombinant vectors. Such vectors include expression vectors that express a portion of, or all of, the transporter polypeptides. Vectors also include insertion vectors, used to integrate into another polynucleotide sequence, such as into the cellular genome, to alter in situ expression of transporter genes and gene products. For example, an endogenous transporter coding sequence can be replaced via homologous recombination with all or part of the coding region containing one or more specifically introduced mutations.

Transporter polynucleotides are also useful for expressing antigenic portions of transporter proteins.

Transporter polynucleotides are also useful as probes for determining the chromosomal positions of transporter polynucleotides by means of in situ hybridization methods, such as FISH. (For a review of this technique, see Verma et al. (1988) Human Chromosomes: A Manual of Basic Techniques (Pergamon Press, New York), and PCR mapping of somatic cell hybrids. The mapping of the sequences to chromosomes is an important first step in correlating these sequences with genes associated with disease.

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between a gene and a disease mapped to the same chromosomal region, can then be identified through linkage analysis (co-inheritance of physically adjacent genes), described in, for example, Egeland et al. ((1987) Nature 325:783-787).

Moreover, differences in the DNA sequences between individuals affected and unaffected with a disease associated with a specified gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations, that are visible from chromosome spreads, or detectable using PCR based on that DNA sequence. Ultimately, complete sequencing of genes from several individuals can be performed to confirm the presence of a mutation and to distinguish mutations from polymorphisms.

Transporter polynucleotide probes are also useful to determine patterns of the presence of the gene encoding transporters and their variants with respect to tissue distribution, for example, whether gene duplication has occurred and whether the duplication occurs in all or only a subset of tissues. The genes can be naturally occurring or can have been introduced into a cell, tissue, or organism exogenously.

Transporter polynucleotides are also useful for designing ribozymes corresponding to all, or a part, of the mRNA produced from genes encoding the polynucleotides described herein.

Transporter polynucleotides are also useful for constructing host cells expressing a part, or all, of a transporter polynucleotide or polypeptide.

Transporter polynucleotides are also useful for constructing transgenic animals expressing all, or a part, of a transporter polynucleotide or polypeptide.

Transporter polynucleotides are also useful for making vectors that express part, or all, of a transporter polypeptide.

Transporter polynucleotides are also useful as hybridization probes for determining the level of transporter nucleic acid expression. Accordingly, the probes can be used to detect the presence of, or to determine levels of, transporter nucleic acid in cells, tissues, and in organisms. The nucleic acid whose level is determined can be DNA or RNA. Accordingly, probes corresponding to the polypeptides described herein can be used to assess gene copy number in a given cell, tissue, or organism. This is particularly relevant in cases in which there has been an amplification of a transporter gene.

Alternatively, the probe can be used in an in situ hybridization context to assess the position of extra copies of a transporter gene, as on extrachromosomal elements or as integrated into chromosomes in which the transporter gene is not normally found, for example, as a homogeneously staining region.

These uses are relevant for diagnosis of disorders involving an increase or decrease in transporter expression relative to normal, such as a proliferative disorder, a differentiative or developmental disorder, or a hematopoietic disorder. Disorders in which transporter expression is relevant include, but are not limited to, those disclosed herein above.

Disorders in which transporter expression is relevant include, but are not limited to, those involving cells and tissues in which the gene is expressed, as disclosed herein.

Thus, the present invention provides a method for identifying a disease or disorder associated with aberrant expression or activity of a transporter nucleic acid, in which a test sample is obtained from a subject and nucleic acid (e.g., mRNA, genomic DNA) is detected, wherein the presence of the nucleic acid is diagnostic for a subject having or at risk of developing a disease or disorder associated with aberrant expression or activity of the nucleic acid.

One aspect of the invention relates to diagnostic assays for determining nucleic acid expression as well as activity in the context of a biological sample (e.g., blood, serum, cells, tissue) to determine whether an individual has a disease or disorder, or is at risk of developing a disease or disorder, associated with aberrant nucleic acid expression or activity. Such assays can be used for prognostic or predictive purpose to thereby prophylactically treat an individual prior to the onset of a disorder characterized by or associated with expression or activity of the nucleic acid molecules.

In vitro techniques for detection of mRNA include Northern hybridizations and in situ hybridizations. In vitro techniques for detecting DNA includes Southern hybridizations and in situ hybridization.

Probes can be used as a part of a diagnostic test kit for identifying cells or tissues that express a transporter, such as by measuring the level of a transporter-encoding nucleic acid in a sample of cells from a subject e.g., mRNA or genomic DNA, or determining if the transporter gene has been mutated.

Nucleic acid expression assays are useful for drug screening to identify compounds that modulate transporter nucleic acid expression (e.g., antisense, polypeptides, peptidomimetics, small molecules or other drugs). A cell is contacted with a candidate compound and the expression of mRNA determined. The level of expression of the mRNA in the presence of the candidate compound is compared to the level of expression of the mRNA in the absence of the candidate compound. The candidate compound can then be identified as a modulator of nucleic acid expression based on this comparison and be used, for example to treat a disorder characterized by aberrant nucleic acid expression. The modulator can bind to the nucleic acid or indirectly modulate expression, such as by interacting with other cellular components that affect nucleic acid expression.

Modulatory methods can be performed in vitro (e.g., by culturing the cell with the agent) or, alternatively, in vivo (e.g., by administering the gent to a subject) in patients or in transgenic animals. The invention thus provides a method for identifying a compound that can be used to treat a disorder associated with nucleic acid expression of a transporter gene. The method typically includes assaying the ability of the compound to modulate the expression of the transporter nucleic acid and thus identifying a compound that can be used to treat a disorder characterized by undesired transporter nucleic acid expression.

The assays can be performed in cell-based and cell-free systems. Cell-based assays include cells naturally expressing the transporter nucleic acid or recombinant cells genetically engineered to express specific nucleic acid sequences. Alternatively, candidate compounds can be assayed in vivo in patients or in transgenic animals.

The assay for transporter nucleic acid expression can involve direct assay of nucleic acid levels, such as mRNA levels, or on collateral compounds (such as substrate transport). Further, the expression of genes that are up- or down-regulated in response to transporter activity can also be assayed. In this embodiment the regulatory regions of these genes can be operably linked to a reporter gene such as luciferase.

Thus, modulators of transporter gene expression can be identified in a method wherein a cell is contacted with a candidate compound and the expression of mRNA determined. The level of expression of transporter mRNA in the presence of the candidate compound is compared to the level of expression of transporter mRNA in the absence of the candidate compound. The candidate compound can then be identified as a modulator of nucleic acid expression based on this comparison and be used, for example to treat a disorder characterized by aberrant nucleic acid expression. When expression of mRNA is statistically significantly greater in the presence of the candidate compound than in its absence, the candidate compound is identified as a stimulator of nucleic acid expression. When nucleic acid expression is statistically significantly less in the presence of the candidate compound than in its absence, the candidate compound is identified as an inhibitor of nucleic acid expression.

Accordingly, the invention provides methods of treatment, with the nucleic acid as a target, using a compound identified through drug screening as a gene modulator to modulate transporter nucleic acid expression. Modulation includes both up-regulation (i.e. activation or agonization) or down-regulation (suppression or antagonization) or effects on nucleic acid activity (e.g. when nucleic acid is mutated or improperly modified). Treatment is of disorders characterized by aberrant expression or activity of the nucleic acid.

Alternatively, a modulator for transporter nucleic acid expression can be a small molecule or drug identified using the screening assays described herein as long as the drug or small molecule inhibits transporter nucleic acid expression.

Transporter polynucleotides are also useful for monitoring the effectiveness of modulating compounds on the expression or activity of a transporter gene in clinical trials or in a treatment regimen. Thus, the gene expression pattern can serve as a barometer for the continuing effectiveness of treatment with the compound, particularly with compounds to which a patient can develop resistance. The gene expression pattern can also serve as a marker indicative of a physiological response of the affected cells to the compound. Accordingly, such monitoring would allow either increased administration of the compound or the administration of alternative compounds to which the patient has not become resistant. Similarly, if the level of nucleic acid expression falls below a desirable level, administration of the compound could be commensurately decreased.

Monitoring can be, for example, as follows: (i) obtaining a pre-administration sample from a subject prior to administration of the agent; (ii) detecting the level of expression of a specified mRNA or genomic DNA of the invention in the pre-administration sample; (iii) obtaining one or more post-administration samples from the subject; (iv) detecting the level of expression or activity of the mRNA or genomic DNA in the post-administration samples; (v) comparing the level of expression or activity of the mRNA or genomic DNA in the pre-administration sample with the mRNA or genomic DNA in the post-administration sample or samples; and (vi) increasing or decreasing the administration of the agent to the subject accordingly.

Transporter polynucleotides are also useful in diagnostic assays for qualitative changes in transporter nucleic acid, and particularly in qualitative changes that lead to pathology. The polynucleotides can be used to detect mutations in transporter genes and gene expression products such as mRNA. The polynucleotides can be used as hybridization probes to detect naturally-occurring genetic mutations in a transporter gene and thereby to determine whether a subject with the mutation is at risk for a disorder caused by the mutation. Mutations include deletion, addition, or substitution of one or more nucleotides in the gene, chromosomal rearrangement, such as inversion or transposition, modification of genomic DNA, such as aberrant methylation patterns or changes in gene copy number, such as amplification. Detection of a mutated form of a transporter gene associated with a dysfunction provides a diagnostic tool for an active disease or susceptibility to disease when the disease results from overexpression, underexpression, or altered expression of a transporter.

Mutations in a transporter gene can be detected at the nucleic acid level by a variety of techniques. Genomic DNA can be analyzed directly or can be amplified by using PCR prior to analysis. RNA or cDNA can be used in the same way.

In certain embodiments, detection of the mutation involves the use of a probe/primer in a polymerase chain reaction (PCR) (see, e.g. U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) PNAS 91:360-364), the latter of which can be particularly useful for detecting point mutations in the gene (see Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to a gene under conditions such that hybridization and amplification of the gene (if present) occurs, and detecting the presence or absence of an amplification product, or detecting the size of the amplification product and comparing the length to a control sample. Deletions and insertions can be detected by a change in size of the amplified product compared to the normal genotype. Point mutations can be identified by hybridizing amplified DNA to normal RNA or antisense DNA sequences.

It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well-known to those of skill in the art. These detection schemes are especially useful for the detection of nucleic acid molecules if such molecules are present in very low numbers.

Alternatively, mutations in a transporter gene can be directly identified, for example, by alterations in restriction enzyme digestion patterns determined by gel electrophoresis.

Further, sequence-specific ribozymes (U.S. Pat. No. 5,498,531) can be used to score for the presence of specific mutations by development or loss of a ribozyme cleavage site.

Perfectly matched sequences can be distinguished from mismatched sequences by nuclease cleavage digestion assays or by differences in melting temperature.

Sequence changes at specific locations can also be assessed by nuclease protection assays such as RNase and S1 protection or the chemical cleavage method.

Furthermore, sequence differences between a mutant transporter gene and a wild-type gene can be determined by direct DNA sequencing. A variety of automated sequencing procedures can be utilized when performing the diagnostic assays ((1995) Biotechniques 19:448), including sequencing by mass spectrometry (see, e.g., PCT International Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in the gene include methods in which protection from cleavage agents is used to detect mismatched bases in RNA/RNA or RNA/DNA duplexes (Myers et al. (1985) Science 230:1242); Cotton et al. (1988) PNAS 85:4397; Saleeba et al. (1992) Meth. Enzymol. 217:286-295), electrophoretic mobility of mutant and wild type nucleic acid is compared (Orita et al. (1989) PNAS 86:2766; Cotton et al. (1993) Mutat. Res. 285:125-144; and Hayashi et al. (1992) Genet. Anal. Tech. Appl. 9:73-79), and movement of mutant or wild-type fragments in polyacrylamide gels containing a gradient of denaturant is assayed using denaturing gradient gel electrophoresis (Myers et al. (1985) Nature 313:495). The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility (Keen et al. (1991) Trends Genet. 7:5). Examples of other techniques for detecting point mutations include, selective oligonucleotide hybridization, selective amplification, and selective primer extension.

In other embodiments, genetic mutations can be identified by hybridizing a sample and control nucleic acids, e.g., DNA or RNA, to high density arrays containing hundreds or thousands of oligonucleotide probes (Cronin et al. (1996) Human Mutation 7:244-255; Kozal et al. (1996) Nature Medicine 2:753-759). For example, genetic mutations can be identified in two dimensional arrays containing light-generated DNA probes as described in Cronin et al. supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

Transporter polynucleotides are also useful for testing an individual for a genotype that while not necessarily causing the disease, nevertheless affects the treatment modality. Thus, the polynucleotides can be used to study the relationship between an individual's genotype and the individual's response to a compound used for treatment (pharmacogenomic relationship). In the present case, for example, a mutation in the transporter gene that results in altered affinity for a substrate-related compound could result in an excessive or decreased drug effect with standard concentrations of the compound. Accordingly, the transporter polynucleotides described herein can be used to assess the mutation content of the gene in an individual in order to select an appropriate compound or dosage regimen for treatment.

Thus polynucleotides displaying genetic variations that affect treatment provide a diagnostic target that can be used to tailor treatment in an individual. Accordingly, the production of recombinant cells and animals containing these polymorphisms allow effective clinical design of treatment compounds and dosage regimens.

The methods can involve obtaining a control biological sample from a control subject, contacting the control sample with a compound or agent capable of detecting mRNA, or genomic DNA, such that the presence of mRNA or genomic DNA is detected in the biological sample, and comparing the presence of mRNA or genomic DNA in the control sample with the presence of mRNA or genomic DNA in the test sample.

Transporter polynucleotides are also useful for chromosome identification when the sequence is identified with an individual chromosome and to a particular location on the chromosome. First, the DNA sequence is matched to the chromosome by in situ or other chromosome-specific hybridization. Sequences can also be correlated to specific chromosomes by preparing PCR primers that can be used for PCR screening of somatic cell hybrids containing individual chromosomes from the desired species. Only hybrids containing the chromosome containing the gene homologous to the primer will yield an amplified fragment. Sublocalization can be achieved using chromosomal fragments. Other strategies include prescreening with labeled flow-sorted chromosomes and preselection by hybridization to chromosome-specific libraries. Further mapping strategies include fluorescence in situ hybridization, which allows hybridization with probes shorter than those traditionally used. Reagents for chromosome mapping can be used individually to mark a single chromosome or a single site on the chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Transporter polynucleotides can also be used to identify individuals from small biological samples. This can be done for example using restriction fragment-length polymorphism (RFLP) to identify an individual. Thus, the polynucleotides described herein are useful as DNA markers for RFLP (See U.S. Pat. No. 5,272,057).

Furthermore, the transporter sequences can be used to provide an alternative technique, which determines the actual DNA sequence of selected fragments in the genome of an individual. Thus, the transporter sequences described herein can be used to prepare two PCR primers from the 5′ and 3′ ends of the sequences. These primers can then be used to amplify DNA from an individual for subsequent sequencing.

Panels of corresponding DNA sequences from individuals prepared in this manner can provide unique individual identifications, as each individual will have a unique set of such DNA sequences. It is estimated that allelic variation in humans occurs with a frequency of about once per each 500 bases. Allelic variation occurs to some degree in the coding regions of these sequences, and to a greater degree in the noncoding regions. Transporter sequences can be used to obtain such identification sequences from individuals and from tissue. The sequences represent unique fragments of the human genome. Each of the sequences described herein can, to some degree, be used as a standard against which DNA from an individual can be compared for identification purposes.

If a panel of reagents from the sequences is used to generate a unique identification database for an individual, those same reagents can later be used to identify tissue from that individual. Using the unique identification database, positive identification of the individual, living or dead, can be made from extremely small tissue samples.

Transporter polynucleotides can also be used in forensic identification procedures. PCR technology can be used to amplify DNA sequences taken from very small biological samples, such as a single hair follicle, body fluids (e.g. blood, saliva, or semen). The amplified sequence can then be compared to a standard allowing identification of the origin of the sample.

Transporter polynucleotides can thus be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As described above, actual base sequence information can be used for identification as an accurate alternative to patterns formed by restriction enzyme generated fragments. Sequences targeted to the noncoding region are particularly useful since greater polymorphism occurs in the noncoding regions, making it easier to differentiate individuals using this technique.

Transporter polynucleotides can further be used to provide polynucleotide reagents, e.g., labeled or labelable probes which can be used in, for example, an in situ hybridization technique, to identify a specific tissue. This is useful in cases in which a forensic pathologist is presented with a tissue of unknown origin. Panels of transporter probes can be used to identify tissue by species and/or by organ type.

In a similar fashion, these primers and probes can be used to screen tissue culture for contamination (i.e. screen for the presence of a mixture of different types of cells in a culture).

Alternatively, transporter polynucleotides can be used directly to block transcription or translation of transporter gene sequences by means of antisense or ribozyme constructs. Thus, in a disorder characterized by abnormally high or undesirable transporter gene expression, nucleic acids can be directly used for treatment.

Transporter polynucleotides are thus useful as antisense constructs to control transporter gene expression in cells, tissues, and organisms. A DNA antisense polynucleotide is designed to be complementary to a region of the gene involved in transcription, preventing transcription and hence production of transporter protein. An antisense RNA or DNA polynucleotide would hybridize to the mRNA and thus block translation of mRNA into transporter protein.

Examples of antisense molecules useful to inhibit nucleic acid expression include antisense molecules complementary to a fragment of the 5′ untranslated region of SEQ ID NOS:1, 4, 7, 10, 13, or 16, which also includes the start codon and antisense molecules which are complementary to a fragment of the 3′ untranslated region of SEQ ID NOS:1, 4, 7, 10, 13, or 16.

Alternatively, a class of antisense molecules can be used to inactivate mRNA in order to decrease expression of transporter nucleic acid. Accordingly, these molecules can treat a disorder characterized by abnormal or undesired transporter nucleic acid expression. This technique involves cleavage by means of ribozymes containing nucleotide sequences complementary to one or more regions in the mRNA that attenuate the ability of the mRNA to be translated. Possible regions include coding regions and particularly coding regions corresponding to the catalytic and other functional activities of the transporter protein.

Transporter polynucleotides also provide vectors for gene therapy in patients containing cells that are aberrant in transporter gene expression. Thus, recombinant cells, which include the patient's cells that have been engineered ex vivo and returned to the patient, are introduced into an individual where the cells produce the desired transporter protein to treat the individual.

The invention also encompasses kits for detecting the presence of a transporter nucleic acid in a biological sample. For example, the kit can comprise reagents such as a labeled or labelable nucleic acid or agent capable of detecting transporter nucleic acid in a biological sample; means for determining the amount of transporter nucleic acid in the sample; and means for comparing the amount of transporter nucleic acid in the sample with a standard. The compound or agent can be packaged in a suitable container. The kit can further comprise instructions for using the kit to detect transporter mRNA or DNA.

Computer Readable Means

The nucleotide or amino acid sequences of the invention are also provided in a variety of mediums to facilitate use thereof. As used herein, “provided” refers to a manufacture, other than an isolated nucleic acid or amino acid molecule, which contains a nucleotide or amino acid sequence of the present invention. Such a manufacture provides the nucleotide or amino acid sequences, or a subset thereof (e.g., a subset of open reading frames (ORFs)) in a form which allows a skilled artisan to examine the manufacture using means not directly applicable to examining the nucleotide or amino acid sequences, or a subset thereof, as they exists in nature or in purified form.

In one application of this embodiment, a nucleotide or amino acid sequence of the present invention can be recorded on computer readable media. As used herein, “computer readable media” refers to any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. The skilled artisan will readily appreciate how any of the presently known computer readable mediums can be used to create a manufacture comprising computer readable medium having recorded thereon a nucleotide or amino acid sequence of the present invention.

As used herein, “recorded” refers to a process for storing information on computer readable medium. The skilled artisan can readily adopt any of the presently known methods for recording information on computer readable medium to generate manufactures comprising the nucleotide or amino acid sequence information of the present invention.

A variety of data storage structures are available to a skilled artisan for creating a computer readable medium having recorded thereon a nucleotide or amino acid sequence of the present invention. The choice of the data storage structure will generally be based on the means chosen to access the stored information. In addition, a variety of data processor programs and formats can be used to store the nucleotide sequence information of the present invention on computer readable medium. The sequence information can be represented in a word processing text file, formatted in commercially-available software such as WordPerfect and Microsoft Word, or represented in the form of an ASCII file, stored in a database application, such as DB2, Sybase, Oracle, or the like. The skilled artisan can readily adapt any number of dataprocessor structuring formats (e.g., text file or database) in order to obtain computer readable medium having recorded thereon the nucleotide sequence information of the present invention.

By providing the nucleotide or amino acid sequences of the invention in computer readable form, the skilled artisan can routinely access the sequence information for a variety of purposes. For example, one skilled in the art can use the nucleotide or amino acid sequences of the invention in computer readable form to compare a target sequence or target structural motif with the sequence information stored within the data storage means. Search means are used to identify fragments or regions of the sequences of the invention which match a particular target sequence or target motif.

As used herein, a “target sequence” can be any DNA or amino acid sequence of six or more nucleotides or two or more amino acids. A skilled artisan can readily recognize that the longer a target sequence is, the less likely a target sequence will be present as a random occurrence in the database. The most preferred sequence length of a target sequence is from about 10 to 100 amino acids or from about 30 to 300 nucleotide residues. However, it is well recognized that commercially important fragments, such as sequence fragments involved in gene expression and protein processing, may be of shorter length.

As used herein, “a target structural motif,” or “target motif,” refers to any rationally selected sequence or combination of sequences in which the sequence(s) are chosen based on a three-dimensional configuration which is formed upon the folding of the target motif. There are a variety of target motifs known in the art. Protein target motifs include, but are not limited to, enzyme active sites and signal sequences. Nucleic acid target motifs include, but are not limited to, promoter sequences, hairpin structures and inducible expression elements (protein binding sequences).

Computer software is publicly available which allows a skilled artisan to access sequence information provided in a computer readable medium for analysis and comparison to other sequences. A variety of known algorithms are disclosed publicly and a variety of commercially available software for conducting search means are and can be used in the computer-based systems of the present invention. Examples of such software includes, but is not limited to, MacPattern (EMBL), BLASTN and BLASTX (NCBIA).

For example, software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) of the sequences of the invention which contain homology to ORFs or proteins from other libraries. Such ORFs are protein encoding fragments and are useful in producing commercially important proteins such as enzymes used in various reactions and in the production of commercially useful metabolites.

Vectors/Host Cells

The invention also provides vectors containing transporter polynucleotides. The term “vector” refers to a vehicle, preferably a nucleic acid molecule that can transport transporter polynucleotides. When the vector is a nucleic acid molecule, the transporter polynucleotides are covalently linked to the vector nucleic acid. With this aspect of the invention, the vector includes a plasmid, single or double stranded phage, a single or double stranded RNA or DNA viral vector, or artificial chromosome, such as a BAC, PAC, YAC, OR MAC.

A vector can be maintained in the host cell as an extrachromosomal element where it replicates and produces additional copies of transporter polynucleotides. Alternatively, the vector may integrate into the host cell genome and produce additional copies of transporter polynucleotides when the host cell replicates.

The invention provides vectors for the maintenance (cloning vectors) or vectors for expression (expression vectors) of transporter polynucleotides. The vectors can function in procaryotic or eukaryotic cells or in both (shuttle vectors).

Expression vectors contain cis-acting regulatory regions that are operably linked in the vector to transporter polynucleotides such that transcription of the polynucleotides is allowed in a host cell. The polynucleotides can be introduced into the host cell with a separate polynucleotide capable of affecting transcription. Thus, the second polynucleotide may provide a trans-acting factor interacting with the cis-regulatory control region to allow transcription of transporter polynucleotides from the vector. Alternatively, a trans-acting factor may be supplied by the host cell. Finally, a trans-acting factor can be produced from the vector itself.

It is understood, however, that in some embodiments, transcription and/or translation of transporter polynucleotides can occur in a cell-free system.

The regulatory sequence to which the polynucleotides described herein can be operably linked include promoters for directing mRNA transcription. These include, but are not limited to, the left promoter from bacteriophage λ, the lac, TRP, and TAC promoters from E. Coli, the early and late promoters from SV40, the CMV immediate early promoter, the adenovirus early and late promoters, and retrovirus long-terminal repeats.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, and retrovirus LTR enhancers.

In addition to containing sites for transcription initiation and control, expression vectors can also contain sequences necessary for transcription termination and, in the transcribed region a ribosome binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al. (1989) Molecular Cloning: A Laboratory Manual 2nd. ed., Cold Spring Harbor Laboratory Press, Cold Spring Harbor, N.Y.).

A variety of expression vectors can be used to express a transporter polynucleotide. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g. cosmids and phagemids. Appropriate cloning and expression vectors for prokaryotic and eukaryotic hosts are described in Sambrook et al. (1989) Molecular Cloning: A Laboratory Manual 2nd. ed., Cold Spring Harbor Laboratory Press, Cold Spring Harbor, N.Y.

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are well known to those of ordinary skill in the art.

Transporter polynucleotides can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are well known to those of ordinary skill in the art.

The vector containing the appropriate polynucleotide can be introduced into an appropriate host cell for propagation or expression using well-known techniques. Bacterial cells include, but are not limited to, E. Coli, Streptomyces, and Salmonella typhimurium. Eukaryotic cells include, but are not limited to, yeast, insect cells such as Drosophila, animal cells such as COS and CHO cells, and plant cells.

It is further recognized that the nucleic acid sequences of the invention can be altered to contain codons, which are preferred, or non preferred, for a particular expression system. For example, the nucleic acid can be one in which at least one altered codon, and preferably at least 10%, or 20% of the codons have been altered such that the sequence is optimized for expression in E. coli, yeast, human, insect, or CHO cells. Methods for determining such codon usage are well known in the art.

As described herein, it may be desirable to express the polypeptide as a fusion protein. Accordingly, the invention provides fusion vectors that allow for the production of transporter polypeptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired polypeptide can ultimately be separated from the fusion moiety. Proteolytic enzymes include, but are not limited to, factor Xa, thrombin, and enterokinase. Typical fusion expression vectors include pGEX (Smith et al. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. Coli expression vectors include pTrc (Amann et al. (1988) Gene 69:301-315) and pET 11d (Studier et al. (1990) Gene Expression Technology: Methods in Enzymology 185:60-89).

Recombinant protein expression can be maximized in a host bacteria by providing a genetic background wherein the host cell has an impaired capacity to proteolytically cleave the recombinant protein. (Gottesman, S. (1990) Gene Expression Technology: Methods in Enzymology 185, Academic Press, San Diego, Calif. 119-128). Alternatively, the sequence of the polynucleotide of interest can be altered to provide preferential codon usage for a specific host cell, for example E. Coli. (Wada et al. (1992) Nucleic Acids Res. 20:2111-2118).

Transporter polynucleotides can also be expressed by expression vectors that are operative in yeast. Examples of vectors for expression in yeast e.g., S. cerevisiae include pYepSec1 (Baldari et al. (1987) EMBO J. 6:229-234), pMFa (Kurjan et al. (1982) Cell 30:933-943), pJRY88 (Schultz et al. (1987) Gene 54:113-123), and pYES2 (Invitrogen Corporation, San Diego, Calif.).

Transporter polynucleotides can also be expressed in insect cells using, for example, baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow et al. (1989) Virology 170:31-39).

In certain embodiments of the invention, the polynucleotides described herein are expressed in mammalian cells using mammalian expression vectors. Examples of mammalian expression vectors include pCDM8 (Seed, B. (1987) Nature 329:840) and pMT2PC (Kaufman et al. (1987) EMBO J. 6:187-195).

The expression vectors listed herein are provided by way of example only of the well-known vectors available to those of ordinary skill in the art that would be useful to express transporter polynucleotides. The person of ordinary skill in the art would be aware of other vectors suitable for maintenance propagation or expression of the polynucleotides described herein. These are found for example in Sambrook et al. (1989) Molecular Cloning: A Laboratory Manual 2nd, ed., Cold Spring Harbor Laboratory, Cold Spring Harbor Laboratory Press, Cold Spring Harbor, N.Y.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the polynucleotide sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include prokaryotic cells, lower eukaryotic cells such as yeast, other eukaryotic cells such as insect cells, and higher eukaryotic cells such as mammalian cells.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook et al. (Molecular Cloning: A Laboratory Manual, 2d ed., Cold Spring Harbor Laboratory, Cold Spring Harbor Laboratory Press, Cold Spring Harbor, N.Y.).

Host cells can contain more than one vector. Thus, different nucleotide sequences can be introduced on different vectors of the same cell. Similarly, transporter polynucleotides can be introduced either alone or with other polynucleotides that are not related to transporter polynucleotides such as those providing trans-acting factors for expression vectors. When more than one vector is introduced into a cell, the vectors can be introduced independently, co-introduced or joined to the transporter polynucleotide vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. The marker can be contained in the same vector that contains the polynucleotides described herein or may be on a separate vector. Markers include tetracycline or ampicillin-resistance genes for prokaryotic host cells and dihydrofolate reductase or neomycin resistance for eukaryotic host cells. However, any marker that provides selection for a phenotypic trait will be effective.

While the mature proteins can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the polypeptide is desired, appropriate secretion signals are incorporated into the vector. The signal sequence can be endogenous to the transporter polypeptides or heterologous to these polypeptides.

Where the polypeptide is not secreted into the medium, the protein can be isolated from the host cell by standard disruption procedures, including freeze thaw, sonication, mechanical disruption, use of lysing agents and the like. The polypeptide can then be recovered and purified by well-known purification methods including ammonium sulfate precipitation, acid extraction, anion or cationic exchange chromatography, phosphocellulose chromatography, hydrophobic-interaction chromatography, affinity chromatography, hydroxylapatite chromatography, lectin chromatography, or high performance liquid chromatography.

It is also understood that depending upon the host cell in recombinant production of the polypeptides described herein, the polypeptides can have various glycosylation patterns, depending upon the cell, or maybe non-glycosylated as when produced in bacteria. In addition, the polypeptides may include an initial modified methionine in some cases as a result of a host-mediated process.

Uses of Vectors and Host Cells

It is understood that “host cells” and “recombinant host cells” refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A “purified preparation of cells”, as used herein, refers to, in the case of plant or animal cells, an in vitro preparation of cells and not an entire intact plant or animal. In the case of cultured cells or microbial cells, it consists of a preparation of at least 10% and more preferably 50% of the subject cells.

The host cells expressing the polypeptides described herein, and particularly recombinant host cells, have a variety of uses. First, the cells are useful for producing transporter proteins or polypeptides that can be further purified to produce desired amounts of transporter protein or fragments. Thus, host cells containing expression vectors are useful for polypeptide production.

Host cells are also useful for conducting cell-based assays involving transporter or transporter fragments. Thus, a recombinant host cell expressing a native transporter is useful to assay for compounds that stimulate or inhibit transporter function, gene expression at the level of transcription or translation, and interaction with other cellular components.

Host cells are also useful for identifying transporter mutants in which these functions are affected. If the mutants naturally occur and give rise to a pathology, host cells containing the mutations are useful to assay compounds that have a desired effect on the mutant transporter (for example, stimulating or inhibiting function) which may not be indicated by their effect on the native transporter.

Recombinant host cells are also useful for expressing the chimeric polypeptides described herein to assess compounds that activate or suppress activation by means of a heterologous domain, segment, site, and the like, as disclosed herein.

Further, mutant transporters can be designed in which one or more of the various functions is engineered to be increased or decreased and used to augment or replace transporter proteins in an individual. Thus, host cells can provide a therapeutic benefit by replacing an aberrant transporter or providing an aberrant transporter that provides a therapeutic result. In one embodiment, the cells provide transporters that are abnormally active.

In another embodiment, the cells provide transporters that are abnormally inactive. These transporters can compete with endogenous transporters in the individual.

In another embodiment, cells expressing transporters that cannot be activated, are introduced into an individual in order to compete with endogenous transporters for substrate. For example, in the case in which excessive substrate or substrate analog is part of a treatment modality, it may be necessary to effectively inactivate the substrate or substrate analog at a specific point in treatment. Providing cells that compete for the molecule, but which cannot be affected by transporter activation would be beneficial.

Homologously recombinant host cells can also be produced that allow the in situ alteration of endogenous transporter polynucleotide sequences in a host cell genome. The host cell includes, but is not limited to, a stable cell line, cell in vivo, or cloned microorganism. This technology is more fully described in WO 93/09222, WO 91/12650, WO 91/06667, U.S. Pat. No. 5,272,071, and U.S. Pat. No. 5,641,670. Briefly, specific polynucleotide sequences corresponding to the transporter polynucleotides or sequences proximal or distal to a transporter gene are allowed to integrate into a host cell genome by homologous recombination where expression of the gene can be affected. In one embodiment, regulatory sequences are introduced that either increase or decrease expression of an endogenous sequence. Accordingly, a transporter protein can be produced in a cell not normally producing it. Alternatively, increased expression of transporter protein can be effected in a cell normally producing the protein at a specific level. Further, expression can be decreased or eliminated by introducing a specific regulatory sequence. The regulatory sequence can be heterologous to the transporter protein sequence or can be a homologous sequence with a desired mutation that affects expression. Alternatively, the entire gene can be deleted. The regulatory sequence can be specific to the host cell or capable of functioning in more than one cell type. Still further, specific mutations can be introduced into any desired region of the gene to produce mutant transporter proteins. Such mutations could be introduced, for example, into the specific functional regions such as the peptide substrate-binding site.

In one embodiment, the host cell can be a fertilized oocyte or embryonic stem cell that can be used to produce a transgenic animal containing the altered transporter gene. Alternatively, the host cell can be a stem cell or other early tissue precursor that gives rise to a specific subset of cells and can be used to produce transgenic tissues in an animal. See also Thomas et al., Cell 51:503 (1987) for a description of homologous recombination vectors. The vector is introduced into an embryonic stem cell line (e.g., by electroporation) and cells in which the introduced gene has homologously recombined with the endogenous transporter gene is selected (see e.g., Li, E. et al. (1992) Cell 69:915). The selected cells are then injected into a blastocyst of an animal (e.g., a mouse) to form aggregation chimeras (see e.g., Bradley, A. in Teratocarcinomas and Embryonic Stem Cells: A Practical Approach, E. J. Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley, A. (1991) Current Opinions in Biotechnology 2:823-829 and in PCT International Publication Nos. WO 90/11354; WO 91/01140; and WO 93/04169.

The genetically engineered host cells can be used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of a transporter protein and identifying and evaluating modulators of transporter protein activity.

Other examples of transgenic animals include non-human primates, sheep, dogs, cows, goats, chickens, and amphibians.

In one embodiment, a host cell is a fertilized oocyte or an embryonic stem cell into which transporter polynucleotide sequences have been introduced.

A transgenic animal can be produced by introducing nucleic acid into the male pronuclei of a fertilized oocyte, e.g., by microinjection, retroviral infection, and allowing the oocyte to develop in a pseudopregnant female foster animal. Any of the transporter nucleotide sequences can be introduced as a transgene into the genome of a non-human animal, such as a mouse.

Any of the regulatory or other sequences useful in expression vectors can form part of the transgenic sequence. This includes intronic sequences and polyadenylation signals, if not already included. A tissue-specific regulatory sequence(s) can be operably linked to the transgene to direct expression of the transporter protein to particular cells.

Methods for generating transgenic animals via embryo manipulation and microinjection, particularly animals such as mice, have become conventional in the art and are described, for example, in U.S. Pat. Nos. 4,736,866 and 4,870,009, both by Leder et al., U.S. Pat. No. 4,873,191 by Wagner et al. and in Hogan, B., Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of transgenic mRNA in tissues or cells of the animals. A transgenic founder animal can then be used to breed additional animals carrying the transgene. Moreover, transgenic animals carrying a transgene can further be bred to other transgenic animals carrying other transgenes. A transgenic animal also includes animals in which the entire animal or tissues in the animal have been produced using the homologously recombinant host cells described herein.

In another embodiment, transgenic non-human animals can be produced which contain selected systems, which allow for regulated expression of the transgene. One example of such a system is the cre/loxP recombinase system of bacteriophage P1. For a description of the cre/loxP recombinase system, see, e.g., Lakso et al. (1992) PNAS 89:6232-6236. Another example of a recombinase system is the FLP recombinase system of S. cerevisiae (O'Gorman et al. (1991) Science 251:1351-1355. If a cre/loxP recombinase system is used to regulate expression of the transgene, animals containing transgenes encoding both the Cre recombinase and a selected protein is required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT International Publication Nos. WO 97/07668 and WO 97/07669. In brief, a cell, e.g., a somatic cell, from the transgenic animal can be isolated and induced to exit the growth cycle and enter Gₒ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyst and then transferred to a pseudopregnant female foster animal. The offspring born of this female animal will be a clone of the animal from which the cell, e.g., the somatic cell, is isolated.

Transgenic animals containing recombinant cells that express the polypeptides described herein are useful to conduct the assays described herein in an in vivo context. Accordingly, the various physiological factors that are present in vivo and that could affect binding or activation, may not be evident from in vitro cell-free or cell-based assays. Accordingly, it is useful to provide non-human transgenic animals to assay in vivo transporter function, including peptide interaction, the effect of specific mutant transporters on transporter function and peptide interaction, and the effect of chimeric transporters. It is also possible to assess the effect of null mutations, that is mutations that substantially or completely eliminate one or more transporter functions.

In general, methods for producing transgenic animals include introducing a nucleic acid sequence according to the present invention, the nucleic acid sequence capable of expressing the protein in a transgenic animal, into a cell in culture or in vivo. When introduced in vivo, the nucleic acid is introduced into an intact organism such that one or more cell types and, accordingly, one or more tissue types, express the nucleic acid encoding the protein. Alternatively, the nucleic acid can be introduced into virtually all cells in an organism by transfecting a cell in culture, such as an embryonic stem cell, as described herein for the production of transgenic animals, and this cell can be used to produce an entire transgenic organism. As described, in a further embodiment, the host cell can be a fertilized oocyte. Such cells are then allowed to develop in a female foster animal to produce the transgenic organism.

Pharmaceutical Compositions

Transporter nucleic acid molecules, proteins, modulators of the protein, and antibodies (also referred to herein as “active compounds”) can be incorporated into pharmaceutical compositions suitable for administration to a subject, e.g., a human. Such compositions typically comprise the nucleic acid molecule, protein, modulator, or antibody and a pharmaceutically acceptable carrier.

The term “administer” is used in its broadest sense and includes any method of introducing the compositions of the present invention into a subject. This includes producing polypeptides or polynucleotides in vivo by in vivo transcription or translation of polynucleotides that have been exogenously introduced into a subject. Thus, polypeptides or nucleic acids produced in the subject from the exogenous compositions are encompassed in the term “administer.”

As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions. A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a transporter protein or anti-transporter antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. “Dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

As defined herein, a therapeutically effective amount of protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The pharmaceutical compositions can be included in a container, pack, or dispenser together with instructions for administration.

This invention may be embodied in many different forms and should not be construed as limited to the embodiments set forth herein; rather, these embodiments are provided so that this disclosure will fully convey the invention to those skilled in the art. Many modifications and other embodiments of the invention will come to mind in one skilled in the art to which this invention pertains having the benefit of the teachings presented in the foregoing description. Although specific terms are employed, they are used as in the art unless otherwise indicated.

### Other Embodiments

In another aspect, the invention features, a method of analyzing a plurality of capture probes. The method can be used, e.g., to analyze gene expression. The method includes: providing a two dimensional array having a plurality of addresses, each address of the plurality being positionally distinguishable from each other address of the plurality, and each address of the plurality having a unique capture probe, e.g., a nucleic acid or peptide sequence; contacting the array with a transporter, preferably purified, nucleic acid, preferably purified, polypeptide, preferably purified, or antibody, and thereby evaluating the plurality of capture probes. Binding, e.g., in the case of a nucleic acid, hybridization with a capture probe at an address of the plurality, is detected, e.g., by signal generated from a label attached to the transporter nucleic acid, polypeptide, or antibody.

The capture probes can be a set of nucleic acids from a selected sample, e.g., a sample of nucleic acids derived from a control or non-stimulated tissue or cell.

The method can include contacting the transporter nucleic acid, polypeptide, or antibody with a first array having a plurality of capture probes and a second array having a different plurality of capture probes. The results of each hybridization can be compared, e.g., to analyze differences in expression between a first and second sample. The first plurality of capture probes can be from a control sample, e.g., a wild type, normal, or non-diseased, non-stimulated, sample, e.g., a biological fluid, tissue, or cell sample. The second plurality of capture probes can be from an experimental sample, e.g., a mutant type, at risk, disease-state or disorder-state, or stimulated, sample, e.g., a biological fluid, tissue, or cell sample.

The plurality of capture probes can be a plurality of nucleic acid probes each of which specifically hybridizes, with an allele of a transporter of the invention. Such methods can be used to diagnose a subject, e.g., to evaluate risk for a disease or disorder, to evaluate suitability of a selected treatment for a subject, to evaluate whether a subject has a disease or disorder. The transporter molecules of the invention are associated with transporter activity, thus they are useful for disorders associated with abnormal transport of molecules across cell membranes.

The method can be used to detect SNPs, as described above.

In another aspect, the invention features, a method of analyzing a plurality of probes. The method is useful, e.g., for analyzing gene expression. The method includes: providing a two dimensional array having a plurality of addresses, each address of the plurality being positionally distinguishable from each other address of the plurality having a unique capture probe, e.g., wherein the capture probes are from a cell or subject which express or misexpress a transporter of the invention or from a cell or subject in which a transporter-mediated response has been elicited, e.g., by contact of the cell with transporter nucleic acid or protein, or administration to the cell or subject transporter nucleic acid or protein; contacting the array with one or more inquiry probe, wherein an inquiry probe can be a nucleic acid, polypeptide, or antibody (which is preferably other than transporter nucleic acid, polypeptide, or antibody); providing a two dimensional array having a plurality of addresses, each address of the plurality being positionally distinguishable from each other address of the plurality, and each address of the plurality having a unique capture probe, e.g., wherein the capture probes are from a cell or subject which does not express transporter (or does not express as highly as in the case of the transporter positive plurality of capture probes) or from a cell or subject which in which a transporter mediated response has not been elicited (or has been elicited to a lesser extent than in the first sample); contacting the array with one or more inquiry probes (which is preferably other than a transporter nucleic acid, polypeptide, or antibody), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

In another aspect, the invention features, a method of analyzing transporters of the invention, e.g., analyzing structure, function, or relatedness to other nucleic acid or amino acid sequences. The method includes: providing a transporter nucleic acid or amino acid sequence; comparing the transporter sequence with one or more preferably a plurality of sequences from a collection of sequences, e.g., a nucleic acid or protein sequence database; to thereby analyze the transporter.

Preferred databases include GenBank™. The method can include evaluating the sequence identity between a transporter sequence and a database sequence. The method can be performed by accessing the database at a second site, e.g., over the internet.

In another aspect, the invention features, a set of oligonucleotides, useful, e.g., for identifying SNP's, or identifying specific alleles of a transporter. The set includes a plurality of oligonucleotides, each of which has a different nucleotide at an interrogation position, e.g., an SNP or the site of a mutation. In a preferred embodiment, the oligonucleotides of the plurality are identical in sequence with one another (except for differences in length). The oligonucleotides can be provided with different labels, such that an oligonucleotide that hybridizes to one allele provides a signal that is distinguishable from an oligonucleotide which hybridizes to a second allele.

This invention is further illustrated by the following examples which should not be construed as limiting. The contents of all references, patents and published patent applications cited throughout this application are incorporated herein by reference.

### EXAMPLES

### Example 1

### Identification and Characterization of Human Transporter cDNAs

The human transporter sequence (FIG. 1A-B; SEQ ID NO:1), which is approximately 1734 nucleotides long including untranslated regions, contains a predicted methionine-initiated coding sequence of about 7308 nucleotides (nucleotides 96-1463 of SEQ ID NO:1; SEQ ID NO:3). The coding sequence encodes a 456 amino acid protein (SEQ ID NO:2).

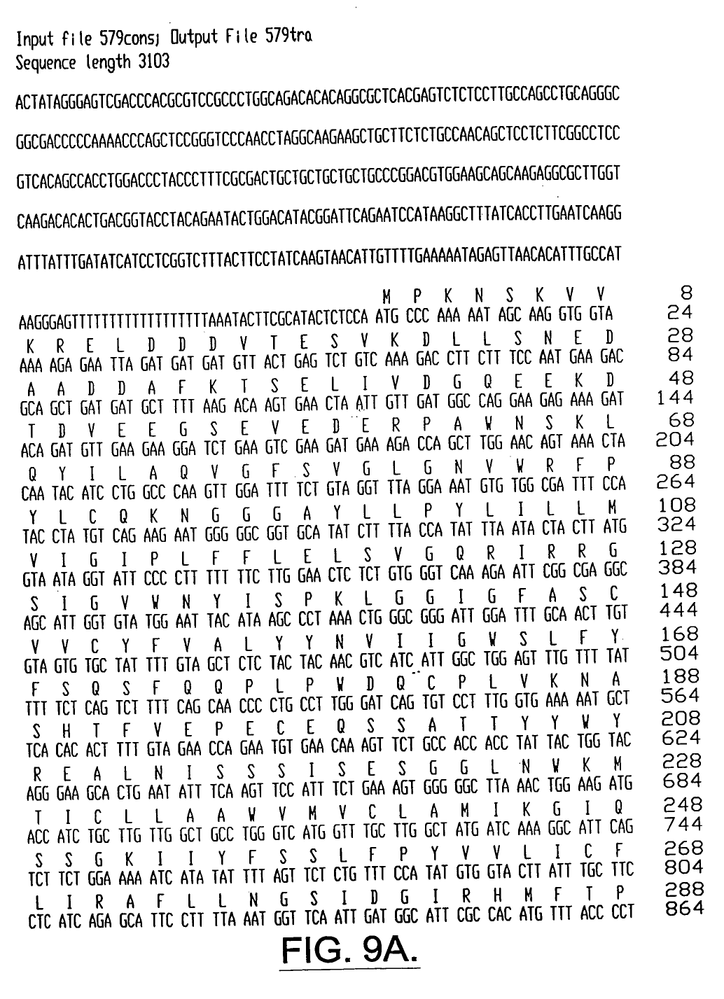

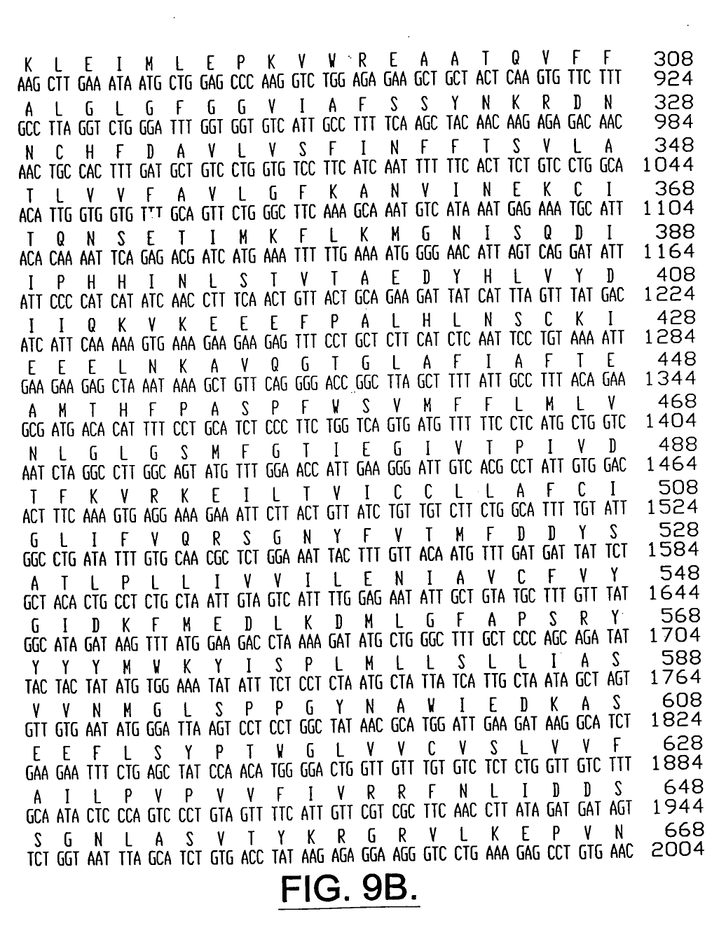

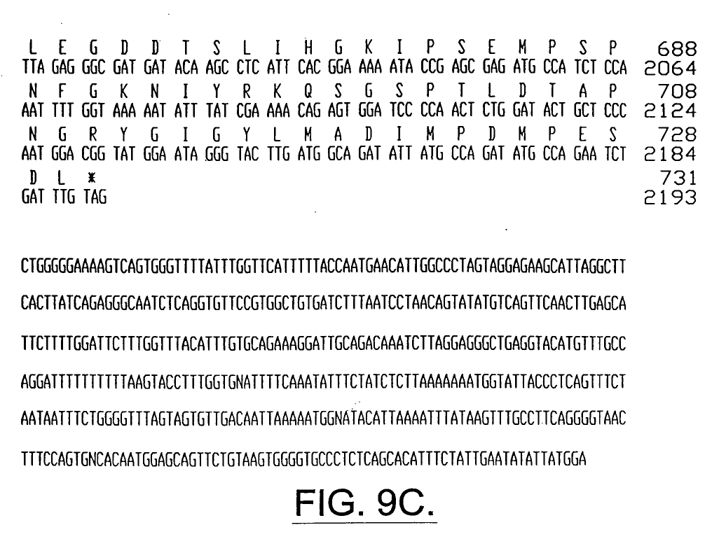

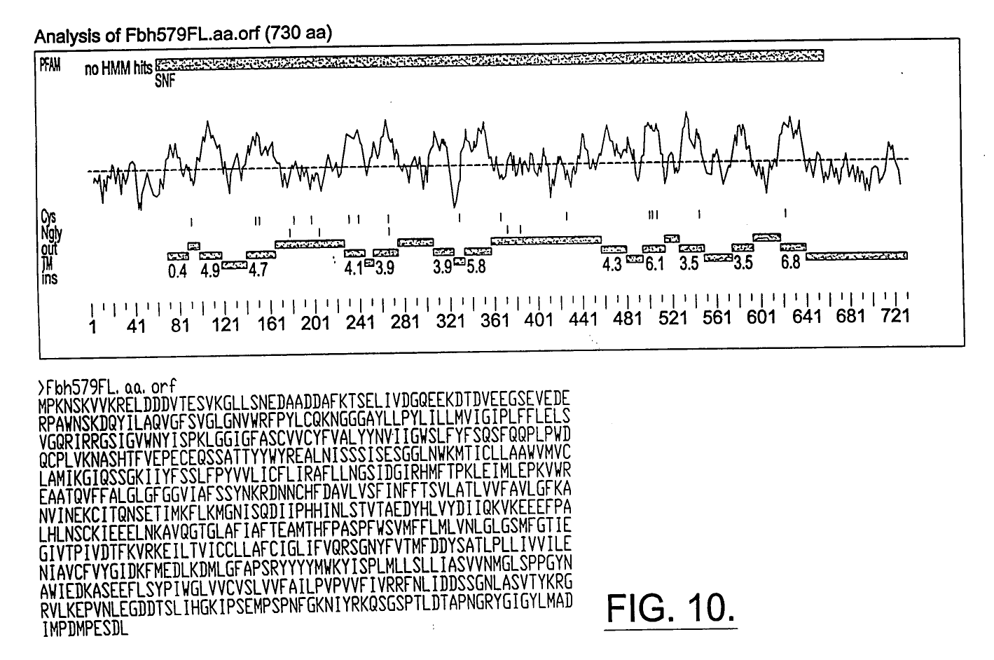

The human transporter sequence (FIG. 9A-C; SEQ ID NO:4), which is approximately 3103 nucleotides long including untranslated regions, contains a predicted methionine-initiated coding sequence of about 2190 nucleotides (nucleotides 442-2631 of SEQ ID NO:4; SEQ ID NO:6). The coding sequence encodes a 730 amino acid protein (SEQ ID NO:5).

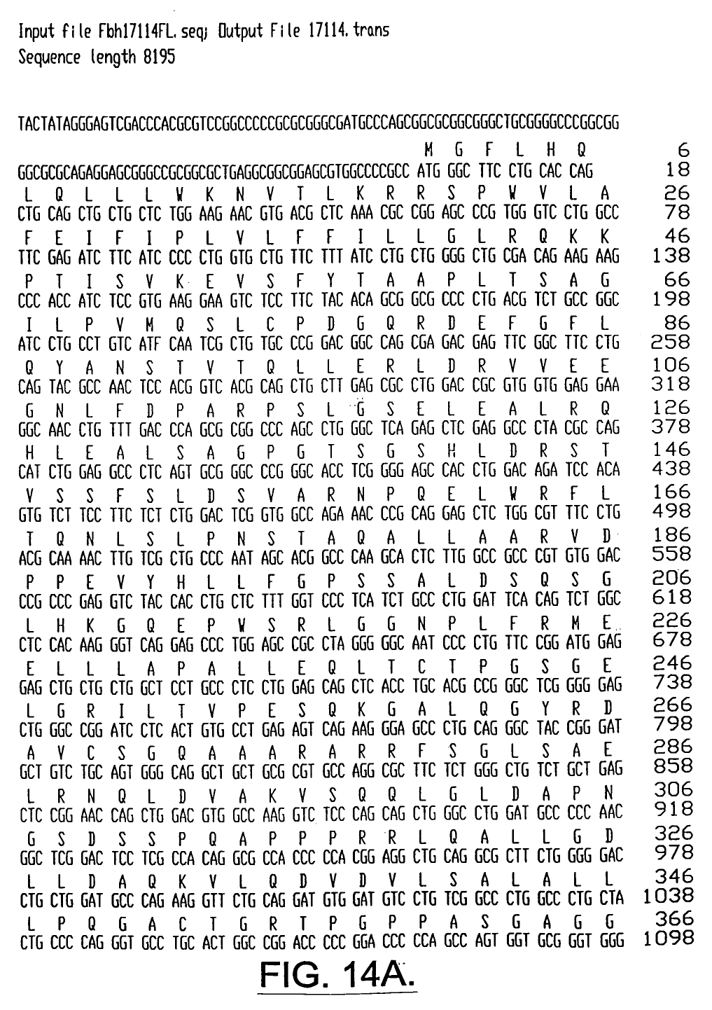

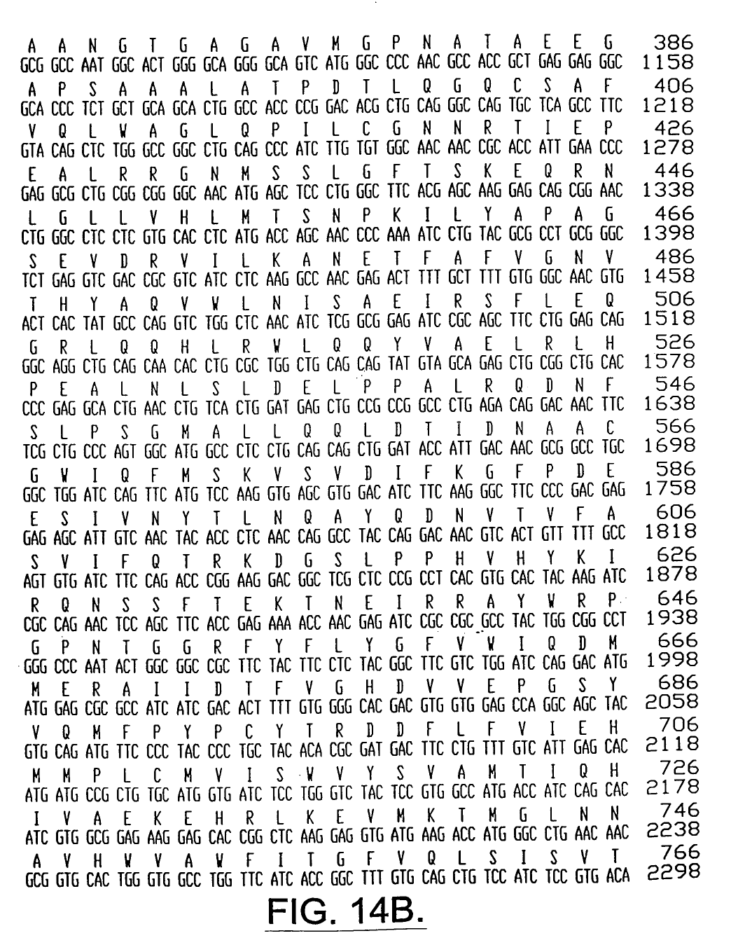

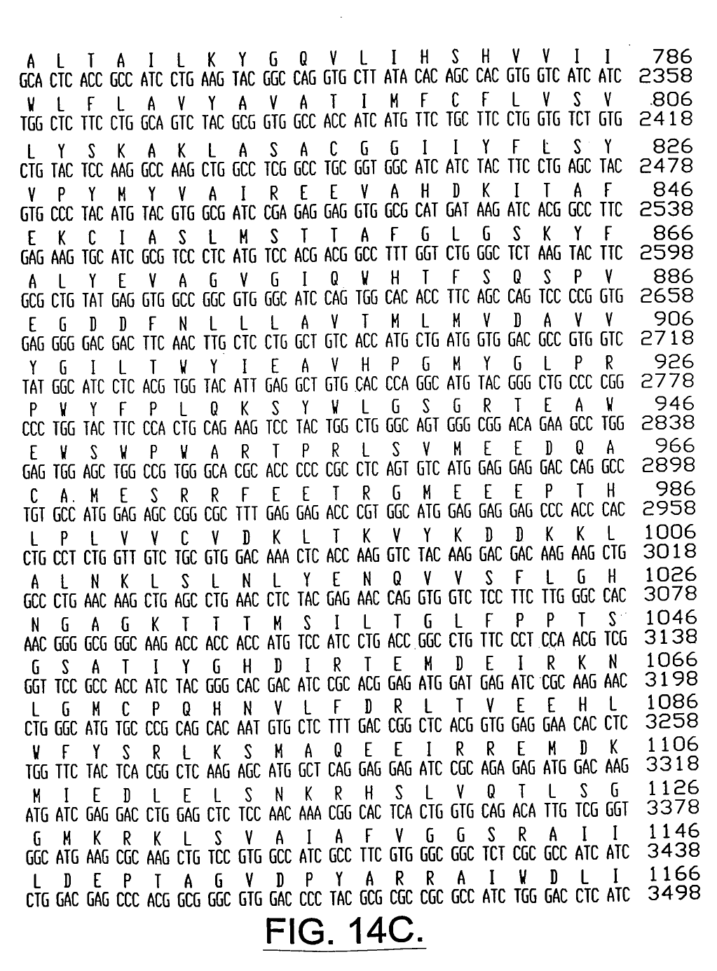

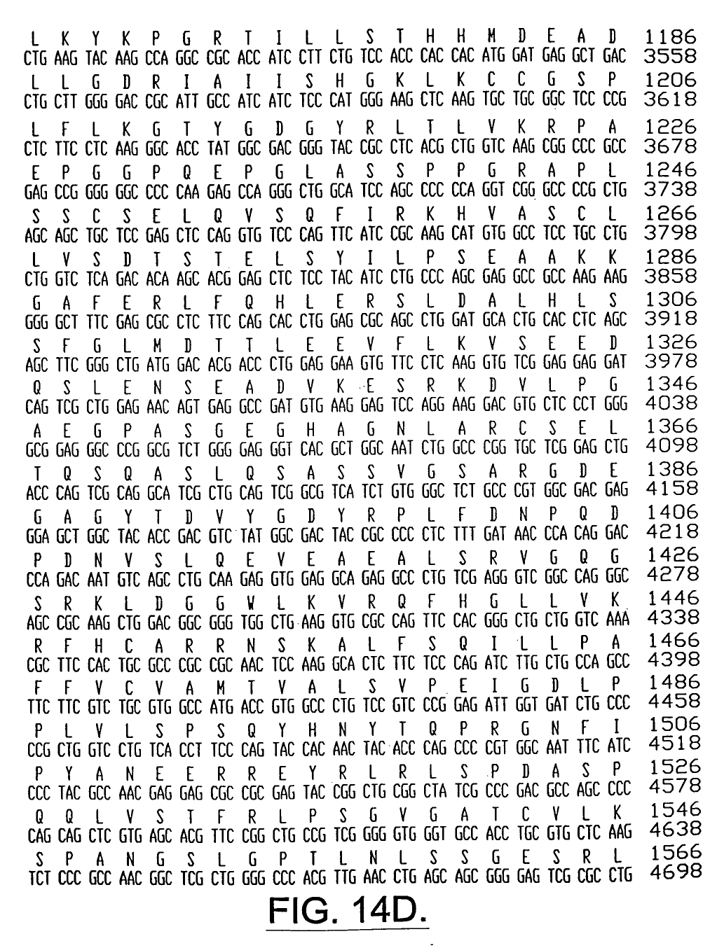

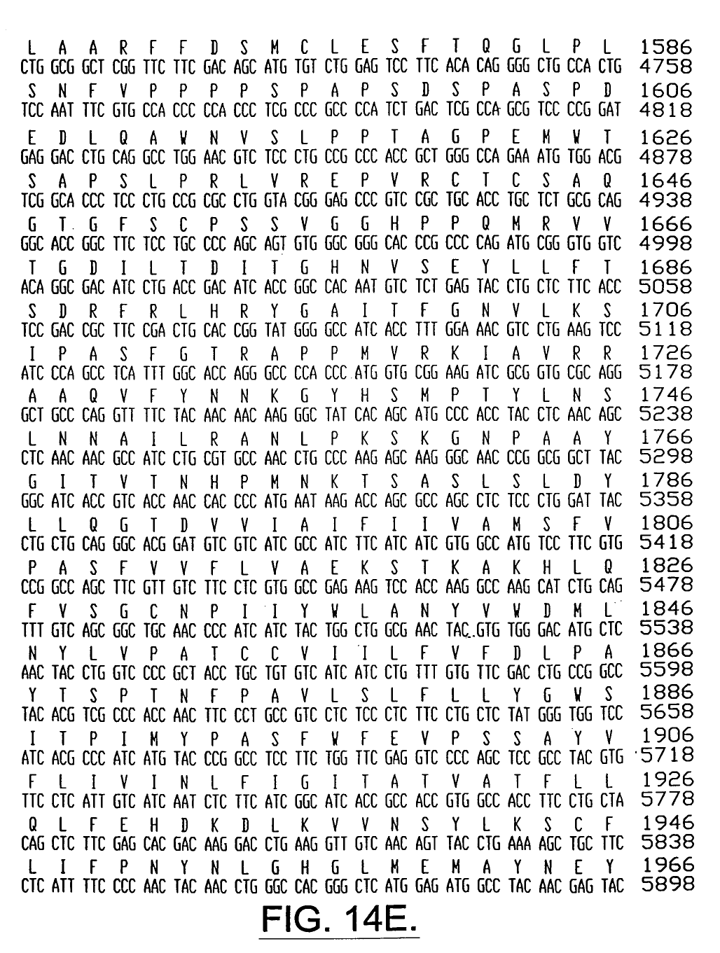

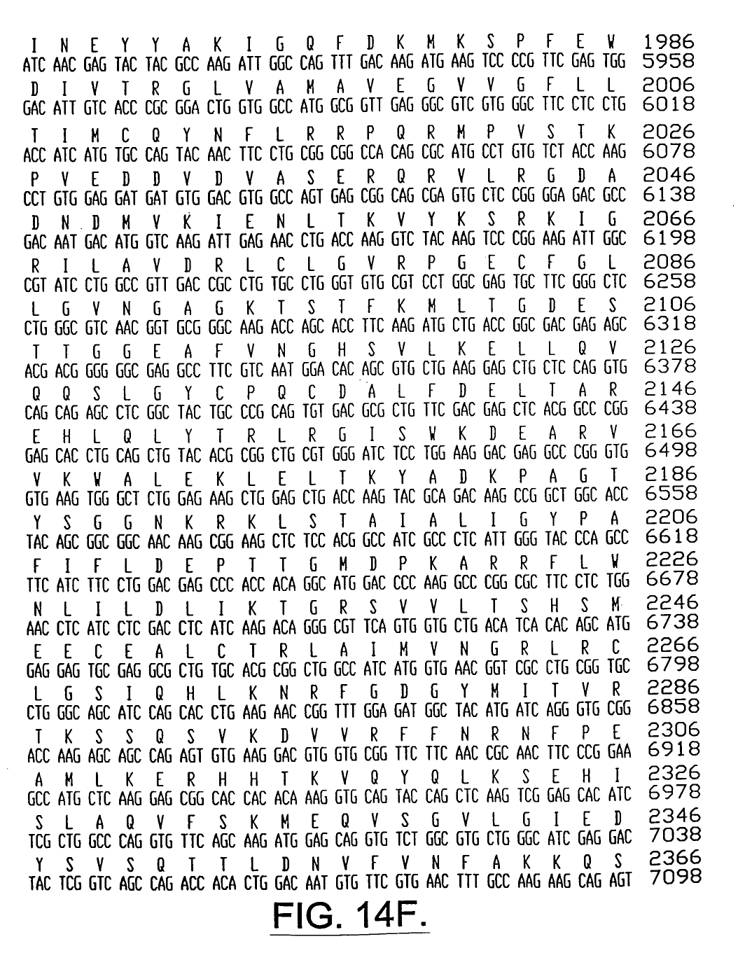

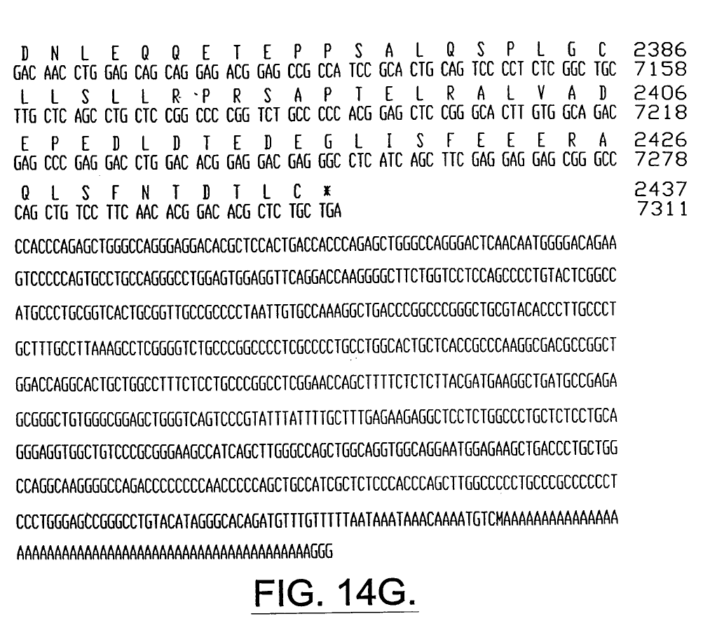

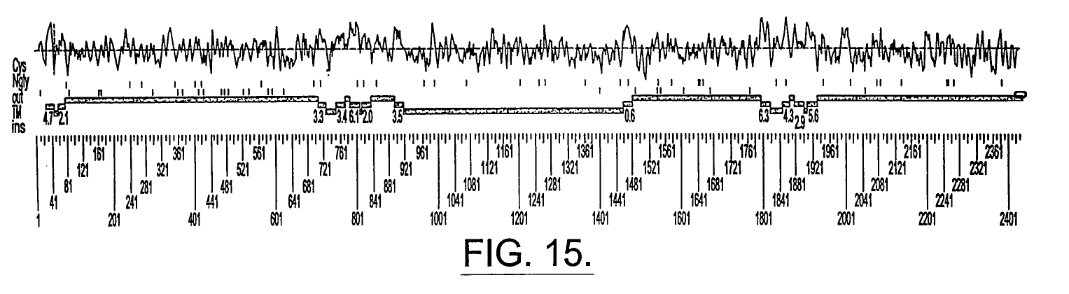

The human transporter sequence (FIG. 14A-G; SEQ ID NO:7), which is approximately 8195 nucleotides long including untranslated regions, contains a predicted methionine-initiated coding sequence of about 7308 nucleotides (nucleotides 132-7439 of SEQ ID NO:7; SEQ ID NO:9). The coding sequence encodes a 2436 amino acid protein (SEQ ID NO:8).

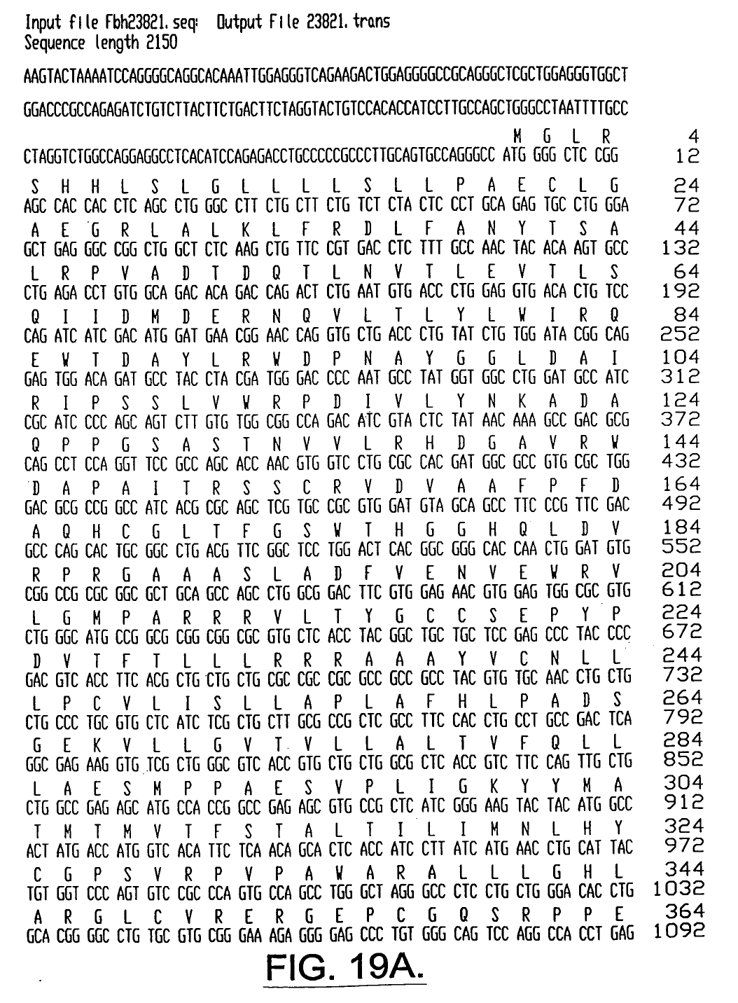

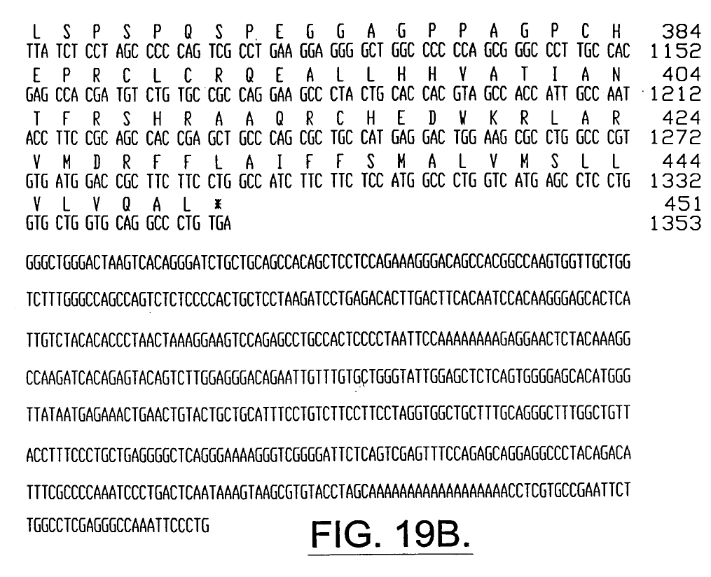

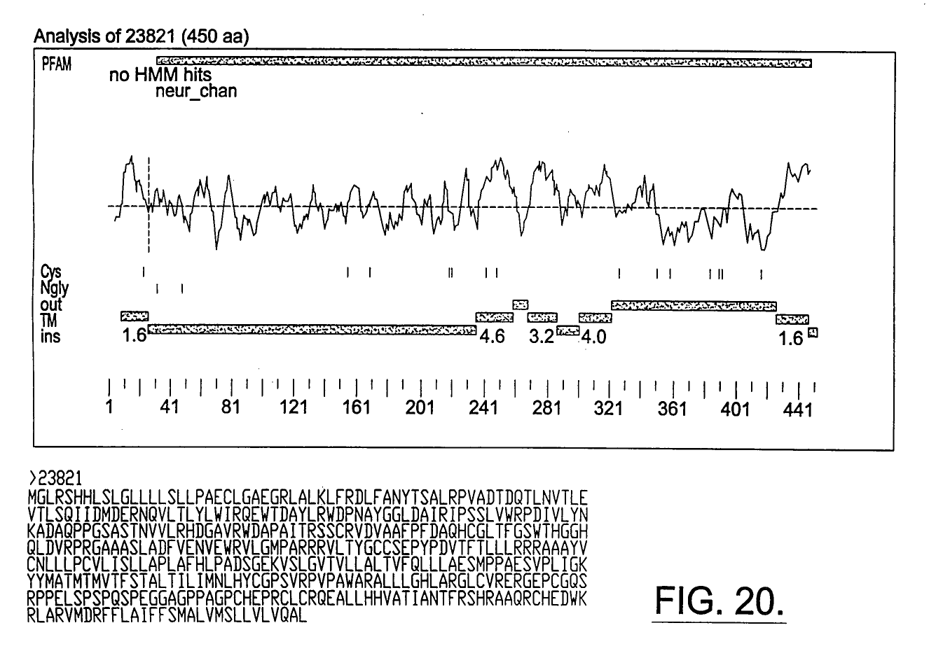

The human transporter sequence (FIG. 19A-B; SEQ ID NO:10), which is approximately 2150 nucleotides long including untranslated regions, contains a predicted methionine-initiated coding sequence of about 1350 nucleotides (nucleotides 221-1570 of SEQ ID NO:10; SEQ ID NO:12). The coding sequence encodes a 450 amino acid protein (SEQ ID NO:111).

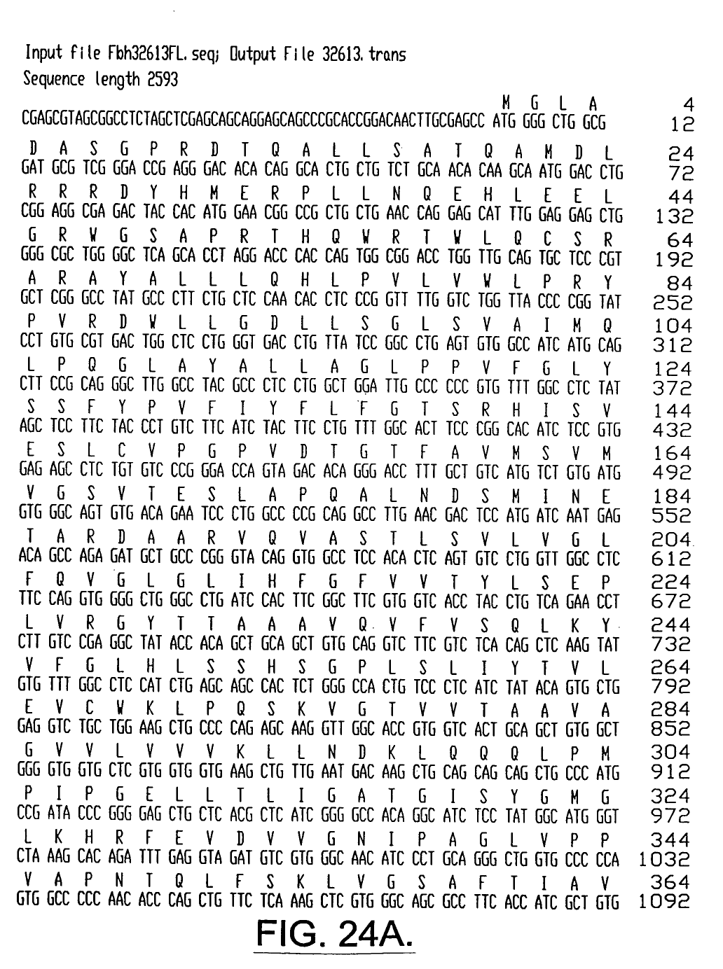

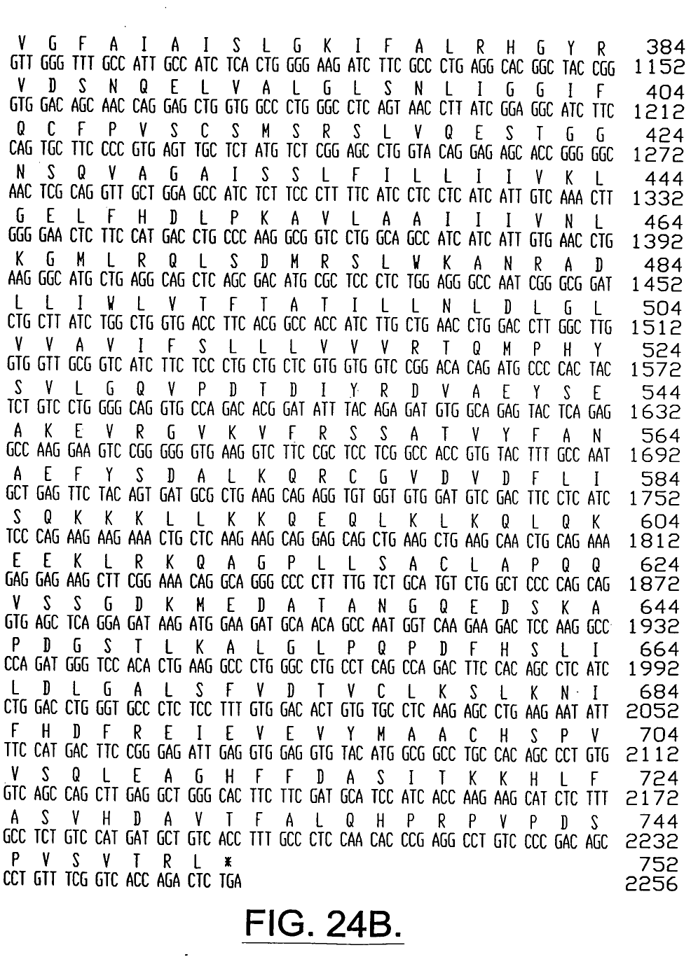

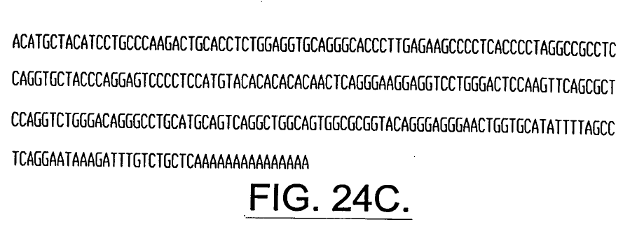

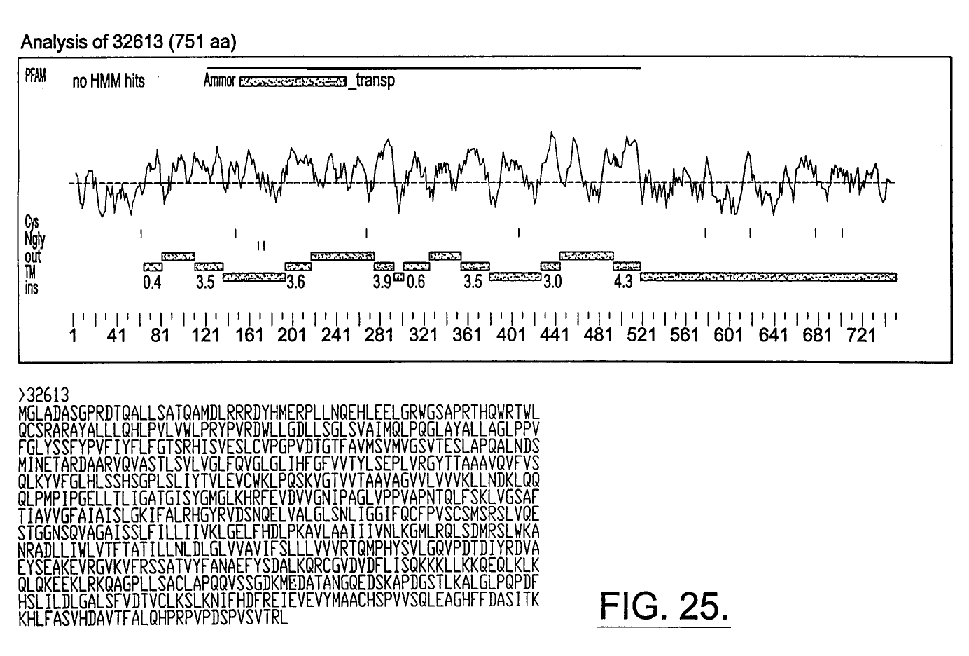

The human transporter sequence (FIG. 24A-C; SEQ ID NO:13), which is approximately 2593 nucleotides long including untranslated regions, contains a predicted methionine-initiated coding sequence of about 2253 nucleotides (nucleotides 62-2314 of SEQ ID NO:13; SEQ ID NO:15). The coding sequence encodes a 751 amino acid protein (SEQ ID NO:14).

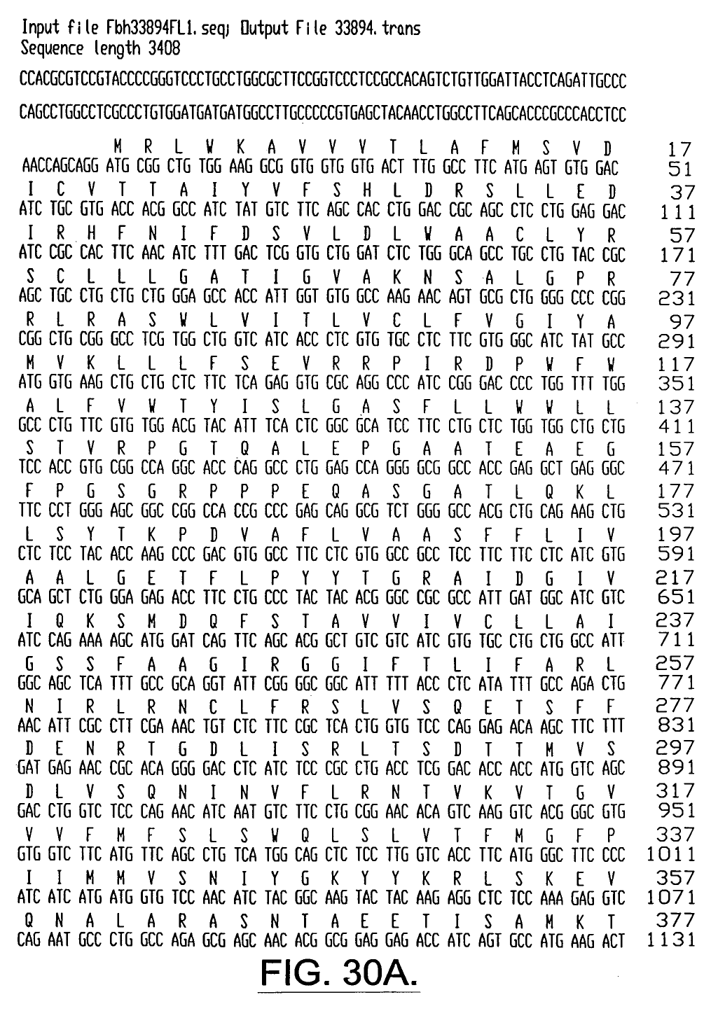

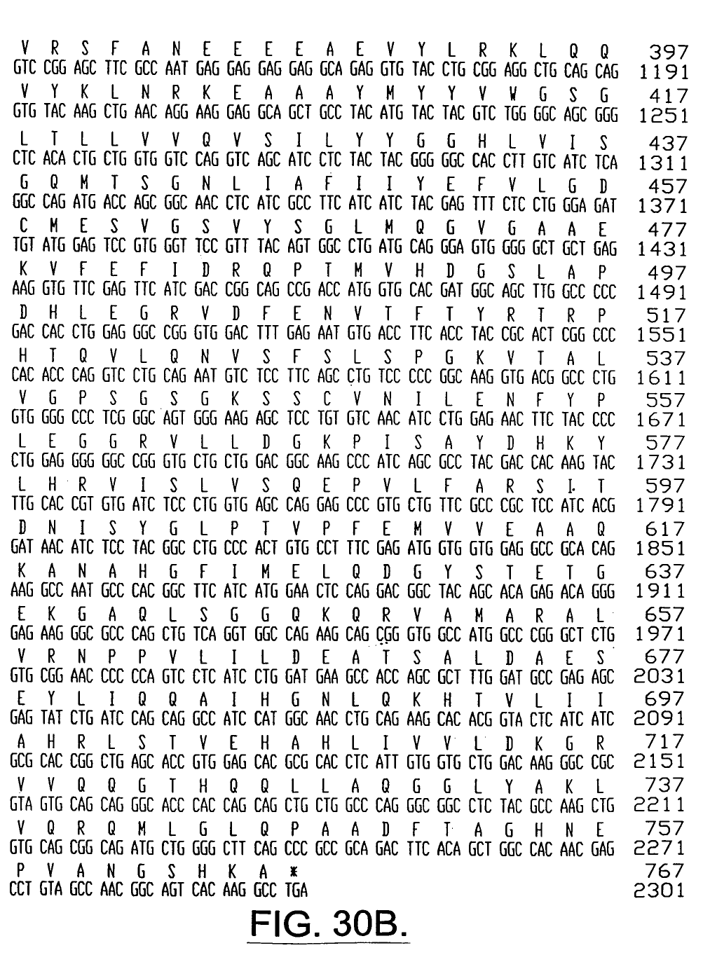

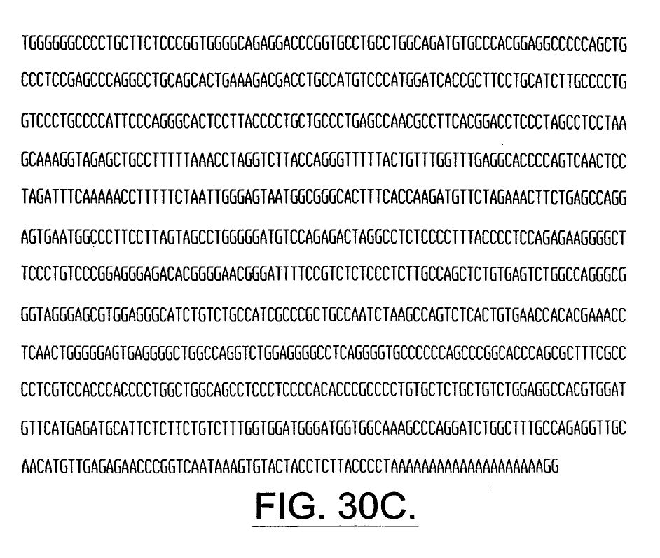

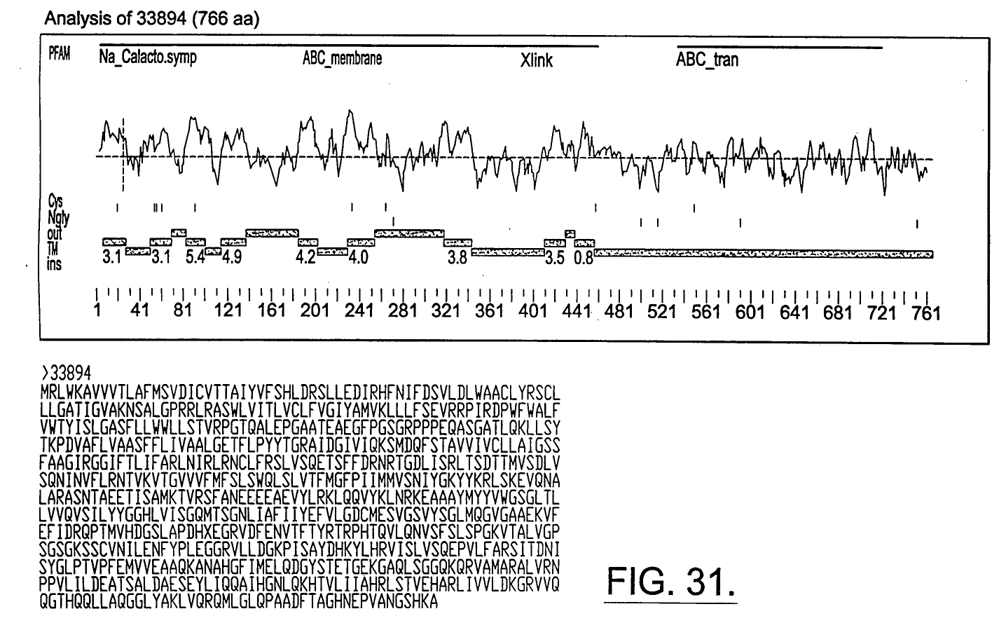

The human transporter sequence (FIG. 30A-C; SEQ ID NO:16), which is approximately 3408 nucleotides long including untranslated regions, contains a predicted methionine-initiated coding sequence of about 2298 nucleotides (nucleotides 169-2469 of SEQ ID NO:16; SEQ ID NO:18). The coding sequence encodes a 766 amino acid protein (SEQ ID NO:17).

### Example 2

### Tissue Distribution of Transporter mRNA in Human Tissue

Expression levels of transporters in various human tissue and cell types were determined by quantitative RT-PCR (Reverse Transcriptase Polymerase Chain Reaction; Taqman® brand PCR kit, Applied Biosystems). The quantitative RT-PCR reactions were performed according to the kit manufacturer's instructions. The results of the Taqman® analysis are shown in: FIGS. 7 and 36A & B (20685-transporter); FIG. 35 (33894-transporter); FIGS. 37A & B and 39 (579-transporter); and FIG. 38 (17114-transporter).

Northern blot hybridizations with various RNA samples are performed under standard conditions and washed under stringent conditions, i.e., 0.2×SSC at 65° C. A DNA probe corresponding to all or a portion of the transporter cDNA (SEQ ID NO:1) can be used. The DNA is radioactively labeled with ³²P-dCTP using the Prime-It Kit (Stratagene, La Jolla, Calif.) according to the instructions of the supplier. Filters containing mRNA from mouse hematopoietic and endocrine tissues, and cancer cell lines (Clontech, Palo Alto, Calif.) are probed in ExpressHyb hybridization solution (Clontech) and washed at high stringency according to manufacturer's recommendations.

### Example 3

### Tissue Distribution of the 579 Transporter mRNA Rat Ortholog in Rat Tissue

Expression levels of the r16854 rat ortholog of 579 in various rat tissue and cell types were determined by quantitative RT-PCR (Reverse Transcriptase Polymerase Chain Reaction; Taqman® brand PCR kit, Applied Biosystems). The quantitative RT-PCR reactions were performed according to the kit manufacturer's instructions. FIG. 40 shows the results of Taqman® analysis for a rat pain panel phase I experiment. FIG. 41 shows the results of Taqman® analysis for a rat pain panel phase II experiment.

### Example 4

### Recombinant Expression of Transporters in Bacterial Cells

In this example, a transporter is expressed as a recombinant glutathione-S-transferase (GST) fusion polypeptide in E. coli and the fusion polypeptide is isolated and characterized. Specifically, a transporter is fused to GST and this fusion polypeptide is expressed in E. coli, e.g., strain PEB199. Expression of the GST-transporter fusion protein in PEB199 is induced with IPTG. The recombinant fusion polypeptide is purified from crude bacterial lysates of the induced PEB199 strain by affinity chromatography on glutathione beads. Using polyacrylamide gel electrophoretic analysis of the polypeptide purified from the bacterial lysates, the molecular weight of the resultant fusion polypeptide is determined.

### Example 5

### Expression of Recombinant Transporter Protein in COS Cells

To express a transporter gene in COS cells, the pcDNA/Amp vector by Invitrogen Corporation (San Diego, Calif.) is used. This vector contains an SV40 origin of replication, an ampicillin resistance gene, an E. coli replication origin, a CMV promoter followed by a polylinker region, and an SV40 intron and polyadenylation site. A DNA fragment encoding the entire transporter protein and an HA tag (Wilson et al. (1984) Cell 37:767) or a FLAG tag fused in-frame to its 3′ end of the fragment is cloned into the polylinker region of the vector, thereby placing the expression of the recombinant protein under the control of the CMV promoter.

To construct the plasmid, the transporter DNA sequence is amplified by PCR using two primers. The 5′ primer contains the restriction site of interest followed by approximately twenty nucleotides of the transporter coding sequence starting from the initiation codon; the 3′ end sequence contains complementary sequences to the other restriction site of interest, a translation stop codon, the HA tag or FLAG tag and the last 20 nucleotides of the transporter coding sequence. The PCR amplified fragment and the pcDNA/Amp vector are digested with the appropriate restriction enzymes and the vector is dephosphorylated using the CIAP enzyme (New England Biolabs, Beverly, Mass.). Preferably the two restriction sites chosen are different so that the transporter gene is inserted in the correct orientation. The ligation mixture is transformed into E. coli cells (strains HB101, DH5α, SURE, available from Stratagene Cloning Systems, La Jolla, Calif., can be used), the transformed culture is plated on ampicillin media plates, and resistant colonies are selected. Plasmid DNA is isolated from transformants and examined by restriction analysis for the presence of the correct fragment.

COS cells are subsequently transfected with the transporter-pcDNA/Amp plasmid DNA using the calcium phosphate or calcium chloride co-precipitation methods, DEAE-dextran-mediated transfection, lipofection, or electroporation. Other suitable methods for transfecting host cells can be found in Sambrook et al., T. Molecular Cloning: A Laboratory Manual. 2nd, ed., Cold Spring Harbor Laboratory, Cold Spring Harbor Laboratory Press, Cold Spring Harbor, N.Y., 1989. The expression of the transporter polypeptide is detected by radiolabelling (³⁵S-methionine or ³⁵S-cysteine available from NEN, Boston, Mass., can be used) and immunoprecipitation (Harlow et al., Antibodies: A Laboratory Manual, Cold Spring Harbor Laboratory Press, Cold Spring Harbor, N.Y., 1988) using an HA specific monoclonal antibody. Briefly, the cells are labeled for 8 hours with ³⁵S-methionine (or ³⁵S-cysteine). The culture media are then collected and the cells are lysed using detergents (RIPA buffer, 150 mM NaCl, 1% NP-40, 0.1% SDS, 0.5% DOC, 50 mM Tris, pH 7.5). Both the cell lysate and the culture media are precipitated with an HA specific monoclonal antibody. Precipitated polypeptides are then analyzed by SDS-PAGE.

Alternatively, DNA containing the transporter coding sequence is cloned directly into the polylinker of the pcDNA/Amp vector using the appropriate restriction sites. The resulting plasmid is transfected into COS cells in the manner described above, and the expression of the transporter polypeptide is detected by radiolabelling and immunoprecipitation using a transporter specific monoclonal antibody.

That which is claimed:

1. An isolated nucleic acid molecule selected from the group consisting of:
a) a nucleic acid molecule comprising a nucleotide sequence which is at least 60% identical to the nucleotide sequence of SEQ ID NOS:1, 3, 4, 6, 7, 9, 10, 12, 13, 15, 16, or 18, or the nucleotide sequence of the cDNA insert of the plasmid deposited with ATCC as Accession Number PTA-2016, wherein said nucleotide sequence encodes a polypeptide having biological activity;
b) a nucleic acid molecule comprising a fragment of at least 30 nucleotides of the nucleotide sequence of SEQ ID NOS:1, 3, 4, 6, 7, 9, 10, 12, 13, 15, 16, or 18 or the nucleotide sequence of the cDNA insert of the plasmid deposited with ATCC as Accession Number PTA-2016;
c) a nucleic acid molecule which encodes a polypeptide comprising the amino acid sequence of SEQ ID NOS:2, 5, 8, 11, 14, or 17, or the amino acid sequence encoded by the cDNA insert of the plasmid deposited with the ATCC as Accession Number PTA-2016;
d) a nucleic acid molecule which encodes a fragment of a polypeptide comprising the amino acid sequence of SEQ ID NOS:2, 5, 8, 11, 14, or 17, or the amino acid sequence encoded by the cDNA insert of the plasmid deposited with the ATCC as Accession Number PTA-2016, wherein the fragment comprises at least 30 contiguous amino acids of SEQ ID NOS:2, 5, 8, 11, 14, or 17, or the amino acid sequence encoded by the cDNA insert of the plasmid deposited with the ATCC as Accession Number PTA-2016;
e) a nucleic acid molecule which encodes a naturally occurring allelic variant of a biologically active polypeptide comprising the amino acid sequence of SEQ ID NOS:2, 5, 8, 11, 14, or 17, or the amino acid sequence encoded by the cDNA insert of the plasmid deposited with the ATCC as Accession Number PTA-2016, wherein the nucleic acid molecule hybridizes to a nucleic acid molecule comprising the complement of SEQ ID NOS:1, 3, 4, 6, 7, 9, 10, 12, 13, 15, 16, or 18 under stringent conditions; and
f) a nucleic acid molecule comprising the complement of a), b), c), d), or e).

2. The isolated nucleic acid molecule of claim 1, which is selected from the group consisting of:
a) a nucleic acid comprising the nucleotide sequence of SEQ ID NOS:1, 3, 4, 6, 7, 9, 10, 12, 13, 15, 16, or 18 or a compliment thereof, or the nucleotide sequence of the cDNA insert of the plasmid deposited with ATCC as Accession Number PTA-2016, or a complement thereof; and
b) a nucleic acid molecule which encodes a polypeptide comprising the amino acid sequence of SEQ ID NOS:2, 5, 8, 11, 14, or 17 or a complement thereof, or the amino acid sequence encoded by the cDNA insert of the plasmid deposited with the ATCC as Accession Number PTA-2016, or a complement thereof.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a biologically active polypeptide which is encoded by a nucleic acid molecule comprising a nucleotide sequence which is at least 60% identical to a nucleic acid comprising the nucleotide sequence of SEQ ID NOS:1, 3, 4, 6, 7, 9, 10, 12, 13, 15, 16, or 18, or the nucleotide sequence of the cDNA insert of the plasmid deposited with the ATCC as Accession Number PTA-2016;
b) a naturally occurring allelic variant of a polypeptide comprising the amino acid sequence of SEQ ID NOS:2, 5, 8, 11, 14, or 17, or the amino acid sequence encoded by the cDNA insert of the plasmid deposited with the ATCC as Accession Number PTA-2016, wherein the polypeptide is encoded by a nucleic acid molecule which hybridizes to a nucleic acid molecule comprising the complement of SEQ ID NOS:1, 3, 4, 6, 7, 9, 10, 12, 13, 15, 16, or 18 under stringent conditions;
c) a fragment of a polypeptide comprising the amino acid sequence of SEQ ID NOS:2, 5, 8, 11, 14, or 17, or the amino acid sequence encoded by the cDNA insert of the plasmid deposited with the ATCC as Accession Number PTA-2016, wherein the fragment comprises at least 30 contiguous amino acids of SEQ ID NOS:2, 5, 8, 11, 14, or 17; and
d) a polypeptide having at least 60% sequence identity to the amino acid sequence of SEQ ID NOS:2, 5, 8, 11, 14, or 17, wherein the polypeptide has biological activity.

9. The isolated polypeptide of claim 8 comprising the amino acid sequence of SEQ ID NOS:2, 5, 8, 11, 14, or 17.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of SEQ ID NOS:2, 5, 8, 11, 14, or 17, or the amino acid sequence encoded by the cDNA insert of the plasmid deposited with the ATCC as Accession Number PTA-2016;
b) a polypeptide comprising a fragment of the amino acid sequence of SEQ ID NOS:2, 5, 8, 11, 14, or 17, or the amino acid sequence encoded by the cDNA insert of the plasmid deposited with the ATCC as Accession Number PTA-2016, wherein the fragment comprises at least 30 contiguous amino acids of SEQ ID NOS:2, 5, 8, 11, 14, or 17, or the amino acid sequence encoded by the cDNA insert of the plasmid deposited with the ATCC as Accession Number PTA-2016;
c) a biologically active naturally occurring allelic variant of a polypeptide comprising the amino acid sequence of SEQ ID NOS:2, 5, 8, 11, 14, or 17, or the amino acid sequence encoded by the cDNA insert of the plasmid deposited with the ATCC as Accession Number PTA-2016, wherein the polypeptide is encoded by a nucleic acid molecule which hybridizes to a nucleic acid molecule comprising the complement of SEQ ID NOS:1, 3, 4, 6, 7, 9, 10, 12, 13, 15, 16, or 18; and
d) a polypeptide having at least 60% sequence identity to the amino acid sequence of SEQ ID NOS:2, 5, 8, 11, 14, or 17, wherein said polypeptide has biological activity;
comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

20. The method of claim 19, wherein the binding of the test compound to the polypeptide is detected by a method selected from the group consisting of:
a) detection of binding by direct detecting of test compound/polypeptide binding;
b) detection of binding using a competition binding assay; and
c) detection of binding using an assay for transporter-like mediated transport of a substrate molecule or ion across a cell membrane.

21. The method of claim 19 wherein said cell is a virus-infected hepatocyte and said polypeptide corresponds to SEQ ID NO:2.

22. The method of claim 21 wherein said cell is in a biopsy.

23. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

24. The method of claim 23 wherein said cell is derived from virally-infected liver tissue and said polypeptide corresponds to SEQ ID NO:2.

25. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound that modulates the activity of the polypeptide.

26. A method of treating a patient afflicted with a pain disorder related to a polypeptide, said method comprising administering to the patient a compound which modulates the activity of said polypeptide in an amount effective to modulate the activity of said polypeptide in the patient, whereby at least one symptom of the disorder is alleviated, wherein said polypeptide is selected from the group consisting of:
a) a biologically active polypeptide which is encoded by a nucleic acid molecule comprising a nucleotide sequence which is at least 60% identical to the nucleotide sequence of SEQ ID NO:6, or the nucleotide sequence of the cDNA insert of the plasmid deposited with the ATCC as Accession Number PTA-2016;
b) a naturally occurring allelic variant of a polypeptide comprising the amino acid sequence of SEQ ID NO:5, or the amino acid sequence encoded by the cDNA insert of the plasmid deposited with the ATCC as Accession Number PTA-2016, wherein the polypeptide is encoded by a nucleic acid molecule which hybridizes to a nucleic acid molecule comprising the complement of SEQ ID NO:6 under stringent conditions;
c) a fragment of a polypeptide comprising the amino acid sequence of SEQ ID NO:5, or the amino acid sequence encoded by the cDNA insert of the plasmid deposited with the ATCC as Accession Number PTA-2016, wherein the fragment comprises at least 30 contiguous amino acids of SEQ ID NO:5; and
d) a polypeptide having at least 60% sequence identity to the amino acid sequence of SEQ ID NO:5, wherein the polypeptide has biological activity.

27. A method of treating a patient afflicted with a pain disorder related to a polypeptide, said method comprising administering to the patient, in an amount effective to modulate the activity of the protein in the patient, a compound selected from the group consisting of said polypeptide, a nucleic acid encoding said polypeptide, and an antisense nucleic acid which is capable of annealing with either of an mRNA encoding said polypeptide and a portion of a genomic DNA encoding said polypeptide, whereby at least one symptom of the disorder is alleviated, wherein said polypeptide is selected from the group consisting of:
a) a biologically active polypeptide which is encoded by a nucleic acid molecule comprising a nucleotide sequence which is at least 60% identical to the nucleotide sequence of SEQ ID NO:6, or the nucleotide sequence of the cDNA insert of the plasmid deposited with the ATCC as Accession Number PTA-2016;
b) a naturally occurring allelic variant of a polypeptide comprising the amino acid sequence of SEQ ID NO:5, or the amino acid sequence encoded by the cDNA insert of the plasmid deposited with the ATCC as Accession Number PTA-2016, wherein the polypeptide is encoded by a nucleic acid molecule which hybridizes to a nucleic acid molecule comprising the complement of SEQ ID NO:6 under stringent conditions;
c) a fragment of a polypeptide comprising the amino acid sequence of SEQ ID NO:5, or the amino acid sequence encoded by the cDNA insert of the plasmid deposited with the ATCC as Accession Number PTA-2016, wherein the fragment comprises at least 30 contiguous amino acids of SEQ ID NO:5; and
d) a polypeptide having at least 60% sequence identity to the amino acid sequence of SEQ ID NO:5, wherein the polypeptide has biological activity.


SEQUENCE LISTING (42 sequences)

SEQ ID NO: 1 - DNA, 1734 bp, Homo sapiens
  CDS (96)...(1466)
cggccgcccgccagagtgccagtggtccttggaggtcgagtccaaggacgtggcttgaagccgggagctggggcgccggagtccacgcaccggggatggaggcgctgggtgacctggagggaccacgcgcaccaggaggtgatgatcctgcaggaagtgcaggagagacccccgggtggctttcgagagaacaggtttttgtactgatatcggcagcttcggcgaacttaggttccatgatgtgctattctatacttggaccgtttttccccaaagaggctgaaaagaagggggccagcaatacaattatcggtatgatctttggatgttttgctttgttcgagttgctggcatccttggtatttggaaactatcttgtacatattggagcaaaatttatgtttgtagcaggaatgtttgtctcaggaggagttacaattctctttggtgtattggaccgagttccagatgggccagtatttattgctatgtgttttctagtgagagtaatggatgcagttagctttgctgcagcaatgactgcatcttcttctatcctggcaaaggcttttccaaataacgtggctacggtattgggaagtcttgagactttttctggactggggctaatactaggtcctcctgtaggtggctttttgtatcaatcctttggctatgaagtgccttttattgttctgggatgcgtcgttttgctgatggtaccactcaatatgtatattttacccaattacgagtctgatccaggtgaacactcattctggaaactgatcgctttacccaaagttggccttatagccttcgtcatcaactcactcagctcgtgttttggcttcctcgatcctactctgtctctctttgttttggagaagttcaatttaccagctggatatgtgggactagtattcctgggtatggcactgtcctatgccatctcttcaccactatttggtctcctaagtgataaaaggccacctctaaggaaatggcttctggtgtttggcaacttaatcacagccgggtgctacatgctcttagggcctgtcccaatcttgcatattaaaagtcagctctggctgctggtgctgatattagttgtaagtggcctctctgctggaatgagtataattccaactttcccggaaattctcagttgtgcacatgaaaatgggtttgaagagggattaagtacattgggacttgtatcaggtctttttagtgcaatgtggtcaattggtgcttttatgggaccaacgctgggtggatttctgtatgagaaaattggttttgaatgggcagcagctatacaaggtctatgggctctgataagtggattagccatgggcttgttttatctactggagtattcaaggagaaaaaggtctaaatctcaaaacatcctcagcacagaggaggaacgaactactctcttgcctaatgaaacctagtccgatggatcctggattgatacaaggttgagaaatgaatgctcctggccttaaacatcaccgtaggaagggtttttaaaattttacgcgcaaaactccgtggaccccgtgccagtgtcttggaagtgtcaacgtgtttttggatgatcctgtattgggctgtacttactgtgatactgaaaagctgtcctgctgaagcagctatatttgaaatattaagtatgaaaggagtaattaaaaacaagcaaaaaaaaaaaaaaaagggcgg

SEQ ID NO: 2 - protein, 456 aa, Homo sapiens
MEALGDLEGPRAPGGDDPAGSAGETPGWLSREQVFVLISAASANLGSMMCYSILGPFFPKEAEKKGASNTIIGMIFGCFALFELLASLVFGNYLVHIGAKFMFVAGMFVSGGVTILFGVLDRVPDGPVFIAMCFLVRVMDAVSFAAAMTASSSILAKAFPNNVATVLGSLETFSGLGLILGPPVGGFLYQSFGYEVPFIVLGCVVLLMVPLNMYILPNYESDPGEHSFWKLIALPKVGLIAFVINSLSSCFGFLDPTLSLFVLEKFNLPAGYVGLVFLGMALSYAISSPLFGLLSDKRPPLRKWLLVFGNLITAGCYMLLGPVPILHIKSQLWLLVLILVVSGLSAGMSIIPTFPEILSCAHENGFEEGLSTLGLVSGLFSAMWSIGAFMGPTLGGFLYEKIGFEWAAAIQGLWALISGLAMGLFYLLEYSRRKRSKSQNILSTEEERTTLLPNET

SEQ ID NO: 3 - DNA, 1368 bp, Homo sapiens
atggaggcgctgggtgacctggagggaccacgcgcaccaggaggtgatgatcctgcaggaagtgcaggagagacccccgggtggctttcgagagaacaggtttttgtactgatatcggcagcttcggcgaacttaggttccatgatgtgctattctatacttggaccgtttttccccaaagaggctgaaaagaagggggccagcaatacaattatcggtatgatctttggatgttttgctttgttcgagttgctggcatccttggtatttggaaactatcttgtacatattggagcaaaatttatgtttgtagcaggaatgtttgtctcaggaggagttacaattctctttggtgtattggaccgagttccagatgggccagtatttattgctatgtgttttctagtgagagtaatggatgcagttagctttgctgcagcaatgactgcatcttcttctatcctggcaaaggcttttccaaataacgtggctacggtattgggaagtcttgagactttttctggactggggctaatactaggtcctcctgtaggtggctttttgtatcaatcctttggctatgaagtgccttttattgttctgggatgcgtcgttttgctgatggtaccactcaatatgtatattttacccaattacgagtctgatccaggtgaacactcattctggaaactgatcgctttacccaaagttggccttatagccttcgtcatcaactcactcagctcgtgttttggcttcctcgatcctactctgtctctctttgttttggagaagttcaatttaccagctggatatgtgggactagtattcctgggtatggcactgtcctatgccatctcttcaccactatttggtctcctaagtgataaaaggccacctctaaggaaatggcttctggtgtttggcaacttaatcacagccgggtgctacatgctcttagggcctgtcccaatcttgcatattaaaagtcagctctggctgctggtgctgatattagttgtaagtggcctctctgctggaatgagtataattccaactttcccggaaattctcagttgtgcacatgaaaatgggtttgaagagggattaagtacattgggacttgtatcaggtctttttagtgcaatgtggtcaattggtgcttttatgggaccaacgctgggtggatttctgtatgagaaaattggttttgaatgggcagcagctatacaaggtctatgggctctgataagtggattagccatgggcttgttttatctactggagtattcaaggagaaaaaggtctaaatctcaaaacatcctcagcacagaggaggaacgaactactctcttgcctaatgaaacc

SEQ ID NO: 4 - DNA, 3103 bp, Homo sapiens
  CDS (442)...(2634)
  misc_feature (1)...(3103): n = A,T,C or G
actatagggagtcgacccacgcgtccgccctggcagacacacaggcgctcacgagtctctccttgccagcctgcagggcggcgacccccaaaacccagctccgggtcccaacctaggcaagaagctgcttctctgccaacagctcctcttcggcctccgtcacagccacctggaccctaccctttcgcgactgctgctgctgctgcccggacgtggaagcagcaagaggcgcttggtcaagacacactgacggtacctacagaatactggacatacggattcagaatccataaggctttatcaccttgaatcaaggatttatttgatatcatcctcggtctttacttcctatcaagtaacattgttttgaaaaatagagttaacacatttgccataagggagttttttttttttttttttaaatacttcgcatactctccaatgcccaaaaatagcaaggtggtaaaaagagaattagatgatgatgttactgagtctgtcaaagaccttctttccaatgaagacgcagctgatgatgcttttaagacaagtgaactaattgttgatggccaggaagagaaagatacagatgttgaagaaggatctgaagtcgaagatgaaagaccagcttggaacagtaaactacaatacatcctggcccaagttggattttctgtaggtttaggaaatgtgtggcgatttccatacctatgtcagaagaatgggggcggtgcatatcttttaccatatttaatactacttatggtaataggtattcccctttttttcttggaactctctgtgggtcaaagaattcggcgaggcagcattggtgtatggaattacataagccctaaactgggcgggattggatttgcaagttgtgtagtgtgctattttgtagctctctactacaacgtcatcattggctggagtttgttttatttttctcagtcttttcagcaacccctgccttgggatcagtgtcctttggtgaaaaatgcttcacacacttttgtagaaccagaatgtgaacaaagttctgccaccacctattactggtacagggaagcactgaatatttcaagttccatttctgaaagtgggggcttaaactggaagatgaccatctgcttgttggctgcctgggtcatggtttgcttggctatgatcaaaggcattcagtcttctggaaaaatcatatattttagttctctgtttccatatgtggtacttatttgcttcctcatcagagcattccttttaaatggttcaattgatggcattcgccacatgtttacccctaagcttgaaataatgctggagcccaaggtctggagagaagctgctactcaagtgttctttgccttaggtctgggatttggtggtgtcattgccttttcaagctacaacaagagagacaacaactgccactttgatgctgtcctggtgtccttcatcaattttttcacttctgtcctggcaacattggtggtgtttgcagttctgggcttcaaagcaaatgtcataaatgagaaatgcattacacaaaattcagagacgatcatgaaatttttgaaaatggggaacattagtcaggatattattccccatcatatcaacctttcaactgttactgcagaagattatcatttagtttatgacatcattcaaaaagtgaaagaagaagagtttcctgctcttcatctcaattcctgtaaaattgaagaagagctaaataaagctgttcaggggaccggcttagcttttattgcctttacagaagcgatgacacattttcctgcatctcccttctggtcagtgatgtttttcctcatgctggtcaatctaggccttggcagtatgtttggaaccattgaagggattgtcacgcctattgtggacactttcaaagtgaggaaagaaattcttactgttatctgttgtcttctggcattttgtattggcctgatatttgtgcaacgctctggaaattactttgttacaatgtttgatgattattctgctacactgcctctgctaattgtagtcattttggagaatattgctgtatgctttgtttatggcatagataagtttatggaagacctaaaagatatgctgggctttgctcccagcagatattactactatatgtggaaatatatttctcctctaatgctattatcattgctaatagctagtgttgtgaatatgggattaagtcctcctggctataacgcatggattgaagataaggcatctgaagaatttctgagctatccaacatggggactggttgtttgtgtctctctggttgtctttgcaatactcccagtccctgtagttttcattgttcgtcgcttcaaccttatagatgatagttctggtaatttagcatctgtgacctataagagaggaagggtcctgaaagagcctgtgaacttagagggcgatgatacaagcctcattcacggaaaaataccgagcgagatgccatctccaaattttggtaaaaatatttatcgaaaacagagtggatccccaactctggatactgctcccaatggacggtatggaatagggtacttgatggcagatattatgccagatatgccagaatctgatttgtagctgggggaaaagtcagtgggttttatttggttcatttttaccaatgaacattggccctagtaggagaagcattaggcttcacttatcagagggcaatctcaggtgttccgtggctgtgatctttaatcctaacagtatatgtcagttcaacttgagcattcttttggattctttggtttacatttgtgcagaaaggattgcagacaaatcttaggagggctgaggtacatgtttgccaggattttttttttaagtacctttggtgnattttcaaatatttctatctcttaaaaaaatggtattaccctcagtttctaataatttctggggtttagtagtgttgacaattaaaaatggnatacattaaaatttataagtttgccttcaggggtaactttccagtgncacaatggagcagttctgtaagtggggtgccctctcagcacatttctattgaatatattatgga

SEQ ID NO: 5 - protein, 730 aa, Homo sapiens
MPKNSKVVKRELDDDVTESVKDLLSNEDAADDAFKTSELIVDGQEEKDTDVEEGSEVEDERPAWNSKLQYILAQVGFSVGLGNVWRFPYLCQKNGGGAYLLPYLILLMVIGIPLFFLELSVGQRIRRGSIGVWNYISPKLGGIGFASCVVCYFVALYYNVIIGWSLFYFSQSFQQPLPWDQCPLVKNASHTFVEPECEQSSATTYYWYREALNISSSISESGGLNWKMTICLLAAWVMVCLAMIKGIQSSGKIIYFSSLFPYVVLICFLIRAFLLNGSIDGIRHMFTPKLEIMLEPKVWREAATQVFFALGLGFGGVIAFSSYNKRDNNCHFDAVLVSFINFFTSVLATLVVFAVLGFKANVINEKCITQNSETIMKFLKMGNISQDIIPHHINLSTVTAEDYHLVYDIIQKVKEEEFPALHLNSCKIEEELNKAVQGTGLAFIAFTEAMTHFPASPFWSVMFFLMLVNLGLGSMFGTIEGIVTPIVDTFKVRKEILTVICCLLAFCIGLIFVQRSGNYFVTMFDDYSATLPLLIVVILENIAVCFVYGIDKFMEDLKDMLGFAPSRYYYYMWKYISPLMLLSLLIASVVNMGLSPPGYNAWIEDKASEEFLSYPTWGLVVCVSLVVFAILPVPVVFIVRRFNLIDDSSGNLASVTYKRGRVLKEPVNLEGDDTSLIHGKIPSEMPSPNFGKNIYRKQSGSPTLDTAPNGRYGIGYLMADIMPDMPESDL

SEQ ID NO: 6 - DNA, 2190 bp, Homo sapiens
atgcccaaaaatagcaaggtggtaaaaagagaattagatgatgatgttactgagtctgtcaaagaccttctttccaatgaagacgcagctgatgatgcttttaagacaagtgaactaattgttgatggccaggaagagaaagatacagatgttgaagaaggatctgaagtcgaagatgaaagaccagcttggaacagtaaactacaatacatcctggcccaagttggattttctgtaggtttaggaaatgtgtggcgatttccatacctatgtcagaagaatgggggcggtgcatatcttttaccatatttaatactacttatggtaataggtattcccctttttttcttggaactctctgtgggtcaaagaattcggcgaggcagcattggtgtatggaattacataagccctaaactgggcgggattggatttgcaagttgtgtagtgtgctattttgtagctctctactacaacgtcatcattggctggagtttgttttatttttctcagtcttttcagcaacccctgccttgggatcagtgtcctttggtgaaaaatgcttcacacacttttgtagaaccagaatgtgaacaaagttctgccaccacctattactggtacagggaagcactgaatatttcaagttccatttctgaaagtgggggcttaaactggaagatgaccatctgcttgttggctgcctgggtcatggtttgcttggctatgatcaaaggcattcagtcttctggaaaaatcatatattttagttctctgtttccatatgtggtacttatttgcttcctcatcagagcattccttttaaatggttcaattgatggcattcgccacatgtttacccctaagcttgaaataatgctggagcccaaggtctggagagaagctgctactcaagtgttctttgccttaggtctgggatttggtggtgtcattgccttttcaagctacaacaagagagacaacaactgccactttgatgctgtcctggtgtccttcatcaattttttcacttctgtcctggcaacattggtggtgtttgcagttctgggcttcaaagcaaatgtcataaatgagaaatgcattacacaaaattcagagacgatcatgaaatttttgaaaatggggaacattagtcaggatattattccccatcatatcaacctttcaactgttactgcagaagattatcatttagtttatgacatcattcaaaaagtgaaagaagaagagtttcctgctcttcatctcaattcctgtaaaattgaagaagagctaaataaagctgttcaggggaccggcttagcttttattgcctttacagaagcgatgacacattttcctgcatctcccttctggtcagtgatgtttttcctcatgctggtcaatctaggccttggcagtatgtttggaaccattgaagggattgtcacgcctattgtggacactttcaaagtgaggaaagaaattcttactgttatctgttgtcttctggcattttgtattggcctgatatttgtgcaacgctctggaaattactttgttacaatgtttgatgattattctgctacactgcctctgctaattgtagtcattttggagaatattgctgtatgctttgtttatggcatagataagtttatggaagacctaaaagatatgctgggctttgctcccagcagatattactactatatgtggaaatatatttctcctctaatgctattatcattgctaatagctagtgttgtgaatatgggattaagtcctcctggctataacgcatggattgaagataaggcatctgaagaatttctgagctatccaacatggggactggttgtttgtgtctctctggttgtctttgcaatactcccagtccctgtagttttcattgttcgtcgcttcaaccttatagatgatagttctggtaatttagcatctgtgacctataagagaggaagggtcctgaaagagcctgtgaacttagagggcgatgatacaagcctcattcacggaaaaataccgagcgagatgccatctccaaattttggtaaaaatatttatcgaaaacagagtggatccccaactctggatactgctcccaatggacggtatggaatagggtacttgatggcagatattatgccagatatgccagaatctgatttg

SEQ ID NO: 7 - DNA, 8195 bp, Homo sapiens
  CDS (132)...(7442)
tactatagggagtcgacccacgcgtccggcccccgcgcgggcgatgcccagcggcgcggcgggctgcggggcccggcggggcgcgcagaggagcgggccgcggcgctgaggcggcggagcgtggccccgccatgggcttcctgcaccagctgcagctgctgctctggaagaacgtgacgctcaaacgccggagcccgtgggtcctggccttcgagatcttcatccccctggtgctgttctttatcctgctggggctgcgacagaagaagcccaccatctccgtgaaggaagtctccttctacacagcggcgcccctgacgtctgccggcatcctgcctgtcatgcaatcgctgtgcccggacggccagcgagacgagttcggcttcctgcagtacgccaactccacggtcacgcagctgcttgagcgcctggaccgcgtggtggaggaaggcaacctgtttgacccagcgcggcccagcctgggctcagagctcgaggccctacgccagcatctggaggccctcagtgcgggcccgggcacctcggggagccacctggacagatccacagtgtcttccttctctctggactcggtggccagaaacccgcaggagctctggcgtttcctgacgcaaaacttgtcgctgcccaatagcacggcccaagcactcttggccgcccgtgtggacccgcccgaggtctaccacctgctctttggtccctcatctgccctggattcacagtctggcctccacaagggtcaggagccctggagccgcctagggggcaatcccctgttccggatggaggagctgctgctggctcctgccctcctggagcagctcacctgcacgccgggctcgggggagctgggccggatcctcactgtgcctgagagtcagaagggagccctgcagggctaccgggatgctgtctgcagtgggcaggctgctgcgcgtgccaggcgcttctctgggctgtctgctgagctccggaaccagctggacgtggccaaggtctcccagcagctgggcctggatgcccccaacggctcggactcctcgccacaggcgccacccccacggaggctgcaggcgcttctgggggacctgctggatgcccagaaggttctgcaggatgtggatgtcctgtcggccctggccctgctactgccccagggtgcctgcactggccggacccccggacccccagccagtggtgcgggtggggcggccaatggcactggggcaggggcagtcatgggccccaacgccaccgctgaggagggcgcaccctctgctgcagcactggccaccccggacacgctgcagggccagtgctcagccttcgtacagctctgggccggcctgcagcccatcttgtgtggcaacaaccgcaccattgaacccgaggcgctgcggcggggcaacatgagctccctgggcttcacgagcaaggagcagcggaacctgggcctcctcgtgcacctcatgaccagcaaccccaaaatcctgtacgcgcctgcgggctctgaggtcgaccgcgtcatcctcaaggccaacgagacttttgcttttgtgggcaacgtgactcactatgcccaggtctggctcaacatctcggcggagatccgcagcttcctggagcagggcaggctgcagcaacacctgcgctggctgcagcagtatgtagcagagctgcggctgcaccccgaggcactgaacctgtcactggatgagctgccgccggccctgagacaggacaacttctcgctgcccagtggcatggccctcctgcagcagctggataccattgacaacgcggcctgcggctggatccagttcatgtccaaggtgagcgtggacatcttcaagggcttccccgacgaggagagcattgtcaactacaccctcaaccaggcctaccaggacaacgtcactgtttttgccagtgtgatcttccagacccggaaggacggctcgctcccgcctcacgtgcactacaagatccgccagaactccagcttcaccgagaaaaccaacgagatccgccgcgcctactggcggcctgggcccaatactggcggccgcttctacttcctctacggcttcgtctggatccaggacatgatggagcgcgccatcatcgacacttttgtggggcacgacgtggtggagccaggcagctacgtgcagatgttcccctacccctgctacacacgcgatgacttcctgtttgtcattgagcacatgatgccgctgtgcatggtgatctcctgggtctactccgtggccatgaccatccagcacatcgtggcggagaaggagcaccggctcaaggaggtgatgaagaccatgggcctgaacaacgcggtgcactgggtggcctggttcatcaccggctttgtgcagctgtccatctccgtgacagcactcaccgccatcctgaagtacggccaggtgcttatacacagccacgtggtcatcatctggctcttcctggcagtctacgcggtggccaccatcatgttctgcttcctggtgtctgtgctgtactccaaggccaagctggcctcggcctgcggtggcatcatctacttcctgagctacgtgccctacatgtacgtggcgatccgagaggaggtggcgcatgataagatcacggccttcgagaagtgcatcgcgtccctcatgtccacgacggcctttggtctgggctctaagtacttcgcgctgtatgaggtggccggcgtgggcatccagtggcacaccttcagccagtccccggtggagggggacgacttcaacttgctcctggctgtcaccatgctgatggtggacgccgtggtctatggcatcctcacgtggtacattgaggctgtgcacccaggcatgtacgggctgccccggccctggtacttcccactgcagaagtcctactggctgggcagtgggcggacagaagcctgggagtggagctggccgtgggcacgcaccccccgcctcagtgtcatggaggaggaccaggcctgtgccatggagagccggcgctttgaggagacccgtggcatggaggaggagcccacccacctgcctctggttgtctgcgtggacaaactcaccaaggtctacaaggacgacaagaagctggccctgaacaagctgagcctgaacctctacgagaaccaggtggtctccttcttgggccacaacggggcgggcaagaccaccaccatgtccatcctgaccggcctgttccctccaacgtcgggttccgccaccatctacgggcacgacatccgcacggagatggatgagatccgcaagaacctgggcatgtgcccgcagcacaatgtgctctttgaccggctcacggtggaggaacacctctggttctactcacggctcaagagcatggctcaggaggagatccgcagagagatggacaagatgatcgaggacctggagctctccaacaaacggcactcactggtgcagacattgtcgggtggcatgaagcgcaagctgtccgtggccatcgccttcgtgggcggctctcgcgccatcatcctggacgagcccacggcgggcgtggacccctacgcgcgccgcgccatctgggacctcatcctgaagtacaagccaggccgcaccatccttctgtccacccaccacatggatgaggctgacctgcttggggaccgcattgccatcatctcccatgggaagctcaagtgctgcggctccccgctcttcctcaagggcacctatggcgacgggtaccgcctcacgctggtcaagcggcccgccgagccggggggcccccaagagccagggctggcatccagccccccaggtcgggccccgctgagcagctgctccgagctccaggtgtcccagttcatccgcaagcatgtggcctcctgcctgctggtctcagacacaagcacggagctctcctacatcctgcccagcgaggccgccaagaagggggctttcgagcgcctcttccagcacctggagcgcagcctggatgcactgcacctcagcagcttcgggctgatggacacgaccctggaggaagtgttcctcaaggtgtcggaggaggatcagtcgctggagaacagtgaggccgatgtgaaggagtccaggaaggatgtgctccctggggcggagggcccggcgtctggggagggtcacgctggcaatctggcccggtgctcggagctgacccagtcgcaggcatcgctgcagtcggcgtcatctgtgggctctgcccgtggcgacgagggagctggctacaccgacgtctatggcgactaccgccccctctttgataacccacaggacccagacaatgtcagcctgcaagaggtggaggcagaggccctgtcgagggtcggccagggcagccgcaagctggacggcgggtggctgaaggtgcgccagttccacgggctgctggtcaaacgcttccactgcgcccgccgcaactccaaggcactcttctcccagatcttgctgccagccttcttcgtctgcgtggccatgaccgtggccctgtccgtcccggagattggtgatctgcccccgctggtcctgtcaccttcccagtaccacaactacacccagccccgtggcaatttcatcccctacgccaacgaggagcgccgcgagtaccggctgcggctatcgcccgacgccagcccccagcagctcgtgagcacgttccggctgccgtcgggggtgggtgccacctgcgtgctcaagtctcccgccaacggctcgctggggcccacgttgaacctgagcagcggggagtcgcgcctgctggcggctcggttcttcgacagcatgtgtctggagtccttcacacaggggctgccactgtccaatttcgtgccacccccaccctcgcccgccccatctgactcgccagcgtccccggatgaggacctgcaggcctggaacgtctccctgccgcccaccgctgggccagaaatgtggacgtcggcaccctccctgccgcgcctggtacgggagcccgtccgctgcacctgctctgcgcagggcaccggcttctcctgccccagcagtgtgggcgggcacccgccccagatgcgggtggtcacaggcgacatcctgaccgacatcaccggccacaatgtctctgagtacctgctcttcacctccgaccgcttccgactgcaccggtatggggccatcacctttggaaacgtcctgaagtccatcccagcctcatttggcaccagggccccacccatggtgcggaagatcgcggtgcgcagggctgcccaggttttctacaacaacaagggctatcacagcatgcccacctacctcaacagcctcaacaacgccatcctgcgtgccaacctgcccaagagcaagggcaacccggcggcttacggcatcaccgtcaccaaccaccccatgaataagaccagcgccagcctctccctggattacctgctgcagggcacggatgtcgtcatcgccatcttcatcatcgtggccatgtccttcgtgccggccagcttcgttgtcttcctcgtggccgagaagtccaccaaggccaagcatctgcagtttgtcagcggctgcaaccccatcatctactggctggcgaactacgtgtgggacatgctcaactacctggtccccgctacctgctgtgtcatcatcctgtttgtgttcgacctgccggcctacacgtcgcccaccaacttccctgccgtcctctccctcttcctgctctatgggtggtccatcacgcccatcatgtacccggcctccttctggttcgaggtccccagctccgcctacgtgttcctcattgtcatcaatctcttcatcggcatcaccgccaccgtggccaccttcctgctacagctcttcgagcacgacaaggacctgaaggttgtcaacagttacctgaaaagctgcttcctcattttccccaactacaacctgggccacgggctcatggagatggcctacaacgagtacatcaacgagtactacgccaagattggccagtttgacaagatgaagtccccgttcgagtgggacattgtcacccgcggactggtggccatggcggttgagggcgtcgtgggcttcctcctgaccatcatgtgccagtacaacttcctgcggcggccacagcgcatgcctgtgtctaccaagcctgtggaggatgatgtggacgtggccagtgagcggcagcgagtgctccggggagacgccgacaatgacatggtcaagattgagaacctgaccaaggtctacaagtcccggaagattggccgtatcctggccgttgaccgcctgtgcctgggtgtgcgtcctggcgagtgcttcgggctcctgggcgtcaacggtgcgggcaagaccagcaccttcaagatgctgaccggcgacgagagcacgacggggggcgaggccttcgtcaatggacacagcgtgctgaaggagctgctccaggtgcagcagagcctcggctactgcccgcagtgtgacgcgctgttcgacgagctcacggcccgggagcacctgcagctgtacacgcggctgcgtgggatctcctggaaggacgaggcccgggtggtgaagtgggctctggagaagctggagctgaccaagtacgcagacaagccggctggcacctacagcggcggcaacaagcggaagctctccacggccatcgccctcattgggtacccagccttcatcttcctggacgagcccaccacaggcatggaccccaaggcccggcgcttcctctggaacctcatcctcgacctcatcaagacagggcgttcagtggtgctgacatcacacagcatggaggagtgcgaggcgctgtgcacgcggctggccatcatggtgaacggtcgcctgcggtgcctgggcagcatccagcacctgaagaaccggtttggagatggctacatgatcacggtgcggaccaagagcagccagagtgtgaaggacgtggtgcggttcttcaaccgcaacttcccggaagccatgctcaaggagcggcaccacacaaaggtgcagtaccagctcaagtcggagcacatctcgctggcccaggtgttcagcaagatggagcaggtgtctggcgtgctgggcatcgaggactactcggtcagccagaccacactggacaatgtgttcgtgaactttgccaagaagcagagtgacaacctggagcagcaggagacggagccgccatccgcactgcagtcccctctcggctgcttgctcagcctgctccggccccggtctgcccccacggagctccgggcacttgtggcagacgagcccgaggacctggacacggaggacgagggcctcatcagcttcgaggaggagcgggcccagctgtccttcaacacggacacgctctgctgaccacccagagctgggccagggaggacacgctccactgaccacccagagctgggccagggactcaacaatggggacagaagtcccccagtgcctgccagggcctggagtggaggttcaggaccaaggggcttctggtcctccagcccctgtactcggccatgccctgcggtcactgcggttgccgcccctaattgtgccaaaggctgacccggcccgggctgcgtacacccttgccctgctttgccttaaagcctcggggtctgcccggcccctcgcccctgcctggcactgctcaccgcccaaggcgacgccggctggaccaggcactgctggcctttctcctgcccggcctcggaaccagcttttctctcttacgatgaaggctgatgccgagagcgggctgtgggcggagctgggtcagtcccgtatttattttgctttgagaagaggctcctctggccctgctctcctgcagggaggtggctgtcccgcgggaagccatcagcttgggccagctggcaggtggcaggaatggagaagctgaccctgctggccaggcaaggggccagaccccccccaacccccagctgccatcgctctcccacccagcttggccccctgcccgcccccctccctgggagccgggcctgtacatagggcacagatgtttgtttttaataaataaacaaaatgtcmaaaaaaaaaaaaaaaaaaaaaaaaaaaaaaaaaaaaaaaaaaaaaaaaaaaaaaggg

SEQ ID NO: 8 - protein, 2436 aa, Homo sapiens
MGFLHQLQLLLWKNVTLKRRSPWVLAFEIFIPLVLFFILLGLRQKKPTISVKEVSFYTAAPLTSAGILPVMQSLCPDGQRDEFGFLQYANSTVTQLLERLDRVVEEGNLFDPARPSLGSELEALRQHLEALSAGPGTSGSHLDRSTVSSFSLDSVARNPQELWRFLTQNLSLPNSTAQALLAARVDPPEVYHLLFGPSSALDSQSGLHKGQEPWSRLGGNPLFRMEELLLAPALLEQLTCTPGSGELGRILTVPESQKGALQGYRDAVCSGQAAARARRFSGLSAELRNQLDVAKVSQQLGLDAPNGSDSSPQAPPPRRLQALLGDLLDAQKVLQDVDVLSALALLLPQGACTGRTPGPPASGAGGAANGTGAGAVMGPNATAEEGAPSAAALATPDTLQGQCSAFVQLWAGLQPILCGNNRTIEPEALRRGNMSSLGFTSKEQRNLGLLVHLMTSNPKILYAPAGSEVDRVILKANETFAFVGNVTHYAQVWLNISAEIRSFLEQGRLQQHLRWLQQYVAELRLHPEALNLSLDELPPALRQDNFSLPSGMALLQQLDTIDNAACGWIQFMSKVSVDIFKGFPDEESIVNYTLNQAYQDNVTVFASVIFQTRKDGSLPPHVHYKIRQNSSFTEKTNEIRRAYWRPGPNTGGRFYFLYGFVWIQDMMERAIIDTFVGHDVVEPGSYVQMFPYPCYTRDDFLFVIEHMMPLCMVISWVYSVAMTIQHIVAEKEHRLKEVMKTMGLNNAVHWVAWFITGFVQLSISVTALTAILKYGQVLIHSHVVIIWLFLAVYAVATIMFCFLVSVLYSKAKLASACGGIIYFLSYVPYMYVAIREEVAHDKITAFEKCIASLMSTTAFGLGSKYFALYEVAGVGIQWHTFSQSPVEGDDFNLLLAVTMLMVDAVVYGILTWYIEAVHPGMYGLPRPWYFPLQKSYWLGSGRTEAWEWSWPWARTPRLSVMEEDQACAMESRRFEETRGMEEEPTHLPLVVCVDKLTKVYKDDKKLALNKLSLNLYENQVVSFLGHNGAGKTTTMSILTGLFPPTSGSATIYGHDIRTEMDEIRKNLGMCPQHNVLFDRLTVEEHLWFYSRLKSMAQEEIRREMDKMIEDLELSNKRHSLVQTLSGGMKRKLSVAIAFVGGSRAIILDEPTAGVDPYARRAIWDLILKYKPGRTILLSTHHMDEADLLGDRIAIISHGKLKCCGSPLFLKGTYGDGYRLTLVKRPAEPGGPQEPGLASSPPGRAPLSSCSELQVSQFIRKHVASCLLVSDTSTELSYILPSEAAKKGAFERLFQHLERSLDALHLSSFGLMDTTLEEVFLKVSEEDQSLENSEADVKESRKDVLPGAEGPASGEGHAGNLARCSELTQSQASLQSASSVGSARGDEGAGYTDVYGDYRPLFDNPQDPDNVSLQEVEAEALSRVGQGSRKLDGGWLKVRQFHGLLVKRFHCARRNSKALFSQILLPAFFVCVAMTVALSVPEIGDLPPLVLSPSQYHNYTQPRGNFIPYANEERREYRLRLSPDASPQQLVSTFRLPSGVGATCVLKSPANGSLGPTLNLSSGESRLLAARFFDSMCLESFTQGLPLSNFVPPPPSPAPSDSPASPDEDLQAWNVSLPPTAGPEMWTSAPSLPRLVREPVRCTCSAQGTGFSCPSSVGGHPPQMRVVTGDILTDITGHNVSEYLLFTSDRFRLHRYGAITFGNVLKSIPASFGTRAPPMVRKIAVRRAAQVFYNNKGYHSMPTYLNSLNNAILRANLPKSKGNPAAYGITVTNHPMNKTSASLSLDYLLQGTDVVIAIFIIVAMSFVPASFVVFLVAEKSTKAKHLQFVSGCNPIIYWLANYVWDMLNYLVPATCCVIILFVFDLPAYTSPTNFPAVLSLFLLYGWSITPIMYPASFWFEVPSSAYVFLIVINLFIGITATVATFLLQLFEHDKDLKVVNSYLKSCFLIFPNYNLGHGLMEMAYNEYINEYYAKIGQFDKMKSPFEWDIVTRGLVAMAVEGVVGFLLTIMCQYNFLRRPQRMPVSTKPVEDDVDVASERQRVLRGDADNDMVKIENLTKVYKSRKIGRILAVDRLCLGVRPGECFGLLGVNGAGKTSTFKMLTGDESTTGGEAFVNGHSVLKELLQVQQSLGYCPQCDALFDELTAREHLQLYTRLRGISWKDEARVVKWALEKLELTKYADKPAGTYSGGNKRKLSTAIALIGYPAFIFLDEPTTGMDPKARRFLWNLILDLIKTGRSVVLTSHSMEECEALCTRLAIMVNGRLRCLGSIQHLKNRFGDGYMITVRTKSSQSVKDVVRFFNRNFPEAMLKERHHTKVQYQLKSEHISLAQVFSKMEQVSGVLGIEDYSVSQTTLDNVFVNFAKKQSDNLEQQETEPPSALQSPLGCLLSLLRPRSAPTELRALVADEPEDLDTEDEGLISFEEERAQLSFNTDTLC

SEQ ID NO: 9 - DNA, 7305 bp, Homo sapiens
atgggcttcctgcaccagctgcagctgctgctctggaagaacgtgacgctcaaacgccggagcccgtgggtcctggccttcgagatcttcatccccctggtgctgttctttatcctgctggggctgcgacagaagaagcccaccatctccgtgaaggaagtctccttctacacagcggcgcccctgacgtctgccggcatcctgcctgtcatgcaatcgctgtgcccggacggccagcgagacgagttcggcttcctgcagtacgccaactccacggtcacgcagctgcttgagcgcctggaccgcgtggtggaggaaggcaacctgtttgacccagcgcggcccagcctgggctcagagctcgaggccctacgccagcatctggaggccctcagtgcgggcccgggcacctcggggagccacctggacagatccacagtgtcttccttctctctggactcggtggccagaaacccgcaggagctctggcgtttcctgacgcaaaacttgtcgctgcccaatagcacggcccaagcactcttggccgcccgtgtggacccgcccgaggtctaccacctgctctttggtccctcatctgccctggattcacagtctggcctccacaagggtcaggagccctggagccgcctagggggcaatcccctgttccggatggaggagctgctgctggctcctgccctcctggagcagctcacctgcacgccgggctcgggggagctgggccggatcctcactgtgcctgagagtcagaagggagccctgcagggctaccgggatgctgtctgcagtgggcaggctgctgcgcgtgccaggcgcttctctgggctgtctgctgagctccggaaccagctggacgtggccaaggtctcccagcagctgggcctggatgcccccaacggctcggactcctcgccacaggcgccacccccacggaggctgcaggcgcttctgggggacctgctggatgcccagaaggttctgcaggatgtggatgtcctgtcggccctggccctgctactgccccagggtgcctgcactggccggacccccggacccccagccagtggtgcgggtggggcggccaatggcactggggcaggggcagtcatgggccccaacgccaccgctgaggagggcgcaccctctgctgcagcactggccaccccggacacgctgcagggccagtgctcagccttcgtacagctctgggccggcctgcagcccatcttgtgtggcaacaaccgcaccattgaacccgaggcgctgcggcggggcaacatgagctccctgggcttcacgagcaaggagcagcggaacctgggcctcctcgtgcacctcatgaccagcaaccccaaaatcctgtacgcgcctgcgggctctgaggtcgaccgcgtcatcctcaaggccaacgagacttttgcttttgtgggcaacgtgactcactatgcccaggtctggctcaacatctcggcggagatccgcagcttcctggagcagggcaggctgcagcaacacctgcgctggctgcagcagtatgtagcagagctgcggctgcaccccgaggcactgaacctgtcactggatgagctgccgccggccctgagacaggacaacttctcgctgcccagtggcatggccctcctgcagcagctggataccattgacaacgcggcctgcggctggatccagttcatgtccaaggtgagcgtggacatcttcaagggcttccccgacgaggagagcattgtcaactacaccctcaaccaggcctaccaggacaacgtcactgtttttgccagtgtgatcttccagacccggaaggacggctcgctcccgcctcacgtgcactacaagatccgccagaactccagcttcaccgagaaaaccaacgagatccgccgcgcctactggcggcctgggcccaatactggcggccgcttctacttcctctacggcttcgtctggatccaggacatgatggagcgcgccatcatcgacacttttgtggggcacgacgtggtggagccaggcagctacgtgcagatgttcccctacccctgctacacacgcgatgacttcctgtttgtcattgagcacatgatgccgctgtgcatggtgatctcctgggtctactccgtggccatgaccatccagcacatcgtggcggagaaggagcaccggctcaaggaggtgatgaagaccatgggcctgaacaacgcggtgcactgggtggcctggttcatcaccggctttgtgcagctgtccatctccgtgacagcactcaccgccatcctgaagtacggccaggtgcttatacacagccacgtggtcatcatctggctcttcctggcagtctacgcggtggccaccatcatgttctgcttcctggtgtctgtgctgtactccaaggccaagctggcctcggcctgcggtggcatcatctacttcctgagctacgtgccctacatgtacgtggcgatccgagaggaggtggcgcatgataagatcacggccttcgagaagtgcatcgcgtccctcatgtccacgacggcctttggtctgggctctaagtacttcgcgctgtatgaggtggccggcgtgggcatccagtggcacaccttcagccagtccccggtggagggggacgacttcaacttgctcctggctgtcaccatgctgatggtggacgccgtggtctatggcatcctcacgtggtacattgaggctgtgcacccaggcatgtacgggctgccccggccctggtacttcccactgcagaagtcctactggctgggcagtgggcggacagaagcctgggagtggagctggccgtgggcacgcaccccccgcctcagtgtcatggaggaggaccaggcctgtgccatggagagccggcgctttgaggagacccgtggcatggaggaggagcccacccacctgcctctggttgtctgcgtggacaaactcaccaaggtctacaaggacgacaagaagctggccctgaacaagctgagcctgaacctctacgagaaccaggtggtctccttcttgggccacaacggggcgggcaagaccaccaccatgtccatcctgaccggcctgttccctccaacgtcgggttccgccaccatctacgggcacgacatccgcacggagatggatgagatccgcaagaacctgggcatgtgcccgcagcacaatgtgctctttgaccggctcacggtggaggaacacctctggttctactcacggctcaagagcatggctcaggaggagatccgcagagagatggacaagatgatcgaggacctggagctctccaacaaacggcactcactggtgcagacattgtcgggtggcatgaagcgcaagctgtccgtggccatcgccttcgtgggcggctctcgcgccatcatcctggacgagcccacggcgggcgtggacccctacgcgcgccgcgccatctgggacctcatcctgaagtacaagccaggccgcaccatccttctgtccacccaccacatggatgaggctgacctgcttggggaccgcattgccatcatctcccatgggaagctcaagtgctgcggctccccgctcttcctcaagggcacctatggcgacgggtaccgcctcacgctggtcaagcggcccgccgagccggggggcccccaagagccagggctggcatccagccccccaggtcgggccccgctgagcagctgctccgagctccaggtgtcccagttcatccgcaagcatgtggcctcctgcctgctggtctcagacacaagcacggagctctcctacatcctgcccagcgaggccgccaagaagggggctttcgagcgcctcttccagcacctggagcgcagcctggatgcactgcacctcagcagcttcgggctgatggacacgaccctggaggaagtgttcctcaaggtgtcggaggaggatcagtcgctggagaacagtgaggccgatgtgaaggagtccaggaaggatgtgctccctggggcggagggcccggcgtctggggagggtcacgctggcaatctggcccggtgctcggagctgacccagtcgcaggcatcgctgcagtcggcgtcatctgtgggctctgcccgtggcgacgagggagctggctacaccgacgtctatggcgactaccgccccctctttgataacccacaggacccagacaatgtcagcctgcaagaggtggaggcagaggccctgtcgagggtcggccagggcagccgcaagctggacggcgggtggctgaaggtgcgccagttccacgggctgctggtcaaacgcttccactgcgcccgccgcaactccaaggcactcttctcccagatcttgctgccagccttcttcgtctgcgtggccatgaccgtggccctgtccgtcccggagattggtgatctgcccccgctggtcctgtcaccttcccagtaccacaactacacccagccccgtggcaatttcatcccctacgccaacgaggagcgccgcgagtaccggctgcggctatcgcccgacgccagcccccagcagctcgtgagcacgttccggctgccgtcgggggtgggtgccacctgcgtgctcaagtctcccgccaacggctcgctggggcccacgttgaacctgagcagcggggagtcgcgcctgctggcggctcggttcttcgacagcatgtgtctggagtccttcacacaggggctgccactgtccaatttcgtgccacccccaccctcgcccgccccatctgactcgccagcgtccccggatgaggacctgcaggcctggaacgtctccctgccgcccaccgctgggccagaaatgtggacgtcggcaccctccctgccgcgcctggtacgggagcccgtccgctgcacctgctctgcgcagggcaccggcttctcctgccccagcagtgtgggcgggcacccgccccagatgcgggtggtcacaggcgacatcctgaccgacatcaccggccacaatgtctctgagtacctgctcttcacctccgaccgcttccgactgcaccggtatggggccatcacctttggaaacgtcctgaagtccatcccagcctcatttggcaccagggccccacccatggtgcggaagatcgcggtgcgcagggctgcccaggttttctacaacaacaagggctatcacagcatgcccacctacctcaacagcctcaacaacgccatcctgcgtgccaacctgcccaagagcaagggcaacccggcggcttacggcatcaccgtcaccaaccaccccatgaataagaccagcgccagcctctccctggattacctgctgcagggcacggatgtcgtcatcgccatcttcatcatcgtggccatgtccttcgtgccggccagcttcgttgtcttcctcgtggccgagaagtccaccaaggccaagcatctgcagtttgtcagcggctgcaaccccatcatctactggctggcgaactacgtgtgggacatgctcaactacctggtccccgctacctgctgtgtcatcatcctgtttgtgttcgacctgccggcctacacgtcgcccaccaacttccctgccgtcctctccctcttcctgctctatgggtggtccatcacgcccatcatgtacccggcctccttctggttcgaggtccccagctccgcctacgtgttcctcattgtcatcaatctcttcatcggcatcaccgccaccgtggccaccttcctgctacagctcttcgagcacgacaaggacctgaaggttgtcaacagttacctgaaaagctgcttcctcattttccccaactacaacctgggccacgggctcatggagatggcctacaacgagtacatcaacgagtactacgccaagattggccagtttgacaagatgaagtccccgttcgagtgggacattgtcacccgcggactggtggccatggcggttgagggcgtcgtgggcttcctcctgaccatcatgtgccagtacaacttcctgcggcggccacagcgcatgcctgtgtctaccaagcctgtggaggatgatgtggacgtggccagtgagcggcagcgagtgctccggggagacgccgacaatgacatggtcaagattgagaacctgaccaaggtctacaagtcccggaagattggccgtatcctggccgttgaccgcctgtgcctgggtgtgcgtcctggcgagtgcttcgggctcctgggcgtcaacggtgcgggcaagaccagcaccttcaagatgctgaccggcgacgagagcacgacggggggcgaggccttcgtcaatggacacagcgtgctgaaggagctgctccaggtgcagcagagcctcggctactgcccgcagtgtgacgcgctgttcgacgagctcacggcccgggagcacctgcagctgtacacgcggctgcgtgggatctcctggaaggacgaggcccgggtggtgaagtgggctctggagaagctggagctgaccaagtacgcagacaagccggctggcacctacagcggcggcaacaagcggaagctctccacggccatcgccctcattgggtacccagccttcatcttcctggacgagcccaccacaggcatggaccccaaggcccggcgcttcctctggaacctcatcctcgacctcatcaagacagggcgttcagtggtgctgacatcacacagcatggaggagtgcgaggcgctgtgcacgcggctggccatcatggtgaacggtcgcctgcggtgcctgggcagcatccagcacctgaagaaccggtttggagatggctacatgatcacggtgcggaccaagagcagccagagtgtgaaggacgtggtgcggttcttcaaccgcaacttcccggaagccatgctcaaggagcggcaccacacaaaggtgcagtaccagctcaagtcggagcacatctcgctggcccaggtgttcagcaagatggagcaggtgtctggcgtgctgggcatcgaggactactcggtcagccagaccacactggacaatgtgttcgtgaactttgccaagaagcagagtgacaacctggagcagcaggagacggagccgccatccgcactgcagtcccctctcggctgcttgctcagcctgctccggccccggtctgcccccacggagctccgggcacttgtggcagacgagcccgaggacctggacacggaggacgagggcctcatcagcttcgaggaggagcgggcccagctgtccttcaacacggacacgctc

SEQ ID NO: 10 - DNA, 2150 bp, Homo sapiens
  CDS (221)...(1573)
aagtactaaaatccaggggcaggcacaaattggagggtcagaagactggaggggccgcagggctcgctggagggtggctggacccgccagagatctgtcttacttctgacttctaggtactgtccacaccatccttgccagctgggcctaattttgccctaggtctggccaggaggcctcacatccagagacctgcccccgcccttgcagtgccagggccatggggctccggagccaccacctcagcctgggccttctgcttctgtctctactccctgcagagtgcctgggagctgagggccggctggctctcaagctgttccgtgacctctttgccaactacacaagtgccctgagacctgtggcagacacagaccagactctgaatgtgaccctggaggtgacactgtcccagatcatcgacatggatgaacggaaccaggtgctgaccctgtatctgtggatacggcaggagtggacagatgcctacctacgatgggaccccaatgcctatggtggcctggatgccatccgcatccccagcagtcttgtgtggcggccagacatcgtactctataacaaagccgacgcgcagcctccaggttccgccagcaccaacgtggtcctgcgccacgatggcgccgtgcgctgggacgcgccggccatcacgcgcagctcgtgccgcgtggatgtagcagccttcccgttcgacgcccagcactgcggcctgacgttcggctcctggactcacggcgggcaccaactggatgtgcggccgcgcggcgctgcagccagcctggcggacttcgtggagaacgtggagtggcgcgtgctgggcatgccggcgcggcggcgcgtgctcacctacggctgctgctccgagccctaccccgacgtcaccttcacgctgctgctgcgccgccgcgccgccgcctacgtgtgcaacctgctgctgccctgcgtgctcatctcgctgcttgcgccgctcgccttccacctgcctgccgactcaggcgagaaggtgtcgctgggcgtcaccgtgctgctggcgctcaccgtcttccagttgctgctggccgagagcatgccaccggccgagagcgtgccgctcatcgggaagtactacatggccactatgaccatggtcacattctcaacagcactcaccatccttatcatgaacctgcattactgtggtcccagtgtccgcccagtgccagcctgggctagggccctcctgctgggacacctggcacggggcctgtgcgtgcgggaaagaggggagccctgtgggcagtccaggccacctgagttatctcctagcccccagtcgcctgaaggaggggctggccccccagcgggcccttgccacgagccacgatgtctgtgccgccaggaagccctactgcaccacgtagccaccattgccaataccttccgcagccaccgagctgcccagcgctgccatgaggactggaagcgcctggcccgtgtgatggaccgcttcttcctggccatcttcttctccatggccctggtcatgagcctcctggtgctggtgcaggccctgtgagggctgggactaagtcacagggatctgctgcagccacagctcctccagaaagggacagccacggccaagtggttgctggtctttgggccagccagtctctccccactgctcctaagatcctgagacacttgacttcacaatccacaagggagcactcattgtctacacaccctaactaaaggaagtccagagcctgccactcccctaattccaaaaaaaagaggaactctacaaaggccaagatcacagagtacagtcttggagggacagaattgtttgtgctgggtattggagctctcagtggggagcacatgggttataatgagaaactgaactgtactgctgcatttcctgtcttccttcctaggtggctgctttgcagggctttggctgttacctttccctgctgaggggctcagggaaaagggtcggggattctcagtcgagtttccagagcaggaggccctacagacatttcgccccaaatccctgactcaataaagtaagcgtgtacctagcaaaaaaaaaaaaaaaaaacctcgtgccgaattcttggcctcgagggccaaattccctg

SEQ ID NO: 11 - protein, 450 aa, Homo sapiens
MGLRSHHLSLGLLLLSLLPAECLGAEGRLALKLFRDLFANYTSALRPVADTDQTLNVTLEVTLSQIIDMDERNQVLTLYLWIRQEWTDAYLRWDPNAYGGLDAIRIPSSLVWRPDIVLYNKADAQPPGSASTNVVLRHDGAVRWDAPAITRSSCRVDVAAFPFDAQHCGLTFGSWTHGGHQLDVRPRGAAASLADFVENVEWRVLGMPARRRVLTYGCCSEPYPDVTFTLLLRRRAAAYVCNLLLPCVLISLLAPLAFHLPADSGEKVSLGVTVLLALTVFQLLLAESMPPAESVPLIGKYYMATMTMVTFSTALTILIMNLHYCGPSVRPVPAWARALLLGHLARGLCVRERGEPCGQSRPPELSPSPQSPEGGAGPPAGPCHEPRCLCRQEALLHHVATIANTFRSHRAAQRCHEDWKRLARVMDRFFLAIFFSMALVMSLLVLVQAL

SEQ ID NO: 12 - DNA, 1350 bp, Homo sapiens
atggggctccggagccaccacctcagcctgggccttctgcttctgtctctactccctgcagagtgcctgggagctgagggccggctggctctcaagctgttccgtgacctctttgccaactacacaagtgccctgagacctgtggcagacacagaccagactctgaatgtgaccctggaggtgacactgtcccagatcatcgacatggatgaacggaaccaggtgctgaccctgtatctgtggatacggcaggagtggacagatgcctacctacgatgggaccccaatgcctatggtggcctggatgccatccgcatccccagcagtcttgtgtggcggccagacatcgtactctataacaaagccgacgcgcagcctccaggttccgccagcaccaacgtggtcctgcgccacgatggcgccgtgcgctgggacgcgccggccatcacgcgcagctcgtgccgcgtggatgtagcagccttcccgttcgacgcccagcactgcggcctgacgttcggctcctggactcacggcgggcaccaactggatgtgcggccgcgcggcgctgcagccagcctggcggacttcgtggagaacgtggagtggcgcgtgctgggcatgccggcgcggcggcgcgtgctcacctacggctgctgctccgagccctaccccgacgtcaccttcacgctgctgctgcgccgccgcgccgccgcctacgtgtgcaacctgctgctgccctgcgtgctcatctcgctgcttgcgccgctcgccttccacctgcctgccgactcaggcgagaaggtgtcgctgggcgtcaccgtgctgctggcgctcaccgtcttccagttgctgctggccgagagcatgccaccggccgagagcgtgccgctcatcgggaagtactacatggccactatgaccatggtcacattctcaacagcactcaccatccttatcatgaacctgcattactgtggtcccagtgtccgcccagtgccagcctgggctagggccctcctgctgggacacctggcacggggcctgtgcgtgcgggaaagaggggagccctgtgggcagtccaggccacctgagttatctcctagcccccagtcgcctgaaggaggggctggccccccagcgggcccttgccacgagccacgatgtctgtgccgccaggaagccctactgcaccacgtagccaccattgccaataccttccgcagccaccgagctgcccagcgctgccatgaggactggaagcgcctggcccgtgtgatggaccgcttcttcctggccatcttcttctccatggccctggtcatgagcctcctggtgctggtgcaggccctg

SEQ ID NO: 13 - DNA, 2593 bp, Homo sapiens
  CDS (62)...(2317)
cgagcgtagcggcctctagctcgagcagcaggagcagcccgcaccggacaacttgcgagccatggggctggcggatgcgtcgggaccgagggacacacaggcactgctgtctgcaacacaagcaatggacctgcggaggcgagactaccacatggaacggccgctgctgaaccaggagcatttggaggagctggggcgctggggctcagcacctaggacccaccagtggcggacctggttgcagtgctcccgtgctcgggcctatgcccttctgctccaacacctcccggttttggtctggttaccccggtatcctgtgcgtgactggctcctgggtgacctgttatccggcctgagtgtggccatcatgcagcttccgcagggcttggcctacgccctcctggctggattgccccccgtgtttggcctctatagctccttctaccctgtcttcatctacttcctgtttggcacttcccggcacatctccgtggagagcctctgtgtcccgggaccagtagacacagggacctttgctgtcatgtctgtgatggtgggcagtgtgacagaatccctggccccgcaggccttgaacgactccatgatcaatgagacagccagagatgctgcccgggtacaggtggcctccacactcagtgtcctggttggcctcttccaggtggggctgggcctgatccacttcggcttcgtggtcacctacctgtcagaacctcttgtccgaggctataccacagctgcagctgtgcaggtcttcgtctcacagctcaagtatgtgtttggcctccatctgagcagccactctgggccactgtccctcatctatacagtgctggaggtctgctggaagctgccccagagcaaggttggcaccgtggtcactgcagctgtggctggggtggtgctcgtggtggtgaagctgttgaatgacaagctgcagcagcagctgcccatgccgatacccggggagctgctcacgctcatcggggccacaggcatctcctatggcatgggtctaaagcacagatttgaggtagatgtcgtgggcaacatccctgcagggctggtgcccccagtggcccccaacacccagctgttctcaaagctcgtgggcagcgccttcaccatcgctgtggttgggtttgccattgccatctcactggggaagatcttcgccctgaggcacggctaccgggtggacagcaaccaggagctggtggccctgggcctcagtaaccttatcggaggcatcttccagtgcttccccgtgagttgctctatgtctcggagcctggtacaggagagcaccgggggcaactcgcaggttgctggagccatctcttcccttttcatcctcctcatcattgtcaaacttggggaactcttccatgacctgcccaaggcggtcctggcagccatcatcattgtgaacctgaagggcatgctgaggcagctcagcgacatgcgctccctctggaaggccaatcgggcggatctgcttatctggctggtgaccttcacggccaccatcttgctgaacctggaccttggcttggtggttgcggtcatcttctccctgctgctcgtggtggtccggacacagatgccccactactctgtcctggggcaggtgccagacacggatatttacagagatgtggcagagtactcagaggccaaggaagtccggggggtgaaggtcttccgctcctcggccaccgtgtactttgccaatgctgagttctacagtgatgcgctgaagcagaggtgtggtgtggatgtcgacttcctcatctcccagaagaagaaactgctcaagaagcaggagcagctgaagctgaagcaactgcagaaagaggagaagcttcggaaacaggcagggccccttttgtctgcatgtctggctccccagcaggtgagctcaggagataagatggaagatgcaacagccaatggtcaagaagactccaaggccccagatgggtccacactgaaggccctgggcctgcctcagccagacttccacagcctcatcctggacctgggtgccctctcctttgtggacactgtgtgcctcaagagcctgaagaatattttccatgacttccgggagattgaggtggaggtgtacatggcggcctgccacagccctgtggtcagccagcttgaggctgggcacttcttcgatgcatccatcaccaagaagcatctctttgcctctgtccatgatgctgtcacctttgccctccaacacccgaggcctgtccccgacagccctgtttcggtcaccagactctgaacatgctacatcctgcccaagactgcacctctggaggtgcagggcacccttgagaagcccctcacccctaggccgcctccaggtgctacccaggagtcccctccatgtacacacacacaactcagggaaggaggtcctgggactccaagttcagcgctccaggtctgggacagggcctgcatgcagtcaggctggcagtggcgcggtacagggagggaactggtgcatattttagcctcaggaataaagatttgtctgctcaaaaaaaaaaaaaaa

SEQ ID NO: 14 - protein, 751 aa, Homo sapiens
MGLADASGPRDTQALLSATQAMDLRRRDYHMERPLLNQEHLEELGRWGSAPRTHQWRTWLQCSRARAYALLLQHLPVLVWLPRYPVRDWLLGDLLSGLSVAIMQLPQGLAYALLAGLPPVFGLYSSFYPVFIYFLFGTSRHISVESLCVPGPVDTGTFAVMSVMVGSVTESLAPQALNDSMINETARDAARVQVASTLSVLVGLFQVGLGLIHFGFVVTYLSEPLVRGYTTAAAVQVFVSQLKYVFGLHLSSHSGPLSLIYTVLEVCWKLPQSKVGTVVTAAVAGVVLVVVKLLNDKLQQQLPMPIPGELLTLIGATGISYGMGLKHRFEVDVVGNIPAGLVPPVAPNTQLFSKLVGSAFTIAVVGFAIAISLGKIFALRHGYRVDSNQELVALGLSNLIGGIFQCFPVSCSMSRSLVQESTGGNSQVAGAISSLFILLIIVKLGELFHDLPKAVLAAIIIVNLKGMLRQLSDMRSLWKANRADLLIWLVTFTATILLNLDLGLVVAVIFSLLLVVVRTQMPHYSVLGQVPDTDIYRDVAEYSEAKEVRGVKVFRSSATVYFANAEFYSDALKQRCGVDVDFLISQKKKLLKKQEQLKLKQLQKEEKLRKQAGPLLSACLAPQQVSSGDKMEDATANGQEDSKAPDGSTLKALGLPQPDFHSLILDLGALSFVDTVCLKSLKNIFHDFREIEVEVYMAACHSPVVSQLEAGHFFDASITKKHLFASVHDAVTFALQHPRPVPDSPVSVTRL

SEQ ID NO: 15 - DNA, 2253 bp, Homo sapiens
atggggctggcggatgcgtcgggaccgagggacacacaggcactgctgtctgcaacacaagcaatggacctgcggaggcgagactaccacatggaacggccgctgctgaaccaggagcatttggaggagctggggcgctggggctcagcacctaggacccaccagtggcggacctggttgcagtgctcccgtgctcgggcctatgcccttctgctccaacacctcccggttttggtctggttaccccggtatcctgtgcgtgactggctcctgggtgacctgttatccggcctgagtgtggccatcatgcagcttccgcagggcttggcctacgccctcctggctggattgccccccgtgtttggcctctatagctccttctaccctgtcttcatctacttcctgtttggcacttcccggcacatctccgtggagagcctctgtgtcccgggaccagtagacacagggacctttgctgtcatgtctgtgatggtgggcagtgtgacagaatccctggccccgcaggccttgaacgactccatgatcaatgagacagccagagatgctgcccgggtacaggtggcctccacactcagtgtcctggttggcctcttccaggtggggctgggcctgatccacttcggcttcgtggtcacctacctgtcagaacctcttgtccgaggctataccacagctgcagctgtgcaggtcttcgtctcacagctcaagtatgtgtttggcctccatctgagcagccactctgggccactgtccctcatctatacagtgctggaggtctgctggaagctgccccagagcaaggttggcaccgtggtcactgcagctgtggctggggtggtgctcgtggtggtgaagctgttgaatgacaagctgcagcagcagctgcccatgccgatacccggggagctgctcacgctcatcggggccacaggcatctcctatggcatgggtctaaagcacagatttgaggtagatgtcgtgggcaacatccctgcagggctggtgcccccagtggcccccaacacccagctgttctcaaagctcgtgggcagcgccttcaccatcgctgtggttgggtttgccattgccatctcactggggaagatcttcgccctgaggcacggctaccgggtggacagcaaccaggagctggtggccctgggcctcagtaaccttatcggaggcatcttccagtgcttccccgtgagttgctctatgtctcggagcctggtacaggagagcaccgggggcaactcgcaggttgctggagccatctcttcccttttcatcctcctcatcattgtcaaacttggggaactcttccatgacctgcccaaggcggtcctggcagccatcatcattgtgaacctgaagggcatgctgaggcagctcagcgacatgcgctccctctggaaggccaatcgggcggatctgcttatctggctggtgaccttcacggccaccatcttgctgaacctggaccttggcttggtggttgcggtcatcttctccctgctgctcgtggtggtccggacacagatgccccactactctgtcctggggcaggtgccagacacggatatttacagagatgtggcagagtactcagaggccaaggaagtccggggggtgaaggtcttccgctcctcggccaccgtgtactttgccaatgctgagttctacagtgatgcgctgaagcagaggtgtggtgtggatgtcgacttcctcatctcccagaagaagaaactgctcaagaagcaggagcagctgaagctgaagcaactgcagaaagaggagaagcttcggaaacaggcagggccccttttgtctgcatgtctggctccccagcaggtgagctcaggagataagatggaagatgcaacagccaatggtcaagaagactccaaggccccagatgggtccacactgaaggccctgggcctgcctcagccagacttccacagcctcatcctggacctgggtgccctctcctttgtggacactgtgtgcctcaagagcctgaagaatattttccatgacttccgggagattgaggtggaggtgtacatggcggcctgccacagccctgtggtcagccagcttgaggctgggcacttcttcgatgcatccatcaccaagaagcatctctttgcctctgtccatgatgctgtcacctttgccctccaacacccgaggcctgtccccgacagccctgtttcggtcaccagactc

SEQ ID NO: 16 - DNA, 3408 bp, Homo sapiens
  CDS (169)...(2469)
ccacgcgtccgtaccccgggtccctgcctggcgcttccggtccctccgccacagtctgttggattacctcagattgccccagcctggcctcgccctgtggatgatgatggccttgcccccgtgagctacaacctggccttcagcacccgcccacctccaaccagcaggatgcggctgtggaaggcggtggtggtgactttggccttcatgagtgtggacatctgcgtgaccacggccatctatgtcttcagccacctggaccgcagcctcctggaggacatccgccacttcaacatctttgactcggtgctggatctctgggcagcctgcctgtaccgcagctgcctgctgctgggagccaccattggtgtggccaagaacagtgcgctggggccccggcggctgcgggcctcgtggctggtcatcaccctcgtgtgcctcttcgtgggcatctatgccatggtgaagctgctgctcttctcagaggtgcgcaggcccatccgggacccctggttttgggccctgttcgtgtggacgtacatttcactcggcgcatccttcctgctctggtggctgctgtccaccgtgcggccaggcacccaggccctggagccaggggcggccaccgaggctgagggcttccctgggagcggccggccaccgcccgagcaggcgtctggggccacgctgcagaagctgctctcctacaccaagcccgacgtggccttcctcgtggccgcctccttcttcctcatcgtggcagctctgggagagaccttcctgccctactacacgggccgcgccattgatggcatcgtcatccagaaaagcatggatcagttcagcacggctgtcgtcatcgtgtgcctgctggccattggcagctcatttgccgcaggtattcggggcggcatttttaccctcatatttgccagactgaacattcgccttcgaaactgtctcttccgctcactggtgtcccaggagacaagcttctttgatgagaaccgcacaggggacctcatctcccgcctgacctcggacaccaccatggtcagcgacctggtctcccagaacatcaatgtcttcctgcggaacacagtcaaggtcacgggcgtggtggtcttcatgttcagcctctcatggcagctctccttggtcaccttcatgggcttccccatcatcatgatggtgtccaacatctacggcaagtactacaagaggctctccaaagaggtccagaatgccctggccagagcgagcaacacggcggaggagaccatcagtgccatgaagactgtccggagcttcgccaatgaggaggaggaggcagaggtgtacctgcggaagctgcagcaggtgtacaagctgaacaggaaggaggcagctgcctacatgtactacgtctggggcagcgggctcacactgctggtggtccaggtcagcatcctctactacgggggccaccttgtcatctcaggccagatgaccagcggcaacctcatcgccttcatcatctacgagtttgtcctgggagattgtatggagtccgtgggttccgtttacagtggcctgatgcagggagtgggggctgctgagaaggtgttcgagttcatcgaccggcagccgaccatggtgcacgatggcagcttggcccccgaccacytggagggccgggtggactttgagaatgtgaccttcacytaccgcactcggccccacacccaggtcctgcagaatgtctccttcagcctgtcccccggcaaggtgacggccctggtggggccctcgggcagtgggaagagctcctgtgtcaacatcctggagaacttctaccccctggaggggggccgggtgctgctggacggcaagcccatcagcgcctacgaccacaagtacttgcaccgtgtgatctccctggtgagccaggagcccgtgctgttcgcccgctccatcacggataacatctcctacggcctgcccactgtgcctttcgagatggtggtggaggccgcacagaaggccaatgcccacggcttcatcatggaactccaggacggctacagcacagagacaggggagaagggcgcccagctgtcaggtggccagaagcagcgggtggccatggcccgggctctggtgcggaaccccccagtcctcatcctggatgaagccaccagcgctttggatgccgagagcgagtatctgatccagcaggccatccatggcaacctgcagaagcacacggtactcatcatcgcgcaccggctgagcaccgtggagcacgcgcacctcattgtggtgctggacaagggccgcgtagtgcagcagggcacccaccagcagctgctggcccagggcggcctctacgccaagctggtgcagcggcagatgctggggcttcagcccgccgcagacttcacagctggccacaacgagcctgtagccaacggcagtcacaaggcctgatggggggcccctgcttctcccggtggggcagaggacccggtgcctgcctggcagatgtgcccacggaggcccccagctgccctccgagcccaggcctgcagcactgaaagacgacctgccatgtcccatggatcaccgcttcctgcatcttgcccctggtccctgccccattcccagggcactccttacccctgctgccctgagccaacgccttcacggacctccctagcctcctaagcaaaggtagagctgcctttttaaacctaggtcttaccagggtttttactgtttggtttgaggcaccccagtcaactcctagatttcaaaaacctttttctaattgggagtaatggcgggcactttcaccaagatgttctagaaacttctgagccaggagtgaatggcccttccttagtagcctgggggatgtccagagactaggcctctcccctttacccctccagagaaggggcttccctgtcccggagggagacacggggaacgggattttccgtctctccctcttgccagctctgtgagtctggccagggcgggtagggagcgtggagggcatctgtctgccatcgcccgctgccaatctaagccagtctcactgtgaaccacacgaaacctcaactgggggagtgaggggctggccaggtctggaggggcctcaggggtgcccccagcccggcacccagcgctttcgcccctcgtccacccacccctggctggcagcctccctccccacacccgcccctgtgctctgctgtctggaggccacgtggatgttcatgagatgcattctcttctgtctttggtggatgggatggtggcaaagcccaggatctggctttgccagaggttgcaacatgttgagagaacccggtcaataaagtgtactacctcttacccctaaaaaaaaaaaaaaaaaaaagg

SEQ ID NO: 17 - protein, 766 aa, Homo sapiens
  VARIANT (1)...(766): Xaa = Any Amino Acid
MRLWKAVVVTLAFMSVDICVTTAIYVFSHLDRSLLEDIRHFNIFDSVLDLWAACLYRSCLLLGATIGVAKNSALGPRRLRASWLVITLVCLFVGIYAMVKLLLFSEVRRPIRDPWFWALFVWTYISLGASFLLWWLLSTVRPGTQALEPGAATEAEGFPGSGRPPPEQASGATLQKLLSYTKPDVAFLVAASFFLIVAALGETFLPYYTGRAIDGIVIQKSMDQFSTAVVIVCLLAIGSSFAAGIRGGIFTLIFARLNIRLRNCLFRSLVSQETSFFDENRTGDLISRLTSDTTMVSDLVSQNINVFLRNTVKVTGVVVFMFSLSWQLSLVTFMGFPIIMMVSNIYGKYYKRLSKEVQNALARASNTAEETISAMKTVRSFANEEEEAEVYLRKLQQVYKLNRKEAAAYMYYVWGSGLTLLVVQVSILYYGGHLVISGQMTSGNLIAFIIYEFVLGDCMESVGSVYSGLMQGVGAAEKVFEFIDRQPTMVHDGSLAPDHXEGRVDFENVTFXYRTRPHTQVLQNVSFSLSPGKVTALVGPSGSGKSSCVNILENFYPLEGGRVLLDGKPISAYDHKYLHRVISLVSQEPVLFARSITDNISYGLPTVPFEMVVEAAQKANAHGFIMELQDGYSTETGEKGAQLSGGQKQRVAMARALVRNPPVLILDEATSALDAESEYLIQQAIHGNLQKHTVLIIAHRLSTVEHAHLIVVLDKGRVVQQGTHQQLLAQGGLYAKLVQRQMLGLQPAADFTAGHNEPVANGSHKA

SEQ ID NO: 18 - DNA, 2298 bp, Homo sapiens
atgcggctgtggaaggcggtggtggtgactttggccttcatgagtgtggacatctgcgtgaccacggccatctatgtcttcagccacctggaccgcagcctcctggaggacatccgccacttcaacatctttgactcggtgctggatctctgggcagcctgcctgtaccgcagctgcctgctgctgggagccaccattggtgtggccaagaacagtgcgctggggccccggcggctgcgggcctcgtggctggtcatcaccctcgtgtgcctcttcgtgggcatctatgccatggtgaagctgctgctcttctcagaggtgcgcaggcccatccgggacccctggttttgggccctgttcgtgtggacgtacatttcactcggcgcatccttcctgctctggtggctgctgtccaccgtgcggccaggcacccaggccctggagccaggggcggccaccgaggctgagggcttccctgggagcggccggccaccgcccgagcaggcgtctggggccacgctgcagaagctgctctcctacaccaagcccgacgtggccttcctcgtggccgcctccttcttcctcatcgtggcagctctgggagagaccttcctgccctactacacgggccgcgccattgatggcatcgtcatccagaaaagcatggatcagttcagcacggctgtcgtcatcgtgtgcctgctggccattggcagctcatttgccgcaggtattcggggcggcatttttaccctcatatttgccagactgaacattcgccttcgaaactgtctcttccgctcactggtgtcccaggagacaagcttctttgatgagaaccgcacaggggacctcatctcccgcctgacctcggacaccaccatggtcagcgacctggtctcccagaacatcaatgtcttcctgcggaacacagtcaaggtcacgggcgtggtggtcttcatgttcagcctctcatggcagctctccttggtcaccttcatgggcttccccatcatcatgatggtgtccaacatctacggcaagtactacaagaggctctccaaagaggtccagaatgccctggccagagcgagcaacacggcggaggagaccatcagtgccatgaagactgtccggagcttcgccaatgaggaggaggaggcagaggtgtacctgcggaagctgcagcaggtgtacaagctgaacaggaaggaggcagctgcctacatgtactacgtctggggcagcgggctcacactgctggtggtccaggtcagcatcctctactacgggggccaccttgtcatctcaggccagatgaccagcggcaacctcatcgccttcatcatctacgagtttgtcctgggagattgtatggagtccgtgggttccgtttacagtggcctgatgcagggagtgggggctgctgagaaggtgttcgagttcatcgaccggcagccgaccatggtgcacgatggcagcttggcccccgaccacytggagggccgggtggactttgagaatgtgaccttcacytaccgcactcggccccacacccaggtcctgcagaatgtctccttcagcctgtcccccggcaaggtgacggccctggtggggccctcgggcagtgggaagagctcctgtgtcaacatcctggagaacttctaccccctggaggggggccgggtgctgctggacggcaagcccatcagcgcctacgaccacaagtacttgcaccgtgtgatctccctggtgagccaggagcccgtgctgttcgcccgctccatcacggataacatctcctacggcctgcccactgtgcctttcgagatggtggtggaggccgcacagaaggccaatgcccacggcttcatcatggaactccaggacggctacagcacagagacaggggagaagggcgcccagctgtcaggtggccagaagcagcgggtggccatggcccgggctctggtgcggaaccccccagtcctcatcctggatgaagccaccagcgctttggatgccgagagcgagtatctgatccagcaggccatccatggcaacctgcagaagcacacggtactcatcatcgcgcaccggctgagcaccgtggagcacgcgcacctcattgtggtgctggacaagggccgcgtagtgcagcagggcacccaccagcagctgctggcccagggcggcctctacgccaagctggtgcagcggcagatgctggggcttcagcccgccgcagacttcacagctggccacaacgagcctgtagccaacggcagtcacaaggcc

SEQ ID NO: 19 - protein, 14 aa, Artificial Sequence
  Pfam consensus sequence
LKEGFDAIPVASRL

SEQ ID NO: 20 - protein, 535 aa, Artificial Sequence
  Pfam consensus sequence
VALVAALGGGFLFGYDTGVIGGFLALIDFLFRFGLLTSSGALAELGYSTVLTGLVVSIFFLGRLIGSLFAGKLGDRFGRKKSLLIALVLFVIGALLSSNTIIGMIFGCFALFELLASLVFGNYLVHIGAKFMFVAGMFVSGGVTIGAAPGYTTIGLWAFYLLIVGRVLVGLGVGGASVLVPMYISEIAPKALRGALGSLYQLAITIGILVAAIIGLGLNKTNNDSALNSWGWRIPLGLQLVPALLLLIGLLFLPESPRWLVEKGKLEEAREVLAKLRGVEDVDQEIQEIKAELEAGVEEEKAGKASWGELFRGRTRPKVRQRLLMGVMLQAFQQLTGINAIFYYSPTIFKSVGVSDSRASLLVTIIVGVVNFVFTLVALIFLVDRFGRRPLLLLGAAGMAICFLILGASIGVALLLLNKPKDPLSKAAGIVAIVFILLFIAFFALGWGPIPWVILSELFPTKVRSKALALATAANWLANFIIGFLFPYITGAIGLALGGYVFLVFAGLLVLFILFVFFFVPETKGRTLEEIEELF

SEQ ID NO: 21 - protein, 412 aa, Artificial Sequence
  Pfam consensus sequence
LIPFKNTNFWRFGLFFFFISMSAYFPFFPIWLKEVNGLTKTETGIVFSCISLFSILFQPLFGLISDKLGLKKHLIWCISLLLVLFAPFFIYVFEPLLQLNILAGALVGGVFLGLVYSAGAGAIEAYIEKVSRNSHFEYGKARMFGCVGWALCASIAGILFSIDPHIVFWLGSGFALILLLLLLLSKPDKSHSAIVADALGANKSAFSLRLAIELFKMRKFWVFVLYVVGVASVYDVFDQQLFAVFFAGFFESPQVGTRVFGYVTTFGELLNALIMFCAPFIVNRIGAKNALLIAGVIMSVRILGSAFATTALEVVILKLLHAFEVPFLLVGVFKYITSNFDKRLSATIFLIGFQFSKQLAIVLLSTLAGKLYDHVGFQTAYLVLGIIVLSFTLISIFTLSGSREQIVLPTPE

SEQ ID NO: 22 - protein, 602 aa, Artificial Sequence
  Pfam consensus sequence
AALTKLDAAKRERSKRISAALQEPRNQRKLVLVIVSIALLLDNMLYMVIVPIIPDYLRDIENEEESELQIAKAGESPHTLAPPAFSNIFSYYDNETSAPLNSTDSLIAALVDEASTIHMATERSILEKNDCLSERKDLENEDVQVGVLFASKAILQLLVNPFSGPLIDRIGYPIPMLIGLTIMFFSTVMFAFGESYAVLFFARSLQGLGSAFADTAGLAMIADRYTEENERSRALGIALAFISFGCLVAPPFGSVLYEFAGKEVPFLILAFVCLLDGLLLLMVLKPSKEAARVSPESQKGVTPIWRLLMDPYIAVVAGALTMANVGLAFLEPTISIWMKETMCDTSKWQLGVVWLPAFVPHVLGVYVTVKLARKYPHHQWLCAAVGLAVVGVSCLCIPLCRNIKGLIIPLCGLCFGIALVDTSLLPTLGYLVDVRHVSVYGSVYAIADISYSVAYALGPIIAGAIVKAIGFTALNLIIGLINILYAPVLFLLRNVYSLKPAKEEKDILLMDQPNPEYQTYVMHDSKPVEGGVKNHLEYGQQYNQKQEATLYDSQYEIMEERQYAKEGYQQDQAYQPNYAVSFGTSSPQGEFPAGEDDEEEQD

SEQ ID NO: 23 - protein, 382 aa, Artificial Sequence
  Pfam consensus sequence
SIFALGIMPYITASIIVQLLTVDVIPALEELQKEGGEAGRRKLNQYTRYLLTLVLAFIQSLGIVFIARRGPLLGPADTALGYLVFNPNFFFYLLIVLALTTGSMLVMWLGEQITEKGIGNGISLLIFAGIAAGLPSGLLKQIFEQANLGSGDLFGSIVLLIVLAIVLLLVIFGVVFVQEAVRKIPVQYAKRQLGRRRVGGQSTYLPLKVNQAGVIPVIFASSILLLPATLGQFLNSQDGSIPAFLSNVGWVRWIANYLSPNSINVISILPTSILYLLLYLILIIFSYFYVSTIQLNPEEIAENLKKMGSFIPGIRPGVKATEKYLEKVLNRLTFVGSLFLALIAILPSILEALLGVGLPVFFGLGGTSLLIVVGVAIDTVKQ

SEQ ID NO: 24 - protein, 624 aa, Artificial Sequence
  Pfam consensus sequence
RETWSGKLDFVLSVVGFAVGLGNVWRFPYLCYKNGGGAFLIPYLIFLIVAGIPLFFLELALGQYTREGSITVWRKKILDKGKGICPLFKGIGYASIVIAFYIGIYYNVIIAWALYYLFSSFTTELPWATCNNSWNTPNCVEEREAENSTNGSLAALSSKNLTDYTLERTSPVEEFWERGVLKLSESSGIEDLGELRWELTLCLLLAWIVVYFCLWKGVKSGSGKVVYFTATFPYVVLIVLLIRGVTLPGAADGIKFYLTPDFSKLLDPQVWIDAATQIFFSLGIGFGVLIALASYNKFHNNCYRDAIIVSFINSITSFLAGFVIFSILGFMANIVQEQGVPENEKILLLSVLSRDLIPHVNLSALTADYSVYDVISEVAESEFVLGLACLEDELDKVQAGPGLAFIAYPEAVTMLPLSPFWAVLFFLMLLTLGLDSQFGGVEGIITALVDEFPILLRKVRRELFILLVCVISFLLGLFMVTEGGIYVFTLFDYYAASGFSLLFVVFFECIAVAWVYGIDRFYDDITEMLGFRPGLYWKLCWKFVSPLILLFLFIFSIVQYGLKPLTYNNWIKEAEDYVYPNWANALGWLLALSSMLCVPLYIIYKLLSTEGDSLLERLQKATTP

SEQ ID NO: 25 - protein, 196 aa, Artificial Sequence
  Pfam consensus sequence
GEVLALVGPNGAGKSTLLKLISGLLPPTEGTILLDGARDLRLSKLKERLERLRKNIGVVFQDPTLFPNVELTVRENIAFGLRLSLGLSKDEQRARLKKAGAEELLERLGLGYDHLLDRRPGTLSGGQKQRVAIARALLTKPKLLLLDEPTAGLDPASRAQLLELLRELRQQGGTVLLITHDLDLLDRLADRILVLE

SEQ ID NO: 26 - protein, 23 aa, Artificial Sequence
  Pfam consensus sequence
NDDSIRSIGLILNLLARAVLEGR

SEQ ID NO: 27 - protein, 487 aa, Artificial Sequence
  Pfam consensus sequence
VALVAALGGGFLFGYDTGVIGGFLALIDFLFRFGLLTSSGALAELGYSTVLTGLVVSIFFLGRLIGSLFAGKLGDRFGRKKSLLIALVLFVIGALLSGAAPGYTTIGLWAFYLLIVGRVLVGLGVGGASVLVPMYISEIAPKALRGALGSLYQLAITIGILVAAIIGLGLNKTNNDSALNSWGWRIPLGLQLVPALLLLIGLLFLPESPRWLVEKGKLEEAREVLAKLRGVEDVDQEIQEIKAELEAGVEEEKAGKASWGELFRGRTRPKVRQRLLMGVMLQAFQQLTGINAIFYYSPTIFKSVGVSDSRASLLVTIIVGVVNFVFTLVALIFLVDRFGRRPLLLLGAAGMAICFLILGASIGVALLLLNKPKDPLSKAAGIVAIVFILLFIAFFALGWGPIPWVILSELFPTKVRSKALALATAANWLANFIIGFLFPYITGAIGLALGGYVFLVFAGLLVLFILFVFFFVPETKGRTLEEIEELF

SEQ ID NO: 28 - protein, 198 aa, Artificial Sequence
  Pfam consensus sequence
GEVLALVGPNGAGKSTLLKLISGLLPPTEGTILLDGARDLRLSKLKERLERLRKNIGVVFQDPTLFPNVELTVRENIAFGLRLSLGLSKDEQRARLKKAGAEELLERLGLGYDHLLDRRPGTLSGGQKQRVAIARALLTKPKLLLLDEPTAGLDPASRAQLLELLRELRQQGGTVLLITHDLDLLDRLADRILVLEDG

SEQ ID NO: 29 - protein, 92 aa, Artificial Sequence
  Pfam consensus sequence
PGEVVLLVGPPGSGKTTLARALARLLGPGVIYIDGEGGQRIRLALALARKDVLLLDEITSLLDVTVIATTNDLDPALLRRRFDRRIVLLRIL

SEQ ID NO: 30 - protein, 92 aa, Artificial Sequence; identical to SEQ ID NO: 29
  Pfam consensus sequence

SEQ ID NO: 31 - protein, 529 aa, Artificial Sequence
  Pfam consensus sequence
EERLLDDLLSEDGYNKRLRPVFGGSDPVTVSLGLTLSQLISVNEKNQEMTTNVWLRQYEWTDYRLRWNPEPRLDYGGILTLLRVPSEKIWLPDIVLYNNKDGDFHVTTTTNVLLRYHPDGSVLWLPPAIYKSSCPIDVTYFPFDQQNCSLKFGSWTYDGDEIDLVWKNGDEGEDKDYTVESVEVDLEDFTELGEWDIIHVPGRKNEKEVYYSSCCTGEYPDITFYFILRRKPLFYTINLIIPCVLISFLSWLVFYLPADAGPEKVTLGISVLLTLTVFLLLIREILPKTSLVVPLIGKYLLFTMFVVTASVEYAVVVLNVHHRSPKSTHKMPEWVRKLFLERKLPRLLFMKRPNESLSEPPVKRPLLRRPHSSSSGSSLKAEEYSLSKPRSELMFEKQMSEEEEYCCALHFQGERDGLDSPGTAKGGRGKASPCKCIKVKGGPVPESGRSLSPLSLKRLSPELKKAVEGVSRRFIAEHKPPKEAVKAWLRSKDEDNEVKEDWKYVAMVIDRLFLWIFPIVFVLGTLGIF

SEQ ID NO: 32 - protein, 459 aa, Artificial Sequence
  Pfam consensus sequence
AWILISAALVIFMMQPGFALLESGLVRSKNVLNFILMKNFVDLAIGICVLAYVLFGYSLAFGDSYGEPGNGFIGNGLVWLFLKFLGVSAAGIQDGTLPDGLPFFLFQLMFAAKTAATIISGAVAERIKFSAYLLFSALLGTLVYPPVAHWVWGELVGGWLAKLGVLVLILKTKAIDFAGSTVVHIVGGVAGLAAALVLGPRIGRFPDDETGKPEAIRPHNLPFAVLGTFLLWFGWFGFNAGSALTANGRAAAIGAGWSTVARAAVNTNLAAAAGALTWLLISRLKTGKPTVLGLANGALAGLVAIGTPACGVVSPWGALIIGLVAGVLSVLGVKYLTPKLKEKLGIDDPLDVFPVHGVGGIWGGIAVGIFAAPKVNNIGFPEEYGASTSGISGGLLYGNGGFKQLGVQLIGIAVILAYAFGVTFILAKLLGLTLGGKLRVSEEEEKVGLDLAEHGETAY

SEQ ID NO: 33 - protein, 412 aa, Artificial Sequence
  Pfam consensus sequence
MKSSKVDTFNWRTWLVQVVCFVLMFVNSVVTLIAASFPGLGFPCYYAALVDYSALNLTVRNGVWVRRRAGHLTPTLFLETPELFAYVVFTALVLLAVAVYYIVGAVAIRRAKKKFEFTASLNQLSAWITLVGDPTTLFLGILRMWTLQLFVLLLSYKHVVLAAFVYLLHFACSVAFTVSFITRGYSSAWYSKFVEQLIPPNPLLHRVVGPGRAVVVNLYLLLLALETLVFSLSLMLALGNSFYISVSDTVFGAVNLFLILAVVWLIVTELVLSKYVKVLFGPYLGTLVFVGSLGLALPVYRRYEAIFVSATQAPNLHTGVRINLAVIAILCLAMIVVRLVRAYLYHRKKHTKFFVRMPKSRYKALVSKARVRSSMRSRRRPSPLSPKVRARRRNEPSLNQAPRASYLREEES

SEQ ID NO: 34 - protein, 450 aa, Artificial Sequence
  Pfam consensus sequence
GQTLLLGLQHLLAMFAATVLVPLLVGDALCLGLSAESLAYLISTTLLVSGIGTLLQLLRYGIGRIFGIRLPIVLGSSFAFVTPAIGLIALMIALGSAPADQGPLEPIGIALAGLFGALLVAGVLFILISFTGLRGRLARLFPPVVTGPVVLLIGLSLIPIAVKGVAGGWAAILDGLLGLCPATPPLLVGSLELLGLAVVVLAVILLLSVFTAVLKGFFKSLPILIGIIVGWILALFMGPSIVDLSPEGSEARTDKNSLAVVRDAPWFQLPLPLPFGLPLDALGAFNPGLILTMLAVAIVAIVESIGDITATAKVSGRDLKPGTYKPRLRRGLLADGLATLLAGLFGAGTPTTTFAENIGVVALTRVASRRVGVTAAVILILLGLFPKFAALLSSIPSPVLGGVMLVLFGMIAGSGVSILQSVDLDYSARNLLIIAVSLVLGLGIPTVPEI

SEQ ID NO: 35 - protein, 328 aa, Artificial Sequence
  Pfam consensus sequence
LGLLRLGFLVEFLSRAVISGFMAGAAILILLSQLKGLLGLSNLFTRHSGIVSVLRALFDLVDNLHDFLKWNWATLVIGISFLIFLLIIKLLPNPKKRKKKLFWVPAPAPLVAVILATLISYLFNRHKLADRYGVSIVGEIPSGLPPPSLPRLNLSPSTLLDLLPIALALALVGLLESILTAKSFAKIKGYKIDSNKELVAQGIANIVGSLFGGYPATGSFSRSAVNVKAGAKTQLSGIVMAVVVLLVLLFLTPLLEYIPMAVLAAIIIVALIGMLIDWSELIRLLWKLSKLDFLIWLATFFGTVFVDNLEIGVLVGVAISLLFLILRV

SEQ ID NO: 36 - protein, 271 aa, Artificial Sequence
  Pfam consensus sequence
ALLLKVIYTVGYSLSLVALLLAIFIFLLFRRLHCTRNYIHLNLFISFILRALLFLIGDAVLQNNVGQDADESLHCSTQVGCKVVAVFLHYFFLANFFWMLVEGLYLYTLLVEVFFSERKRLRWYLLIGWGVPAVVVVVWAIVRQIKSPKGYGEDDGCLWLSNEDNTGFWWIIKGPVLLAILVNFIFLINILRILVQKLRESNTGESDQYRLVKSTLVLLPLLGITWILFLFAPENDARGISSVVFLYLFAILNSFQGFFVAVLYCFLNGEV

SEQ ID NO: 37 - protein, 285 aa, Artificial Sequence
  Pfam consensus sequence
QLGYFFFALVLSLAGVVLLWICFFGTKEVYSSSDTRENGQKTTSLLQSLKLLAKNDQLLILCLAALFYLLAINILGGAQLYYVTYVLGDPELFSYLLLYNILVGLIGSLLFPRLVKRFGKKTVFAGCIVLMVLGSLLIFFVAGSSLALILVLIFLAGILQQLVTLLVWVLQVIMVSDTVDYGEWKTGVRLEGLVYSVFLFVLKLGLALSGALVGWILGYIGYVANASQSTSTALGQLVFILALFALPPALLLLAAFIMLRFYKLTEKKLAEIVEELEKWRTRKRK

SEQ ID NO: 38 - protein, 23 aa, Artificial Sequence
  Pfam consensus sequence
GEVFHYRAPSGRYKLTFEEAQAA

SEQ ID NO: 39 - protein, 285 aa, Artificial Sequence
  Pfam consensus sequence
LLIAILLLILAGATALVTFPLLLGRFLDSGFPLSDGNDDHARSSLISLAILSLFAVFVLQGLLLQGSFYLLAGERLGQRLRKRLFRALLRQILGLFDSFFDTNSVGELTSRLTNDVEKIRDGLGEKLGLLFQSLATVVGGLIVMFYYSWKLTLVLLAILPLLILVSAVLAKKLRKLSRKEQKAYAKAGSVAEESLSGIRTVKAFGREEYELERFDKALEDAEKAGIKKAIIAGLLFGITQLISYLSYALALWFGGYLVASVISGGLSVGTLFAFLSLLGQLIGPL

SEQ ID NO: 40 - protein, 162 aa, Artificial Sequence
  Pfam consensus sequence
RGCTVWFTGLSGSGKSTIANALERKLFAQGISVYLLDGDNVRHGLNKDLGFSEEDREENIRRVGEVAKLFADAGLIVLTSFISPYRADRDQARELHEDGEEAGLKFIEVFVDTPLEVCEQRDPKGLYKKARAGEIKGFTGIDSPYEAPENPELVLDTTKQSV

SEQ ID NO: 41 - protein, 198 aa, Artificial Sequence; identical to SEQ ID NO: 28
  Pfam consensus sequence

SEQ ID NO: 42 - protein, 92 aa, Artificial Sequence; identical to SEQ ID NO: 29
  Pfam consensus sequence